(19) United States
(12) Patent Application Publication
(10) Pub. No.: US 2017/0164878 A1
(43) Pub. Date: Jun. 15, 2017

(54) Wearable Technology for Non-Invasive Glucose Monitoring

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 15/431,769

(22) Filed: Feb. 14, 2017

Related U.S. Application Data
(63) Continuation-in-part of application No. 14/330,649, filed on Jul. 14, 2014.
Application No. 14/330,649 is a continuation-in-part of application No. 14/992,073, filed on Jan. 11, 2016.
Application No. 14/992,073 is a continuation-in-part of application No. 15/206,215, filed on Jul. 8, 2016.
Application No. 15/206,215 is a continuation-in-part of application No. 15/236,401, filed on Aug. 13, 2016.
Application No. 15/236,401 is a continuation-in-part of application No. 15/294,746, filed on Oct. 16, 2016.
Application No. 14/330,649 is a continuation-in-part of application No. 13/523,739, filed on Jun. 14, 2012, now Pat. No. 9,042,596.
Application No. 13/523,739 is a continuation-in-part of application No. 13/797,955, filed on Mar. 12, 2013, now Pat. No. 9,456,916.
Application No. 15/206,215 is a continuation-in-part of application No. 14/948,308, filed on Nov. 21, 2015.
Application No. 14/948,308 is a continuation-in-part of application No. 14/951,475, filed on Nov. 24, 2015.
Application No. 15/236,401 is a continuation-in-part of application No. 14/599,522, filed on Jan. 18, 2015.
Application No. 14/599,522 is a continuation-in-part of application No. 15/136,948, filed on Apr. 24, 2016.
Application No. 15/294,746 is a continuation-in-part of application No. 14/623,337, filed on Feb. 16, 2015, now Pat. No. 9,582,035.
Application No. 14/623,337 is a continuation-in-part of application No. 14/951,475, filed on Nov. 24, 2015.
(60) Provisional application No. 62/311,462, filed on Mar. 22, 2016.
(60) Provisional application No. 62/349,277, filed on Jun. 13, 2016.
(60) Provisional application No. 62/439,147, filed on Dec. 26, 2016.
(60) Provisional application No. 62/349,277, filed on Jun. 13, 2016.
(60) Provisional application No. 62/160,172, filed on May 12, 2015.
(60) Provisional application No. 62/169,661, filed on Jun. 2, 2015.
(60) Provisional application No. 62/303,126, filed on Mar. 3, 2016.
(60) Provisional application No. 62/322,594, filed on Apr. 14, 2016.
(60) Provisional application No. 62/245,311, filed on Oct. 23, 2015.
(60) Provisional application No. 62/349,277, filed on Jun. 13, 2016.

(51) Int. Cl. A61B 5/145; A61B 5/1455; A61B 5/0476; A61B 5/00

(52) U.S. Cl. CPC A61B 5/14532; A61B 5/6826; A61B 5/6824; A61B 5/681; A61B 5/1455; A61B 5/0476; A61B 5/6817

(57) ABSTRACT

This invention is a wearable device for non-invasive glucose monitoring. It can be embodied in a finger ring, wrist band, wrist watch, or ear-worn device. In an example, it can include a circumferential biosensor array which spans at least 25% of the circumference of the device. A biosensor array can include an array of electromagnetic energy emitters and receivers and/or an array of light emitters and receivers.

### CROSS-REFERENCE TO RELATED APPLICATIONS

This application:

(a) is a CIP of application Ser. No. 14/330,649 “Eyewear System for Monitoring and Modifying Nutritional Intake” filed Jul. 14, 2014 which was: (1) a CIP of application Ser. No. 13/523,739 “The Willpower Watch™: A Wearable Food Consumption Monitor” filed Jun. 14, 2012; and (2) a CIP of application Ser. No. 13/797,955 “Device for Selectively Reducing Absorption of Unhealthy Food” filed Mar. 12, 2013 which claimed the priority benefit of the priority benefit of provisional application 61/729,494 “Device for Selectively Reducing Absorption of Unhealthy Food” filed Nov. 23, 2012;

(b) is a CIP of application Ser. No. 14/992,073 “Wearable Device for the Ear with Electroencephalographic and Spectroscopic Sensors” filed Jan. 11, 2016 which was: (1) a CIP of application Ser. No. 14/599,522 “Mobile Wearable Electromagnetic Brain Activity Monitor” filed Jan. 18, 2015 which in turn was: a CIP of application Ser. No. 14/562,719 “Willpower Glasses™: A Wearable Food Consumption Monitor” filed Dec. 7, 2014 which claimed the priority benefit of provisional application 61/932,517 “Nutrode™: Wearable EEG Monitor for Modifying Food Consumption” filed Jan. 28, 2014; claimed the priority benefit of provisional application 61/932,517 “Nutrode™: Wearable EEG Monitor for Modifying Food Consumption” filed Jan. 28, 2014; claimed the priority benefit of provisional application 61/939,244 “Brainwave-Controlled Eyewear” filed Feb. 12, 2014; claimed the priority benefit of provisional application 62/017,615 “Nervision™ Integrated Eyewear and EEG Monitor” filed Jun. 26, 2014; and claimed the priority benefit of provisional application 62/089,696 “Electroencephalographic Eyewear” filed Dec. 6, 2014; (2) a CIP of application Ser. No. 14/550,953 “Wearable Food Consumption Monitor” filed Nov. 22, 2014; and (3) a CIP of application Ser. No. 13/616,238 “Interactive Voluntary and Involuntary Caloric Intake Monitor” filed Sep. 14, 2012;

(c) is a CIP of application Ser. No. 15/206,215 “Finger Ring with Electromagnetic Energy Sensor for Monitoring Food Consumption” filed Jul. 8, 2016 which (1) was a CIP of application Ser. No. 14/948,308 “Spectroscopic Finger Ring for Compositional Analysis of Food or Other Environmental Objects” filed Nov. 21, 2015 which, in turn, was: a CIP of application Ser. No. 13/901,099 “Smart Watch and Food-Imaging Member for Monitoring Food Consumption” filed May 23, 2013; a CIP of application Ser. No. 14/132,292 “Caloric Intake Measuring System using Spectroscopic and 3D Imaging Analysis” filed Dec. 18, 2013; and a CIP of application Ser. No. 14/449,387 “Wearable Imaging Member and Spectroscopic Optical Sensor for Food Identification and Nutrition Modification” filed Aug. 1, 2014; (2) was a CIP of application Ser. No. 14/951,475 “Wearable Spectroscopic Sensor to Measure Food Consumption Based on Interaction Between Light and the Human Body” filed Nov. 24, 2015 which, in turn, was: a CIP of application Ser. No. 13/901,131 “Smart Watch and Food Utensil for Monitoring Food Consumption” filed May 23, 2013; a CIP of application Ser. No. 14/071,112 “Wearable Spectroscopy Sensor to Measure Food Consumption” filed Nov. 4, 2013; a CIP of application Ser. No. 14/623,337 “Wearable Computing Devices and Methods for the Wrist and/or Forearm” filed Feb. 16, 2015; and claimed the priority benefit of provisional application 62/245,311 “Wearable Device for the Arm with Close-Fitting Biometric Sensors” filed Oct. 23, 2015; and (3) claimed the priority benefit of provisional application 62/349,277 “Glucowear™ System for Monitoring and Managing Intra-body Glucose Levels” filed Jun. 13, 2016;

(d) is a CIP of application Ser. No. 15/236,401 “Wearable Brain Activity Monitor” filed Aug. 13, 2016 which: (1) was a CIP of application Ser. No. 15/136,948 “Wearable and Mobile Brain Computer Interface (BCI) Device and Method” filed Apr. 24, 2016 which: was a CIP of application Ser. No. 14/599,522 “Mobile Wearable Electromagnetic Brain Activity Monitor” filed Jan. 18, 2015 which: was a CIP of application Ser. No. 14/562,719 “Willpower Glasses™: A Wearable Food Consumption Monitor” filed Dec. 7, 2014 which claimed the priority benefit of provisional application 61/932,517 “Nutrode™: Wearable EEG Monitor for Modifying Food Consumption” filed Jan. 28, 2014; claimed the priority benefit of provisional application 61/932,517 “Nutrode™: Wearable EEG Monitor for Modifying Food Consumption” filed Jan. 28, 2014; claimed the priority benefit of provisional application 61/939,244 “Brainwave-Controlled Eyewear” filed Feb. 12, 2014; claimed the priority benefit of provisional application 62/017,615 “Nervision™ Integrated Eyewear and EEG Monitor” filed Jun. 26, 2014; and claimed the priority benefit of provisional application 62/089,696 “Electroencephalographic Eyewear” filed Dec. 9, 2014; (2) claimed the priority benefit of provisional application 62/160,172 “Hair-Engaging Mobile Brain Activity Monitor” filed May 12, 2015; (3) claimed the priority benefit of provisional application 62/169,661 “Internet of Thinks (IoT): A Brain Computer Interface (BCI) Using EEG Patterns Associated with the Same Command Across Different Action Modes” filed Jun. 2, 2015; (4) claimed the priority benefit of provisional application 62/303,126 “Undulating Mobile EEG Monitor Spanning a Portion of the Forehead” filed Mar. 3, 2016; and (5) claimed the priority benefit of provisional application 62/322,594 “Halo-Style Mobile Electroencephalographic (EEG) Monitor” filed Apr. 14, 2016; and (6) was a CIP of application Ser. No. 14/599,522 “Mobile Wearable Electromagnetic Brain Activity Monitor” filed Jan. 18, 2015 which: was a CIP of application Ser. No. 14/562,719 “Willpower Glasses™: A Wearable Food Consumption Monitor” filed Dec. 7, 2014 which claimed the priority benefit of provisional application 61/932,517 “Nutrode™: Wearable EEG Monitor for Modifying Food Consumption” filed Jan. 28, 2014; claimed the priority benefit of provisional application 61/932,517 “Nutrode™: Wearable EEG Monitor for Modifying Food Consumption” filed Jan. 28, 2014; claimed the priority benefit of provisional application 61/939,244 “Brainwave-Controlled Eyewear” filed Feb. 12, 2014; claimed the priority benefit of provisional application 62/017,615 “Nervision™ Integrated Eyewear and EEG Monitor” filed Jun. 26, 2014; and claimed the priority benefit of provisional application 62/089,696 “Electroencephalographic Eyewear” filed Dec. 9, 2014;

(e) is a CIP of application Ser. No. 15/294,746 “Wearable Device for the Arm with Close-Fitting Biometric Sensors” filed Oct. 16, 2016 which: (1) was a CIP of application Ser. No. 14/623,337 “Wearable Computing Devices and Methods for the Wrist and/or Forearm” filed Feb. 16, 2015 which, in turn, claimed the priority benefit of: provisional application 61/944,090 “Wearable Computing Device for the Wrist and/or Arm” filed Feb. 25, 2014; provisional application 61/948,124 “Wearable Computing Device for the Wrist and/or Arm” filed Mar. 5, 2014; provisional application 62/100,217 “Forearm Wearable Device with Distal-to-Proximal Flexibly-Connected Display Modules” filed Jan. 6, 2015; provisional application 62/106,856 “Forearm Wearable Computing Device with Proximal and Distal Arcuate Bands” filed Jan. 23, 2015; provisional application 62/111,163 “Forearm-Wearable Computing Device with Large Display Area” filed Feb. 3, 2015; provisional application 62/113,423 “Sensor-Informed Modification of the Interface Modality Between a Human and a Wearable Computing Device” filed Feb. 7, 2015; and provisional application 62/115,691 “Adjustment of Wearable Computer-to-Human Interface Based on Environmental and/or Physiological Sensors” filed Feb. 13, 2015; (2) was a CIP of application Ser. No. 14/951,475 “Wearable Spectroscopic Sensor to Measure Food Consumption Based on Interaction Between Light and the Human Body” filed Nov. 24, 2015 which, in turn: (a) is a CIP of application Ser. No. 13/901,131 “Smart Watch and Food Utensil for Monitoring Food Consumption” filed May 23, 2013; (b) is a CIP of application Ser. No. 14/071,112 “Wearable Spectroscopy Sensor to Measure Food Consumption” filed Nov. 4, 2013; (c) is a CIP of application Ser. No. 14/623,337 “Wearable Computing Devices and Methods for the Wrist and/or Forearm” filed Feb. 16, 2015; and (d) claims the priority benefit of provisional application 62/245,311 “Wearable Device for the Arm with Close-Fitting Biometric Sensors” filed Oct. 23, 2015; (3) claimed the priority benefit of provisional application 62/245,311 “Wearable Device for the Arm with Close-Fitting Biometric Sensors” filed Oct. 23, 2015; and (4) claimed the priority benefit of provisional application 62/349,277 “Glucowear™ System for Monitoring and Managing Intra-body Glucose Levels” filed Jun. 13, 2016;

(f) claims the priority benefit of provisional application 62/311,462 “Glucowear™ Automated Closed-Loop System for Glycemic Control” filed Mar. 22, 2016;

(g) claims the priority benefit of provisional application 62/349,277 “Glucowear™ System for Monitoring and Managing Intra-body Glucose Levels” filed Jun. 13, 2016; and

(h) claims the priority benefit of provisional application 62/439,147 “Arcuate Wearable Device for Measuring Body Hydration and/or Glucose Level” filed Dec. 26, 2016.

The entire contents of these related applications are incorporated herein by reference.

### FEDERALLY SPONSORED RESEARCH

Not Applicable

### SEQUENCE LISTING OR PROGRAM

Not Applicable

### BACKGROUND

Field of Invention

This invention relates to non-invasive glucose monitoring

### INTRODUCTION

The prevalence and harmful effects of diabetes continue to increase around the world. A key part of reducing the harmful effects of diabetes is monitoring and managing body glucose levels. Repeated finger pricking to obtain blood samples for testing is one way to measure body glucose levels, but it is invasive and can be uncomfortable. There has been considerable work toward the development of wearable technology with biosensors to enable non-invasive monitoring of body glucose levels, but challenges remain. For example, wearable devices can slip, rotate, and otherwise move relative to a person's body. Such movement can change the locations and proximity of biosensors relative to the person's body which can interfere with accurate measurement of glucose levels. Also, tissue characteristics vary between people. The invention disclosed herein can help to address these problems. It discloses novel wearable technology that can enable more accurate non-invasive measurement of body glucose levels despite body motion and variation in tissue characteristics between people.

### REVIEW OF THE PRIOR ART

As mentioned above, there has already been considerable work toward the development of wearable technology with biosensors to enable non-invasive monitoring of body glucose levels. For example, U.S. Pat. No. 6,614,238 (Jean et al., Sep. 2, 2003, “A Microwave Sensor Having Improved Sensitivity”) discloses the use of a microwave sensor for measuring the permittivity of tested material. U.S. Pat. No. 6,987,393 (Jean et al., Jan. 17, 2006, “Ultra Wideband Pulse Dispersion Spectrometry Method and Apparatus Providing Multi-Component Composition Analysis”) and U.S. Patent Application 20120310055 (Jean, B., Dec. 6, 2012, “Ultra-Wide Band Non-Invasive Biological Sensor and Method”) disclose the use of microwave spectroscopy for measuring the permittivity of tested material. U.S. Pat. No. 7,371,217 (Kim et al., May 13, 2008, “Device for the Non-Invasive Measurement of Blood Glucose Concentrations by Millimeter Waves and Method Thereof”) discloses the use of a rectangular waveguide for measuring glucose concentration. U.S. Pat. No. 7,627,357 (Zribi et al., Dec. 1, 2009, “System and Method for Non-Invasive Glucose Monitoring”) discloses a method for determining analyte concentration levels by scattering radiation off, or through, a target.

U.S. Pat. No. 7,680,522 (Andersohn et al., Mar. 16, 2010, “Method and Apparatus for Detecting Misapplied Sensors”) discloses a method and system for determining whether a spectrophotometric sensor is misapplied. U.S. Pat. No. 8,199,007 (Coakley et al., Jun. 12, 2012, “Flex Circuit Snap Track for a Biometric Sensor”) discloses a sensor assembly configured to house an optical component. U.S. Pat. No. 8,515,517 (Hayter et al., Aug. 20, 2013, “Method and System for Dynamically Updating Calibration Parameters for an Analyte Sensor”) discloses methods and apparatuses for determining and dynamically updating a calibration parameter. U.S. Pat. No. 8,613,892 (Stafford, Dec. 24, 2013, “Analyte Meter with a Moveable Head and Methods of Using the Same”) discloses in vitro analyte meters with moveable meter portions.

U.S. Pat. No. 8,868,147 (Stippick et al., Oct. 21, 2014, “Method and Apparatus for Controlling Positioning of a Noninvasive Analyzer Sample Probe”) discloses a probe interface method and apparatus for use in conjunction with an optical based noninvasive analyzer. U.S. Pat. No. 8,930,145 (Li et al., Jan. 6, 2015, “Light Focusing Continuous Wave Photoacoustic Spectroscopy and Its Applications to Patient Monitoring”) discloses systems and methods that use spatial modulation to focus continuous wave light into a localized region of interest such as an individual blood vessel. U.S. Pat. No. 8,961,415 (LeBoeuf et al., Feb. 24, 2015, “Methods and Apparatus for Assessing Physiological Conditions”) discloses monitoring apparatuses and methods to detect physiological information from a subject such as heart rate, subject activity level, subject tympanic membrane temperature, and subject breathing rate.

U.S. Pat. No. 8,989,230 (Dummer et al., Mar. 24, 2015, “Method and Apparatus Including Movable-Mirror MEMS-Tuned Surface-Emitting Lasers”) discloses an apparatus having a substrate, a solid-state gain medium, a reflective mirror on one side of the medium, a movable reflective mirror on an opposite side of the medium, and a mechanism configured to move the movable mirror to tune a characteristic wavelength. U.S. Pat. No. 9,037,204 (Schlottau, May 19, 2015, “Filtered Detector Array for Optical Patient Sensors”) discloses optical patient monitoring systems including a broadband emitter configured to emit two or more wavelengths of light into the tissue of a patient. U.S. Pat. No. 9,061,899 (Rowe et al., Jun. 23, 2015, “Apparatus and Method of Biometric Determination Using Specialized Optical Spectroscopy Systems”) discloses methods and apparatuses for performing biometric determinations using optical spectroscopy of tissue including include determination or verifications of identity, estimation of age, estimation of sex, determination of sample liveness and sample authenticity.

U.S. Pat. No. 9,134,175 (Matsushita, Sep. 15, 2015, “Measurement Device”) discloses a spectrometry device including a wavelength-tunable interference filter that is provided with a stationary reflection film, a movable reflection film and an electrostatic actuator which changes a gap dimension between the stationary reflection film and the movable reflection film. U.S. Patent Application 20080200790 (Kim et al., Aug. 21, 2008, “Apparatus for Measuring Blood Sugar and Apparatus for Monitoring Blood Sugar Comprising the Same”) discloses the use of finger-contact microwave sensor to measure blood sugar. U.S. Patent Application 20080319299 (Stippick et al., Dec. 25, 2008, “Method and Apparatus for Controlling Positioning of a Noninvasive Analyzer Sample Probe”) discloses a probe interface method and apparatus for use in conjunction with an optical based noninvasive analyzer, wherein an algorithm controls a sample probe position and attitude relative to a skin sample site before and/or during sampling.

U.S. Patent Application 20090018420 (White, Jan. 15, 2009, “Apparatus for Non-Invasive Spectroscopic Measurement of Analytes, and Method of Using the Same”) discloses an apparatus for spectroscopic evaluation of a subject's body fluids at the interstitial region adjacent to or in between a subject's extremities. U.S. Patent Application 20090105605 (Abreu, Apr. 23, 2009, “Apparatus and Method for Measuring Biologic Parameters”) discloses a sensor fitted on support structures using a special geometry for acquiring continuous and undisturbed data on the physiology of the body. U.S. Patent Application 20100249546 (White, Sep. 30, 2010, “Apparatus for Non-Invasive Spectroscopic Measurement of Analytes, and Method of Using the Same”) discloses an apparatus for spectroscopic evaluation of a subject's body fluids at the interstitial region adjacent to or in between a subject's extremities.

U.S. Patent Application 20120056289 (Tian et al., Mar. 8, 2012, “Materials, Systems and Methods for Optoelectronic Devices”) discloses a photodetector comprising an integrated circuit and at least two optically sensitive layers. U.S. Patent Application 20130041235 (Rogers et al., Feb. 14, 2013, “Flexible and Stretchable Electronic Systems for Epidermal Electronics”) discloses skin-mounted biomedical devices and methods of making and using biomedical devices for sensing and actuation applications. U.S. Patent Application 20130197319 (Monty et al., Aug. 1, 2013, “Flexible Electrode for Detecting Changes in Temperature, Humidity, and Sodium Ion Concentration in Sweat”) discloses a flexible sensor suitable for contact with skin comprising: a nanocomposite; and a top layer; where the sensor provides in-situ detection in sweat or other aqueous body fluids at the skin surface of at least one physiological parameter selected from the group consisting of a physiological salt component, temperature, moisture, humidity, or combinations thereof.

U.S. Patent Application 20130199822 (Fan et al., Aug. 8, 2013, “Flexible, Permeable, Electrically Conductive and Transparent Textiles and Methods for Making Them”) discloses methods for forming a flexible, permeable, electrically conductive and substantially transparent textile utilizing vapor phase deposition. U.S. Patent Application 20130248380 (Cui, Sep. 26, 2013, “Flexible Graphene Biosensor”) discloses a biosensor comprising a graphene electrode linked to a biosensing element a linker, wherein the biosensing element is bonded to a flexible substrate. U.S. Patent Application 20140009638 (Baraniuk et al., Jan. 9, 2014, “Dual-Port Measurements of Light Reflected from Micromirror Array”) discloses an imaging system and method that captures compressive sensing (CS) measurements of a received light stream and also obtains samples of background light level (BGLL).

U.S. Patent Application 20140058220 (LeBoeuf et al., Feb. 7, 2014, “Apparatus, Systems and Methods for Obtaining Cleaner Physiological Information Signals”) discloses real-time, noninvasive health and environmental monitors including a plurality of compact sensors integrated within small, low-profile devices, such as earpiece modules. U.S. Patent Application 20140064315 (Dummer et al., Mar. 6, 2014, “Method and Apparatus Including Movable-Mirror MEMS-Tuned Surface-Emitting Lasers”) discloses a VCSEL apparatus having a substrate, a solid-state gain medium, a reflective mirror on one side of the medium, a movable reflective minor on an opposite side of the medium, and a mechanism configured to move the movable minor to tune a characteristic wavelength. U.S. Patent Application 20140148658 (Zalevsky et al., May 29, 2014, “Method and System for Non-Invasively Monitoring Biological or Biochemical Parameters of Individual”) discloses a system and method which measures speckle patterns generated by a portion of a person's body.

U.S. patent application 20140316230 (Denison et al., Oct. 23, 2014, “Methods and Devices for Brain Activity Monitoring Supporting Mental State Development and Training”) discloses an electromagnetic brain activity sensors in contact with a person's head via an arcuate member which loops around the rear portion of a person's head, from one side to the other side. U.S. Patent Application 20140339438 (Correns et al., Nov. 20, 2014, “Devices and Methods for Spectroscopic Analysis”) discloses devices and methods for spectrometric analysis of light-emitting samples. U.S. Patent Application 20150005644 (Rhoads, Jan. 1, 2015, “Dermoscopic Data Acquisition Employing Display Illumination”) discloses use of a smartphone camera to gather skin imagery while controlled spectral illumination is emitted from the smartphone display.

U.S. Patent Application 20150015888 (Gulati et al., Jan. 15, 2015, “Dynamic Radially Controlled Light Input to a Noninvasive Analyzer Apparatus and Method of Use Thereof”) discloses an analyzer apparatus and method to dynamically irradiate a sample with incident light where the incident light is varied in time in terms of any of: position, radial position relative to a point of the skin of a subject, solid angle, incident angle, depth of focus, energy, and/or intensity. U.S. patent application 20150036138 (Watson et al., Feb. 5, 2015, “Analyzing and Correlating Spectra, Identifying Samples and Their Ingredients, and Displaying Related Personalized Information”) describes obtaining two spectra from the same sample under two different conditions at about the same time for comparison. U.S. Patent Application 20150073723 (Mulligan et al., Mar. 12, 2015, “Noninvasive Hydration Monitoring”) discloses novel tools and techniques for assessing, predicting and/or estimating the hydration of a patient and/or an amount of fluid needed for effective hydration of the patient.

U.S. Patent Application 20150094551 (Frix et al., Apr. 2, 2015, “Continuous Transdermal Monitoring System and Method”) discloses methods and systems for continuous transdermal monitoring (“CTM”) with a pulse oximetry sensor having a plurality of light detectors arranged as an array. U.S. Patent Application 20150099943 (Russell, Apr. 9, 2015, “Wearable Physiological Sensing Device with Optical Pathways”) discloses a wearable physiological sensing device may with at least one light source; a first light pipe coupled with the at least one light source, the first light pipe at least partially circumscribing an extremity of a patient, and at least one aperture for radiating light from the light source into the extremity. U.S. Patent Application 20150112170 (Amerson et al., Apr. 23, 2015, “Device and Method for Non-Invasive Glucose Monitoring”) discloses a device and method for non-invasively measuring analytes and physiological parameters measuring terahertz radiation emitted though biological tissue.

U.S. Patent Application 20150126824 (LeBoeuf et al., May 7, 2015, “Apparatus for Assessing Physiological Conditions”) discloses monitoring apparatuses and methods for assessing a physiological condition of a subject including at least two types of physiological information and possibly also environmental information. U.S. Patent Application 20150126825 (LeBoeuf et al., May 7, 2015, “Physiological Monitoring Apparatus”) discloses wearable apparatuses including a plurality of compact sensors integrated within small, low-profile devices such as earpiece modules for monitoring various physiological and environmental factors. U.S. Patent Application 20150130633 (Grubstein et al., May 14, 2015) and 20150130634 (Grubstein et al., May 14, 2015, “Indicator and Analytics for Sensor Insertion in a Continuous Analyte Monitoring System and Related Methods”) disclose systems and methods for tracking sensor insertion locations in a continuous analyte monitoring system.

U.S. Patent Application 20150135118 (Grubstein et al., May 14, 2015, “Indicator and Analytics for Sensor Insertion in a Continuous Analyte Monitoring System and Related Methods”) discloses systems and methods for, among others, tracking sensor insertion locations in a continuous analyte monitoring system. U.S. Patent Application 20150141769 (Mulligan et al., May 21, 2015, “Noninvasive Monitoring for Fluid Resuscitation”) discloses novel tools and techniques for assessing, predicting and/or estimating the effectiveness of fluid resuscitation of a patient and/or an amount of fluid needed for effective resuscitation of the patient. U.S. Patent Application 20150148623 (Benaron, May 28, 2015, “Hydration Monitoring Sensor and Method for Cell Phones, Smart Watches, Occupancy Sensors, and Wearables”) discloses a sensor for hydration monitoring in mobile devices, wearables, security, illumination, photography, and other devices and systems using an optional phosphor-coated broadband white LED to produce broadband light which is then transmitted along with any ambient light to a body target.

U.S. Patent Application 20150148624 (Benaron, May 28, 2015, “Method for Detecting Physiology at Distance or During Movement for Mobile Devices, Illumination, Security, Occupancy Sensors, and Wearables”) discloses a sensor which uses broadband light transmitted to a target such as the ear, face, or wrist of a living subject. U.S. patent application 20150148632 (Benaron, May 28, 2015, “Calorie Monitoring Sensor and Method for Cell Phones, Smart Watches, Occupancy Sensors, and Wearables”) discloses a sensor for calorie monitoring in mobile devices, wearables, security, illumination, photography, and other devices and systems which uses an optional phosphor-coated broadband white LED to produce broadband light, which is then transmitted along with any ambient light to a target such as the ear, face, or wrist of a living subject. Calorie monitoring systems incorporating the sensor as well as methods are also disclosed.

U.S. patent application 20150148636 (Benaron, May 28, 2015, “Ambient Light Method for Cell Phones, Smart Watches, Occupancy Sensors, and Wearables”) discloses a sensor for respiratory and metabolic monitoring in mobile devices, wearables, security, illumination, photography, and other devices and systems that uses a broadband ambient light. The sensor can provide identifying features of type or status of a tissue target, such calories used or ingested. U.S. Patent Application 20150168217 (Englund et al., Jun. 18, 2015, “Methods and Apparatus for Spectrometry”) discloses how multimode interference can be used to achieve ultra-high resolving powers and a broad spectroscopy range within a monolithic, millimeter-scale device. U.S. Patent Application 20150192462 (Schiering et al., Jul. 9, 2015, “Dual Spectrometer”) discloses systems and techniques for optical spectrometer detection using IR spectroscopy components and Raman spectroscopy components.

U.S. Patent Application 20150216454 (Kasahara et al., Aug. 6, 2015, “Biological Information Measurement Apparatus and Biological Information Measurement Method”) discloses a blood glucose level measurement apparatus which is mounted on the wrist or the like of a user and performs a measurement using light. U.S. Patent Application 20150216479 (Abreu, Aug. 6, 2015, “Apparatus and Method for Measuring Biologic Parameters”) discloses support structures for positioning sensors on a physiologic tunnel for measuring physical, chemical and biological parameters of the body. U.S. Patent Application 20150216484 (Kasahara et al., Aug. 6, 2015, “Biological Information Processing Apparatus, and Biological Information Processing Method”) discloses a measurement method selection unit which selects one measurement method on the basis of a detection result from a body motion detection unit, from among a plurality of measurement methods of measuring a blood glucose level applying irradiation waves toward a living body of the subject.

U.S. Patent Application 20150224275 (Pastoor et al., Aug. 13, 2015, “Customisation or Adjustment of Patient Interfaces”) discloses a sensor device in the form of a patient interface which has a sensor arrangement for determining a degree of fitting of a contact surface to the patient. U.S. Patent Application 20150233762 (Goldring et al., Aug. 20, 2015, “Spectrometry System with Illuminator”) discloses a spectrometer comprising a plurality of isolated optical channels. U.S. Patent Application 20150238083 (Faubert et al., Aug. 27, 2015, “Method and System for Optically Investigating a Tissue of a Subject”) discloses a probe device for optically investigating a tissue of a subject, comprising: a first probe element, a second probe element, and a third probe element each to be positioned at a respective vertex of a triangle for sensing the tissue, the first probe element each comprising a first light source for emitting light having a first wavelength, the second probe element each comprising a second light source for emitting light having a second wavelength and a first photodetector for detecting light having the first wavelength and scattered the tissue, and the third probe element comprising a second photodetector for detecting light having the first and second wavelengths and scattered the tissue. U.S. Patent Application 20150260573 (Ishimaru, Sep. 17, 2015, “Spectroscopic Measurement Device”) discloses a spectroscopic measurement device including a dark filter that is arranged on an optical path between an imaging optical system and a light detection unit and includes a plurality of regions having different transmittances.

The AIRO wristband raised money on Kickstarter in 2013. It was generally described in an article entitled “Wearable Tech Company Revolutionizes Health Monitoring” by Nicole Fallon in Business News Daily on Oct. 29, 2013. This article generally describes the wristband as “using light wavelengths to monitor nutrition, exercise, stress and sleep patterns,” but does not provide many details on device structure or function. A search did not show any related patent applications. The company appears to have subsequently refunded money contributed to it by crowd-funding supporters. The Healbe GoBe™ raised money on Indiegogo in 2014. It appears to be a wristband that is intended to measure caloric intake using electromagnetic energy. Thus far, there do not yet appear to be results published in a peer-reviewed journal. The BioRing™ launched a campaign on Indiegogo in 2016. It appears to be a ring intended to measure caloric intake using a biosensor.

(Green, et al., 2005, “Design of a Microwave Sensor for Non-Invasive Determination of Blood-Glucose Concentration,” MS Thesis, Dept. of Electrical and Computer Engineering, Baylor University, 2005) discloses the use of microwave sensors and resonators for measuring glucose concentrations. (Jean et al., 2008, “A Microwave Frequency Sensor for Non-Invasive Blood-Glucose Measurement,” IEEE Sensors Applications Symposium, Atlanta, Ga., Feb. 12-14, 2008, 4-7) and (Jean, B., 2016, “Non-Invasive Blood Glucose Analysis Through Use of High Frequency Measurement of the Dielectric Constant,” http://web.ecs.baylor.edu/faculty/jean/research/blood.htm) disclose the use of microwaves to measure glucose concentration. (Kim et al., 2008, “Microwave Dielectric Resonator Biosensor for Aqueous Glucose Solution,” Review of Scientific Instruments, 79(8), 2008, 1-3) discloses the use of a microwave resonator to measure glucose level.

(Caduff et al., 2009, “Non-Invasive Glucose Monitoring in Patients with Type 1 Diabetes: A Multisensor System Combining Sensors for Dielectric and Optical Characterisation of Skin,” Biosensors and Bioelectronics, 24, 2009, 2778-2784) and (Caduff et al., 2011, “Characteristics of a Multisensor System for Non-Invasive Glucose Monitoring with External Validation and Prospective Evaluation,” Biosensors and Bioelectronics, 26, 2011, 3794-3800) disclose a device with a combination of dielectric and optical sensors for monitoring glucose levels. (Babajanyan et al., 2010, “Real-Time Noninvasive Measurement of Glucose Concentration Using a Microwave Biosensor,” Journal of Sensors, Vol. 2010, Dec. 29, 2010) discloses a real-time glucose sensor using a microwave resonator with a probe.

(Tao et al., 2011, “Metamaterials on Paper as a Sensing Platform,” Advanced Materials, 23(28), Jul. 26, 2011, 3197-3201) discloses the use of split-ring resonators in biosensors. (Guarin et al., 2013, “Microwave-Based Noninvasive Concentration Measurements for Biomedical Applications,” IEEE Newsletter, July, 2013, http://lifesciences.ieee.org/publications/newsletter/july-2013/374-microwave-based-noninvasive-sensors-for-biomedical-applications) discloses the use of microwave spectroscopy for measuring glucose concentration levels. (Kumar et al., 2013, “Measuring Blood Glucose Levels with Microwave Sensor,” International Journal of Computer Applications, 72(15), June, 2013, 4-9) discloses the use of a spiral resonator to measure glucose levels.

(Choi et al., 2014, “Design of Continuous Non-Invasive Blood Glucose Monitoring Sensor Based on a Microwave Split Ring Resonator,” RF Wireless Technology Biomedical Healthcare Applications; IEEE MTT-S International Microwave Workshop Series, London, Dec. 8-10, 2014, 1-3) and (Choi et al., 2015, “Design and In Vitro Interference Test of Microwave Noninvasive Blood Glucose Monitoring Sensor,” IEEE Transactions on Microwave Theory and Techniques, 63(10), October, 2015) disclose a glucose monitor that includes a pair of microwave split-ring resonators. (Kim et al., 2014, “A Reusable Robust Radio Frequency Biosensor Using Microwave Resonator by Integrated Passive Device Technology for Quantitative Detection of Glucose Level,” Biosensors and Bioelectronics, Vol. 67, May 15, 2015, 687-693) discloses the use of a rectangular microwave resonator to measure glucose level. (Mazady, A., 2014, “Non-Invasive Glucose Meter,” Electrical and Computer Engineering Department, University of Connecticut, Nov. 15, 2014, http://www.engr.uconn.edu/˜mam10069/Docs/NonInvasiveGlucoseMeasurement.pdf) discloses the use of impedance spectroscopy for measuring glucose levels.

(Caduff et al., 2015, “Glucose Detection from Skin Dielectric Measurements,” Dielectric Relaxation in Biological Systems, Oxford University Press, 2015) discusses the theory and selected evidence concerning the use of dielectric sensors for measuring glucose levels. (Chien et al., 2015, “A Microwave Reconfigurable Dielectric-Based Glucose Sensor with 20 mg/dL Sensitivity at Sub-nL Sensing Volume in CMOS,” IEEE, 2015) discloses a dielectric-based glucose sensor. (Fu et al., 2015, “Study on a Glucose Concentration Measurement System Based on Microwave Perturbation Technique,” Journal of Microwave Power and Electromagnetic Energy, 49(4), 2015, 215-224) discloses measurement of glucose concentration using microwave perturbation. (Gadawe et al., 2015, “Non Invasive Microwave Sensor for Near Field Biological Applications,” Intl. Journal of Innovative Research in Computer and Communication Engineering, 3(5), May, 2015, 4641-4647) discloses a spiral microwave sensor for glucose monitoring.

(Hadkar et al., 2015, “Design of Square Shaped Miniaturized Split Ring Resonators,” Int. Journal of Engineering Research and Applications, 5(5), Part 4, May, 2015, 11-14) discloses the use of square split-ring resonators. (Kim et al., 2015, “Rapid, Sensitive, and Reusable Detection of Glucose by a Robust Radiofrequency Integrated Passive Device Biosensor Chip,” Scientific Reports, 5, 7807, Jan. 15, 2015) discloses the use of an intertwined spiral inductor to measure glucose concentration. (VRBA et al., 2015, “A Microwave Metamaterial Inspired Sensor for Non-Invasive Blood Glucose Monitoring,” Radioengineering, 24(4), December, 2015, 877) discloses the use of inter-digitated sensors for measuring glucose levels. (Wellenzohn et al., 2015, “A Theoretical Design of a Biosensor Device Based on Split Ring Resonators for Operation in the Microwave Regime,” Procedia Engineering, 120, 2015, 865-869) discloses the use of nested split-ring resonators for measuring glucose levels.

### SUMMARY OF THE INVENTION

This invention is a wearable device for non-invasive glucose monitoring. In an example, it can be a finger ring, wrist band, or watch with a circumferential biosensor array including at least one electromagnetic energy emitter and at least one electromagnetic energy receiver. Data from the at least one electromagnetic energy receiver is analyzed to measure the person's body glucose level. The circumferential biosensor array can span at least 25% of the circumference of the finger ring, wrist band, or watch. In an example, the circumferential biosensor array can have at least two sensor pairs, wherein each sensor pair includes an electromagnetic energy emitter and an electromagnetic energy receiver. In an example, the circumferential biosensor array can have at least two sensor triads, wherein each sensor triad includes an electromagnetic energy emitter, an electromagnetic energy resonator, and an electromagnetic energy receiver.

In another example, a circumferential biosensor array can include at least one light emitter (which is configured to emit energy toward the person's body) and at least one light receiver (which is configured to receive light that has passed through and/or been reflected from the person's body). A first light emitter can emit light with a first frequency and/or spectrum and a second light emitter can emit light with a second frequency and/or spectrum. A first light emitter can emit light at a first angle and a second light emitter can emit light at a second angle.

In another example, this invention can be a wearable device that is configured to be worn on a person's ear, wherein the device includes one or more electroencephalographic sensors. In an example, at least one electroencephalographic sensor can be located on a portion of the ear-worn device which is configured to project onto the person's forehead. In an example, a first electroencephalographic sensor can be located on a portion of the ear-worn device which is configured to be inserted into the person's ear and a second electroencephalographic sensor can be located on a portion of the ear-worn device which is configured to project onto the person's forehead.

### INTRODUCTION TO THE FIGURES

FIG. 1 shows a device with sensors at different circumferential locations.

FIG. 2 shows a device projecting light at different angles.

FIG. 3 shows a device with a rotating sensor.

FIG. 4 shows a device with a two-dimensional array of sensors.

FIG. 5 shows a device with sensors which press toward the body from a flat enclosure.

FIG. 6 shows a device with sensors which press toward the body from a curved enclosure.

FIG. 7 shows a device with a sensor pushed toward the body by an inflated member.

FIG. 8 shows a device with sensors pushed toward the body by an inflated member.

FIG. 9 shows a device with a sensor pushed toward the body by an adjustable inflated member.

FIG. 10 shows a device with sensors pushed toward the body by inflated members.

FIG. 11 shows a device with sensors pushed toward the body by toroidal inflated members.

FIG. 12 shows a device with sensors pushed toward the body by connected toroidal members.

FIG. 13 shows a device with a sensor on a rotating ball.

FIG. 14 shows a first device with sensors on a radially-undulating strap.

FIG. 15 shows a second device with sensors on a radially-undulating strap.

FIG. 16 shows a device with sensors on a laterally-undulating strap.

FIG. 17 shows a first device with sensors on a strap with elastic portions.

FIG. 18 shows a second device with sensors on a strap with elastic portions.

FIG. 19 shows a device with sensors and a band with a side buckle or clasp.

FIG. 20 shows a device with sensors and a “clam-shell” band.

FIG. 21 shows a device with sensors, a “clam-shell” band, and a compressible member.

FIG. 22 shows a device with sensors, an outer rigid bracelet, and an inner elastic half-band.

FIG. 23 shows a device with sensors, an outer rigid bracelet, and an inner elastic band.

FIG. 24 shows a device with sensors, a clam-shell outer band, and an upper inner elastic half-band.

FIG. 25 defines “proximal-to-distal” and “circumferential” axes on a wearable band.

FIG. 26 shows a device with an energy emitter and receiver along the same circumference.

FIG. 27 shows a device with an energy emitter and receiver along the same proximal-to-distal line.

FIG. 28 shows a first device with an emitter and two receivers along the same circumference.

FIG. 29 shows a first device with two emitters and a receiver along the same circumference.

FIG. 30 shows a second device with an emitter and two receivers along the same circumference.

FIG. 31 shows a second device with two emitters and a receiver along the same circumference.

FIG. 32 shows a device with a central emitter and receivers around it.

FIG. 33 shows a device with a central receiver and emitters around it.

FIG. 34 shows a device with a central emitter and eight receivers around it.

FIG. 35 shows a device with a central receiver and eight emitters around it.

FIG. 36 shows a device with an emitter and receivers on either side along the same circumference.

FIG. 37 shows a device with a receiver and emitters on either side along the same circumference.

FIG. 38 shows a device with pairs of emitters and receivers along the same circumference.

FIG. 39 shows a first device with emitters and receivers along different circumferences.

FIG. 40 shows a second device with emitters and receivers along different circumferences.

FIG. 41 shows a device with an emitter, and receiver, and a barrier around the receiver.

FIG. 42 shows a device with an emitter, and receiver, and barriers around both.

FIG. 43 shows a device with an emitter, and receiver, and a straight barrier between them.

FIG. 44 shows a device with an emitter, and receiver, and a straight barriers on both sides of them.

FIG. 45 shows a device with an emitter, two receivers, and barriers around both receivers.

FIG. 46 shows a device with an emitter, two receivers, and straight barriers on both sides of them.

FIG. 47 shows a device with an emitter with a barrier around it and receivers around the barrier.

FIG. 48 shows a device with an emitter, barrier around it, receivers around the barrier, and another barrier.

FIG. 49 shows a device with emitters and receivers along three circumferences.

FIG. 50 shows a device with emitters and receivers along three circumferences with barriers between them.

FIG. 51 shows a device with an emitter, a receiver, and a split-ring resonator between them.

FIG. 52 shows a device with an emitter, two receivers, and two split-ring resonators between them.

FIG. 53 shows a device with a receiver, two emitters, and two split-ring resonators between them.

FIG. 54 shows a device with an emitter, a receiver, and nested split-ring resonators between them.

FIG. 55 shows a device with an emitter, a receiver, and stacked split-ring resonators between them.

FIG. 56 shows a device with pairs of emitters and receivers with split-ring resonators between them.

FIG. 57 shows a first device with a housing with an emitter and receiver.

FIG. 58 shows a second device with a housing with an emitter and receiver.

FIG. 59 shows a device with a housing with emitters and a receiver in a line.

FIG. 60 shows a device with a housing with emitters in a circle around a receiver.

FIG. 61 shows multiple housings each with an emitter and receiver.

FIG. 62 shows a device with an emitter, a receiver, and a fluid-based glucose sensor.

FIG. 63 shows a glucose managing system with a wrist band and an implanted pump.

FIG. 64 shows a glucose managing system with a wrist band with a split-ring resonator and implanted pump.

FIGS. 65 and 66 show a device worn like a finger ring with an emitter and receiver.

FIGS. 67 and 68 show a device worn like a finger ring with an emitter, a receiver, and a split-ring resonator.

FIG. 69 shows a glucose managing system with an ear-worn EEG sensor and an implanted pump.

FIG. 70 shows a first EEG sensor device worn around the rear portion of a person's head.

FIG. 71 shows a second EEG sensor device worn around the rear portion of a person's head.

FIG. 72 shows a third EEG sensor device worn around the rear portion of a person's head.

### DETAILED DESCRIPTION OF THE FIGURES

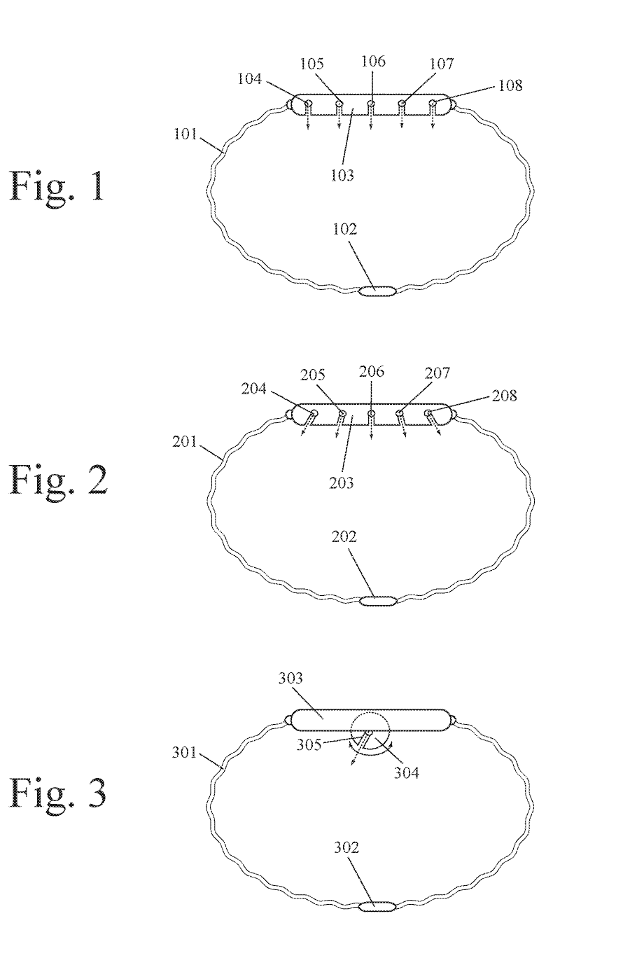

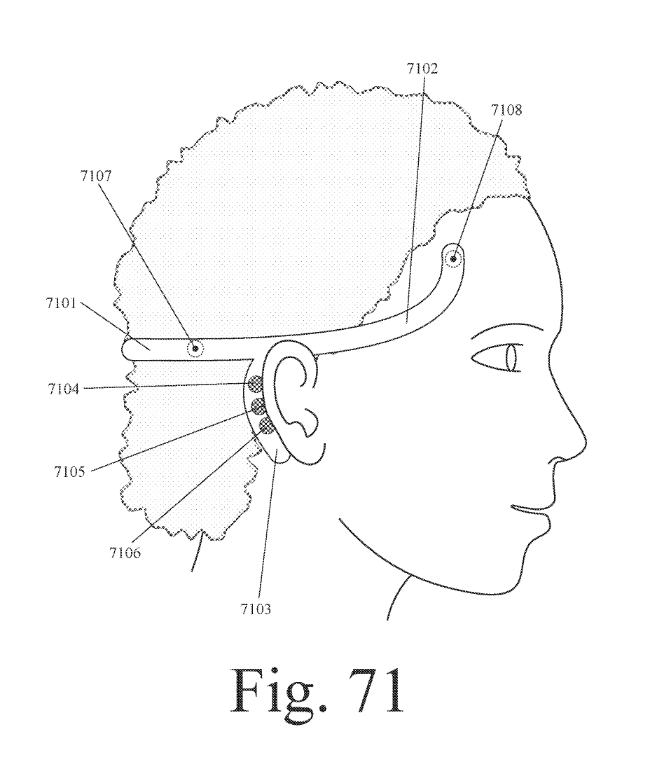

FIGS. 1 through 71 show examples of how this invention can be embodied in a wearable device for non-invasive glucose monitoring. However, it is useful to provide an introductory section before discussing these specific figures and examples. Design and component variations discussed in this introductory section can be applied to the examples in specific figures where relevant, but are not repeated in the narratives accompanying each figure in order to avoid redundant content. This introductory section now follows. After this introductory section, FIGS. 1 through 71 are discussed.

In an example, a wearable device for non-invasive glucose monitoring can comprise: an attachment member which spans at least a portion of the circumference of a person's arm; an enclosure which is part of and/or attached to the attachment member; a first spectroscopic sensor in the enclosure which projects a beam of light onto the arm surface at a first angle relative to the enclosure; and a second spectroscopic sensor in the enclosure which projects a beam of light onto the arm surface at a second angle relative to the enclosure, wherein the first angle differs from the second angle by at least 10 degrees. Data from the spectroscopic sensors can be analyzed to measure a person's glucose levels.

In an example, a wearable device for non-invasive glucose monitoring can comprise: an attachment member which spans at least a portion of the circumference of a person's arm; an enclosure which is part of and/or attached to the attachment member; an elastic member filled with a fluid, gel, or gas which is attached to and/or part of the enclosure; and one or more biometric sensors which record biometric data concerning the person's arm tissue, wherein these one or more biometric sensors are attached to a circumference-center-facing wall of the elastic member. A biometric sensor can be a spectroscopic sensor which measures the spectrum of light energy reflected from and/or absorbed by tissue of the person's arm. Alternatively, a biometric sensor can be an electromagnetic energy sensor which measures parameters and/or patterns of electromagnetic energy passing through and/or emitted by tissue of the person's arm. In an example, measured parameters and/or patterns of electromagnetic energy can be selected from the group consisting of: impedance, resistance, conductivity, permittivity, and electromagnetic wave pattern. Data from the one or more sensors can be analyzed to measure a person's glucose levels.

In an example, a wearable device for non-invasive glucose monitoring can comprise: a circumferentially-undulating attachment member which spans at least a portion of the circumference of a person's arm; and a plurality of biometric sensors which collect data concerning arm tissue, wherein each biometric sensor is located at the proximal portion of an undulation, and wherein the proximal portion of an undulation is the portion of an undulating wave which is closest to the circumferential center of the device. Data from the one or more sensors can be analyzed to measure a person's glucose levels.

In an example, a wearable device for non-invasive glucose monitoring can comprise: an attachment member which is configured to span at least a portion of the circumference of a person's arm; an enclosure which is part of and/or attached to the attachment member; a first spectroscopic sensor in the enclosure which is configured to project a beam of light onto the arm surface at a first angle relative to the enclosure; and a second spectroscopic sensor in the enclosure which is configured to project a beam of light onto the arm surface at a second angle relative to the enclosure, wherein the first angle differs from the second angle by at least 10 degrees.

In an example, an attachment member can be selected from the group consisting of: strap, band, bracelet, ring, armlet, cuff, and sleeve. In an example, an attachment member can be configured to be attached to the person's arm by connecting two ends of the attachment member with a clasp, clip, buckle, hook, pin, plug, or hook-and-eye mechanism. In an example, an attachment member can be configured to be attached to the person's arm by stretching and sliding it over the person's hand onto the arm. In an example, data from first and/or second spectroscopic sensors can be analyzed to measure a person's glucose levels.

In an example, a wearable device for non-invasive glucose monitoring can comprise: an attachment member which is configured to span at least a portion of the circumference of a person's arm; an enclosure which is part of and/or attached to the attachment member; an elastic member filled with a fluid, gel, or gas which is attached to and/or part of the enclosure; and one or more biometric sensors which are configured to record biometric data concerning the person's arm tissue, wherein these one or more biometric sensors are attached to a circumference-center-facing wall of the elastic member.

In an example, a biometric sensor can be a spectroscopic sensor which is configured to measure the spectrum of light energy reflected from and/or absorbed by tissue of the person's arm. In an example, a biometric sensor can be an electromagnetic energy sensor which is configured to measure parameters and/or patterns of electromagnetic energy passing through and/or emitted by tissue of the person's arm. In an example, measured parameters and/or patterns of electromagnetic energy can be selected from the group consisting of: impedance, resistance, conductivity, permittivity, and electromagnetic wave pattern. In an example, data from a biometric sensor can be analyzed to measure a person's glucose levels.

In an example, a wearable device for non-invasive glucose monitoring can comprise: a circumferentially-undulating attachment member which is configured to span at least a portion of the circumference of a person's arm; and a plurality of biometric sensors which collect data concerning arm tissue, wherein each biometric sensor is located at the proximal portion of an undulation, and wherein the proximal portion of an undulation is the portion of an undulating wave which is closest to the circumferential center of the device. In an example, data from the plurality of biometric sensors can be analyzed to measure a person's glucose levels.

In an example, a wearable device for non-invasive glucose monitoring can comprise: an arcuate band which is configured to span (some or all of) the circumferential perimeter of a part of a person's body (such as a wrist, arm, finger, ankle, and/or leg); an energy emitter which is configured to emit energy toward the part of the person's body; an energy receiver which is configured to receive energy from the energy emitter after that energy has passed through and/or been reflected from the part of the person's body; a data processor which receives data from the energy receiver which is analyzed in order to measure the person's body glucose level; and an energy source which provides energy to the energy emitter and/or to the data processor.

In an example, a wearable device for non-invasive glucose monitoring can comprise: an arcuate band which is configured to span (some or all of) the circumferential perimeter of a part of a person's body (such as a wrist, arm, finger, ankle, and/or leg); an energy emitter which is configured to emit energy toward the part of the person's body; an energy receiver which is configured to receive energy from the energy emitter after that energy has passed through and/or been reflected from the part of the person's body; a data processor which receives data from the energy receiver which is analyzed in order to measure the person's body glucose level; an energy source which provides energy to the energy emitter and/or to the data processor; a data transmitter which transmits data from the data processor to a remote device and/or remote location; and an energy barrier between the energy emitter and the energy receiver which reduces transmission of energy from the energy emitter to the energy receiver.

In an example, a wearable device for non-invasive glucose monitoring can comprise: an arcuate band which is configured to span (some or all of) the circumferential perimeter of a part of a person's body (such as a wrist, arm, finger, ankle, and/or leg); an energy emitter which is configured to emit energy toward the part of the person's body; an energy receiver which is configured to receive energy from the energy emitter after that energy has passed through and/or been reflected from the part of the person's body; a data processor which receives data from the energy receiver which is analyzed in order to measure the person's body glucose level; an energy source which provides energy to the energy emitter and/or to the data processor; a data transmitter which transmits data from the data processor to a remote device and/or remote location; and an energy conductor between the energy emitter and the energy receiver which increases transmission of energy from the energy emitter to the energy receiver.

In an example, a wearable device for non-invasive glucose monitoring can comprise: an arcuate band which is configured to span (some or all of) the circumferential perimeter of a part of a person's body (such as a wrist, arm, finger, ankle, and/or leg); an energy emitter which is configured to emit energy toward the part of the person's body; an energy receiver which is configured to receive energy from the energy emitter after that energy has passed through and/or been reflected from the part of the person's body; a data processor which receives data from the energy receiver which is analyzed in order to measure the person's body glucose level; an energy source which provides energy to the energy emitter and/or to the data processor; a data transmitter which transmits data from the data processor to a remote device and/or remote location; and a housing which is held on the person's body by the arcuate band, wherein the energy emitter and the energy receiver are part of the housing.

In an example, a band can be flexible. In an example, a band can be rigid. In an example, portions of a band can be flexible and portions can be rigid. In an example, a band can be elastic or stretchable. In an example, a band can be made from a metal, a polymer, a fabric, or a combination thereof. In an example, a band can be a bracelet, bangle, armlet, or arm band. In an example, a band can be a fitness band or smart watch. In an example, a band can be the strap portion of a fitness monitor or smart watch. In an example, a band can be a finger ring. In an example, a band can be a finger sleeve. In an example, a band can be the cuff of a shirt, pants, shorts, or sock. In an example, a band can include electronic components such as biometric sensors and a display. In an example, a band can further comprise a housing which includes electronic components such as biometric sensors and a display. In an example, a band can be selected from the group consisting of: arm band, armlet, bangle, bracelet, finger ring, finger sleeve, fitness band, pants cuff, shirt cuff, smart watch, and sock cuff.

In an example, a band can span the full circumference of the cross-sectional perimeter of a part of a person's body. In an example, a band can fully encircle a person's wrist, arm, finger, ankle, and/or leg. In an example, a band can span between 50% and 95% of the cross-sectional perimeter of a part of a person's body. In an example, a band can span between 50% and 95% of the cross-sectional perimeter of a part of a person's wrist, arm, finger, ankle, and/or leg. In an example, a band can span between 75% and 95% of the cross-sectional perimeter of a part of a person's wrist, arm, finger, ankle, and/or leg. In an example, a band can comprise a single, continuous piece of material. In an example, a band can comprise two or more pieces and/or segments. In an example, a band can comprise a (partially) circumferential series of flexibly-connected segments.

In an example, the term “proximal” when applied to a person's wrist or arm can mean “closer to the person's shoulder when the arm is extended” and the term “distal” can mean “farther from the person's shoulder when the arm is extended.” In an example, a proximal-to-distal axis can be defined for a band worn on the wrist and/or arm using this shoulder-based definition of proximal and distal. In an example, the term “proximal” when applied to a person's ankle or leg means “closer to the person's hip when the leg is extended” and the term “distal” means “farther from the person's hip when the leg is extended.” In an example, a proximal-to-distal axis can be defined for a band worn on the ankle and/or leg using this hip-based definition of proximal and distal. In an example, the cross-sectional perimeter and/or circumference of an arm or leg can be perpendicular to the central proximal-to-distal axis of that arm or leg, respectively. In an example, the cross-sectional perimeter and/or circumference of a forearm or lower leg can be perpendicular to the central proximal-to-distal axis of that forearm or lower leg, respectively.

In an example, a band can have two ends which can be connected to each other around a part of a person's body by a mechanism selected from the group consisting of: buckle, clasp, hook, pin, knob, button, zipper, magnet, adhesive, and hook-and-eye fabric. In an example, a band can be stretched or expanded so as to slip over a person's hand onto their wrist and/or arm or over a person's foot onto their ankle and/or leg. In an example, a band can have two moveable portions connected by a hinge or joint, wherein these two moveable portions can be locked into position around a person's wrist, arm, ankle, or leg. In an example, a band can have two ends which can be pulled apart from each other by application of an external force to slip the band around a person's wrist, arm, ankle, or leg and then these two ends move back together when the force is removed so as to hold the band around the person's wrist, arm, ankle, or leg.

In an example, an energy emitter can be located on the inward-facing (i.e. body-facing) side of a band. In an example, an energy receiver can also be located on the inward-facing (i.e. body-facing) side of a band. In an example, an energy emitter and an energy receiver can emit and receive light energy, respectively. In an example, a light energy emitter can emit light energy toward body tissue and an energy receiver can receive a portion of that light energy, after the light energy has passed through and/or been reflected from the body tissue.

In an example, a light energy emitter and a light energy receiver together can comprise a spectroscopic (or “spectroscopy”) sensor. In an example, the spectrum of light energy is changed when the light energy passes through body tissue and/or is reflected from body tissue. In an example, changes in the spectrum of light energy which has passed through and/or been reflected from body tissue can be analyzed to detect the composition and/or configuration of body tissue. In an example, these changes in the spectrum of light energy can be analyzed to provide information on the composition and/or configuration of body tissue which, in turn, enables measurement of body glucose level. In an example, a light energy emitter and a light energy receiver together can comprise a sensor selected from the group consisting of: backscattering spectrometry sensor, infrared spectroscopy sensor, ion mobility spectroscopic sensor, mass spectrometry sensor, Near Infrared Spectroscopy sensor (NIS), Raman spectroscopy sensor, spectrometry sensor, spectrophotometer, spectroscopy sensor, ultraviolet spectroscopy sensor, and white light spectroscopy sensor.

In an example, a light energy emitter can emit coherent light. In an example, a light energy emitter can be a laser. In an example, a light energy emitter can be a Light Emitting Diode (LED). In an example, a light energy emitter can emit infrared or near-infrared light. In an example, a light energy emitter can emit ultraviolet light. In an example, a light energy emitter emit red light and/or be a red-light laser. In an example, a light energy emitter emit green light and/or be a green-light laser. In an example, a light energy emitter can emit white light and/or be a white-light laser. In an example, a light energy emitter can emit light with frequency and/or spectrum changes over time. In an example, a light energy emitter can emit a sequence of light pulses at different selected frequencies. In an example, a light energy emitter can emit polarized light. In an example, the polarization of light can change after the light passes through and/or is reflected from body tissue and these changes can be used to measure body glucose level.

In an example, portions of the spectrum of light emitted by a light energy emitter can be absorbed by body tissue and spectral analysis of these absorbed portions can enable measurement of body glucose level. In an example, portions of the spectrum of light emitted by a light energy emitter can be amplified by body tissue and spectral analysis of these amplified portions can enable measurement of body glucose level. In an example, portions of the spectrum of light emitted by a light energy emitter can be shifted by interaction with body tissue and spectral analysis of these shifted portions can enable measurement of body glucose level.

In an example, the depth, breadth, location, and/or type of body tissue or fluid from which light from a light energy emitter is reflected can be changed by adjusting the frequency, color, and/or spectrum of light emitted from the light energy emitter. In an example, the frequency, color, and/or spectrum of light emitted from the light energy emitter can be adjusted in order to more accurately measure body glucose level. In an example, the frequency, color, and/or spectrum of light emitted from the light energy emitter can be adjusted automatically (in an iterative manner) by a device in order to more accurately measure body glucose level for a specific person, for a specific type of activity, or for a specific configuration of the device relative to the person's body surface. In an example, the frequency, color, and/or spectrum of light emitted from the light energy emitter can be adjusted automatically to maintain accurate measurement of body glucose level even if the device shifts and/or moves relative to the person's body surface. In an example, a device can automatically vary the frequency, color, and/or spectrum of light from a light emitter to scan through a range of tissue depths, locations, and/or types in order to obtain more accurate measurement of body glucose level. In an example, a wearable device for non-invasive glucose monitoring can further comprise one or more optical filters or lenses which change the frequency, color, and/or spectrum of light emitted by a light energy emitter.

In an example, the depth, breadth, location, and/or type of body tissue or fluid from which light from a light energy emitter is reflected can be changed by adjusting the power and/or intensity of light emitted from the light energy emitter. In an example, the power and/or intensity of light emitted from the light energy emitter can be adjusted in order to more accurately measure body glucose level. In an example, the power and/or intensity of light emitted from the light energy emitter can be adjusted automatically (in an iterative manner) by a device in order to more accurately measure body glucose level for a specific person, for a specific type of activity, or for a specific configuration of the device relative to the person's body surface. In an example, the power and/or intensity of light emitted from the light energy emitter can be adjusted automatically to maintain accurate measurement of body glucose level even if the device shifts and/or moves relative to the person's body surface. In an example, a device can automatically vary the power and/or intensity of light from a light emitter to scan through a range of tissue depths, locations, and/or types in order to obtain more accurate measurement of body glucose level.

In an example, the depth, breadth, location, and/or type of body tissue or fluid from which light from a light energy emitter is reflected can be changed by adjusting the angle of light emitted from the light energy emitter. In an example, the angle of light emitted from the light energy emitter can be adjusted in order to more accurately measure body glucose level. In an example, the angle of light emitted from the light energy emitter can be adjusted automatically (in an iterative manner) by a device in order to more accurately measure body glucose level for a specific person, for a specific type of activity, or for a specific configuration of the device relative to the person's body surface. In an example, the angle of light emitted from the light energy emitter can be adjusted automatically to maintain accurate measurement of body glucose level even if the device shifts and/or moves relative to the person's body surface. In an example, a device can automatically vary the angle of light from a light emitter to scan through a range of tissue depths, locations, and/or types in order to obtain more accurate measurement of body glucose level. In an example, a wearable device for non-invasive glucose monitoring can further comprise one or more optical filters or lenses which change the projection and/or body incidence angle of a light beam emitted by a light energy emitter.

In an example, the depth, breadth, location, and/or type of body tissue or fluid from which light from a light energy emitter is reflected can be changed by adjusting the coherence, polarization, and/or phase of light emitted from the light energy emitter. In an example, the coherence, polarization, and/or phase of light emitted from the light energy emitter can be adjusted in order to more accurately measure body glucose level. In an example, the coherence, polarization, and/or phase of light emitted from the light energy emitter can be adjusted automatically (in an iterative manner) by a device in order to more accurately measure body glucose level for a specific person, for a specific type of activity, or for a specific configuration of the device relative to the person's body surface. In an example, the coherence, polarization, and/or phase of light emitted from the light energy emitter can be adjusted automatically to maintain accurate measurement of body glucose level even if the device shifts and/or moves relative to the person's body surface. In an example, a device can automatically vary the coherence, polarization, and/or phase of light from a light emitter to scan through a range of tissue depths, locations, and/or types in order to obtain more accurate measurement of body glucose level. In an example, a wearable device for non-invasive glucose monitoring can further comprise one or more optical filters or lenses which change the coherence, polarization, and/or phase of light emitted by a light energy emitter.

In an example, a wearable device for non-invasive glucose monitoring can comprise a first light energy emitter and a second light energy emitter. In an example, the first light energy emitter can emit light with a first light frequency, color, and/or spectrum and the second light energy emitter can emit light with a second light frequency, color, and/or spectrum. In an example, light from the first light energy emitter can reflect primarily from a first depth, breadth, location, and/or type of body tissue and light from the second light energy emitter can reflect primarily from a first depth, breadth, location, and/or type of body tissue. In an example, first and second light energy emitters can emit light simultaneously. In an example, first and second light energy emitters can emit light in a selected chronological sequence and/or timing pattern.

In an example, a wearable device for non-invasive glucose monitoring can comprise a first light energy emitter and a second light energy emitter. In an example, the first light energy emitter can emit light with a first light power and/or intensity and the second light energy emitter can emit light with a second light power and/or intensity. In an example, light from the first light energy emitter can reflect primarily from a first depth, breadth, location, and/or type of body tissue and light from the second light energy emitter can reflect primarily from a first depth, breadth, location, and/or type of body tissue. In an example, first and second light energy emitters can emit light simultaneously. In an example, first and second light energy emitters can emit light in a selected chronological sequence and/or timing pattern.

In an example, a wearable device for non-invasive glucose monitoring can comprise a first light energy emitter and a second light energy emitter. In an example, the first light energy emitter can emit light with a first light projection and/or body incidence angle and the second light energy emitter can emit light with a second light projection and/or body incidence angle. In an example, light from the first light energy emitter can reflect primarily from a first depth, breadth, location, and/or type of body tissue and light from the second light energy emitter can reflect primarily from a first depth, breadth, location, and/or type of body tissue. In an example, first and second light energy emitters can emit light simultaneously. In an example, first and second light energy emitters can emit light in a selected chronological sequence and/or timing pattern.

In an example, a wearable device for non-invasive glucose monitoring can comprise a first light energy emitter and a second light energy emitter. In an example, the first light energy emitter can emit light with a first light coherence, polarization, and/or phase and the second light energy emitter can emit light with a second light coherence, polarization, and/or phase. In an example, light from the first light energy emitter can reflect primarily from a first depth, breadth, location, and/or type of body tissue and light from the second light energy emitter can reflect primarily from a first depth, breadth, location, and/or type of body tissue. In an example, first and second light energy emitters can emit light simultaneously. In an example, first and second light energy emitters can emit light in a selected chronological sequence and/or timing pattern.

In an example, the depth, breadth, location, and/or type of body tissue or fluid from which light from a light energy emitter is reflected and received by a light energy receiver can be changed by adjusting the distance between a light energy emitter and a light energy receiver. In an example, the distance between a light energy emitter and a light energy receiver can be adjusted in order to more accurately measure body glucose level. In an example, the distance between a light energy emitter and a light energy receiver can be adjusted automatically (in an iterative manner) by a device in order to more accurately measure body glucose level for a specific person, for a specific type of activity, or for a specific configuration of the device relative to the person's body surface. In an example, the distance between a light energy emitter and a light energy receiver can be adjusted automatically to maintain accurate measurement of body glucose level even if the device shifts and/or moves relative to the person's body surface. In an example, a device can automatically vary the distance between a light energy emitter and a light energy receiver to scan through a range of tissue depths, locations, and/or types in order to obtain more accurate measurement of body glucose level.

In an example, the depths, breadths, locations, and/or types of body tissue or fluid from which light beams from a plurality of light energy emitters are reflected can be determined by a selected geometric configuration of the plurality of light energy emitters and a light energy receiver. In an example, a selected geometric configuration of a plurality of light energy emitters and a light energy receiver can be designed to most accurately measure body glucose level. In an example, the geometric configuration of a plurality of light energy emitters and a light energy receiver can be adjusted automatically (in an iterative manner) by a device in order to more accurately measure body glucose level for a specific person, for a specific type of activity, or for a specific configuration of the device relative to the person's body surface. In an example, the geometric configuration of a plurality of light energy emitters and a light energy receiver can be adjusted automatically to maintain accurate measurement of body glucose level even if the device shifts and/or moves relative to the person's body surface. In an example, a device can automatically vary the geometric configuration of a plurality of light energy emitters and a light energy receiver in order to scan through a range of tissue depths, locations, and/or types in order to measure body glucose level more accurately. In an example, a plurality of light energy emitters can emit light simultaneously. In an example, a plurality of light energy emitters can emit light in a selected chronological sequence and/or timing pattern.

In an example, a plurality of light energy emitters can be configured in a linear array in proximity to a light energy receiver. In an example, a plurality of light energy emitters can be configured in a linear array including a light energy receiver. In an example, a plurality of light energy emitters can be configured in a polygonal array in proximity to a light energy receiver. In an example, a plurality of light energy emitters can be configured in a polygonal array including a light energy receiver. In an example, a plurality of light energy emitters can be configured in a polygonal array around a light energy receiver. In an example, a plurality of light energy emitters can be configured in a circular or other arcuate array in proximity to a light energy receiver. In an example, a plurality of light energy emitters can be configured in a circular or other arcuate array including a light energy receiver. In an example, a plurality of light energy emitters can be configured in a circular or other arcuate array around a light energy receiver. In an example, a plurality of light energy emitters can emit light in a circular sequence around a central light energy receiver.

In an example, the depths, breadths, locations, and/or types of body tissue or fluid from which light beams are reflected and received by a plurality of light energy receivers can be determined by a selected geometric configuration of a light energy emitter and the plurality of light energy receivers. In an example, a selected geometric configuration of a light energy emitter and a plurality of light energy receivers can be designed to most accurately measure body glucose level. In an example, the geometric configuration of a light energy emitter and a plurality of light energy receivers can be adjusted automatically (in an iterative manner) by a device in order to more accurately measure body glucose level for a specific person, for a specific type of activity, or for a specific configuration of the device relative to the person's body surface. In an example, the geometric configuration of a light energy emitter and a plurality of light energy receivers can be adjusted automatically to maintain accurate measurement of body glucose level even if the device shifts and/or moves relative to the person's body surface. In an example, a device can automatically vary the geometric configuration of a light energy emitter and a plurality of light energy receivers in order to scan through a range of tissue depths, locations, and/or types in order to measure body glucose level more accurately.

In an example, a plurality of light energy receivers can be configured in a linear array in proximity to a light energy emitter. In an example, a plurality of light energy receivers can be configured in a linear array including a light energy emitter. In an example, a plurality of light energy receivers can be configured in a polygonal array in proximity to a light energy emitter. In an example, a plurality of light energy receivers can be configured in a polygonal array including a light energy emitter. In an example, a plurality of light energy receivers can be configured in a polygonal array around a light energy emitter. In an example, a plurality of light energy receivers can be configured in a circular or other arcuate array in proximity to a light energy emitter. In an example, a plurality of light energy receivers can be configured in a circular or other arcuate array including a light energy emitter. In an example, a plurality of light energy receivers can be configured in a circular or other arcuate array around a light energy emitter.

In an example, a light energy emitter can be part of an arcuate band. In an example, a light energy emitter can be part of a housing which is held on a person's body by an arcuate band. In an example, a wearable device for non-invasive glucose monitoring can comprise an array, grid, and/or matrix of two or more light energy emitters with a proximal-to-distal orientation. In an example, a wearable device for non-invasive glucose monitoring can comprise an array, grid, and/or matrix of two or more light energy emitters along a proximal-to-distal axis. In an example, a wearable device for non-invasive glucose monitoring can comprise an array, grid, and/or matrix of two or more light energy emitters with a circumferential orientation. In an example, a wearable device for non-invasive glucose monitoring can comprise an array, grid, and/or matrix of two or more light energy emitters along a circumferential axis.

In an example, a wearable device for non-invasive glucose monitoring can comprise a linear array, grid, and/or matrix of light energy emitters. In an example, a wearable device for non-invasive glucose monitoring can comprise a rectangular array, grid, and/or matrix of light energy emitters. In an example, a wearable device for non-invasive glucose monitoring can comprise a circular or elliptical array, grid, and/or matrix of light energy emitters. In an example, a wearable device for non-invasive glucose monitoring can comprise a checkerboard array, grid, and/or matrix of light energy emitters. In an example, a wearable device for non-invasive glucose monitoring can comprise a three-dimensional stacked array, grid, and/or matrix of light energy emitters. In an example, a wearable device for non-invasive glucose monitoring can comprise a sunburst and/or radial-spoke array, grid, and/or matrix of light energy emitters. In an example, a wearable device for non-invasive glucose monitoring can comprise a sinusoidal array, grid, and/or matrix of light energy emitters.

In an example, an array, grid, and/or matrix of two or more light energy emitters can span up to 10% of the cross-sectional circumference of a part of a person's body such as a wrist, arm, finger, ankle, or leg. In an example, an array, grid, and/or matrix of two or more light energy emitters can span between 10% and 25% of the cross-sectional circumference of a part of a person's body such as a wrist, arm, finger, ankle, or leg. In an example, an array, grid, and/or matrix of two or more light energy emitters can span between 25% and 50% of the cross-sectional circumference of a part of a person's body such as a wrist, arm, finger, ankle, or leg. In an example, an array, grid, and/or matrix of two or more light energy emitters can span between 50% and 100% of the cross-sectional circumference of a part of a person's body such as a wrist, arm, finger, ankle, or leg.

In an example, a light energy receiver can be part of an arcuate band. In an example, a light energy receiver can be part of a housing which is held on a person's body by an arcuate band. In an example, a wearable device for non-invasive glucose monitoring can comprise an array, grid, and/or matrix of two or more light energy receivers with a proximal-to-distal orientation. In an example, a wearable device for non-invasive glucose monitoring can comprise an array, grid, and/or matrix of two or more light energy receivers along a proximal-to-distal axis. In an example, a wearable device for non-invasive glucose monitoring can comprise an array, grid, and/or matrix of two or more light energy receivers with a circumferential orientation. In an example, a wearable device for non-invasive glucose monitoring can comprise an array, grid, and/or matrix of two or more light energy receivers along a circumferential axis.

In an example, a wearable device for non-invasive glucose monitoring can comprise a linear array, grid, and/or matrix of light energy receivers. In an example, a wearable device for non-invasive glucose monitoring can comprise a rectangular array, grid, and/or matrix of light energy receivers. In an example, a wearable device for non-invasive glucose monitoring can comprise a circular or elliptical array, grid, and/or matrix of light energy receivers. In an example, a wearable device for non-invasive glucose monitoring can comprise a checkerboard array, grid, and/or matrix of light energy receivers. In an example, a wearable device for non-invasive glucose monitoring can comprise a three-dimensional stacked array, grid, and/or matrix of light energy receivers. In an example, a wearable device for non-invasive glucose monitoring can comprise a sunburst and/or radial-spoke array, grid, and/or matrix of light energy receivers. In an example, a wearable device for non-invasive glucose monitoring can comprise a sinusoidal array, grid, and/or matrix of light energy receivers.

In an example, an array, grid, and/or matrix of two or more light energy receivers can span up to 10% of the cross-sectional circumference of a part of a person's body such as a wrist, arm, finger, ankle, or leg. In an example, an array, grid, and/or matrix of two or more light energy receivers can span between 10% and 25% of the cross-sectional circumference of a part of a person's body such as a wrist, arm, finger, ankle, or leg. In an example, an array, grid, and/or matrix of two or more light energy receivers can span between 25% and 50% of the cross-sectional circumference of a part of a person's body such as a wrist, arm, finger, ankle, or leg. In an example, an array, grid, and/or matrix of two or more light energy receivers can span between 50% and 100% of the cross-sectional circumference of a part of a person's body such as a wrist, arm, finger, ankle, or leg.

In an example, a light energy emitter and a light energy receiver can be part of an arcuate band. In an example, a light energy emitter and a light energy receiver can be part of a housing which is held on a person's body by an arcuate band. In an example, a wearable device for non-invasive glucose monitoring can comprise an array, grid, and/or matrix of (alternating) light energy emitters and receivers with a proximal-to-distal orientation. In an example, a wearable device for non-invasive glucose monitoring can comprise an array, grid, and/or matrix of (alternating) light energy emitters and receivers along a proximal-to-distal axis. In an example, a wearable device for non-invasive glucose monitoring can comprise an array, grid, and/or matrix of (alternating) light energy emitters and receivers with a circumferential orientation. In an example, a wearable device for non-invasive glucose monitoring can comprise an array, grid, and/or matrix of (alternating) light energy emitters and receivers along a circumferential axis.

In an example, a wearable device for non-invasive glucose monitoring can comprise a linear array, grid, and/or matrix of (alternating) light energy emitters and receivers. In an example, a wearable device for non-invasive glucose monitoring can comprise a rectangular array, grid, and/or matrix of (alternating) light energy emitters and receivers. In an example, a wearable device for non-invasive glucose monitoring can comprise a circular or elliptical array, grid, and/or matrix of (alternating) light energy emitters and receivers. In an example, a wearable device for non-invasive glucose monitoring can comprise a checkerboard array, grid, and/or matrix of (alternating) light energy emitters and receivers. In an example, a wearable device for non-invasive glucose monitoring can comprise a three-dimensional stacked array, grid, and/or matrix of (alternating) light energy emitters and receivers. In an example, a wearable device for non-invasive glucose monitoring can comprise a sunburst and/or radial-spoke array, grid, and/or matrix of (alternating) light energy emitters and receivers. In an example, a wearable device for non-invasive glucose monitoring can comprise a sinusoidal array, grid, and/or matrix of (alternating) light energy emitters and receivers.

In an example, an array, grid, and/or matrix of (alternating) light energy emitters and receivers can span up to 10% of the circumference of a part of a person's body such as a wrist, arm, finger, ankle, or leg. In an example, an array, grid, and/or matrix of (alternating) light energy emitters and receivers can span between 10% and 25% of the circumference of a part of a person's body such as a wrist, arm, finger, ankle, or leg. In an example, an array, grid, and/or matrix of (alternating) light energy emitters and receivers can span between 25% and 50% of the circumference of a part of a person's body such as a wrist, arm, finger, ankle, or leg. In an example, an array, grid, and/or matrix of (alternating) light energy emitters and receivers can span between 50% and 100% of the circumference of a part of a person's body such as a wrist, arm, finger, ankle, or leg.

In an example, a wearable device for non-invasive glucose monitoring can comprise an array, grid, and/or matrix of light energy emitters which differ in one or more parameters selected from the group consisting of: location and/or distance from a light energy receiver; distance to body surface; light beam frequency, color, and/or spectrum; light beam coherence, polarity, and/or phase; light beam power and/or intensity; light beam projection and/or body incidence angle; light beam duration; light beam size; and light beam focal distance. In an example, a wearable device for non-invasive glucose monitoring can comprise an array, grid, and/or matrix of light energy receivers which differ in: location and/or distance from a light energy emitter; and/or distance to body surface.

In an example, the frequency, color, and/or spectrum of a beam of light emitted from a light energy emitter can be changed over time to create a chronological sequence of beams of light with different frequencies, colors, and/or spectrums. In an example, the angle of a beam of light emitted from a light energy emitter can be changed over time to create a chronological sequence of beams of light with different projection and/or body incidence angles. In an example, the power or intensity of a beam of light emitted from a light energy emitter can be changed over time to create a chronological sequence of beams of light with different power or intensity levels. Such sequences can help to more accurately measure body glucose level.

In an example, the frequency, color, and/or spectrum of a beam of light emitted from a light energy emitter can be changed in response to specific environmental conditions (e.g. temperature or humidity) and/or specific activities in which the person wearing a device is engaged (e.g. high level of movement, eating, sleeping, etc.) in order to more accurately measure body glucose level. In an example, the projection angle of a beam of light emitted from a light energy emitter can be changed in response to specific environmental conditions (e.g. temperature or humidity) and/or specific activities in which the person wearing a device is engaged (e.g. high level of movement, eating, sleeping, etc.) in order to more accurately measure body glucose level. In an example, the power and/or intensity of a beam of light emitted from a light energy emitter can be changed in response to specific environmental conditions (e.g. temperature or humidity) and/or specific activities in which the person wearing a device is engaged (e.g. high level of movement, eating, sleeping, etc.) in order to more accurately measure body glucose level.

In an example, an energy emitter and an energy receiver can emit and receive electromagnetic energy, respectively. In an example, an electromagnetic energy emitter can be an antenna. In an example, an electromagnetic energy emitter can emit electricity. In an example, an electromagnetic energy emitter can emit microwaves. In an example, an electromagnetic energy emitter can emit radio waves. In an example, an electromagnetic energy receiver can be an electromagnetic energy sensor. In an example, an electromagnetic energy receiver can be an electromagnetic antenna. In an example, an electromagnetic energy emitter can emit electromagnetic energy into body tissue and an electromagnetic energy receiver can receive a portion of that electromagnetic energy after the electromagnetic energy has passed through the body tissue.

In an example, changes in electromagnetic energy received by an electromagnetic energy receiver can be analyzed to detect changes in the conductivity, resistance, capacitance, impedance, and/or permittivity of body tissue. In an example, changes in the conductivity, resistance, capacitance, impedance, and/or permittivity of body tissue can be analyzed to detect changes in the composition and/or configuration of the body tissue. In an example, changes in the conductivity, resistance, capacitance, impedance, and/or permittivity of body tissue can be analyzed to detect changes in body glucose level. In an example, changes in electromagnetic energy received by the electromagnetic energy receiver can be analyzed to detect changes in body glucose level.

In an example, the depth, breadth, location, and/or type of body tissue or fluid through which electromagnetic energy passes can be changed by adjusting the power, wavelength, and/or frequency of electromagnetic energy emitted from an electromagnetic energy emitter. In an example, the power, wavelength, and/or frequency of electromagnetic energy emitted from an electromagnetic energy emitter can be adjusted in order to more accurately measure body glucose level. In an example, the power, wavelength, and/or frequency of electromagnetic energy emitted from an electromagnetic energy emitter can be adjusted automatically (in an iterative manner) by a device in order to more accurately measure body glucose level for a specific person, for a specific type of activity, or for a specific configuration of the device relative to the person's body surface. In an example, the power, wavelength, and/or frequency of electromagnetic energy emitted from an electromagnetic energy emitter can be adjusted automatically to maintain accurate measurement of body glucose level even if the device shifts and/or moves relative to the person's body surface. In an example, a device can automatically vary the power, wavelength, and/or frequency of electromagnetic energy from an electromagnetic energy emitter to scan through a range of tissue depths, locations, and/or types in order to obtain more accurate measurement of body glucose level.

In an example, the depth, breadth, location, and/or type of body tissue or fluid through which electromagnetic energy passes can be changed by adjusting the location or shape of an electromagnetic energy emitter. In an example, the location or shape of an electromagnetic energy emitter can be adjusted in order to more accurately measure body glucose level. In an example, the location or shape of an electromagnetic energy emitter can be adjusted automatically (in an iterative manner) by a device in order to more accurately measure body glucose level for a specific person, for a specific type of activity, or for a specific configuration of the device relative to the person's body surface. In an example, the location or shape of an electromagnetic energy emitter can be adjusted automatically to maintain accurate measurement of body glucose level even if the device shifts and/or moves relative to the person's body surface. In an example, a device can automatically vary the location an electromagnetic energy emitter to scan through a range of tissue depths, locations, and/or types in order to obtain more accurate measurement of body glucose level.

In an example, a wearable device for non-invasive glucose monitoring can comprise a first electromagnetic energy emitter and a second electromagnetic energy emitter. In an example, the first electromagnetic energy emitter can emit electromagnetic energy with a first electromagnetic energy frequency and the second electromagnetic energy emitter can emit electromagnetic energy with a second electromagnetic energy frequency. In an example, electromagnetic energy from the first electromagnetic energy emitter can reflect primarily from a first depth, breadth, location, and/or type of body tissue and electromagnetic energy from the second electromagnetic energy emitter can reflect primarily from a first depth, breadth, location, and/or type of body tissue. In an example, first and second electromagnetic energy emitters can emit electromagnetic energy simultaneously. In an example, first and second electromagnetic energy emitters can emit electromagnetic energy in a selected chronological sequence and/or timing pattern.

In an example, a selected geometric configuration of a plurality of electromagnetic energy emitters and an electromagnetic energy receiver can be designed to most accurately measure body glucose level. In an example, the geometric configuration of a plurality of electromagnetic energy emitters and an electromagnetic energy receiver can be adjusted automatically (in an iterative manner) by a device in order to more accurately measure body glucose level for a specific person, for a specific type of activity, or for a specific configuration of the device relative to the person's body surface. In an example, the geometric configuration of a plurality of electromagnetic energy emitters and an electromagnetic energy receiver can be adjusted automatically to maintain accurate measurement of body glucose level even if the device shifts and/or moves relative to the person's body surface. In an example, a device can automatically vary the geometric configuration of a plurality of electromagnetic energy emitters and an electromagnetic energy receiver in order to scan through a range of tissue depths, locations, and/or types in order to measure body glucose level more accurately. In an example, a plurality of electromagnetic energy emitters can emit electromagnetic energy simultaneously. In an example, a plurality of electromagnetic energy emitters can emit electromagnetic energy in a selected chronological sequence and/or timing pattern.

In an example, a selected geometric configuration of an electromagnetic energy emitter and a plurality of electromagnetic energy receivers can be designed to most accurately measure body glucose level. In an example, the geometric configuration of an electromagnetic energy emitter and a plurality of electromagnetic energy receivers can be adjusted automatically (in an iterative manner) by a device in order to more accurately measure body glucose level for a specific person, for a specific type of activity, or for a specific configuration of the device relative to the person's body surface. In an example, the geometric configuration of an electromagnetic energy emitter and a plurality of electromagnetic energy receivers can be adjusted automatically to maintain accurate measurement of body glucose level even if the device shifts and/or moves relative to the person's body surface. In an example, a device can automatically vary the geometric configuration of an electromagnetic energy emitter and a plurality of electromagnetic energy receivers in order to scan through a range of tissue depths, locations, and/or types in order to measure body glucose level more accurately.

In an example, an energy emitter and an energy receiver can emit and receive, respectively, microwave-level electromagnetic energy. In an example, there can be a resonator between a microwave energy emitter and a microwave energy receiver, wherein the resonant frequency of the resonator is changed by changes in body glucose levels of nearby tissue and/or fluid. In an example, a resonator can be a split ring resonator. In an example, changes in body glucose levels of nearby tissue and/or fluid change the permittivity of the tissue and/or fluid which, in turn, changes the resonant frequency of the resonator. In an example, a wearable device for non-invasive glucose monitoring can comprise a microwave energy emitter, a microwave energy receiver, and a resonator, wherein changes in microwave transmission from the microwave energy emitter to the microwave energy receiver are used to measure changes in body glucose levels of nearby tissue and/or fluid.

The permittivity of tissue and/or fluid is the ability of tissue and/or fluid to transmit an electromagnetic energy field. Permittivity depends on the amount of electrical energy that is stored within the tissue and/or fluid when the tissue and/or fluid is exposed to an electromagnetic energy field. In an example, this device can measure body glucose levels in nearby body fluid and/or tissue by measuring the permittivity of that body fluid and/or tissue. In an example, this device can measure changes body glucose levels in nearby body fluid and/or tissue by measuring changes in the permittivity of that body fluid and/or tissue. In an example, changes in body glucose levels of tissue and/or fluid can change the permittivity of that tissue and/or fluid, wherein these changes in turn can be measured by this device. In an example, changes in glucose levels in tissue and/or fluid change the permittivity of cell membranes. In an example, the permittivity and glucose levels of blood can be measured by this device.

In an example, the permittivity of tissue and/or fluid can be different at different microwave energy frequencies. In an example, this device can emit microwave energy at varying frequencies to collect data on the electromagnetic energy interaction between that energy and tissue and/or fluid at different frequencies. In an example, this device can sweep through a selected range of microwave frequencies. In an example, this device can be an electromagnetic energy spectroscopy sensor which collects data on the permittivity of tissue and/or fluid across a selected (sub)spectrum of microwave frequencies. In an example, collecting data on the permittivity of tissue and/or fluid across a range of microwave frequencies can provide more accurate estimation of body glucose levels than collecting data on permittivity at a single microwave frequency.

In an example, a wearable device for non-invasive glucose monitoring can function as a dielectric constant sensor. A dielectric constant is the real part of permittivity. In an example, this device can measure body glucose levels in nearby body fluid and/or tissue by measuring the dielectric constant of that body fluid and/or tissue. In an example, this device can measure changes in body glucose levels in nearby body fluid and/or tissue by measuring changes in the dielectric constant of that body fluid and/or tissue. In an example, there can be an inverse relationship between glucose level in tissue and/or fluid and the dielectric constant of that body fluid and/or tissue.

In an example, this device can collect data concerning the amount and spectral distribution of microwave energy which is reflected from tissue and/or fluid. This can depend on the angle at which energy is reflected as well as the permittivity of the tissue and/or fluid. In an example, this device can measure changes in microwave energy that is reflected from tissue and/or fluid as well as microwave energy that is transmitted through tissue and/or fluid. In an example, changes in body glucose levels can change the reflection coefficient as measured by this device. In an example, changes in body glucose levels can change the resonant frequency of a resonator within this device. In an example, microwave energy emitted near tissue and/or fluid interacts with that tissue and/or fluid, dispersing energy into the transmitted energy and creating a measurable distorted output signal. This distorted output signal can be used to help estimate body glucose levels.

In an example, a microwave energy emitter which is part of this device can receive microwave energy as well as emit microwave energy. In an example, a microwave energy emitter can be configured to measure microwave energy which is reflected from tissue and/or fluid. In an example, changes in the amount and/or spectrum of microwave energy reflected from tissue and/or fluid can be used to measure changes in body glucose levels. In an example, this device can be a spectroscopy sensor which measures changes in the spectrum of electromagnetic energy caused by reflection of that energy from tissue and/or fluid.

In an example, a wearable device for non-invasive glucose monitoring can function as an impedance sensor. In an example, this device can estimate changes in body glucose levels in body fluid and/or tissue by measuring changes in the impedance of that body fluid and/or tissue. In an example, this device can be a resistance sensor. In an example, this device can estimate changes in body glucose levels in body fluid and/or tissue by measuring changes in the resistance of that body fluid and/or tissue. In an example, this device can be an inductance sensor. In an example, this device can estimate changes in body glucose levels in body fluid and/or tissue by measuring changes in the inductance of that body fluid and/or tissue.

In an example, a wearable device for non-invasive glucose monitoring can function as a capacitance sensor. In an example, this device can estimate changes in body glucose levels in body fluid and/or tissue by measuring changes in the capacitance of that body fluid and/or tissue. In an example, this device can be a conductance sensor. In an example, this device can estimate changes in body glucose levels in body fluid and/or tissue by measuring changes in the conductance of that body fluid and/or tissue. In an example, this device can be a conductivity sensor. In an example, this device can estimate changes in body glucose levels in body fluid and/or tissue by measuring changes in the conductivity of that body fluid and/or tissue.

In an example, a wearable device for non-invasive glucose monitoring can function as an impedance sensor. In an example, this device can collect data which is used to estimate changes in body glucose levels in body fluid and/or tissue by collecting data on changes in the impedance of that body fluid and/or tissue. In an example, this device can be a resistance sensor. In an example, this device can collect data which is used to estimate changes in body glucose levels in body fluid and/or tissue by collecting data on changes in the resistance of that body fluid and/or tissue. In an example, this device can be an inductance sensor. In an example, this device can collect data which is used to estimate changes in body glucose levels in body fluid and/or tissue by collecting data on changes in the inductance of that body fluid and/or tissue.

In an example, a wearable device for non-invasive glucose monitoring can function as a capacitance sensor. In an example, this device can collect data which is used to estimate changes in body glucose levels in body fluid and/or tissue by collecting data on changes in the capacitance of that body fluid and/or tissue. In an example, this device can be a conductance sensor. In an example, this device can collect data which is used to estimate changes in body glucose levels in body fluid and/or tissue by collecting data on changes in the conductance of that body fluid and/or tissue. In an example, this device can be a conductivity sensor. In an example, this device can collect data which is used to estimate changes in body glucose levels in body fluid and/or tissue by collecting data on changes in the conductivity of that body fluid and/or tissue.

In an example, an array, grid, and/or matrix of two or more electromagnetic energy emitters can span up to 10% of the circumference of a part of a person's body such as a wrist, arm, finger, ankle, or leg. In an example, an array, grid, and/or matrix of two or more electromagnetic energy emitters can span between 10% and 25% of the circumference of a part of a person's body such as a wrist, arm, finger, ankle, or leg. In an example, an array, grid, and/or matrix of two or more electromagnetic energy emitters can span between 25% and 50% of the circumference of a part of a person's body such as a wrist, arm, finger, ankle, or leg. In an example, an array, grid, and/or matrix of two or more electromagnetic energy emitters can span between 50% and 100% of the circumference of a part of a person's body such as a wrist, arm, finger, ankle, or leg.

In an example, an array, grid, and/or matrix of two or more electromagnetic energy receivers can span up to 10% of the circumference of a part of a person's body such as a wrist, arm, finger, ankle, or leg. In an example, an array, grid, and/or matrix of two or more electromagnetic energy receivers can span between 10% and 25% of the circumference of a part of a person's body such as a wrist, arm, finger, ankle, or leg. In an example, an array, grid, and/or matrix of two or more electromagnetic energy receivers can span between 25% and 50% of the circumference of a part of a person's body such as a wrist, arm, finger, ankle, or leg. In an example, an array, grid, and/or matrix of two or more electromagnetic energy receivers can span between 50% and 100% of the circumference of a part of a person's body such as a wrist, arm, finger, ankle, or leg.

In an example, an array, grid, and/or matrix of (alternating) electromagnetic energy emitters and receivers can span up to 10% of the circumference of a part of a person's body such as a wrist, arm, finger, ankle, or leg. In an example, an array, grid, and/or matrix of (alternating) electromagnetic energy emitters and receivers can span between 10% and 25% of the circumference of a part of a person's body such as a wrist, arm, finger, ankle, or leg. In an example, an array, grid, and/or matrix of (alternating) electromagnetic energy emitters and receivers can span between 25% and 50% of the circumference of a part of a person's body such as a wrist, arm, finger, ankle, or leg. In an example, an array, grid, and/or matrix of (alternating) electromagnetic energy emitters and receivers can span between 50% and 100% of the circumference of a part of a person's body such as a wrist, arm, finger, ankle, or leg.

In an example, the frequency and/or wavelength of electromagnetic energy emitted from an electromagnetic energy emitter can be changed over time to create a chronological sequence of electromagnetic energy with different frequencies and/or wavelengths. In an example, the power and/or current of electromagnetic energy emitted from an electromagnetic energy emitter can be changed over time to create a chronological sequence of electromagnetic energy with different power or current levels. Such sequences can help to more accurately measure body glucose level.

In an example, the frequency and/or wavelength of electromagnetic energy emitted from an electromagnetic energy emitter can be changed in response to specific environmental conditions (e.g. temperature or humidity) and/or specific activities in which the person wearing a device is engaged (e.g. high level of movement, eating, sleeping, etc.) in order to more accurately measure body glucose level. In an example, the power and/or current of electromagnetic energy emitted from an electromagnetic energy emitter can be changed in response to specific environmental conditions (e.g. temperature or humidity) and/or specific activities in which the person wearing a device is engaged (e.g. high level of movement, eating, sleeping, etc.) in order to more accurately measure body glucose level.

In an example, an energy emitter can be separated from an energy receiver by a selected distance. In an example, there can be a selected distance between an energy emitter and an energy receiver. In an example, (an orthogonal component of) this distance can be measured along a circumferential axis. In an example, an energy emitter and an energy receiver can both be along the same circumferential line. In an example, (an orthogonal component of) this distance can be measured along a proximal-to-distal axis. In an example, an energy emitter and an energy receiver can both be along the same proximal-to-distal line. In an example, this selected distance can be expressed in inches and be within the range of 1/16″ to 2″. In an example, this selected distance can be expressed in metric units and be within the range of 2 mm to 5 cm. In an example, if this selected distance is along a circumferential axis, this distance can be expressed in (compass or polar coordinate) degrees and be within the range of 2 degrees to 60 degrees.

In an example, a wearable device for non-invasive glucose monitoring can have two (or more) energy emitters. In an example, two (or more) energy emitters can emit energy in a non-simultaneous (e.g. sequential) manner. In an example, a first energy emitter can be separated from a second energy emitter by a selected distance. In an example, there can be a selected distance between a first energy emitter and a second energy receiver. In an example, (an orthogonal component of) this distance can be measured along a circumferential axis. In an example, a first energy emitter and a second energy emitter can both be along the same circumferential line. In an example, (an orthogonal component of) this distance can be measured along a proximal-to-distal axis. In an example, a first energy emitter and a second energy emitter can both be along the same proximal-to-distal line. In an example, this selected distance can be expressed in inches and be within the range of 1/16″ to 2″. In an example, this selected distance can be expressed in metric units and be within the range of 2 mm to 5 cm. In an example, if this distance is along a circumferential axis, this selected distance can be expressed in (compass or polar coordinate) degrees and be within the range of 2 degrees to 60 degrees.

In an example, a wearable device for non-invasive glucose monitoring can have a circumferential array, matrix, or grid of four or more energy emitters, each of which is separated from the nearest other energy emitter by a distance within the range of 1/16″ to 2″. In an example, a wearable device for non-invasive glucose monitoring can have a circumferential array, matrix, or grid of four or more energy emitters, each of which is separated from the nearest other energy emitter by a distance within the range of 2 mm to 5 cm. In an example, a wearable device for non-invasive glucose monitoring can have a circumferential array, matrix, or grid of four or more energy emitters, each of which is separated from the nearest other energy emitter by a distance within the range of 2 degrees to 60 degrees. In an example, a wearable device for non-invasive glucose monitoring can have a circumferential array of energy emitters which spans between 25% and 100% of the cross-sectional perimeter circumference of a part of the body (e.g. wrist, arm, finger, ankle, or leg) to which the device is attached. In an example, this circumferential array of energy emitters can be even spaced or distributed, with the same pair-wise distance or number of degrees between adjacent energy emitters.

In an example, a wearable device for non-invasive glucose monitoring can have two (or more) energy receivers. In an example, a first energy receiver can be separated from a second energy receiver by a selected distance. In an example, there can be a selected distance between a first energy receiver and a second energy receiver. In an example, (an orthogonal component of) this distance can be measured along a circumferential axis. In an example, a first energy receiver and a second energy receiver can both be along the same circumferential line. In an example, (an orthogonal component of) this distance can be measured along a proximal-to-distal axis. In an example, a first energy receiver and a second energy receiver can both be along the same proximal-to-distal line. In an example, this selected distance can be expressed in inches and be within the range of 1/16″ to 2″. In an example, this selected distance can be expressed in metric units and be within the range of 2 mm to 5 cm. In an example, if this distance is along a circumferential axis, this selected distance can be expressed in (compass or polar coordinate) degrees and be within the range of 2 degrees to 60 degrees.

In an example, a wearable device for non-invasive glucose monitoring can have a circumferential array, matrix, or grid of four or more energy receivers, each of which is separated from the nearest other energy receiver by a distance within the range of 1/16″ to 2″. In an example, a wearable device for non-invasive glucose monitoring can have a circumferential array, matrix, or grid of four or more energy receivers, each of which is separated from the nearest other energy receiver by a distance within the range of 2 mm to 5 cm. In an example, a wearable device for non-invasive glucose monitoring can have a circumferential array, matrix, or grid of four or more energy receivers, each of which is separated from the nearest other energy receiver by a distance within the range of 2 degrees to 60 degrees. In an example, a wearable device for non-invasive glucose monitoring can have a circumferential array of energy receivers which spans between 25% and 100% of the cross-sectional perimeter circumference of a part of the body (e.g. wrist, arm, finger, ankle, or leg) to which the device is attached. In an example, this circumferential array of energy receivers can be even spaced or distributed, with the same pair-wise distance or number of degrees between adjacent energy receivers.

In an example, an energy emitter can be within the circumferential section bounded by 330 degrees and 0 degrees and an energy receiver can be within the circumferential section bounded by 0 degrees and 30 degrees, or vice versa. In an example, an energy emitter can be within the circumferential section bounded by 300 degrees and 0 degrees and an energy receiver can be within the circumferential section bounded by 0 degrees and 60 degrees, or vice versa. In an example, an energy emitter can be within the circumferential section bounded by 330 degrees and 30 degrees, a first energy receiver can be within the circumferential section bounded by 270 degrees and 330 degrees, and a second energy receiver can be within the circumferential section bounded by 30 degrees and 90 degrees. In an example, an energy receiver can be within the circumferential section bounded by 330 degrees and 30 degrees, a first energy emitter can be within the circumferential section bounded by 270 degrees and 330 degrees, and a second energy emitter can be within the circumferential section bounded by 30 degrees and 90 degrees.

In an example, a wearable device for non-invasive glucose monitoring can comprise an array of energy emitters and energy receivers which is part of a wearable arcuate band or one or more segments (or housings) which are attached to a wearable arcuate band. In an example, a wearable device for non-invasive glucose monitoring can comprise a two-dimensional array of energy emitters and energy receivers which is part of a wearable arcuate band or one or more segments (or housings) which are attached to a wearable arcuate band. In an example, a wearable device for non-invasive glucose monitoring can comprise a three-dimensionally stacked array of energy emitters and energy receivers which is part of a wearable arcuate band or one or more segments (or housings) which are attached to a wearable arcuate band. In an example, data from this array can be analyzed to measure a person's body glucose level.

In an example, the type of energy emitted by the energy emitters and received by the energy receivers can be light energy. In an example, the type of energy emitted by the energy emitters and received by the energy receivers can be (non-light-spectrum) electromagnetic energy. In an example, the type of energy emitted by the energy emitters and received by the energy receivers can be microwave energy.

In an example, an array of energy emitters and/or energy receivers can have a circumferential axis and a proximal-to-distal axis. In an example, this array can have at least three energy emitters and/or energy receivers along a circumferential axis and at least two energy emitters and/or energy receivers along a proximal-to-distal axis. In an example, an array can be formed from a plurality of sets of energy emitters and energy receivers, wherein each set forms the vertexes of a square or rectangle. In an example, an array can be formed from a plurality of sets of energy emitters and energy receivers, wherein each set forms the vertexes of a hexagon. In an example, an array can be formed from a plurality of sets of energy emitters and energy receivers, wherein each set forms a circle.

In an example, an array of energy emitters and energy receivers can have a square or rectangular shape. In an example, an array of energy emitters and energy receivers can have a hexagonal shape. In an example, an array of energy emitters and energy receivers can have a circular shape. In an example, an array of energy emitters and energy receivers can have a sunburst (e.g. radial spoke) shape. In an example, an array of energy emitters and energy receivers can have a cylindrical and/or ring shape. In an example, an array of energy emitters and energy receivers can have a conic section shape. In an example, an array of energy emitters and energy receivers can have a saddle shape. In an example, an array of energy emitters and energy receivers can have a helical shape.

In an example, a wearable device for non-invasive glucose monitoring can further comprise a track, channel, or slot along which an energy emitter, an energy receiver, or both can be moved. In an example, this movement can be done manually. In an example, this movement can be done automatically by one or more actuators. In an example, this track, channel, or slot can have a circumferential orientation. In an example, this track, channel, or slot can have a proximal-to-distal orientation. In an example, the distance between an energy emitter and an energy receiver can be adjusted by moving the energy emitter, the energy receiver, or both along such a track, channel, or slot. In an example, the location of an energy emitter and/or an energy receiver relative to a person's body can be adjusted by moving the energy emitter, the energy receiver, or both along such a track, channel, or slot. In an example, movement of an energy emitter, an energy receiver, or both along a track, channel, or slot can enable more accurate measurement of body glucose level. In an example, movement of an energy emitter, an energy receiver, or both along a track, channel, or slot can enable customization of a device to the anatomy of a specific person for more accurate measurement of that person's body glucose level.

In an example, a wearable device for non-invasive glucose monitoring can further comprise a rotating member which holds an energy emitter, an energy receiver, or both. In an example, rotation of this member can be done manually. In an example, this rotation can be done automatically by one or more actuators. In an example, the distance between an energy emitter and an energy receiver can be adjusted by rotating the rotating member. In an example, the location of an energy emitter and/or an energy receiver relative to a person's body can be adjusted by rotating the rotating member. In an example, movement of an energy emitter, an energy receiver, or both by a rotating member can enable more accurate measurement of body glucose level. In an example, such movement of an energy emitter, an energy receiver, or both can enable customization of a device to the anatomy of a specific person for more accurate measurement of that person's body glucose level.

In an example, a wearable device for non-invasive glucose monitoring can include a data processor which receives data from an energy receiver. In an example, a data processor can be selected from the group consisting of: central processing unit, computer, microchip, and microprocessor. In an example, data from an energy receiver can be analyzed to measure body glucose level using one or more methods selected from the group consisting of: Analysis of Variance (ANOVA), Artificial Neural Network (ANN), Auto-Regressive (AR) Modeling, Bayesian Analysis, Bonferroni Analysis (BA), Centroid Analysis, Chi-Squared Analysis, Cluster Analysis, Correlation, Covariance, Data Normalization (DN), Decision Tree Analysis (DTA), Discrete Fourier transform (DFT), Discriminant Analysis (DA), Empirical Mode Decomposition (EMD), Factor Analysis (FA), Fast Fourier Transform (FFT), Feature Vector Analysis (FVA), Fisher Linear Discriminant, Fourier Transformation (FT) Method, Fuzzy Logic (FL) Modeling, Gaussian Model (GM), Generalized Auto-Regressive Conditional Heteroscedasticity (GARCH) Modeling, Hidden Markov Model (HMM), Independent Components Analysis (ICA), Inter-Band Power Ratio, Inter-Channel Power Ratio, Inter-Montage Power Mean, Inter-Montage Ratio, Kalman Filter (KF), Kernel Estimation, Krysto Fur Analysis, Laplacian Filter, Laplacian Montage Analysis, Least Squares Estimation, Linear Regression, Linear Transform, Logit Model, Machine Learning (ML), Markov Model, Maximum Entropy Modeling, Maximum Likelihood, Mean Power, Multi-Band Covariance Analysis, Multi-Channel Covariance Analysis, Multivariate Linear Regression, Multivariate Logit, Multivariate Regression, Naive Bayes Classifier, Neural Network, Non-Linear Programming, Non-negative Matrix Factorization (NMF), Power Spectral Density, Power Spectrum Analysis, Principal Components Analysis (PCA), Probit Model, Quadratic Minimum Distance Classifier, Random Forest (RF), Random Forest Analysis (RFA), Regression Model, Signal Amplitude (SA), Signal Averaging, Signal Decomposition, Sine Wave Compositing, Singular Value Decomposition (SVD), Spine Function, Support Vector and/or Machine (SVM), Time Domain Analysis, Time Frequency Analysis, Time Series Model, Trained Bayes Classifier, Variance, Waveform Identification, Wavelet Analysis, and Wavelet Transformation.

In an example, a wearable device for non-invasive glucose monitoring can further comprise an energy source which powers an energy emitter, an energy receiver, a data processor, and/or a data transmitter. In an example, an energy source can be a battery. In an example, an energy source can transduce, harvest, and/or generate energy from body motion or kinetic energy. In an example, an energy source can transduce, harvest, and/or generate energy from ambient light energy. In an example, an energy source can transduce, harvest, and/or generate energy from body thermal energy. In an example, an energy source can transduce, harvest, and/or generate energy from ambient electromagnetic energy.

In an example, a wearable device for non-invasive glucose monitoring can further comprise a wireless data transmitter and/or data receiver. In various examples, this device can be in wireless communication with an external device selected from the group consisting of: a cell phone, an electronic tablet, electronically-functional eyewear, a home electronics portal, an implanted medical device, an internet portal, a laptop computer, a mobile computer, a mobile phone, a remote computer, a remote control unit, a smart phone, a smart utensil, a television set, and a wearable data processing hub. In an example, additional data processing and analysis can be done within an external device.

In an example, a wearable device for non-invasive glucose monitoring can further comprise an energy barrier between an energy emitter and an energy receiver which reduces the transmission of energy from the emitter to the receiver. In an example, an energy barrier between a light energy emitter and a light energy receiver can be opaque. In an example, an energy barrier between an electromagnetic energy emitter and an electromagnetic energy receiver can be non-conductive and/or radio-opaque. In an example, an energy barrier between a light energy emitter and a light energy receiver can be compressible, flexible, and/or elastic. In an example, an energy barrier can comprise compressible foam. In an example, an energy barrier can be an inflatable member (such as a balloon) which is filled with a gas or liquid. In an example, an energy barrier can have a linear shape. In an example, an energy barrier can have a circular, elliptical, sinusoidal, or other arcuate shape. In an example, an energy barrier can surround an energy receiver. In an example, an energy barrier can surround an energy emitter.

In an example, a wearable device for non-invasive glucose monitoring can further comprise an energy conductor between an energy emitter and an energy receiver which increases the transmission of energy from the emitter to the receiver. In an example, an energy conductor between a light energy emitter and a light energy receiver can be an optical lens and/or fiber optic conduit. In an example, an energy conductor between an electromagnetic energy emitter and an electromagnetic energy receiver can be a split-ring resonator. In an example, an energy conductor can be a circular split-ring resonator. In an example, an energy conductor can be a quadrilateral split-ring resonator. In an example, an energy conductor can comprise a plurality of split-ring resonators with a configuration selected from the group consisting of: nested or concentric; arrayed in series; arrayed in parallel; stacked three-dimensionally; symmetric; asymmetric; spiral and/or helical; and interdigitated or interlocking. In an example, an energy conductor between an electromagnetic energy emitter and an electromagnetic energy receiver can be a capacitor.

In an example, a wearable device for non-invasive glucose monitoring can further comprise a housing which is held on a person's body by an arcuate band. In an example, this housing can be more rigid (less flexible) than the band. In an example, this housing can hold an energy emitter and an energy receiver. In an example, a housing can be the display (and/or primary processing) component of a smart watch, fitness band, wearable body hydration monitor, or wearable glucose monitor.

In an example, a wearable device for non-invasive glucose monitoring can further comprise one or more other types of biometric or environmental sensors in addition to the primary energy emitters and receivers discussed above. In an example, the primary energy emitter and the primary energy receiver of this device, discussed above, can be a light energy emitter and a light energy receiver, but a wearable device for non-invasive glucose monitoring can also include a (non-light-spectrum) electromagnetic energy emitter and a (non-light-spectrum) electromagnetic energy receiver. In an example, the primary energy emitter and the primary energy receiver of this device, discussed above, can be a (non-light-spectrum) electromagnetic energy emitter and a (non-light-spectrum) electromagnetic energy receiver, but a wearable device for non-invasive glucose monitoring can also include a light energy emitter and a light energy receiver. In an example, a wearable device for non-invasive glucose monitoring can comprise both light energy and electromagnetic energy sensors for measuring body glucose levels. In an example, a wearable device for non-invasive glucose monitoring can comprise both spectroscopic and microwave energy sensors for measuring body glucose levels.

In an example, a wearable device for non-invasive glucose monitoring can further comprise one or more other types of biometric or environmental sensors selected from the group consisting of: accelerometer, action potential sensor, ballistocardiographic sensor, biochemical sensor, blood flow sensor, blood pressure sensor, camera, capacitance hygrometry sensor, chemiluminescence sensor, chemoreceptor sensor, chromatography sensor, conductivity sensor, electrical resistance sensor, electrocardiographic (ECG) sensor or other sensor measuring electromagnetic energy from (or transmitted through) a person's heart, electromagnetic resistance sensor, electromyographic (EMG) sensor or other sensor measuring electromagnetic energy from (or transmitted through) a person's muscles, electroporation sensor, galvanic skin response (GSR) sensor, glucose sensor, gyroscope, Hall-effect sensor, heart rate sensor, humidity sensor, impedance sensor, inertial sensor, infrared light sensor, infrared spectroscopy sensor, ion mobility spectroscopic sensor, laser sensor, light intensity sensor, light-spectrum-analyzing sensor, magnetic field sensor, magnetometer, and microphone or other sound sensor.

In an example, a wearable device for non-invasive glucose monitoring can further comprise one or more other types of biometric or environmental sensors selected from the group consisting of: motion sensor, muscle function monitor, near-infrared spectroscopic sensor, neural impulse monitor, neurosensor, optical sensor, optoelectronic sensor, oximetry sensor, pH level sensor, photochemical sensor, photoelectric sensor, photoplethysmographic (PPG) sensor, piezocapacitive sensor, piezoelectric sensor, piezoresistive sensor, plethysmographic sensor, pressure sensor, pulse sensor, Raman spectroscopy sensor, respiratory or pulmonary function sensor, sensor measuring the quantity or spectrum of light absorbed by a person's body, sensor measuring the quantity or spectrum of light reflected from a person's body, skin conductance sensor, skin moisture sensor, sound sensor, spectral analysis sensor, spectrometry sensor, spectrophotometer sensor, spectroscopic sensor, strain gauge, stretch sensor, sweat sensor, sympathetic nerve activity sensor, systolic blood pressure sensor, thermal energy sensor, thermistor or other body temperature sensor, tissue impedance sensor, ultrasonic sensor, ultraviolet light sensor, ultraviolet spectroscopy sensor, variable impedance sensor, variable resistance sensor, variable translucence sensor, and voltmeter.

In an example, a glucose monitoring and managing system can comprise: a wearable glucose-monitoring microwave sensor that collects data which is used to measure a person's intra-body glucose levels; and a wearable or implanted pump which delivers a glucose-level-modifying substance into the person's body based on data collected by the wearable glucose-monitoring microwave sensor. In an example, a closed-loop glucose monitoring and managing system can comprise: a wearable glucose-monitoring microwave sensor which automatically collects data which is used to measure a person's intra-body glucose levels; and a wearable or implanted pump which automatically delivers a glucose-level-modifying substance into the person's body based on data collected by the wearable glucose-monitoring microwave sensor. In an example, a closed-loop glucose monitoring and managing system can comprise: a wearable glucose-monitoring microwave sensor which automatically collects data which is used to measure a person's intra-body glucose levels; and a wearable or implanted pump which automatically delivers a glucose-level-modifying substance into the person's body to maintain intra-body glucose levels within a selected range, wherein operation of the pump is based on data collected by the wearable glucose-monitoring microwave sensor.

In an example, a wearable glucose-monitoring microwave sensor can comprise a microwave energy emitter and a microwave energy receiver, wherein transmission of microwave energy from the microwave energy emitter to the microwave energy receiver is changed by changes in the glucose levels of nearby body tissue and/or fluid. In an example, a wearable glucose-monitoring microwave sensor can collect data concerning microwave energy which is transmitted through body tissue and/or fluid, wherein this transmitted microwave energy is changed by changes in the level of glucose in the body tissue and/or fluid. In an example, a wearable glucose-monitoring microwave sensor can collect data concerning microwave energy which is reflected from body tissue and/or fluid, wherein this reflected microwave energy is changed by changes in the level of glucose in the body tissue and/or fluid. In an example, a wearable glucose-monitoring microwave sensor can collect data concerning the electromagnetic interaction between transmitted microwave energy and nearby body tissue and/or fluid, wherein this interaction is changed by changes in the level of glucose in the body tissue and/or fluid.

In an example, a glucose monitoring and managing system can comprise: a wearable glucose-monitoring microwave sensor which collects data which is used to measure a person's intra-body glucose levels, wherein this glucose-monitoring microwave sensor further comprises a microwave energy emitter and a microwave energy receiver; and a wearable or implanted pump which delivers a glucose-level-modifying substance into the person's body based on data collected by the wearable glucose-monitoring microwave sensor. In an example, a closed-loop glucose monitoring and managing system can comprise: a wearable glucose-monitoring microwave sensor which automatically collects data which is used to measure a person's intra-body glucose levels, wherein this glucose-monitoring microwave sensor further comprises a microwave energy emitter and a microwave energy receiver; and a wearable or implanted pump which automatically delivers a glucose-level-modifying substance into the person's body to maintain intra-body glucose levels within a selected range, wherein operation of the pump is based on data collected by the wearable glucose-monitoring microwave sensor.

In an example, a glucose monitoring and managing system can comprise: a wearable glucose-monitoring microwave sensor which collects data which is used to measure a person's intra-body glucose levels; and a wearable or implanted pump which delivers a glucose-level-modifying substance into the person's body based on data collected by the wearable glucose-monitoring microwave sensor. In an example, a glucose monitoring and managing system can comprise: a wearable glucose-monitoring microwave sensor which measures intra-body glucose level; and a wearable or implanted insulin pump, wherein the pump automatically dispenses insulin when needed based on data from the wearable glucose-monitoring microwave sensor. In an example, a glucose monitoring and managing system can comprise: a wearable glucose-monitoring microwave sensor which collects data concerning electromagnetic energy that is transmitted through and/or reflected from body tissue and/or fluid, wherein this data is analyzed to measure a person's intra-body glucose level; and a wearable or implanted insulin pump, wherein the pump automatically dispenses insulin when needed based on analysis of data from the wearable glucose-monitoring microwave sensor.

In an example, a wearable glucose-monitoring microwave sensor can be a permittivity sensor. The permittivity of body tissue and/or fluid is the ability of body tissue and/or fluid to transmit an electromagnetic field. Permittivity depends on the amount of electrical energy that is stored within the body tissue and/or fluid when the body tissue and/or fluid is exposed to an electromagnetic field. In an example, a wearable glucose-monitoring microwave sensor can measure glucose levels in nearby body fluid and/or tissue by measuring the permittivity of that body fluid and/or tissue. In an example, a wearable glucose-monitoring microwave sensor can measure changes glucose levels in nearby body fluid and/or tissue by measuring changes in the permittivity of that body fluid and/or tissue. In an example, changes in the glucose levels of body tissue and/or fluid can change the permittivity of that body tissue and/or fluid, wherein these changes in turn can be measured by a wearable glucose-monitoring microwave sensor. In an example, changes in glucose levels in body tissue and/or fluid change the permittivity of cell membranes. In an example, the permittivity and glucose levels of blood can be measured by a wearable glucose-monitoring microwave sensor.

In an example, the permittivity of body tissue and/or fluid can be different at different microwave energy frequencies. In an example, a wearable glucose-monitoring microwave sensor can emit microwave energy at varying frequencies to collect data on the electromagnetic interaction between that energy and body tissue and/or fluid at different frequencies. In an example, a wearable glucose-monitoring microwave sensor can sweep through a selected range of microwave frequencies. In an example, a wearable glucose-monitoring microwave sensor can be an electromagnetic spectroscopy sensor which collects data on the permittivity of body tissue and/or fluid across a selected (sub)spectrum of microwave frequencies. In an example, collecting data on the permittivity of body tissue and/or fluid across a range of microwave frequencies can provide more accurate estimation of intra-body glucose levels than collecting data on permittivity at a single microwave frequency.

In an example, a wearable glucose-monitoring microwave sensor can be a dielectric constant sensor. A dielectric constant is the real part of permittivity. In an example, a wearable glucose-monitoring microwave sensor can measure glucose levels in nearby body fluid and/or tissue by measuring the dielectric constant of that body fluid and/or tissue. In an example, a wearable glucose-monitoring microwave sensor can measure changes in glucose levels in nearby body fluid and/or tissue by measuring changes in the dielectric constant of that body fluid and/or tissue. In an example, there can be an inverse relationship between the glucose level in body tissue and/or fluid and the dielectric constant of that body fluid and/or tissue.

In an example, a wearable glucose-monitoring microwave sensor can collect data concerning the amount and spectral distribution of microwave energy which is reflected from body tissue and/or fluid. This can depend on the angle at which energy is reflected as well as the permittivity of the body tissue and/or fluid. In an example, a wearable glucose-monitoring microwave sensor can measure changes in microwave energy that is reflected from body tissue and/or fluid as well as microwave energy that is transmitted through body tissue and/or fluid. In an example, changes in intra-body glucose levels can change the reflection coefficient as measured by a wearable glucose-monitoring microwave sensor. In an example, changes in intra-body glucose levels can change the resonant frequency of a resonator within a wearable glucose-monitoring microwave sensor. In an example, microwave energy emitted near body tissue and/or fluid interacts with that body tissue and/or fluid, dispersing energy into the transmitted energy and creating a measurable distorted output signal. This distorted output signal can be used to help estimate intra-body glucose levels.

In an example, a microwave energy emitter which is part of a wearable glucose-monitoring microwave sensor can receive microwave energy as well as emit microwave energy. In an example, a microwave energy emitter can be configured to measure microwave energy which is reflected from body tissue and/or fluid. In an example, changes in the amount and/or spectrum of microwave energy reflected from body tissue and/or fluid can be used to measure changes in intra-body glucose levels. In an example, a wearable glucose-monitoring microwave sensor can be a spectroscopy sensor which measures changes in the spectrum of electromagnetic energy caused by reflection of that energy from body tissue and/or fluid.

In an example, a wearable glucose-monitoring microwave sensor can be an impedance sensor. In an example, a wearable glucose-monitoring microwave sensor can estimate changes in glucose levels in body fluid and/or tissue by measuring changes in the impedance of that body fluid and/or tissue. In an example, a wearable glucose-monitoring microwave sensor can be a resistance sensor. In an example, a wearable glucose-monitoring microwave sensor can estimate changes in glucose levels in body fluid and/or tissue by measuring changes in the resistance of that body fluid and/or tissue. In an example, a wearable glucose-monitoring microwave sensor can be an inductance sensor. In an example, a wearable glucose-monitoring microwave sensor can estimate changes in glucose levels in body fluid and/or tissue by measuring changes in the inductance of that body fluid and/or tissue.

In an example, a wearable glucose-monitoring microwave sensor can be a capacitance sensor. In an example, a wearable glucose-monitoring microwave sensor can estimate changes in glucose levels in body fluid and/or tissue by measuring changes in the capacitance of that body fluid and/or tissue. In an example, a wearable glucose-monitoring microwave sensor can be a conductance sensor. In an example, a wearable glucose-monitoring microwave sensor can estimate changes in glucose levels in body fluid and/or tissue by measuring changes in the conductance of that body fluid and/or tissue. In an example, a wearable glucose-monitoring microwave sensor can be a conductivity sensor. In an example, a wearable glucose-monitoring microwave sensor can estimate changes in glucose levels in body fluid and/or tissue by measuring changes in the conductivity of that body fluid and/or tissue.

In an example, a wearable glucose-monitoring microwave sensor can be an impedance sensor. In an example, a wearable glucose-monitoring microwave sensor can collect data which is used to estimate changes in glucose levels in body fluid and/or tissue by collecting data on changes in the impedance of that body fluid and/or tissue. In an example, a wearable glucose-monitoring microwave sensor can be a resistance sensor. In an example, a wearable glucose-monitoring microwave sensor can collect data which is used to estimate changes in glucose levels in body fluid and/or tissue by collecting data on changes in the resistance of that body fluid and/or tissue. In an example, a wearable glucose-monitoring microwave sensor can be an inductance sensor. In an example, a wearable glucose-monitoring microwave sensor can collect data which is used to estimate changes in glucose levels in body fluid and/or tissue by collecting data on changes in the inductance of that body fluid and/or tissue.

In an example, a wearable glucose-monitoring microwave sensor can be a capacitance sensor. In an example, a wearable glucose-monitoring microwave sensor can collect data which is used to estimate changes in glucose levels in body fluid and/or tissue by collecting data on changes in the capacitance of that body fluid and/or tissue. In an example, a wearable glucose-monitoring microwave sensor can be a conductance sensor. In an example, a wearable glucose-monitoring microwave sensor can collect data which is used to estimate changes in glucose levels in body fluid and/or tissue by collecting data on changes in the conductance of that body fluid and/or tissue. In an example, a wearable glucose-monitoring microwave sensor can be a conductivity sensor. In an example, a wearable glucose-monitoring microwave sensor can collect data which is used to estimate changes in glucose levels in body fluid and/or tissue by collecting data on changes in the conductivity of that body fluid and/or tissue.

In an example, a wearable glucose-monitoring microwave sensor can comprise: a first component which is configured to emit microwave energy in proximity to body tissue and/or fluid; and a second component which receives this microwave energy after it has interacted with the body tissue and/or fluid. In an example, a wearable glucose-monitoring microwave sensor can comprise: a first component which is configured to transmit microwave energy into body tissue and/or fluid; and a second component which receives this microwave energy after it has been transmitted through the body tissue and/or fluid. In an example, a wearable glucose-monitoring microwave sensor can comprise: a first component which is configured to emit microwave energy in proximity to body tissue and/or fluid and to receive microwave energy which is reflected from this body tissue and/or fluid; and a second component which receives this microwave energy after it has been transmitted through the body tissue and/or fluid.

In an example, a wearable glucose-monitoring microwave sensor can comprise: a microwave energy emitter which is configured to emit microwave energy in proximity to body tissue and/or fluid; and a microwave energy receiver which receives this microwave energy after it has interacted with the body tissue and/or fluid. In an example, a wearable glucose-monitoring microwave sensor can comprise: a microwave energy emitter which is configured to transmit microwave energy into body tissue and/or fluid; and a microwave energy receiver which receives this microwave energy after it has been transmitted through the body tissue and/or fluid. In an example, a wearable glucose-monitoring microwave sensor can comprise: a microwave energy emitter which is configured to emit microwave energy in proximity to body tissue and/or fluid and to receive microwave energy which is reflected from this body tissue and/or fluid; and a microwave energy receiver which receives this microwave energy after it has been transmitted through the body tissue and/or fluid.

In an example, a wearable glucose-monitoring microwave sensor can be configured to measure microwave energy which has been transmitted across a distance in proximity to body tissue and/or fluid. In an example, changes in microwave energy transmitted through body tissue and/or fluid can be used to measure changes in intra-body glucose levels. In an example, a wearable glucose-monitoring microwave sensor can collect data which is used to measure intra-body glucose levels by transmitting electromagnetic energy through body tissue and/or fluid and receiving that electromagnetic energy after it has been transmitted through the body tissue and/or fluid. In an example, intra-body glucose levels can be estimated by analysis of: transmitted microwave energy received by a microwave energy receiver (which is affected by the electromagnetic properties of nearby body tissue and/or fluid): reflected microwave energy received by a microwave energy emitter (which is affected by the electromagnetic properties of nearby body tissue and/or fluid); or both.

In an example, a wearable glucose-monitoring microwave sensor can collect data which is used to measure intra-body glucose levels by emitting electromagnetic energy in proximity to body tissue and/or fluid and by receiving that electromagnetic energy after it has been reflected from the body tissue and/or fluid. In an example, intra-body glucose levels can be estimated by collecting data on microwave energy reflected from body tissue and/or fluid, collecting data on microwave energy transmitted through body tissue and/or fluid, or both.

In an example, a wearable glucose-monitoring microwave sensor can collect data concerning the interaction between an electromagnetic field and body tissue and/or fluid. In an example, intra-body glucose levels can be estimated by analysis of the permittivity or dielectric constant of nearby body tissue and/or fluid. In an example, a wearable glucose-monitoring microwave sensor can measure intra-body glucose levels by measuring how microwave energy emitted by a microwave energy emitter interacts with body tissue and/or fluid between the microwave energy emitter and a microwave energy receiver. In an example, a wearable glucose-monitoring microwave sensor can collect data which is used to measure intra-body glucose levels by creating an electromagnetic field which interacts with body tissue and/or fluid and by collecting data concerning the interaction between that electromagnetic field and the body tissue and/or fluid.

In an example, there can be a gap between a first (microwave-emitting) component and a second (microwave-receiving) component. In an example, there can be an insulator between a first (microwave-emitting) component and a second (microwave-receiving) component. In an example, there can be body tissue and/or fluid directly between a first (microwave-emitting) component and a second (microwave-receiving) component. In an example, there can be body tissue and/or fluid in proximity to a gap or insulator between a first (microwave-emitting) component and a second (microwave-receiving) component. In an example, a wearable glucose-monitoring microwave sensor can comprise: a microwave energy emitter and a microwave energy receiver, wherein the transmitter and receiver are separated and worn in proximity to body tissue and/or fluid.

In an example, a wearable glucose-monitoring microwave sensor can comprise a microwave energy emitting pole and a microwave energy receiving pole. In an example, a wearable glucose-monitoring microwave sensor can comprise a microwave energy emitting antenna and a microwave energy receiving antenna. In an example, a wearable glucose-monitoring microwave sensor can comprise a microwave energy emitting surface and a microwave energy receiving surface. In an example, a wearable glucose-monitoring microwave sensor can be selected from the group consisting of: defected ground structure, electromagnetic resonator, microwave antenna, microwave probe, microstrip, microwave spectrometer, and wave guide.

In an example, a wearable glucose-monitoring microwave sensor can comprise a microwave energy emitter and a microwave energy receiver which are co-planar. In an example, the microwave energy emitter and the microwave energy receiver can both lie within a common flat plane. In an example, a wearable glucose-monitoring microwave sensor can comprise a microwave energy emitter and a microwave energy receiver which are co-linear. In an example, the longitudinal axis of a microwave energy emitter and the longitudinal axis of a microwave energy receiver can be aligned along a common (virtual) straight line, with a gap or insulator between them. In an example, a wearable glucose-monitoring microwave sensor can comprise a microwave energy emitter and a microwave energy receiver which are parallel. In an example, the longitudinal axis of a microwave energy emitter can be parallel to the longitudinal axis of a microwave energy receiver. In an example, the ends of a microwave energy emitter and a microwave energy receiver which are closest to each other can be parallel. In an example, a microwave energy emitter and a microwave energy receiver can both be “T” shaped and in a symmetric configuration wherein their closest ends are parallel.

In an example, a wearable glucose-monitoring microwave sensor can comprise a microwave energy emitter and a microwave energy receiver whose closest edges are equidistant. In an example, the portions of the perimeter of a microwave energy emitter and the perimeter of a microwave energy receiver which are closest to each other can be separated by a constant distance. In an example, an equidistant configuration of a microwave energy emitter and microwave energy receiver can be an interlocking or interdigitating configuration. In an example, an equidistant configuration of a microwave energy emitter and microwave energy receiver can be a geometrically complementary configuration. In an example, an equidistant configuration of a microwave energy emitter and microwave energy receiver can be a sinusoidally-complementary configuration. In an example, a wearable glucose-monitoring microwave sensor can comprise two adjacent microwave antennae with interlocking, interdigitating, or intermeshing projections.

In an example, a wearable glucose-monitoring microwave sensor can comprise a microwave energy emitter and a microwave energy receiver which are both within the same curved planar surface. In an example, a wearable glucose-monitoring microwave sensor can comprise an arcuate microwave energy emitter and an arcuate microwave energy receiver which are configured to conform to the curved surface of a person's body. In an example, a wearable glucose-monitoring microwave sensor can comprise an arcuate microwave energy emitter and arcuate microwave energy receiver which are configured to be a uniform distance from the curved surface of a person's body. In an example, a wearable glucose-monitoring microwave sensor can comprise an arcuate microwave energy emitter and arcuate microwave energy receiver which are configured to be equidistant from the curved surface of a person's body. In an example, a wearable glucose-monitoring microwave sensor can comprise an arcuate microwave energy emitter and an arcuate microwave energy receiver which are contained within an arcuate circular band or ring which is worn around a portion of a person's body.

In an example, a wearable glucose-monitoring microwave sensor can comprise an arcuate microwave energy emitter and an arcuate microwave energy receiver which are co-arcuate, being located along the perimeter and/or surface of the same (virtual) circle, ellipse, oval, band, ring, or cylinder. In an example, a wearable glucose-monitoring microwave sensor can comprise an arcuate microwave energy emitter and an arcuate microwave energy receiver which are co-arcuate, being located along the same arc of a common (virtual) circle, ellipse, oval, band, ring, or cylinder. In an example, a wearable glucose-monitoring microwave sensor can comprise an arcuate microwave energy emitter and/or an arcuate microwave energy receiver with a saddle shape. In an example, a wearable glucose-monitoring microwave sensor can comprise an arcuate microwave energy emitter and/or an arcuate microwave energy receiver with a conic section shape.

In an example, a wearable glucose-monitoring microwave sensor can comprise a flexible microwave energy emitter and a flexible arcuate microwave energy receiver which are configured to conform to the curved surface of a person's body when worn on the person's body. In an example, a wearable glucose-monitoring microwave sensor can comprise an arcuate microwave energy emitter and an arcuate microwave energy receiver which are within a flexible, stretchable, and/or elastic wearable device which conforms to the curvature of the surface of a person's body. In an example, a wearable glucose-monitoring microwave sensor can comprise a microwave energy emitter and a microwave energy receiver which are within a flexible, stretchable, and/or elastic wearable device so as to keep the microwave energy emitter and microwave energy receiver a uniform distance from the surface of a person's body.

In an example, a wearable glucose-monitoring microwave sensor can comprise a spiral microwave antenna. In an example, a spiral microwave antenna can be flat. In an example, a spiral microwave antenna can be saddle-shaped. In an example, a spiral microwave antennae can be configured to conform to the curved surface of a person's body. In an example, a wearable glucose-monitoring microwave sensor can include a spiral microwave energy emitter. In an example, a wearable glucose-monitoring microwave sensor can include a spiral microwave energy receiver. In an example, a wearable glucose-monitoring microwave sensor can include two adjacent spiral antennae. In an example, a wearable glucose-monitoring microwave sensor can include two concentric and/or intertwined spiral antennae. In an example, a wearable glucose-monitoring microwave sensor can include two parallel and/or stacked spiral antennae. In an example, a wearable glucose-monitoring microwave sensor can include spiral antennae with opposite clockwise directions. In an example, a spiral microwave antenna can be a circular, oval, square, hexagonal, or octagonal spiral.

In an example, a wearable glucose-monitoring microwave sensor can comprise a microwave energy emitter and a microwave energy receiver which are nested and/or concentric. In an example, a wearable glucose-monitoring microwave sensor can comprise a microwave energy emitter and a microwave energy receiver comprising nested and/or concentric rings which form a target or “bulls eye” configuration. In an example, a microwave energy emitter can further comprise a plurality of nested and/or concentric microwave antennae. In an example, a microwave energy receiver can further comprise a plurality of nested and/or concentric microwave antennae.

In an example, a wearable glucose-monitoring microwave sensor can comprise an open-ended coaxial probe. In an example, a wearable glucose-monitoring microwave sensor can comprise an open-ended coaxial probe which is configured to be substantially perpendicular to the surface of a person's body. In an example, a wearable glucose-monitoring microwave sensor can comprise an open-ended coaxial probe which is configured to be substantially parallel to the surface of a person's body. In an example, a wearable glucose-monitoring microwave sensor can comprise a waveguide. In an example, a waveguide can have a longitudinal axis which is perpendicular to the surface of a person's body. In an example, a waveguide can have a longitudinal axis which is parallel to the surface of a person's body. In an example, a waveguide can be cylindrical, cubic, spherical, or rectangular.

In an example, a wearable glucose-monitoring microwave sensor can have a shape selected from the group consisting of: “bulls eye” pattern, arc of a circle, circle, circular spiral, coaxial probe, comb shape, concentric rings, conic section, convex lens shape, cube shape, cylinder, cylindrical band, dumbbell, ellipse, hemisphere, hexagon, H-shape antenna, microstrip, nested rings, non-equilateral polygon, octagon, Omega-shape, oval, oval spiral, parallel rings, polygon with rounded vertices, polygonal spiral, quadrilateral, rectangle, rectangular cylinder, rectangular spiral, ring, ring between two microwave poles, saddle shape, sawtooth shape, sinusoidal wave, sphere, split ring, square, square spiral, square wave, S-shape, stacked rings, straight co-linear poles, straight line, target pattern, triangle, T-shape, two complementary sinusoidal shapes, two intertwined spirals, two symmetric spirals, U-shape, Y-shape, and zigzag pattern.

In an example, a wearable glucose-monitoring microwave sensor can further comprise a microwave energy emitter with a shape selected from the group consisting of: “bulls eye” pattern, arc of a circle, circle, circular spiral, coaxial probe, comb shape, concentric rings, conic section, convex lens shape, cube shape, cylinder, cylindrical band, dumbbell, ellipse, hemisphere, hexagon, H-shape antenna, microstrip, nested rings, non-equilateral polygon, octagon, Omega-shape, oval, oval spiral, parallel rings, polygon with rounded vertices, polygonal spiral, quadrilateral, rectangle, rectangular cylinder, rectangular spiral, ring, ring between two microwave poles, saddle shape, sawtooth shape, sinusoidal wave, sphere, split ring, square, square spiral, square wave, S-shape, stacked rings, straight co-linear poles, straight line, target pattern, triangle, T-shape, two complementary sinusoidal shapes, two intertwined spirals, two symmetric spirals, U-shape, Y-shape, and zigzag pattern.

In an example, a wearable glucose-monitoring microwave sensor can further comprise a microwave energy receiver with a shape selected from the group consisting of: “bulls eye” pattern, arc of a circle, circle, circular spiral, coaxial probe, comb shape, concentric rings, conic section, convex lens shape, cube shape, cylinder, cylindrical band, dumbbell, ellipse, hemisphere, hexagon, H-shape antenna, microstrip, nested rings, non-equilateral polygon, octagon, Omega-shape, oval, oval spiral, parallel rings, polygon with rounded vertices, polygonal spiral, quadrilateral, rectangle, rectangular cylinder, rectangular spiral, ring, ring between two microwave poles, saddle shape, sawtooth shape, sinusoidal wave, sphere, split ring, square, square spiral, square wave, S-shape, stacked rings, straight co-linear poles, straight line, target pattern, triangle, T-shape, two complementary sinusoidal shapes, two intertwined spirals, two symmetric spirals, U-shape, Y-shape, and zigzag pattern.

In an example, a wearable glucose-monitoring microwave sensor can comprise a plurality of microwave energy emitters and/or receivers. In an example, a wearable glucose-monitoring microwave sensor can comprise an array, matrix, or series of microwave energy emitters and/or receivers. In an example, a wearable glucose-monitoring microwave sensor can comprise a co-planar array, matrix, or series of microwave energy emitters and/or receivers. In an example, a wearable glucose-monitoring microwave sensor can comprise a co-planar repeating array, matrix, or series of microwave energy emitters and/or receivers.

In an example, a wearable glucose-monitoring microwave sensor can comprise an arcuate array, matrix, or series of microwave energy emitters. In an example, a wearable glucose-monitoring microwave sensor can comprise an arcuate array, matrix, or series of microwave energy emitters around an arcuate band that is worn around a portion of a person's body. In an example, a wearable glucose-monitoring microwave sensor can comprise an arcuate array, matrix, or series of microwave energy receivers. In an example, a wearable glucose-monitoring microwave sensor can comprise an arcuate array, matrix, or series of microwave energy receivers around an arcuate band that is worn around a portion of a person's body.

In an example, a wearable glucose-monitoring microwave sensor can comprise an array, matrix, or series of microwave energy emitters and/or receivers of different sizes or configurations. In an example, a wearable glucose-monitoring microwave sensor can comprise an array, matrix, or series of microwave energy emitters and/or receivers with a selected progression of different sizes (e.g. smaller to larger), shapes (e.g. less arcuate to more arcuate), gaps (e.g. closer together to farther apart), material compositions, and/or rotations.

In an example, a wearable glucose-monitoring microwave sensor can comprise an array, matrix, or series of microwave antennae with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of wave guides with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of microwave spectrometers with a selected progression of different sizes (e.g. smaller to larger), shapes (e.g. less arcuate to more arcuate), gaps (e.g. closer together to farther apart), material compositions, and/or rotations.

In an example, a wearable glucose-monitoring microwave sensor can comprise an array, matrix, or series of defected ground structures with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of electromagnetic resonators with a selected progression of different sizes (e.g. smaller to larger), shapes (e.g. less arcuate to more arcuate), gaps (e.g. closer together to farther apart), material compositions, and/or rotations. In an example, a wearable glucose-monitoring microwave sensor can comprise an array, matrix, or series of split ring resonators with a selected progression of different sizes (e.g. smaller to larger), shapes (e.g. less arcuate to more arcuate), gaps (e.g. closer together to farther apart), material compositions, and/or rotations.

In an example, a glucose monitoring and managing system can comprise: a plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a co-planar plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: an arcuate plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a cylindrical plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: an array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors. In an example, a glucose monitoring and managing system can comprise: a co-planar array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors.

In an example, a glucose monitoring and managing system can comprise: an arcuate array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors. In an example, a glucose monitoring and managing system can comprise: a cylindrical or ring-shaped array, matrix, or series of wearable glucose-monitoring microwave sensors which collect data which is used to measure a person's intra-body glucose levels; and a wearable or implanted pump which delivers a glucose-level-modifying substance into the person's body based on data collected by the array, matrix, or series of wearable glucose-monitoring microwave sensors.

In an example, a glucose monitoring and managing system can comprise: a plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a co-planar plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: an arcuate plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors. In an example, a glucose monitoring and managing system can comprise: a cylindrical plurality of wearable glucose-monitoring microwave sensors which collect data which is used to measure a person's intra-body glucose levels; and a wearable or implanted pump which delivers a glucose-level-modifying substance into the person's body based on data collected by the plurality of wearable glucose-monitoring microwave sensors.

In an example, a glucose monitoring and managing system can comprise: a first wearable glucose-monitoring microwave sensor which collects data which is used to measure a person's intra-body glucose levels; a second wearable glucose-monitoring microwave sensor which collects data which is used to measure a person's intra-body glucose levels; and a wearable or implanted pump which delivers a glucose-level-modifying substance into the person's body based on data collected by the plurality of wearable glucose-monitoring microwave sensors. In an example, the first and second wearable glucose-monitoring microwave sensors can be co-linear. In an example, the first and second wearable glucose-monitoring microwave sensors can be co-planar. In an example, the first and second wearable glucose-monitoring microwave sensors can be stacked and/or parallel to each other. In an example, first and second wearable glucose-monitoring microwave sensors can be located along the curved surface of a common (virtual) ring, band, or cylinder. In an example, first and second wearable glucose-monitoring microwave sensors can be located along the curved surface of a finger ring, wrist band, or arm band.

In an example, a glucose monitoring and managing system can comprise: a first wearable glucose-monitoring microwave sensor which collects data which is used to measure a person's intra-body glucose levels; a second wearable glucose-monitoring microwave sensor which collects data which is used to measure a person's intra-body glucose levels, wherein the first and second wearable glucose-monitoring microwave sensors are both located around the circumference of a finger ring, wrist band, bracelet, shirt cuff, or arm band; and a wearable or implanted pump which delivers a glucose-level-modifying substance into the person's body based on data collected by the plurality of wearable glucose-monitoring microwave sensors.

In an example, a glucose monitoring and managing system can comprise: an array, matrix, or series of wearable glucose-monitoring microwave sensors which collect data which is used to measure a person's intra-body glucose levels, wherein this array, matrix, or series of wearable glucose-monitoring microwave sensors is distributed around (a portion of) the circumference of a finger ring, wrist band, bracelet, shirt cuff, or arm band; and a wearable or implanted pump which delivers a glucose-level-modifying substance into the person's body based on data collected by the array, matrix, or series of wearable glucose-monitoring microwave sensors.

In an example, a wearable glucose-monitoring microwave sensor can comprise: a wearable microwave energy emitter; an array, matrix, or series of wearable microwave energy receivers which collect data which is used to measure a person's intra-body glucose levels; and a wearable or implanted pump which delivers a glucose-level-modifying substance into the person's body based on data collected by the plurality of wearable glucose-monitoring microwave sensors. In an example, a wearable glucose-monitoring microwave sensor can comprise: a wearable microwave energy emitter; an array, matrix, or series of wearable microwave energy receivers which collect data which is used to measure a person's intra-body glucose levels, wherein this array, matrix, or series of wearable microwave energy receivers is distributed around (a portion of) the circumference of a finger ring, wrist band, bracelet, shirt cuff, or arm band; and a wearable or implanted pump which delivers a glucose-level-modifying substance into the person's body based on data collected by the plurality of wearable glucose-monitoring microwave sensors.

In an example, a wearable glucose-monitoring microwave sensor can comprise: an array, matrix, or series of wearable microwave energy emitters, wherein this array, matrix, or series of wearable microwave energy emitters is distributed around (a portion of) the circumference of a finger ring, wrist band, bracelet, shirt cuff, or arm band; a wearable microwave energy receiver which collects data which is used to measure a person's intra-body glucose levels; and a wearable or implanted pump which delivers a glucose-level-modifying substance into the person's body based on data collected by the plurality of wearable glucose-monitoring microwave sensors.

In an example, a microwave energy emitter and a microwave energy receiver which comprise a wearable glucose-monitoring microwave sensor can be symmetric with respect to a line perpendicular to the shortest-line distance between them. In an example, a microwave energy emitter and a microwave energy receiver can be symmetric with respect to a line bisecting the gap between them. In an example, the ends of a microwave energy emitter and a microwave energy receiver which face each other can have the same shape. In an example, the ends of a microwave energy emitter and a microwave energy receiver which face each other can have complementary or interlocking shapes.

In an example, a plurality of wearable glucose-monitoring microwave sensors can be configured to confirm to the arcuate surface of a portion of a person's body. In an example, a plurality of wearable glucose-monitoring microwave sensors can be configured to encircle a person's finger, wrist, or arm. In an example, a plurality of wearable glucose-monitoring microwave sensors can be configured to span a portion of the circumference of a person's finger, wrist, or arm. In an example, an array, matrix, or series of wearable glucose-monitoring microwave sensors can be configured to span (a portion of) the circumference of a person's finger, wrist, or arm. In an example, a flexible array, matrix, or series of wearable glucose-monitoring microwave sensors can be configured to conform to (a portion of) the circumference of a person's finger, wrist, or arm.

In an example, a wearable glucose-monitoring microwave sensor can comprise a flexible array of microwave sensors which bends and/or stretches to conform to the arcuate surface of a portion of a person's body. In an example, a wearable glucose-monitoring microwave sensor can comprise a flexible array of microwave antennas which bends and/or stretches to conform to the arcuate surface of a portion of a person's body. In an example, a wearable glucose-monitoring microwave sensor can comprise a flexible array of wave guides which bends and/or stretches to conform to the arcuate surface of a portion of a person's body.

In an example, a wearable glucose-monitoring microwave sensor can further comprise one or more inflatable members whose inflation level can be adjusted to adjust the location of the sensor relative to the surface of a person's body.

In an example, a wearable glucose-monitoring microwave sensor can further comprise one or more inflatable members whose inflation level can be adjusted to adjust the pressure of the sensor on the surface of a person's body. In an example, a wearable glucose-monitoring microwave sensor can further comprise one or more pneumatic or hydraulic members which can be adjusted to adjust the location of the sensor relative to the surface of a person's body. In an example, a wearable glucose-monitoring microwave sensor can further comprise one or more pneumatic or hydraulic members which can be adjusted to adjust the pressure of the sensor on the surface of a person's body.

In an example, a plurality of wearable glucose-monitoring sensors can be three-dimensionally stacked. In an example, a plurality of wearable glucose-monitoring sensors can be stacked in parallel to each other. In an example, a plurality of wearable glucose-monitoring sensors can be stacked along a virtual vector which extends radially outward from the cross-sectional center of a body member. In an example, an array, matrix, or series of wearable glucose-monitoring sensors can be three-dimensionally stacked. In an example, an array, matrix, or series of wearable glucose-monitoring sensors can be stacked in parallel to each other. In an example, an array, matrix, or series of wearable glucose-monitoring sensors can be stacked along a virtual vector which extends radially outward from the cross-sectional center of a body member.

In an example, a plurality of wearable glucose-monitoring sensors can be distributed within a three-dimensional array or matrix along two (polar) coordinate dimensions: radial compass degree or clock-hour position around the circumference of a body part; and distance from the cross-sectional center of the body part. In an example, a plurality of wearable glucose-monitoring sensors on a finger ring can be distributed within a three-dimensional array or matrix along two (polar) coordinate dimensions: radial compass degree or clock-hour position around the circumference of the finger; and distance from the cross-sectional center of the finger. In an example, a plurality of wearable glucose-monitoring sensors on a wrist band can be distributed within a three-dimensional array or matrix along two (polar) coordinate dimensions: radial compass degree or clock-hour position around the circumference of the wrist; and distance from the cross-sectional center of the wrist.

In an example, a stacked array, matrix, or series of wearable glucose-monitoring microwave sensors can have a selected progression of different sizes, shapes, and/or orientations along a vector extending outward from the cross-sectional center of a body part such as a finger, wrist, or arm. In an example, sensors closer to the surface of the finger, wrist, or arm can have a first size, shape, and/or orientation and sensors further from the surface of the finger, wrist, or arm can have a second size, shape, and/or orientation. In an example, a stacked array, matrix, or series of wearable glucose-monitoring microwave sensors can have a selected progression of different sizes, shapes, and/or orientations around the circumference of a body part such as a finger, wrist, or arm. In an example, sensors closer to a ventral surface of the finger, wrist, or arm can have a first size, shape, and/or orientation and sensors further from the ventral surface of the finger, wrist, or arm can have a second size, shape, and/or orientation.

In an example, one or more aspects of the configuration and/or operation of a wearable glucose-monitoring microwave sensor can be automatically adjusted to improve measurement of intra-body glucose levels. In an example, a wearable glucose-monitoring microwave sensor can further comprise one or more actuators which automatically adjust its location, orientation, position, and/or configuration in order to more accurately and consistently measure intra-body glucose levels. In an example, such automatic adjustment occurs when a person first wears the wearable glucose-monitoring microwave sensor as the sensor is calibrated to the anatomy and/or physiology of that specific person. In an example, such automatic adjustment occurs each time the person wears the wearable glucose-monitoring microwave sensor as the sensor is calibrated to the specific placement of the device at different occasions. In an example, such automatic adjustment occurs in real time whenever the person is wearing the device to continually adjust for real-time body motion, sensor shifting, and other variables. In an example, one or more actuators which automatically adjust the configuration and/or operation of a wearable glucose-monitoring microwave sensor can be electromagnetic actuators.

In an example, one or more actuators which automatically adjust the configuration and/or operation of a wearable glucose-monitoring microwave sensor can be MEMS actuators. In an example, one or more actuators which automatically adjust the configuration and/or operation of a wearable glucose-monitoring microwave sensor can be pneumatic actuators. In an example, one or more actuators which automatically adjust the configuration and/or operation of a wearable glucose-monitoring microwave sensor can be hydraulic actuators.

In an example, an actuator can automatically adjust the distance between a wearable glucose-monitoring microwave sensor and the surface of a person's body in order to more accurately and/or consistently measure intra-body glucose levels. In an example, an actuator can automatically adjust the distance between a wearable glucose-monitoring microwave sensor and the surface of a person's body in order to maintain a relatively constant distance for more accurate and/or consistent measurement of intra-body glucose levels. In an example, an actuator can automatically adjust the distance between a wearable glucose-monitoring microwave sensor and the surface of a person's body when this distance would otherwise be changed by body motion and sensor shifting.

In an example, an actuator can automatically adjust the orientation of a wearable glucose-monitoring microwave sensor relative to the surface of a person's body in order to more accurately and/or consistently measure intra-body glucose levels. In an example, an actuator can automatically adjust the orientation of a wearable glucose-monitoring microwave sensor relative to the surface of a person's body in order to maintain a constant energy reflection angle for more accurate and/or consistent measurement of intra-body glucose levels. In an example, an actuator can automatically adjust the orientation of a wearable glucose-monitoring microwave sensor relative to the surface of a person's body when this orientation would otherwise be changed by body motion and sensor shifting.

In an example, an actuator can automatically adjust the position of a wearable glucose-monitoring microwave sensor relative to specific anatomical structures and/or landmarks on a person's body. In an example, an actuator can automatically adjust the position of a wearable glucose-monitoring microwave sensor to maintain proximity to specific anatomical structures and/or landmarks (such as specific portions of body vasculature) for more accurate and/or consistent measurement of intra-body glucose levels. In an example, an actuator can automatically adjust the location of a wearable glucose-monitoring microwave sensor relative to anatomical landmarks of a person's body when this location would otherwise be changed by body motion and sensor shifting. In an example, the location of a wearable glucose-monitoring microwave sensor can be automatically adjusted by one or more actuators in order to position it at the same location on a person's body when its location would otherwise be shifted by movement of the person's body.

In an example, a wearable glucose-monitoring microwave sensor can further comprise one or more actuators which automatically adjust the gap between a microwave energy emitter and a microwave energy receiver. In an example, the size of this gap can be adjusted when a person first wears the sensor in order to calibrate it to the specific anatomy and physiology of that person. In an example, the size of this gap can be adjusted each time a person wears the device in order to calibrate it for differences in placement or other variables from one wearing to the next. In an example, the size of this gap can be adjusted in real time in order to maintain accurate measurement of intra-body glucose levels. In an example, the gap between a microwave energy emitter and a microwave energy receiver can be automatically adjusted based on one or more parameters selected from the group consisting of: changes in the location on a person's body on which they are worn; changes their distance from the surface of the person's body; changes in the pressure and/or force which they apply to the person's body; changes in their angle and/or orientation relative to the person's body; changes in body temperature and/or ambient temperature; changes in body moisture level and/or ambient humidity; and body motion and/or speed.

In an example, a wearable glucose-monitoring microwave sensor can further comprise one or more actuators which automatically adjust the shape of the sensor. In an example, the shape of a sensor can be adjusted when a person first wears the sensor in order to calibrate it to the specific anatomy and physiology of that person. In an example, the shape of a sensor can be adjusted each time a person wears the device in order to calibrate it for differences in placement or other variables from one wearing to the next. In an example, the shape of a sensor can be adjusted in real time in order to maintain accurate measurement of intra-body glucose levels. In an example, the shape of a sensor can be automatically adjusted based on one or more parameters selected from the group consisting of: changes in the location on a person's body on which they are worn; changes their distance from the surface of the person's body; changes in the pressure and/or force which they apply to the person's body; changes in their angle and/or orientation relative to the person's body; changes in body temperature and/or ambient temperature; changes in body moisture level and/or ambient humidity; and body motion and/or speed.

In an example, a wearable glucose-monitoring microwave sensor can have a first level of operation and a second level of operation, wherein the second level of operation provides more accurate measurement of intra-body glucose levels than the first level of operation, and wherein the sensor can be reversibly changed from the first level of operation to the second level of operation. In an example, the second level of operation requires more power than the first level of operation. In an example, the second level of operation activates a larger number of sensors in a sensor array than the first level of operation. In an example, the second level of operation involves more frequent scans than the first level of operation.

In an example, operation of the wearable glucose-monitoring microwave sensor at the second level of operation can require more power and be more accurate than operation of the wearable glucose-monitoring microwave sensor at the first level of operation. In an example, automatic and reversible adjustment of the operation of a wearable glucose-monitoring microwave sensor from a first level of operation to a second level of operation, and vice versa, can help to conserve energy because the microwave sensor only operates at higher power levels when needed. In an example, automatic and reversible adjustment of the operation of a wearable glucose-monitoring microwave sensor from a first level of operation to a second level of operation, and vice versa, can help to reduce a person's exposure to microwave energy because the microwave sensor only operates at higher power levels when needed.

In an example, a wearable glucose-monitoring microwave sensor can be changed from a first level of operation to a second level of operation based on changes in physiological or environmental factors which are detected by other wearable or implanted sensors. In an example, a wearable glucose-monitoring microwave sensor can be changed from a first level of operation to a second level of operation when analysis of data collected by other wearable or implanted sensors indicates that a person is eating or likely to eat soon. In an example, a wearable glucose-monitoring microwave sensor can be changed from a first level of operation to a second level of operation when data indicates a glucose level below or above a normal range. In an example, a wearable glucose-monitoring microwave sensor can be changed from a first level of operation to a second level of operation when analysis of data collected by other wearable or implanted sensors indicates that a person very physically active. In an example, a wearable glucose-monitoring microwave sensor can operate at a first level of operation until an abnormally low or high intra-body glucose level is detected, at which time it can shift to operation at a second level of operation.

In an example, a wearable glucose-monitoring microwave sensor can be changed from a first level of operation to a second level of operation based on one or more factors selected from the group consisting of: detection that a person is eating; detection of an abnormal intra-body glucose level; a change in the position and/or orientation of a microwave sensor relative to a person's body; a change in the degree of contact and/or pressure between a microwave sensor and a person's body; a change in a person's body moisture level and/or environmental moisture level; a change in a person's body temperature and/or environmental temperature; a change in ambient electromagnetic energy level; a change in a person's body movement speed, acceleration, orientation, or direction; and a change in geographic location (e.g. detected by GPS) and/or proximity to a place that is associated with food consumption.

In an example, a wearable glucose-monitoring microwave sensor can emit electromagnetic energy in the range of 100 MHz to 100 GHz. In an example, a wearable glucose-monitoring microwave sensor can emit electromagnetic energy in the range of 100 MHz to 5 GHz. In an example, a wearable glucose-monitoring microwave sensor can emit electromagnetic energy in the range of 300 MHz to 300 GHz. In an example, a wearable glucose-monitoring microwave sensor can emit microwave energy at a constant frequency. In an example, a wearable glucose-monitoring microwave sensor can emit microwave energy at varying frequencies within a selected range in order to collect information about the interaction between that energy and body tissue across a spectrum of frequencies. In an example, a wearable glucose-monitoring microwave sensor can emit microwave energy at frequencies which sweep through a selected range.

In an example, a wearable glucose-monitoring microwave sensor can be a microwave spectroscopy sensor. In an example, a microwave spectroscopy sensor can measure the permittivity of body tissue and/or fluid as a function of microwave energy at varying frequencies. In an example, a microwave spectroscopy sensor can measure multiple resonant frequencies of one or more resonators by sweeping through a range of microwave frequencies. In an example, a wearable glucose-monitoring microwave sensor can be a spectroscopy sensor which collects information about electromagnetic interaction between microwave energy and body tissue and/or fluids at multiple microwave frequencies. In an example, information about electromagnetic interaction throughout a microwave frequency spectrum can provide more accurate information concerning intra-body glucose levels than information about electromagnetic interaction at a single microwave frequency. In an example, a wearable glucose-monitoring microwave sensor can be a microwave spectrometer. In an example, a wearable glucose-monitoring microwave sensor can be an impedance spectroscopy sensor.

In an example, a wearable glucose-monitoring microwave sensor can include a microwave emitter which sweeps through a range of microwave frequencies. In an example, a wearable glucose-monitoring microwave sensor can include a microwave-emitting component which emits pulses of ultra-wideband microwave energy. In an example, a wearable glucose-monitoring microwave sensor can expose body tissue and/or fluid to microwave energy at different frequencies and collect information on the response of that body tissue and/of fluid at each frequency. In an example, a wearable glucose-monitoring microwave sensor can measure intra-body glucose levels via impedance or dielectric spectroscopy.

In an example, a glucose monitoring and managing system can further comprise an electromagnetic resonator. In an example, a wearable glucose-monitoring microwave sensor can further comprise an electromagnetic resonator. One or more resonant frequencies of such a resonator can change with changes in the glucose level of nearby body tissue and/or body fluid. One or more resonant frequencies of such a resonator can be changed by a change in the permittivity of nearby body tissue and/or fluid which, in turn, can be changed by changes in the glucose level of nearby body tissue and/or body fluid. The resonant frequency of a resonator is reduced by dielectric loading of body tissue. In an example, there can be an inverse relationship between the glucose levels of body tissue and/or fluid and the resonant frequency of a resonator. In this manner, intra-body glucose levels can be estimated by measuring one or more resonant frequencies of an electromagnetic resonator.

In an example, an electromagnetic resonator can be located between a microwave energy emitter and a microwave energy receiver. In an example, when the resonator resonates at a resonant frequency, the resonator acts as a partial short-circuit between the microwave energy emitter and the microwave energy receiver. In an example, a resonator creates a drop in impedance in the transmission of microwave energy from the transmitter to the receiver when the resonator resonates. In this manner, one or more resonant frequencies of the resonator can be observed by observing changes in the transmission of microwave energy from the microwave energy emitter to the microwave energy receiver. In an example, multiple resonant frequencies can be observed with a selected range (or spectrum) of microwave energy frequencies.

In an example, a wearable glucose-monitoring microwave sensor can comprise a microwave energy emitter, a microwave energy receiver, and an electromagnetic resonator, wherein one or more resonant frequencies of the resonator are shifted by changes in the glucose levels of nearby body tissue and/or fluid. In an example, changes in the glucose levels of nearby body tissue and/or fluid change the permittivity of the body tissue and/or fluid which, in turn, changes the one or more resonant frequencies of the resonator. In an example, a wearable glucose-monitoring microwave sensor can comprise a microwave energy emitter, a microwave energy receiver, and a resonator, wherein changes in microwave transmission are used to measure changes in the glucose levels of nearby body tissue and/or fluid.

In an example, a wearable glucose-monitoring microwave sensor can comprise: a first component which transmits microwave energy; a second component which receives microwave energy; and a third component between the first and second components which resonates in response to microwave energy transmission. In an example, the resonant frequency of the third component changes based on changes in the glucose levels of nearby body tissue and/or fluid. In an example, the third component can be a microwave resonator. In an example, the resonant frequency of the third component changes based on changes in the permittivity of nearby body tissue and/or fluid which, in turn, depends on the glucose levels in that body tissue and/or fluid.

An electromagnetic resonator does not have to be in direct contact with a person's body in order to have its resonant frequency affected by the electromagnetic properties of body tissue and/or fluid. An electromagnetic field can spill out into nearby body tissue and/or fluid from the side of a resonator which faces the body tissue and/or fluid. This electromagnetic field can penetrate nearby body tissue and/or fluid and measure glucose levels at a greater tissue depth than is possible with measurement methods which rely on reflection of light energy. In an example, changes in the glucose levels of nearby body tissue and/or fluid change the permittivity of that body tissue and/or fluid which change the resonating frequency of the electromagnetic resonator. In this manner, observed changes in one or more resonant frequencies of the resonator can be used to estimate intra-body glucose levels.

In an example, a resonator which is part of a wearable glucose-monitoring microwave sensor can be arcuate. In an example, a resonator which is part of a wearable glucose-monitoring microwave sensor can be circular. In an example, a resonator which is part of a wearable glucose-monitoring microwave sensor can be a ring. In an example, a resonator which is part of a wearable glucose-monitoring microwave sensor can have a shape which is selected from the group consisting of: circular ring or band; concentric ring; target pattern; nested and/or concentric rings; ring with a single split or gap; ring with multiple splits (or gaps); and three-dimensionally stacked or parallel rings. In an example, a resonator which is part of a wearable glucose-monitoring microwave sensor can have a shape which is selected from the group consisting of: cylinder; cylindrical band; ellipse; elliptical ring; and oval.

In an example, a resonator which is part of a wearable glucose-monitoring microwave sensor can be polygonal. In an example, a resonator which is part of a wearable glucose-monitoring microwave sensor can be square or rectangular. In an example, a resonator which is part of a wearable glucose-monitoring microwave sensor can have a shape which is selected from the group consisting of: polygon with rounded vertices; square, rectangle, or other quadrilateral; square or rectangular cylinder; triangle; hexagon or octagon; non-equilateral polygon; other polygon or polygon with rounded vertexes; cube; and stacked or parallel squares.

In an example, a resonator which is part of a wearable glucose-monitoring microwave sensor can be a spiral. In an example, a resonator which is part of a wearable glucose-monitoring microwave sensor can be selected from the group consisting of: circular spiral; oval spiral; square spiral; rectangular spiral; other polygonal spiral; two intertwined spirals; two symmetric spirals; and two stacked spirals. In various examples, a resonator can be selected from the group consisting of: annular Bragg resonator, bow tie shape, co-linear elements, comb shape, conic section shape, convex lens shape, C-shape, dumbbell shape, figure eight shape, hemisphere, H-shape, microstrip, Omega-shape, saddle shape, sawtooth shape, semicircle, sinusoidal shape, sphere, square wave shape, S-shape, straight line, T-shape, U-shape, Y-shape, and zigzag shape.

In an example, a resonator which is part of a wearable glucose-monitoring microwave sensor can have one or more splits (or gaps). In an example, a resonator which is part of a wearable glucose-monitoring microwave sensor can have one or more splits (or gaps) in its perimeter. The capacitance of a split or gap in the perimeter of a resonator can cause a resonator to resonate at a wave length which is larger than the size of the resonator. In an example, a resonator can have a gap in its perimeter which produces large capacitance values and lowers the resonant frequency of the resonator. In an example, a resonator can be a ring with one or more spits or gaps in its circumference. In an example, a resonator can be a Split Ring Resonator (SRR). In an example, a resonator can comprise two or more nested and/or concentric rings with spits or gaps in their circumferences.

In an example, a wearable glucose-monitoring microwave sensor can comprise: a microwave energy emitter; a microwave energy receiver; and a Split Ring Resonator (SRR) between the microwave energy emitter and the microwave energy receiver. In an example, changes in the glucose levels of nearby body tissue and/or fluid change the permittivity of that body tissue and/or fluid, which changes the resonant frequency of the Split Ring Resonator (SRR), which enables measurement of intra-body glucose levels.

In an example, compass or clock-hour coordinates for a resonator can be defined with respect to a “connector line” between a microwave energy emitter and a microwave energy receiver. In an example, the “connector line” can be defined as a virtual straight line which is drawn between the closest points on the microwave energy emitter and the microwave energy receiver. In an alternative example, the “connector line” can be defined as a virtual straight line which is drawn between the centroids of a microwave energy emitter and microwave energy receiver. In an example, virtual compass or clock-hour coordinates can be superimposed on a resonator, centered around the centroid of the resonator, so that the connector line intersects the 270-degree (9 o'clock) and the 90-degree (3 o'clock) locations on the compass or clock-hour coordinate system. In an example, virtual compass or clock-hour coordinates can be centered around the mid-point of the “connector line.”

In an example, a single split or gap of a resonator can be centered at the 360-degree (12 o'clock) or 180-degree (6 o'clock) location using the above-defined compass or clock-hour coordinates. In an example, there can be two splits (or gaps) in a resonator, one centered at the 360-degree (12 o'clock) location and one at the 180-degree (6 o'clock) location. More generally, a resonator can have a number (X) of perimeter splits of gaps whose positions are separated by (360/X) degrees.

In an example, with nested and/or concentric resonators, the location of the split or gap in an outer element (e.g. ring or square) can be rotated 180 degrees relative to the location of the split or gap in the inner element (e.g. ring or square). More generally, a resonator can have a number (Y) of nested and/or concentric rings, wherein the locations of splits (or gaps) in pairs of adjacent rings (e.g. proximal rings separated by an inter-ring gap) vary from each other by 180-degree rotation. More generally, a resonator can have (Y) nested and/or concentric rings, each with (X) splits (or gaps), wherein the locations of splits (or gaps) in pairs of adjacent rings (e.g. proximal rings separated by an inter-ring gap) vary from each other by (360/2X) degrees.

In an example, a wearable glucose-monitoring microwave sensor can comprise a first resonator with a first resonant frequency and a second resonator with a second resonant frequency. In an example, a first resonator can differ from a second resonator in one or more aspects selected from the group consisting of: resonator shape; resonator size; resonator orientation with respect to the connector line; resonator material; resonator distance from the surface of a person's body; resonator symmetry; number of splits (or gaps); size of splits (or gaps); location and/or orientation of splits (or gaps); nested configuration; and stacked configuration. In an example, a wearable glucose-monitoring microwave sensor can comprise a plurality of resonators with different resonant frequencies. In an example, a plurality of resonators with different resonant frequencies can provide more rapid and/or complete information on the permittivity of nearby body tissue and/or fluid (and thus intra-body glucose levels) across a selected range of microwave frequencies than is possible with a single resonator.

In an example, a wearable glucose-monitoring microwave sensor can comprise a first resonator with a first shape and a second resonator with a second shape, wherein the second shape is different than the first shape. In an example, a wearable glucose-monitoring microwave sensor can comprise a first resonator with a first shape and a second resonator with a second shape, wherein the second shape is more arcuate than the first shape. In an example, a wearable glucose-monitoring microwave sensor can comprise a first resonator with a first shape and a second resonator with a second shape, wherein the second shape is more convex than the first shape. In an example, a wearable glucose-monitoring microwave sensor can comprise a first resonator with a first size and a second resonator with a second size, wherein the second size is at least 10% larger than the first size.

In an example, a wearable glucose-monitoring microwave sensor can comprise a first resonator with a first orientation and a second resonator with a second orientation, wherein the second orientation is different than the first orientation. In an example, a wearable glucose-monitoring microwave sensor can comprise a first resonator with a first orientation and a second resonator with a second orientation, wherein the second orientation is rotated by at least 10 degrees relative to the first orientation. In an example, a wearable glucose-monitoring microwave sensor can comprise a first resonator with a split (or gap) with a first position relative to compass coordinates and a second resonator with a split (or gap) with a first position relative to compass coordinates, wherein the second position is rotated by at least 10 degrees relative to the first position.

In an example, a wearable glucose-monitoring microwave sensor can comprise a first resonator with a first number of splits (or gaps) and a second resonator with a second number of splits (or gaps), wherein the second number is larger than the first number. In an example, a wearable glucose-monitoring microwave sensor can comprise a first resonator made from a first material and a second resonator made from a second material, wherein the second material is different than the first material. In an example, a wearable glucose-monitoring microwave sensor can comprise a first resonator made from a first material and a second resonator made from a second material, wherein the second material is more conductive from the first material. In an example, a wearable glucose-monitoring microwave sensor can comprise a first resonator located at a first distance from the surface of a person's body and a second resonator located at a second distance from the surface of a person's body, wherein the second distance is greater than the first distance.

In an example, a wearable glucose-monitoring microwave sensor can comprise a first resonator and a second resonator, wherein the first and second resonators are co-planar. In an example, a wearable glucose-monitoring microwave sensor can comprise a first resonator and a second resonator, wherein the first and second resonators both lie in the same flat plane. In an example, a first resonator and a second resonator can be co-planar and adjacent. In an example, a first resonator and a second resonator can be co-planar and symmetric. In an example, a first resonator and a second resonator can be co-planar and repeated. In an example, a first resonator and a second resonator can be co-planar and nested and/or concentric. In an example, a wearable glucose-monitoring microwave sensor can comprise an array, matrix, or series of co-planar resonators. In an example, a wearable glucose-monitoring microwave sensor can comprise an array, matrix, or series of co-planar resonators with different resonant frequencies.

In an example, a wearable glucose-monitoring microwave sensor can comprise a first Split Ring Resonator (SRR) and a second Split Ring Resonator (SRR), wherein the first and second Split Ring Resonators (SRRs) are co-planar. In an example, a wearable glucose-monitoring microwave sensor can comprise a first Split Ring Resonator (SRR) and a second Split Ring Resonator (SRR), wherein the first and second Split Ring Resonators (SRRs) both lie in the same flat plane. In an example, a first Split Ring Resonator (SRR) and a second Split Ring Resonator (SRR) can be co-planar and adjacent. In an example, a first Split Ring Resonator (SRR) and a second Split Ring Resonator (SRR) can be co-planar and symmetric. In an example, a first Split Ring Resonator (SRR) and a second Split Ring Resonator (SRR) can be co-planar and repeated. In an example, a first Split Ring Resonator (SRR) and a second Split Ring Resonator (SRR) can be co-planar and nested and/or concentric. In an example, a wearable glucose-monitoring microwave sensor can comprise an array, matrix, or series of co-planar Split Ring Resonator (SRR)s. In an example, a wearable glucose-monitoring microwave sensor can comprise an array, matrix, or series of co-planar Split Ring Resonators (SRRs) with different resonant frequencies.

In an example, a wearable glucose-monitoring microwave sensor can comprise a uniform array, matrix, or series of identical co-planar resonators. In an example, a wearable glucose-monitoring microwave sensor can comprise a non-uniform array, matrix, or series of co-planar resonators. In an example, a wearable glucose-monitoring microwave sensor can comprise an array, matrix, or series of co-planar resonators, wherein there is a selected progression in resonator frequency along an axis of the array, matrix, or series. In an example, a wearable glucose-monitoring microwave sensor can comprise an array, matrix, or series of co-planar resonators, wherein there is a selected progression in resonator frequency from a lower resonant frequency to a higher resonant frequency along an axis of the array, matrix, or series. In an example, a wearable glucose-monitoring microwave sensor can comprise an array, matrix, or series of co-planar resonators, wherein there is a selected progression in resonator size along an axis of the array, matrix, or series. In an example, a wearable glucose-monitoring microwave sensor can comprise an array, matrix, or series of co-planar resonators, wherein there is a selected progression in resonator size from a smaller to larger along an axis of the array, matrix, or series.

In an example, a wearable glucose-monitoring microwave sensor can comprise an arcuate array, matrix, or series of resonators. In an example, a wearable glucose-monitoring microwave sensor can comprise an array, matrix, or series of resonators which lie along the surface of a (virtual) ring or cylinder. In an example, a wearable glucose-monitoring microwave sensor can comprise an array, matrix, or series of resonators which lie along the surface of a finger ring, wrist band, or arm band. In an example, a wearable glucose-monitoring microwave sensor can comprise an array, matrix, or series of resonators which are distributed around (a portion of) the circumference of a finger ring, wrist band, or arm band. In an example, a wearable glucose-monitoring microwave sensor can comprise a cylindrical array, matrix, or series of resonators. In an example, a wearable glucose-monitoring microwave sensor can comprise an oval-shaped array, matrix, or series of resonators. In an example, a wearable glucose-monitoring microwave sensor can comprise a saddle-shaped array, matrix, or series of resonators.

In an example, a wearable glucose-monitoring microwave sensor can comprise a flexible, stretchable, and/or elastic arcuate array, matrix, or series of resonators which can bend to conform to the curvature of the surface of a person's body. In an example, a wearable glucose-monitoring microwave sensor can comprise a flexible, stretchable, and/or elastic array, matrix, or series of resonators which can conform to a relatively-uniform distance from the surface of a person's body. In an example, a wearable glucose-monitoring microwave sensor can comprise a flexible, stretchable, and/or elastic array, matrix, or series of resonators which can be incorporated into a wrist band or arm band. In an example, a wearable glucose-monitoring microwave sensor can comprise a flexible arcuate array, matrix, or series of resonators which can be incorporated into a shirt sleeve or cuff. In an example, a wearable glucose-monitoring microwave sensor can comprise a flexible arcuate array, matrix, or series of resonators which can be incorporated into the cuff of a pair of shorts or pants. In an example, a wearable glucose-monitoring microwave sensor can comprise a flexible arcuate array, matrix, or series of resonators which can be incorporated into a sock.

In an example, a wearable glucose-monitoring microwave sensor can comprise a plurality of stacked resonators. In an example, a wearable glucose-monitoring microwave sensor can comprise a plurality of three-dimensionally-stacked resonators. In an example, a wearable glucose-monitoring microwave sensor can comprise a plurality of parallel stacked resonators. In an example, a wearable glucose-monitoring microwave sensor can comprise a plurality of resonators which are stacked along a radial vector which extends outward from the cross-sectional center of a person's finger, wrist, arm, or leg. In an example, a wearable glucose-monitoring microwave sensor can comprise a plurality of resonators which are stacked along a vector which is perpendicular to the surface of a person's body. In an example, a wearable glucose-monitoring microwave sensor for estimating intra-body glucose levels can include stacked or parallel resonators. In an example, there can be two stacked or parallel resonators between a microwave energy emitter and a microwave energy receiver. In an example, there can be three of more stacked or parallel resonators between a microwave energy emitter and a microwave energy receiver.

In an example, a wearable glucose-monitoring microwave sensor can comprise a three-dimensional array, matrix, or series of resonators. In an example, a wearable glucose-monitoring microwave sensor can comprise a three-dimensional array, matrix, or series of resonators wherein at least one dimension of the array, matrix, or series is aligned with a vector which extends radially outward from the cross-sectional center of a person's finger, wrist, arm, or leg. In an example, a wearable glucose-monitoring microwave sensor can comprise a three-dimensional array, matrix, or series of resonators wherein at least one dimension of the array, matrix, or series is aligned with a vector which is perpendicular to the surface of a person's body. In an example, a wearable glucose-monitoring microwave sensor can comprise: a microwave energy emitter; a microwave energy receiver; and a three-dimensional array, matrix, or series of resonators between the microwave energy emitter and the microwave energy receiver.

In an example, a wearable glucose-monitoring microwave sensor can comprise a first resonator whose centroid is a first distance from the surface of a person's body and a second resonator whose centroid is a second distance from the surface of a person's body, wherein the second distance is greater than the first distance. In an example, a wearable glucose-monitoring microwave sensor can comprise a first resonator whose closest point to the surface of a person's body is a first distance and a second resonator whose closest point to the surface of a person's body is a second distance, wherein the second distance is greater than the first distance.

In an example, a wearable glucose-monitoring microwave sensor can comprise a plurality of stacked Split Ring Resonators (SRRs). In an example, a wearable glucose-monitoring microwave sensor can comprise a plurality of three-dimensionally-stacked Split Ring Resonators (SRRs). In an example, a wearable glucose-monitoring microwave sensor can comprise a plurality of parallel stacked Split Ring Resonators (SRRs). In an example, a wearable glucose-monitoring microwave sensor can comprise a plurality of Split Ring Resonators (SRRs) which are stacked along a radial vector which extends outward from the cross-sectional center of a person's finger, wrist, arm, or leg. In an example, a wearable glucose-monitoring microwave sensor can comprise a plurality of Split Ring Resonators (SRRs) which are stacked along a vector which is perpendicular to the surface of a person's body. In an example, a wearable glucose-monitoring microwave sensor for estimating intra-body glucose levels can include stacked or parallel Split Ring Resonators (SRRs). In an example, there can be two stacked or parallel Split Ring Resonators (SRRs) between a microwave energy emitter and a microwave energy receiver. In an example, there can be three of more stacked or parallel Split Ring Resonators (SRRs) between a microwave energy emitter and a microwave energy receiver.

In an example, a wearable glucose-monitoring microwave sensor can comprise a three-dimensional array, matrix, or series of Split Ring Resonators (SRRs). In an example, a wearable glucose-monitoring microwave sensor can comprise a three-dimensional array, matrix, or series of Split Ring Resonators (SRRs) wherein at least one dimension of the array, matrix, or series is aligned with a vector which extends radially outward from the cross-sectional center of a person's finger, wrist, arm, or leg. In an example, a wearable glucose-monitoring microwave sensor can comprise a three-dimensional array, matrix, or series of Split Ring Resonators (SRRs) wherein at least one dimension of the array, matrix, or series is aligned with a vector which is perpendicular to the surface of a person's body. In an example, a wearable glucose-monitoring microwave sensor can comprise: a microwave energy emitter; a microwave energy receiver; and a three-dimensional array, matrix, or series of Split Ring Resonators (SRRs) between the microwave energy emitter and the microwave energy receiver.

In an example, a wearable glucose-monitoring microwave sensor can comprise a first Split Ring Resonator (SRR) whose centroid is a first distance from the surface of a person's body and a second Split Ring Resonator (SRR) whose centroid is a second distance from the surface of a person's body, wherein the second distance is greater than the first distance. In an example, a wearable glucose-monitoring microwave sensor can comprise a first Split Ring Resonator (SRR) whose closest point to the surface of a person's body is a first distance and a second Split Ring Resonator (SRR) whose closest point to the surface of a person's body is a second distance, wherein the second distance is greater than the first distance.

In an example, a wearable glucose-monitoring microwave sensor can comprise a uniform three-dimensional array, matrix, or series of resonators. In an example, a wearable glucose-monitoring microwave sensor can comprise a non-uniform three-dimensional array, matrix, or series of resonators. In an example, resonators in a three-dimensional array, matrix, or series of resonators can vary in size, shape, resonant frequency, or other features. In an example, resonators in a three-dimensional array, matrix, or series which are closer to the surface of a person's body can be smaller and resonators which are farther from the surface of the person's body can be larger, or vice versa. In an example, resonators in a three-dimensional array, matrix, or series which are closer to the surface of a person's body can have a lower resonant frequency and resonators which are farther from the surface of the person's body can have a higher resonant frequency, or vice versa.

In an example, a wearable glucose-monitoring microwave sensor can comprise a first resonator with a first size which is configured to be worn a first distance from the surface of a person's body and a second resonator with a second size which is configured to be worn a second distance from the surface of a person's body, wherein the second size is greater than the first size and wherein the second distance is greater than the first distance. In an example, a wearable glucose-monitoring microwave sensor can comprise a first resonator with a first size which is configured to be worn a first distance from the surface of a person's body and a second resonator with a second size which is configured to be worn a second distance from the surface of a person's body, wherein the second size is at least 10% greater than the first size and wherein the second distance is at least 10% greater than the first distance.

In an example, one or more parameters of a resonator in a wearable glucose-monitoring microwave sensor can be automatically adjusted for greater accuracy in measuring intra-body glucose levels. In an example, a wearable glucose-monitoring microwave sensor can include one or more actuators which automatically adjust parameters of a resonator for greater accuracy in measuring intra-body glucose levels. In an example, automatic adjustment of a resonator can occur when a particular person first wears a device in order to customize the device to the person's specific anatomy and/or physiology. In an example, automatic adjustment of a resonator can occur each time the same person wears a device in order to optimize the device for specific way in which the device is worn (each time that it is worn). In an example, automatic adjustment of a resonator can occur in real time as a person is wearing a device based on changing physiological and environmental factors.

In an example, the position of one or more resonators can be automatically adjusted by one or more actuators in order to maintain a uniform distance from the surface of a person's body. In an example, the position of one or more resonators can be automatically adjusted by one or more actuators in order to maintain a consistent location on the surface of a person's body. In an example, the one or more parameters of a resonator in a wearable glucose-monitoring microwave sensor that are automatically adjusted can be selected from the group consisting of: split (or gap) distance; inter-resonator distance in an array, matrix, or series of resonators; distance between a resonator and the surface of a person's body; angle between a resonator and the surface of a person's body; orientation of a resonator relative to the surface of a person's body; location of a resonator on a person's body (relative to anatomical landmarks such as vasculature); frequency of microwave energy emitted; distance between a resonator and a microwave energy emitter or receiver; orientation of a resonator relative to a microwave energy emitter or receiver; and curvature of an array, matrix, or series of resonators.

In an example, the location, size, or shape of one or more resonators in a wearable glucose-monitoring microwave sensor can be automatically adjusted by one or more actuators in response to changes in temperature. In an example, the location, size, or shape of one or more resonators in a wearable glucose-monitoring microwave sensor can be automatically adjusted by one or more actuators in response to changes in moisture. In an example, the one or more parameters of a resonator in a wearable glucose-monitoring microwave sensor can be automatically adjusted based on one or more physiological and environment factors selected from the group consisting of: body motion or configuration; body moisture level and/or ambient humidity; body temperature and/or ambient temperature; food consumption; exercise; geographic location; and ambient electromagnetic activity.

In an example, a wearable glucose-monitoring microwave sensor can be incorporated into a wrist band, smart watch, or bracelet. In an example, a wearable glucose-monitoring microwave sensor can be incorporated into a finger ring. In an example, a wearable glucose-monitoring microwave sensor can be incorporated into an arm band. In an example, a wearable glucose-monitoring microwave sensor can be incorporated into an ear bud, ear plug, hearing aid, earphone, headphone, or earring. In an example, a wearable glucose-monitoring microwave sensor can be incorporated into the cuff and/or sleeve of a shirt. In an example, a wearable glucose-monitoring microwave sensor can be incorporated into the cuff and/or leg of a pair of pants or shorts. In an example, a wearable glucose-monitoring microwave sensor can be incorporated into a wearable patch or tattoo.

In an example, a wearable glucose-monitoring microwave sensor can be incorporated into the fabric of an article of clothing. In an example, electroconductive threads or yarns can function as microwave energy emitters and/or receivers. In an example, electroconductive thread or yarns in an article of clothing can expose body tissue and/or fluid to an electromagnetic field and the interaction of this body tissue and/or fluid with the electromagnetic fluid can be used to measure intra-body glucose levels. In an example, a wearable microwave sensor can be integrated into the fabric of a shirt, pair of pants or shorts, undershirt, underpants, sock, or belt. In an example, an electromagnetically-functional shirt, pair of pants or short, undershirt, underpants, sock, or belt can function as a wearable glucose-monitoring microwave sensor. In an example, an electromagnetically-functional shirt, pair of pants or short, undershirt, underpants, sock, or belt can function as the glucose-monitoring component of a closed-loop system for monitoring and managing intra-body glucose levels.

In an example, a first electroconductive thread or yarn in a woven fabric can function as a microwave energy emitter and a second electroconductive thread of yarn in a woven fabric can function as a microwave energy receiver. In an example, a series of resonators can be woven into fabric between the first electroconductive thread and the second electroconductive thread. In an example, a fabric woven from first and second electroconductive threads can create an electromagnetic field which can be used to measure the permittivity of nearby body tissue and/or fluid which, in turn, can be used to estimate intra-body glucose levels. In an example, a fabric comprised of electroconductive threads and electromagnetic resonators between electroconductive threads can create an electromagnetic field which can be used to measure the permittivity of nearby body tissue and/or fluid which, in turn, can be used to estimate intra-body glucose levels.

In an example, a wearable glucose-monitoring microwave sensor can be worn on a person's finger, hand, wrist, lower arm, or upper arm. In an example, a wearable glucose-monitoring microwave sensor can be worn on, in, or around a person's ear. In an example, a wearable glucose-monitoring microwave sensor can be worn on, in, or around a person's torso. In an example, a wearable glucose-monitoring microwave sensor can be incorporated into a piece of jewelry or clothing accessory. In an example, a wearable glucose-monitoring microwave sensor can be incorporated into a finger ring, watch, wrist band, bracelet, armband, necklace, or earring. In an example, a wearable glucose-monitoring microwave sensor can be incorporated into an article of clothing. In an example, a wearable glucose-monitoring microwave sensor can be incorporated into a glove, shirt, undershirt, pair of pants, shorts, undershorts, sock, shoe, belt, or eyeglasses.

In an example, a glucose monitoring and managing system can comprise: a wearable glucose-monitoring microwave sensor which collects data which is used to measure a person's intra-body glucose levels; and a wearable or implanted pump which delivers a glucose-level-modifying substance into the person's body based on data collected by the wearable glucose-monitoring microwave sensor. In an example, a wearable glucose-monitoring microwave sensor which monitors intra-body glucose levels and a wearable or implanted insulin pump which modifies intra-body glucose levels can together comprise a closed-loop system for automatically measuring and managing intra-body glucose levels. In an example, a wearable glucose-monitoring microwave sensor which monitors intra-body glucose levels and a wearable or implanted insulin pump which modifies intra-body glucose levels can together comprise a closed-loop system for automatically maintaining glucose levels within a selected range.

In an example, a pump which delivers a glucose-level-modifying substance into a person's body can be worn on the person's body. In an example, a pump which delivers a glucose-level-modifying substance into a person's body can be implanted within the person's body. In an example, a pump can dispense a substance which modifies the metabolism of intra-body glucose. In an example, a pump can dispense a substance which directly modifies the level of intra-body glucose. In an example, a pump can dispense insulin. In an example, a wearable glucose-monitoring microwave sensor and a wearable or implanted pump can be in wireless communication with each other. In an example, a wearable glucose-monitoring microwave sensor and a wearable or implanted insulin pump can be indirectly and wirelessly linked to each other by each having wireless communication with a common (albeit physically separate) data processing component.

In an example, In an example, a glucose monitoring and managing system can further comprise a microwave vector network analyzer which analyzes how the transmission and/or reflection of microwave energy is affected by interaction with nearby body tissue and/or fluid.

In an example, a glucose monitoring and managing system can further comprise one or more other sensors selected from the group consisting of: accelerometer, gyroscope, other inertial sensor, goniometer, bend sensor, stretch sensor, pressure sensor, GPS sensor, jaw motion sensor, and other motion sensor. In an example, one or more of these other sensors can detect when a person is eating food. In an example, detection of food consumption can trigger (a higher level of) operation of a wearable glucose-monitoring microwave sensor. In an example, a glucose monitoring and managing system can further comprise one or more of these other sensors selected from the group consisting of: microphone, swallow sensor, chewing sensor, ultrasound sensor, and other sound energy sensor. In an example, one or more of these other sensors can detect when a person is eating food. In an example, detection of food consumption can trigger (a higher level of) operation of a wearable glucose-monitoring microwave sensor.

In an example, a glucose monitoring and managing system can further comprise one or more of these other sensors selected from the group consisting of: camera, spectroscopic sensor, near-infrared sensor, infrared sensor, optoelectronic sensor, and other light energy sensor. In an example, one or more of these other sensors can detect when a person is eating food. In an example, detection of food consumption can trigger (a higher level of) operation of a wearable glucose-monitoring microwave sensor. In an example, a glucose monitoring and managing system can further comprise one or more of these other sensors selected from the group consisting of: EEG sensor, EMG sensor, other neuromuscular activity sensor, the electromagnetic brain activity sensor, skin impedance sensor, skin conductivity sensor, and other electromagnetic energy sensor. In an example, one or more of these other sensors can detect when a person is eating food. In an example, detection of food consumption can trigger (a higher level of) operation of a wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a motion sensor which collects data which is analyzed to detects changes in the configuration, position, and/or orientation of the microwave sensor relative to the person's body and/or changes in the person's body movement speed, acceleration, orientation, or direction; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a thermal energy sensor which collects data which is analyzed to detect changes in a person's body temperature and/or environmental temperature; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a moisture sensor which collects data which is analyzed to detect changes in a person's body moisture level and/or environmental moisture level; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

In an example, a glucose monitoring and managing system can comprise: (a) a wearable glucose-monitoring microwave sensor which collects data which is analyzed to measure a person's intra-body glucose level; (b) a geographic position sensor which collects data which is analyzed to detect changes in geographic location and/or proximity to a place that is associated with food consumption; and wherein the operation of the wearable glucose-monitoring microwave sensor is changed based on analysis of data from the motion sensor; and (c) a pump; wherein this pump is worn by, or implanted within, the person; wherein the pump dispenses a substance into the person's body which modifies the person's intra-body glucose level; and wherein dispensation of this substance is based on measurement of intra-body glucose level from analysis of data collected by the wearable glucose-monitoring microwave sensor.

A wearable device for non-invasive glucose monitoring can comprise; an electromagnetic energy emitter which emits electromagnetic energy into a person's finger tissue at a first location; an electromagnetic energy receiver which receives electromagnetic energy from the person's finger tissue at a second location, wherein parameters or patterns of the received electromagnetic energy are changed by the person's consumption of food and analyzed to monitor the person's food consumption; a power source; a data processor; and a data transmitter.

In an example, a wearable device for non-invasive glucose monitoring can monitor a person's glucose levels by measuring changes in the electromagnetic impedance, resistance, conductivity, or permittivity of finger tissue. In an example, a finger-worn device can further comprise an electromagnetic resonator between an electromagnetic energy emitter and an electromagnetic energy receiver. In an example, an electromagnetic resonator can comprise a split ring. In an example, an electromagnetic resonator can comprise two or more nested rings. In an example, an electromagnetic resonator can comprise two or more stacked rings. In an example, an electromagnetic resonator can comprise a spiral.

Information from a wearable device for non-invasive glucose monitoring can also be combined with a computer-to-human interface that provides feedback to encourage the person to eat healthy foods and to limit excess consumption of unhealthy foods. In an example, a wearable device for non-invasive glucose monitoring can be in wireless communication with a separate feedback device that modifies the person's eating behavior. In an example, capability for monitoring glucose levels can be combined with capability for providing behavior-modifying feedback within a single device. In an example, a single device can be used to measure glucose levels and to provide visual, auditory, tactile, or other feedback to encourage the person to eat in a healthier manner.

In various examples, a wearable device for non-invasive glucose monitoring can be selected from the group consisting of: neuroelectrical sensor, action potential sensor, ECG sensor, EKG sensor, EEG sensor, EGG sensor, capacitance sensor, conductivity sensor, impedance sensor, galvanic skin response sensor, variable impedance sensor, variable resistance sensor, interferometer, magnetometer, RF sensor, electrophoretic sensor, optoelectronic sensor, piezoelectric sensor, and piezocapacitive sensor. In an example, a wearable device for non-invasive glucose monitoring can include a high-energy sensor based on microwaves or x-rays. In various examples a wearable device for non-invasive glucose monitoring can be selected from the group consisting of: a microwave sensor, a microwave spectrometer, and an x-ray detector.

In various examples, a wearable device for non-invasive glucose monitoring can be selected from the group consisting of: chemical sensor, biochemical sensor, amino acid sensor, chemiresistor, chemoreceptor, photochemical sensor, optical sensor, chromatography sensor, fiber optic sensor, infrared sensor, optoelectronic sensor, spectral analysis sensor, spectrophotometer, olfactory sensor, electronic nose, metal oxide semiconductor sensor, conducting polymer sensor, quartz crystal microbalance sensor, electromagnetic sensor, variable impedance sensor, variable resistance sensor, conductance sensor, neural impulse sensor, EEG sensor, EGG sensor, EMG sensor, interferometer, galvanic skin response sensor, cholesterol sensor, HDL sensor, LDL sensor, electrode, neuroelectrical sensor, neural action potential sensor, Micro Electrical Mechanical System (MEMS) sensor, laboratory-on-a-chip, or medichip, micronutrient sensor, osmolality sensor, protein-based sensor or reagent-based sensor, saturated fat sensor or trans fat sensor, action potential sensor, biological sensor, enzyme-based sensor, protein-based sensor, reagent-based sensor, camera, video camera, fixed focal-length camera, variable focal-length camera, pattern recognition sensor, microfluidic sensor, motion sensor, accelerometer, flow sensor, strain gauge, electrogoniometer, inclinometer, peristalsis sensor, multiple-analyte sensor array, an array of cross-reactive sensors, pH level sensor, sodium sensor, sonic energy sensor, microphone, sound-based chewing sensor, sound-based swallow sensor, ultrasonic sensor, sugar sensor, glucose sensor, temperature sensor, thermometer, and thermistor.

In an example, a wearable device for non-invasive glucose monitoring can be worn directly on a person's body. In an example a wearable device for non-invasive glucose monitoring can be worn on, or incorporated into, a person's clothing.

In various examples, a wearable sensor can be worn on a person in a location selected from the group consisting of: wrist, neck, finger, hand, head, ear, eyes, nose, teeth, mouth, torso, chest, waist, and leg. In various examples, a wearable sensor can be attached to a person or to a person's clothing by a means selected from the group consisting of: strap, clip, clamp, snap, pin, hook and eye fastener, magnet, and adhesive.

In various examples, a wearable sensor can be worn on a person in a manner like a clothing accessory or piece of jewelry selected from the group consisting of: wristwatch, wristphone, wristband, bracelet, cufflink, armband, armlet, and finger ring; necklace, neck chain, pendant, dog tags, locket, amulet, necklace phone, and medallion; eyewear, eyeglasses, spectacles, sunglasses, contact lens, goggles, monocle, and visor; clip, tie clip, pin, brooch, clothing button, and pin-type button; headband, hair pin, headphones, ear phones, hearing aid, earring; and dental appliance, palatal vault attachment, and nose ring.

In an example, a wearable device for non-invasive glucose monitoring can be incorporated into a smart watch or other device that is worn on a person's wrist. In an example, a wearable device for non-invasive glucose monitoring can be worn on, or attached to, other members of a person's body or to a person's clothing. In an example, a wearable device for non-invasive glucose monitoring can be worn on, or attached to, a person's body or clothing. In an example, a device can be worn on, or attached to, a part of a person's body that is selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg.

In an example, a wearable device for non-invasive glucose monitoring can be worn in a manner similar to a piece of jewelry or accessory. In various examples, a wearable device for non-invasive glucose monitoring can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

In an example, a wearable device for non-invasive glucose monitoring can be attached to a person's body or clothing. In an example, a wearable device for non-invasive glucose monitoring can be attached to a person's body or clothing using an attachment means selected from the group consisting of: band, strap, chain, hook and eye fabric, ring, adhesive, bracelet, buckle, button, clamp, clip, elastic band, eyewear, magnet, necklace, piercing, pin, string, suture, tensile member, wrist band, and zipper. In an example, a device can be incorporated into the creation of a specific article of clothing. In an example, a wearable device for non-invasive glucose monitoring can be integrated into a specific article of clothing by a means selected from the group consisting of: adhesive, band, buckle, button, clip, elastic band, hook and eye fabric, magnet, pin, pocket, pouch, sewing, strap, tensile member, and zipper.

In an example, a wearable device for non-invasive glucose monitoring can comprise one or more sensors selected from the group consisting of: motion sensor, accelerometer (single multiple axis), electrogoniometer, or strain gauge; optical sensor, miniature still picture camera, miniature video camera, miniature spectroscopy sensor; sound sensor, miniature microphone, speech recognition software, pulse sensor, ultrasound sensor; electromagnetic sensor, skin galvanic response (Galvanic Skin Response) sensor, EMG sensor, chewing sensor, swallowing sensor; temperature sensor, thermometer, infrared sensor; and chemical sensor, chemical sensor array, miniature spectroscopy sensor, glucose sensor, cholesterol sensor, or sodium sensor.

In an example, a wearable device for non-invasive glucose monitoring can be entirely wearable or include a wearable component. In an example, a wearable device or component can be worn directly on a person's body, can be worn on a person's clothing, or can be integrated into a specific article of clothing. In an example, a wearable device for non-invasive glucose monitoring can be in wireless communication with an external device. In various examples, a wearable device for non-invasive glucose monitoring can be in wireless communication with an external device selected from the group consisting of: a cell phone, an electronic tablet, electronically-functional eyewear, a home electronics portal, an internet portal, a laptop computer, a mobile phone, a remote computer, a remote control unit, a smart phone, a smart utensil, a television set, and a virtual menu system.

In an example, a wearable device for non-invasive glucose monitoring can comprise multiple components selected from the group consisting of: Central Processing Unit (CPU) or microprocessor; food-consumption monitoring component (motion sensor, electromagnetic sensor, optical sensor, and/or chemical sensor); graphic display component (display screen and/or coherent light projection); human-to-computer communication component (speech recognition, touch screen, keypad or buttons, and/or gesture recognition); memory component (flash, RAM, or ROM); power source and/or power-transducing component; time keeping and display component; wireless data transmission and reception component; and strap or band.

In an example, a wearable device for non-invasive glucose monitoring can include a hand-held component in addition to a wearable component. In an example, the combination and integration of a wearable member and a hand-held member can provide advantages that are not possible with either a wearable member alone or a hand-held member alone. In an example, a wearable device for non-invasive glucose monitoring can include a hand-held component that is selected from the group consisting of: smart phone, mobile phone, cell phone, holophone, or application of such a phone; electronic tablet, other flat-surface mobile electronic device, personal Digital Assistant (PDA), or laptop; digital camera; and smart eyewear, electronically-functional eyewear, or augmented reality eyewear. In an example, such a hand-held component can be in wireless communication with a wearable component of such a system. In an example, a wearable device for non-invasive glucose monitoring can include integration with a general-purpose mobile device that is used to collect data concerning food consumption. In an example, the hand-held component can be a general purpose device, of which collecting data for food identification is only one among many functions that it performs. In an example, a wearable device for non-invasive glucose monitoring can comprise: a wearable member that continually monitors for possible food consumption; a hand-held smart phone that is used to take pictures of food that will be consumed; wireless communication between the wearable member and the smart phone; and software that integrates the operation of the wearable member and the smart phone.

In various examples, a wearable device for non-invasive glucose monitoring can provide feedback to the person that is selected from the group consisting of: auditory feedback (such as a voice message, alarm, buzzer, ring tone, or song); feedback via computer-generated speech; mild external electric charge or neural stimulation; periodic feedback at a selected time of the day or week; phantom taste or smell; phone call; pre-recorded audio or video message by the person from an earlier time; television-based messages; and tactile, vibratory, or pressure-based feedback.

In various examples, a wearable device for non-invasive glucose monitoring can provide feedback to the person that is selected from the group consisting of: feedback concerning food consumption (such as types and amounts of foods, ingredients, and nutrients consumed, calories consumed, calories expended, and net energy balance during a period of time); information about good or bad ingredients in nearby food; information concerning financial incentives or penalties associated with acts of food consumption and achievement of health-related goals; information concerning progress toward meeting a weight, energy-balance, and/or other health-related goal; information concerning the calories or nutritional components of specific food items; and number of calories consumed per eating event or time period.

In various examples, a wearable device for non-invasive glucose monitoring can provide feedback to the person that is selected from the group consisting of: augmented reality feedback (such as virtual visual elements superimposed on foods within a person's field of vision); changes in a picture or image of a person reflecting the likely effects of a continued pattern of food consumption; display of a person's progress toward achieving energy balance, weight management, dietary, or other health-related goals; graphical display of foods, ingredients, or nutrients consumed relative to standard amounts (such as embodied in pie charts, bar charts, percentages, color spectrums, icons, emoticons, animations, and morphed images); graphical representations of food items; graphical representations of the effects of eating particular foods; holographic display; information on a computer display screen (such as a graphical user interface); lights, pictures, images, or other optical feedback; touch screen display; and visual feedback through electronically-functional eyewear.

In various examples, a wearable device for non-invasive glucose monitoring can provide feedback to the person that is selected from the group consisting of: advice concerning consumption of specific foods or suggested food alternatives (such as advice from a dietician, nutritionist, nurse, physician, health coach, other health care professional, virtual agent, or health plan); electronic verbal or written feedback (such as phone calls, electronic verbal messages, or electronic text messages); live communication from a health care professional; questions to the person that are directed toward better measurement or modification of food consumption; real-time advice concerning whether to eat specific foods and suggestions for alternatives if foods are not healthy; social feedback (such as encouragement or admonitions from friends and/or a social network); suggestions for meal planning and food consumption for an upcoming day; and suggestions for physical activity and caloric expenditure to achieve desired energy balance outcomes.

In various examples, a wearable device for non-invasive glucose monitoring can be in wireless communication with an external device or system selected from the group consisting of: internet portal; smart phone, mobile phone, cell phone, holophone, or application of such a phone; electronic tablet, other flat-surface mobile electronic device, personal Digital Assistant (PDA), remote control unit, or laptop; smart eyewear, electronically-functional eyewear, or augmented reality eyewear; electronic store display, electronic restaurant menu, or vending machine; and desktop computer, television, or mainframe computer. In various examples, a device can receive food-identifying information from a source selected from the group consisting of: electromagnetic transmissions from a food display or RFID food tag in a grocery store, electromagnetic transmissions from a physical menu or virtual user interface at a restaurant, and electromagnetic transmissions from a vending machine.

In an example, a wearable device for non-invasive glucose monitoring can be tamper resistant. In an example, a wearable device can detect when it has been removed from the person's body by monitoring signals from the body such as pulse, motion, heat, skin electromagnetism, or proximity to an implanted device. In an example, a wearable device for non-invasive glucose monitoring can detect if it has been removed from the person's body by detecting a lack of motion, lack of a pulse, and/or lack of electromagnetic response from skin. In various examples, a wearable device for non-invasive glucose monitoring can continually monitor optical, electromagnetic, temperature, pressure, or motion signals that indicate that the device is properly worn by a person. In an example, a wearable device can trigger feedback if the device is removed from the person and the signals stop.

In an example, a sensor can be selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer.

In an example, a wearable device for non-invasive glucose monitoring can comprise: a ring which is configured to be worn on a person's finger, wherein this ring further comprises—an electromagnetic energy sensor which is configured to measure parameters or patterns of electromagnetic energy transmitted through the person's finger tissue, wherein these parameters or patterns of electromagnetic energy are changed by the person's consumption of food; a power source; a data processor; and a data transmitter. In an example, an electromagnetic energy sensor can be an electromagnetic impedance sensor which measures the impedance of a person's finger tissue. In an example, an electromagnetic energy sensor can be an electromagnetic resistance sensor which measures the resistance, however futile, of a person's finger tissue. In an example, an electromagnetic energy sensor can be an electromagnetic conductivity sensor which measures the conductivity of a person's finger tissue.

In an example, a wearable device for non-invasive glucose monitoring can comprise—a ring which is configured to be worn on a person's finger, wherein this ring further comprises: an electromagnetic energy emitter which is configured to emit electromagnetic energy into the person's finger tissue at a first location; an electromagnetic energy receiver which is configured to receive electromagnetic energy from the person's finger tissue at a second location, wherein parameters or patterns of the received electromagnetic energy are changed by the person's consumption of food; a power source; a data processor; and a data transmitter.

In an example, parameters or patterns of the electromagnetic energy can include the impedance of a person's finger tissue. In an example, parameters or patterns of the electromagnetic energy can include the resistance of a person's finger tissue. In an example, parameters or patterns of the electromagnetic energy can include the conductivity of a person's finger tissue. In an example, parameters or patterns of the electromagnetic energy can include the permittivity of a person's finger tissue. In an example, a wearable device for non-invasive glucose monitoring can further comprise a plurality of electromagnetic energy emitters or electromagnetic energy receivers distributed around the circumference of the finger ring.

In an example, a wearable device for non-invasive glucose monitoring can further comprise an electromagnetic resonator between an electromagnetic energy emitter and an electromagnetic energy receiver. In an example, an electromagnetic resonator can comprise a split ring. In an example, an electromagnetic resonator can comprise two or more nested rings. In an example, an electromagnetic resonator can comprise two or more stacked rings. In an example, an electromagnetic resonator can comprise a spiral. In an example, a wearable device for non-invasive glucose monitoring can comprise—a ring which is configured to be worn on a person's finger, wherein this ring further comprises: an electromagnetic energy emitter which is configured to emit electromagnetic energy at a first location; an electromagnetic energy receiver which is configured to receive electromagnetic energy at a second location, wherein parameters or patterns of the received electromagnetic energy are changed by the person's consumption of food; an electromagnetic energy resonator between the electromagnetic energy emitter and the electromagnetic energy receiver; a power source; a data processor; and a data transmitter.

In an example, a wearable device for non-invasive glucose monitoring can be embodied in an automated system for glycemic control comprising: an ear-worn diagnostic component with multiple biosensors which collect data which is jointly analyzed to measure and/or predict body glucose levels; and a wearable and/or implantable pump which dispenses one or more glycemic control substances into a person's body when needed based on analysis of data from the biosensors in the ear-worn diagnostic component.

In an example, the ear-worn diagnostic component of this system can further comprise: one or more electromagnetic energy sensors which collect data concerning electromagnetic brain activity which is analyzed to detect and/or predict body glucose levels; and one or more light energy sensors which collect data concerning the spectrum of light reflected from or having passed through body tissue which is analyzed to detect and/or predict body glucose levels.

In an example, the therapeutic component of this system can further comprise a wearable and/or implantable pump which dispenses insulin (or some other glycemic control substance) when needed based on joint analysis of data from the electromagnetic energy and light energy biometric sensors. In an example, this system can be a closed-loop system for glycemic control in which the pump is automatically triggered to dispense one or more glycemic control substances when joint analysis of data from the electromagnetic energy and light energy sensors detects and/or predicts a change in body glucose levels. In an example, the timing and amount of glycemic control substances dispensed can depend on the timing and magnitude of changes in body glucose levels predicted by joint analysis of data from the electromagnetic energy and light energy sensors.

In an example, a wearable device for non-invasive glucose monitoring can be embodied in an automated closed-loop system for glycemic control comprising: an ear-worn diagnostic component that is worn on a person's ear, around a person's ear, and/or within a person's ear canal, wherein the ear-worn diagnostic component further comprises at least one electromagnetic energy sensor which collects a first set of biometric data, wherein the first set of biometric data concerns the person's electromagnetic brain activity, wherein the ear-worn diagnostic component further comprises at least one light energy sensor which collects a second set of biometric data, and wherein the second set of biometric data concerns the spectrum of light which has been reflected from or passed through the person's body tissue; a wearable and/or implanted pump which dispenses one or more glycemic control substances into the person's body; and a data processor which jointly analyzes the first set of biometric data and the second set of biometric data in order to determine the person's need for the glycemic control substance, and wherein the wearable and/or implanted pump is automatically triggered to dispense the one or more glycemic control substances into the person's body when needed based on the results of analysis by the data processor.

Analysis of a person's electromagnetic brain activity can detect and/or predict changes in body glucose levels earlier than is possible using other types of wearable biometric sensors in the prior art. In an example, analysis of electromagnetic brain activity can predict changes in body glucose levels when a person first sees and smells food, before actual food consumption. In an example, analysis of electromagnetic brain activity can predict changes in body glucose levels when a person begins to chew and/or swallow food, before the food has been digested and nutrients have entered the blood stream. In this manner, analysis of electromagnetic brain activity can provide earlier measurement and/or prediction of changes in body glucose levels than is possible with biometric sensors in the prior art which measure glucose levels in blood or interstitial fluid. Earlier detection and/or prediction of changes in body glucose levels can enable more accurate dispensation of a glycemic control substance by a pump in anticipation of glucose level changes and thus improves glycemic control.

Although analysis of electromagnetic brain activity provides earlier detection and prediction of changes in glucose levels than biometric sensors which measure glucose concentration in blood and/or interstitial fluid, joint analysis of data concerning electromagnetic brain activity and data concerning the spectrum of light reflected from or having passed through body tissue can provide more accurate measurement and/or prediction of body glucose levels than is possible with either type of data alone. Accordingly, a wearable device for non-invasive glucose monitoring can include both electromagnetic energy sensors and spectroscopic light energy sensors and joint analysis of both types of data for measurement and prediction of body glucose levels.

In an example, data concerning electromagnetic brain activity can provide earlier prediction of changes in body glucose levels and data concerning reflected light spectra can provide lagged, but more accurate, measurement of changes in body glucose levels. Accordingly, joint statistical analysis of these two types of biometric data can enable earlier and more-accurate prediction of changes in body glucose levels than is possible with either type of data alone. Joint statistical analysis of these two types of biometric data as disclosed herein can automatically control the operation of a pump that dispenses insulin (or some other glycemic control substance) for better glycemic control than is possible with devices and systems in the prior art.

In an example, an ear-worn diagnostic component can be partially inserted into the ear canal, curve around the rear of the ear, and also be attached to the earlobe. In various examples, an ear-worn diagnostic component can be worn on a person's ear, around a person's ear, and/or within a person's ear canal in a manner similar to a Bluetooth ear device, an ear bud, an ear hook, an ear plug, an earlobe clip, an earphone or set of earphones, an earpiece, an earring, a forehead-spanning band that rests on top of a person's ears, an electroencephalographic (EEG) sensor, a headband, a headphone, a headset, a hearing aid, an ear-worn oximeter, or a tiara. In various examples, an ear-worn diagnostic component of this system can comprise a wearable accessory selected from the group consisting of: a Bluetooth ear device, an ear bud, an ear hook, an ear plug, an earlobe clip, an earphone or set of earphones, an earpiece, an earring, a forehead-spanning band that rests on top of a person's ears, an electroencephalographic (EEG) sensor, a headband, a headphone, a headset, a hearing aid, an ear-worn oximeter, and a tiara.

In an example, an ear-worn component can span some or all of the perimeter of a person's outer ear and/or the tissue which connects the outer ear to the head. In an example, an ear-worn component can span the rear portion of a person's outer ear and/or the tissue connecting the outer ear to the head. In an example, an ear-worn component can rest on the top of the outer ear and/or the tissue connecting the outer ear to the head. In an example, an ear-worn component can curve around the upper and rear portions of an outer ear and/or the tissue connecting the outer ear to the head. In an example, an ear-worn component can include a portion located behind the ear and a portion which is inserted into the ear canal.

In an example, an ear-worn component can be attached to a person's earlobe. In an example, an ear-worn component can clamp or clip to a person's earlobe like an earring. In an example, an ear-worn component can be held onto a person's earlobe by magnetic components. In an example, an ear-worn component can clamp or clip to a person's earlobe like an ear-worn oximeter. In an example, an ear-worn component can pierce through an opening in a person's earlobe in a manner like a earring. In an example, an ear-worn component can curve around the rear portion of an ear and also be attached to the earlobe. In an example, an ear-worn diagnostic component can include a light energy sensor which collects data concerning the spectrum of light which has been reflected from, or passed through, the earlobe. In an example, an ear-worn diagnostic component can have a shape which is selected from the group consisting of: spiral or partial spiral; semi-circle; C-shape; and S-shape.

In an example, an ear-worn diagnostic component can include an arcuate prong, arm, or other protrusion which extends forward from an ear to a location on the temple and/or forehead. In an example, such a prong, arm, or other protrusion can be wavy and/or sinusoidal. In an example, such a prong, arm, or other protrusion can hold at least one electromagnetic energy sensor against the person's temple and/or forehead. In an example, an ear-worn diagnostic component can further comprise: a right-side ear-worn component which rests on the top portion of the right ear, a left-side ear-worn component which rests on the top portion of the left ear, and a band across the forehead which connects the right and left side components. In an example, this band can include one or more electromagnetic energy sensors. In an example, an ear-worn component of this system can be attached to the side-piece of a pair of eyeglasses. In an example, an ear-worn component of this system can be integrated into the side-piece of a pair of eyeglasses.

In an example, a right-side ear-worn component can be worn on a right ear, a left-side ear-worn component can be worn on a left ear, and a band over the top of the head can connect the right and left side components. In an example, the ear-worn component of this system can be embodied in a pair of headphones or earphones. In an example, the ear-worn component of this system can be embodied in a headset. In an example, the ear-worn component of this system can be embodied in a headband.

In an example, an ear-worn diagnostic component can further comprise: a rear segment which is configured to be worn on the rear-facing surface of an ear; a frontal-ear segment which is configured to be worn on the front-facing surface of the ear; a side segment which is configured to span (on the first side of the head) from the ear to the temple, a side portion of the face, and/or a side portion of the forehead; and a top segment which is configured to span (on the first side of the head) from the side segment to the top of the head. In an example, the side segment can further comprise an electromagnetic energy sensor which collects data concerning electromagnetic brain activity.

In an example, an ear-worn diagnostic component can be an ear-inserted component which is inserted (partially or fully) into an ear canal. In an example, an ear-inserted diagnostic component can be worn like a hearing aid, ear bud, or ear plug. In an example, an ear-inserted diagnostic component can be integrated into a hearing aid, ear bud, or ear plug. In an example, an ear-inserted diagnostic component can include an electromagnetic energy sensor which collects data concerning electromagnetic brain activity. This data can be used to measure and/or predict changes in glucose levels and control the operation of a glycemic control substance pump. In an example, an ear-inserted diagnostic component can include a light energy sensor which collects data concerning the spectrum of light which has been reflected from body tissue within a person's ear canal. This data can be used to measure and/or predict changes in glucose levels and control the operation of a glycemic control substance pump. In an example, data from an electromagnetic energy sensor and data from a light energy sensor can be jointly analyzed in order to measure and/or predict glucose levels and automatically control the operation of a glycemic control substance pump.

In an example, an ear-worn diagnostic component can include an electromagnetic energy sensor which is partially inserted into an ear canal. In an example, an ear-worn diagnostic component can comprise one or more electromagnetic energy sensors which collect data concerning electromagnetic brain activity, wherein this data is analyzed to measure and/or predict body glucose levels and automatically control the operation of a glycemic control substance pump. In an example, an electromagnetic energy sensor can be electroencephalographic (EEG) sensor. In an example, changes in body glucose levels cause changes in electroencephalographic signals. In an example, changes in electroencephalographic signals can be used to measure body glucose levels. In an example, changes in electroencephalographic signals can be used to predict changes in body glucose levels. In an example, changes in electroencephalographic signals can be used to determine the amount of a glycemic control substance that should be automatically dispensed by a pump in order to proactively keep body glucose levels within a normal range.

In an example, an electromagnetic energy sensor can be an electromagnetic energy receiver which receives electromagnetic energy which is naturally generated by the electromagnetic activity of the brain. In an example, an electromagnetic energy sensor can comprise an electromagnetic energy emitter at a first location and an electromagnetic energy receiver at a second location, wherein the electromagnetic energy receiver receives energy which has been transmitted from the electromagnetic energy emitter through body tissue. In an example, the electromagnetic energy receiver can collect data concerning (changes in) the conductivity, resistance, and/or impedance of electromagnetic energy transmitted through body tissue from the electromagnetic energy emitter to the electromagnetic energy receiver due to electromagnetic brain activity. In an example, an electromagnetic energy emitter and an electromagnetic energy receiver can together be referred to as an electromagnetic energy sensor.

In an example, an electromagnetic energy sensor can be a dry sensor. In an example, an electromagnetic energy sensor can be a wet sensor. In an example, an electromagnetic energy sensor can be an inductive sensor. In an example, an electromagnetic energy sensor can be a capacitive sensor. In an example, an electromagnetic energy sensor can comprise only an electromagnetic energy receiver. In an example, an electromagnetic energy sensor can be an EEG sensor which collects data concerning the natural emission of electromagnetic energy by a person's brain. In an example, an electromagnetic energy sensor can comprise both an electromagnetic energy emitter and an electromagnetic energy receiver. In an example, an electromagnetic energy sensor can collect data concerning changes in the transmission of electromagnetic energy from an emitter to a receiver due to changes in electromagnetic brain activity. In an example, an electromagnetic brain activity sensor can measure voltage fluctuations resulting from ionic current within the neurons of the brain.

In an example, this system can comprise one or more electromagnetic energy sensors which are configured to measure electromagnetic brain activity in the frontal lobes of the brain in response to glucose levels. In an example, this system can comprise one or more electromagnetic energy sensors which are configured to measure electromagnetic brain activity in the occipital cortex of the brain in response to glucose levels. In an example, this system can comprise one or more electromagnetic energy sensors which are configured to measure electromagnetic brain activity in the visual cortex of the brain in response to glucose levels.

In an example, this system can comprise one or more electromagnetic energy sensors which are configured to measure electromagnetic brain activity in the parietal cortex of the brain in response to glucose levels. In an example, this system can comprise one or more electromagnetic energy sensors which are configured to measure electromagnetic brain activity along the frontal-occipital mid-line in response to glucose levels. In an example, this system can comprise one or more electromagnetic energy sensors which are configured to measure electromagnetic brain activity in the hypothalamus in response to glucose levels.

In an example, an electromagnetic energy sensor can measure voltage fluctuations between a first electrode (e.g. sensor) and a second (reference) electrode (e.g. sensor) due to electromagnetic brain activity. In an example, voltage differences between a first electrode and a second (reference) electrode can be called a “channel” In an example, a set of channels can be called a “montage.” In an example, a pattern of brain activity can be a change in electromagnetic brain activity measured from one location or channel relative to electromagnetic brain activity measured from one or more different locations or channels.

In an example, one or more electromagnetic energy sensors which collect data concerning brain activities or channels can be placed at one or more placement sites selected from the group of standard EEG placement sites consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, DJC, TP8, T5/P7, P5, P3, P1, Pz, P2, P4, P6, T6/P8, PO7, PO5, PO3, POz, PO4, PO6, PO8, O1, Oz, and O2.

In an example, this system can comprise electromagnetic energy sensors which are configured to measure electromagnetic brain activity from standard EEG locations A1 and/or A2. In an example, this system can comprise electromagnetic energy sensors which are configured to measure electromagnetic brain activity from standard EEG locations C1, C2, C3, C4, C5, C6, Cz, T3, and T4. In an example, this system can comprise electromagnetic energy sensors which are configured to measure electromagnetic brain activity from standard EEG locations C1, C5, CP1, CP3, FC1, and FC3. In an example, this system can comprise electromagnetic energy sensors which are configured to measure electromagnetic brain activity from standard EEG locations C1, C5, CP1, CP5, FC1, FC3, P1, P5, and PO3.

In an example, this system can comprise electromagnetic energy sensors which are configured to measure electromagnetic brain activity from standard EEG locations C1, C5, CP3, and FC3. In an example, this system can comprise electromagnetic energy sensors which are configured to measure electromagnetic brain activity from standard EEG locations C3 and/or C4. In an example, this system can comprise electromagnetic energy sensors which are configured to measure electromagnetic brain activity from standard EEG locations C3 and/or T5. In an example, this system can comprise electromagnetic energy sensors which are configured to measure electromagnetic brain activity from standard EEG locations C3, C4 and Cz. In an example, this system can comprise electromagnetic energy sensors which are configured to measure electromagnetic brain activity from standard EEG locations C3, C4, and Pz.

In an example, this system can comprise electromagnetic energy sensors which are configured to measure electromagnetic brain activity from standard EEG locations CP1, CP2, CP3, CP4, CP5, CP6, CPz, TP7, and TP8. In an example, this system can comprise electromagnetic energy sensors which are configured to measure electromagnetic brain activity from standard EEG locations Cz and/or Pz. In an example, this system can comprise electromagnetic energy sensors which are configured to measure electromagnetic brain activity from standard EEG locations O1 and/or O2. In an example, this system can comprise electromagnetic energy sensors which are configured to measure electromagnetic brain activity from standard EEG locations O1, O2, P3, and P4.

In an example, this system can comprise electromagnetic energy sensors which are configured to measure electromagnetic brain activity from standard EEG locations P1, P2, P3, P4, P5, P6, Pz, T5, and T6. In an example, this system can comprise electromagnetic energy sensors which are configured to measure electromagnetic brain activity from standard EEG locations P3 and/or P4. In an example, this system can comprise electromagnetic energy sensors which are configured to measure electromagnetic brain activity from standard EEG locations P3 and/or T3. In an example, this system can comprise electromagnetic energy sensors which are configured to measure electromagnetic brain activity from standard EEG locations P4 and/or T4. In an example, this system can comprise electromagnetic energy sensors which are configured to measure electromagnetic brain activity from standard EEG locations PO3 and/or PO4.

In an example, a pattern of electromagnetic brain activity which is associated with a body glucose level can be a transient pattern. In an example, a transient pattern of electromagnetic brain activity can be an ERP (Event Related Potential or Evoked Response Potential). In an example, different transient patterns of electromagnetic brain activity can be triggered by different types of food selected from the group consisting of: a selected type of carbohydrate, a class of carbohydrates, or all carbohydrates; a selected type of sugar, a class of sugars, or all sugars; a selected type of fat, a class of fats, or all fats; a selected type of cholesterol, a class of cholesterols, or all cholesterols; a selected type of protein, a class of proteins, or all proteins; a selected type of fiber, a class of fiber, or all fibers; a specific sodium compound, a class of sodium compounds, or all sodium compounds; high-carbohydrate food, high-sugar food, high-fat food, fried food, high-cholesterol food, high-protein food, high-fiber food, and/or high-sodium food.

In an example, a specific transient pattern of electromagnetic brain activity can indicate and/or predict hypoglycemia, hyperglycemia, and/or neuroglycemia. In an example, a specific transient pattern of electromagnetic brain activity can indicate and/or predict a specific range of body glucose levels. In an example, a specific transient pattern of electromagnetic brain activity can indicate and/or predict a specific change in body glucose levels. In an example, a specific transient pattern of electromagnetic brain activity can be used to trigger a specific dose of a glycemic control substance by a pump in order to maintain body glucose levels within a normal range.

In an example, a specific pattern of brain activity associated with food consumption and/or a change in body glucose level can be identified as an ERP (Event Related Potential or Evoked Response Potential). In an example, a specific change in body glucose level can be identified as a transient pattern of brain activity which does not repeat over time. In an example, statistical methods used to associate specific brainwave patterns can include analysis of wave frequency, wave frequency band, wave amplitude, wave phase, and wave form or morphology. In an example, wave form or morphology can be identified from the group consisting of: simple sinusoidal wave, composite sinusoidal wave, simple saw-tooth wave, composite saw-tooth wave, biphasic wave, tri-phasic wave, and spike.

In an example, a pattern of electromagnetic brain activity which is associated with a body glucose level can be a repeating pattern. In an example, a repeating pattern of electromagnetic brain activity can be analyzed and decomposed into different frequency bands using Fourier Transformation methods. In an example, a pattern of electromagnetic brain activity can be a sinusoidal pattern. In an example, a repeating electromagnetic brain activity pattern can be modeled as a combination of multiple sine waves. In an example, a repeating electromagnetic brain activity pattern can be decomposed into sub-patterns in different frequency bands.

In an example, data concerning a person's electromagnetic brain activity from one or more wearable electromagnetic energy sensors can be analyzed to detect and/or predict changes in glucose levels earlier than using other types of wearable biometric sensors. Earlier detection and/or prediction of changes in glucose levels can enable more accurate glycemic control. In an example, analysis of electromagnetic brain activity can predict changes in glucose levels by identifying when a person sees and smells food—before actual food consumption. In an example, analysis of electromagnetic brain activity can predict changes in glucose levels when a person begins to eat food—before digested nutrients enter the blood stream. In an example, analysis of electromagnetic brain activity can predict changes in glucose levels long before food consumption is detected by changes in interstitial fluid.

There can be a phased effect of food consumption on brain activity. A first phase occurs when a person sees and smells food before eating it. A second phase occurs when a person tastes, smells, and feels food as they eat it. A third phase occurs as food is digested within a person's gastrointestinal tract and nutrients from food enter the person's blood stream. Statistical analysis of brain activity can comprise analysis of the separate, sequential, or cumulative effects of these three phases of food consumption for more accurate measurement and/or prediction of changes in body glucose levels.

In an example, a specific repeating pattern of electromagnetic brain activity can indicate and/or predict hypoglycemia, hyperglycemia, and/or neuroglycemia. In an example, a specific repeating pattern of electromagnetic brain can predict a specific range of body glucose levels. In an example, a specific repeating pattern of electromagnetic brain can predict a specific change of body glucose levels. In an example, a specific repeating pattern of electromagnetic brain activity can be used to trigger a specific dose of a glycemic control substance by a pump in order to maintain body glucose levels within a normal range.

In an example, repeating patterns of electromagnetic brain activity can be decomposed into frequency bands. In an example, this frequency decomposition can be done using Fourier Transformation. In an example, frequency bands for repeating patterns of electromagnetic brain activity can be selected from the group consisting of: Delta, Theta, Alpha, Beta, and Gamma. Repeating brain waveforms classified as Delta waves can be within a frequency band selected from the group consisting of: 0.5-3.5 Hz, 0.5-4 Hz, 1-3 Hz, 1-4 Hz, and 2-4 Hz. Repeating brain waveforms classified as Theta waves can be within a frequency band selected from the group consisting of: from the group consisting of: 3.5-7 Hz, 3-7 Hz, 4-7 Hz, 4-7.5 Hz, 4-8 Hz, and 5-7 Hz. Repeating brain waveforms classified as Alpha waves can be within a frequency band selected from the group consisting of: 7-13 Hz, 7-14 Hz, 8-12 Hz, 8-13 Hz, 7-11 Hz, 8-10 Hz, and 8-10 Hz. Repeating brain waveforms classified as Beta waves can be within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. Repeating brain waveforms classified as Gamma waves can be within a frequency band selected from the group consisting of: group consisting of: 30-100 Hz, 35-100 Hz, 40-100 Hz, and greater than 30 Hz.

In an example, this system can analyze repeating patterns of electromagnetic brain activity in one or more specific-frequency bands. In an example, these specific-frequency bands can be selected from the group consisting of: Alpha, Beta and/or sensory Motor Rhythm (SMR), Delta, Gamma, and Theta. In an example, this system can analyze repeating patterns of electromagnetic brain activity in a specific-frequency band in order to detect and/or predict a person's glucose level. In an example, this system can analyze repeating patterns of electromagnetic brain activity in a specific-frequency band in order to detect, predict, reduce, and/or avoid hypoglycemia, hyperglycemia, and/or neuroglycemia. In an example, this system can analyze repeating patterns of electromagnetic brain activity in a specific-frequency band in order to automatically control the operation of a drug pump for glycemic control.

In an example, this system can identify a change in the average power level of electromagnetic brain activity in a specific-frequency band in order to detect and/or predict a person's glucose level. In an example, this system can identify a change in the average power level of electromagnetic brain activity in a specific-frequency band in order to detect, predict, reduce, and/or avoid hypoglycemia, hyperglycemia, and/or neuroglycemia. In an example, this system can identify a change in the average power level of electromagnetic brain activity in a specific-frequency band in order to control the operation of a drug pump for glycemic control.

In an example, this system can identify a slowing or speeding up of electromagnetic brain activity in a specific-frequency band in order to detect and/or predict a person's glucose level. In an example, this system can identify can identify a slowing or speeding up of electromagnetic brain activity in a specific-frequency band in order to detect, predict, reduce, and/or avoid hypoglycemia, hyperglycemia, and/or neuroglycemia. In an example, this system can identify can identify a slowing or speeding up of electromagnetic brain activity in a specific-frequency band in order to control the operation of a drug pump for glycemic control. More generally, this system can identify can identify a lower or higher speed of electromagnetic brain activity in a specific-frequency band in order to control the operation of a drug pump for glycemic control.

In an example, this system can identify a shift in the centroid of electromagnetic brain activity in a specific-frequency band in order to detect and/or predict a person's glucose level. In an example, this system can identify can identify a shift in the centroid of electromagnetic brain activity in a specific-frequency band in order to detect, predict, reduce, and/or avoid hypoglycemia, hyperglycemia, and/or neuroglycemia. In an example, this system can identify can identify a shift in the centroid of electromagnetic brain activity in a specific-frequency band in order to control the operation of a drug pump for glycemic control.

In an example, this system can analyze repeating patterns of electromagnetic brain activity in the Alpha frequency range, wherein the Alpha range can be selected from the group of ranges consisting of: 7-12 Hz, 8-12 Hz, and 8-13 Hz. In an example, this system can analyze repeating patterns of electromagnetic brain activity in the Alpha range in order to detect and/or predict a person's glucose level. In an example, this system can analyze repeating patterns of electromagnetic brain activity in the Alpha range in order to detect, predict, reduce, and/or avoid hypoglycemia, hyperglycemia, and/or neuroglycemia. In an example, this system can analyze repeating patterns of electromagnetic brain activity in the Alpha range in order to control the operation of a drug pump for glycemic control.

In an example, this system can identify decreased (e.g. decreased average power level) of electromagnetic brain activity in the Alpha range in order to detect and/or predict a person's glucose level. In an example, this system can identify decreased (e.g. decreased average power level) of electromagnetic brain activity in the Alpha range in order to detect, predict, reduce, and/or avoid hypoglycemia, hyperglycemia, and/or neuroglycemia. In an example, this system can identify decreased (e.g. decreased average power level) of electromagnetic brain activity in the Alpha range in order to control the operation of a drug pump for glycemic control. More generally, this system can identify decreased or increased power of electromagnetic brain activity in the Alpha range in order to control the operation of a drug pump for glycemic control.

In an example, this system can identify a slowing of electromagnetic brain activity in the Alpha range in order to detect and/or predict a person's glucose level. In an example, this system can identify can identify a slowing of electromagnetic brain activity in the Alpha range in order to detect, predict, reduce, and/or avoid hypoglycemia, hyperglycemia, and/or neuroglycemia. In an example, this system can identify can identify a slowing of electromagnetic brain activity in the Alpha range in order to control the operation of a drug pump for glycemic control. More generally, this system can identify can identify a lower or higher speed of electromagnetic brain activity in the Alpha range in order to control the operation of a drug pump for glycemic control.

In an example, this system can identify a shift in the centroid of electromagnetic brain activity in the Alpha range in order to detect and/or predict a person's glucose level. In an example, this system can identify can identify a shift in the centroid of electromagnetic brain activity in the Alpha range in order to detect, predict, reduce, and/or avoid hypoglycemia, hyperglycemia, and/or neuroglycemia. In an example, this system can identify can identify a shift in the centroid of electromagnetic brain activity in the Alpha range in order to control the operation of a drug pump for glycemic control.

In an example, changes in glucose levels can be associated with a change in wave shape for brainwaves in the Alpha frequency band. In an example, changes in glucose levels can be associated with a change in which brain regions originate or modify brainwaves within the Alpha frequency band. In an example, changes in glucose levels can be associated with a change in brainwave activity within the Alpha band from the anterior vs. posterior a person's brain. In an example, changes in glucose levels can be associated with a change in brainwave activity within the Alpha band for a particular brain lobe or organelle. In an example, changes in glucose levels can be associated with a change in brainwave activity within the Alpha band as measured from a specific electrode site, a specific electrode channel, and/or a specific montage of channels.

In an example, this system can analyze repeating patterns of electromagnetic brain activity in the Delta frequency range, wherein the Delta range is 1-4 Hz. In an example, this system can analyze repeating patterns of electromagnetic brain activity in the Delta range in order to detect and/or predict a person's glucose level. In an example, this system can analyze repeating patterns of electromagnetic brain activity in the Delta range in order to detect, predict, reduce, and/or avoid hypoglycemia, hyperglycemia, and/or neuroglycemia. In an example, this system can analyze repeating patterns of electromagnetic brain activity in the Delta range in order to control the operation of a drug pump for glycemic control.

In an example, this system can identify increased (e.g. increased average power level) of electromagnetic brain activity in the Delta range in order to detect and/or predict a person's glucose level. In an example, this system can identify increased (e.g. increased average power level) of electromagnetic brain activity in the Delta range in order to detect, predict, reduce, and/or avoid hypoglycemia, hyperglycemia, and/or neuroglycemia. In an example, this system can identify increased (e.g. increased average power level) of electromagnetic brain activity in the Delta range in order to control the operation of a drug pump for glycemic control. More generally, this system can identify increased or decreased power of electromagnetic brain activity in the Delta range in order to control the operation of a drug pump for glycemic control.

In an example, this system can identify a slowing of electromagnetic brain activity in the Delta range in order to detect and/or predict a person's glucose level. In an example, this system can identify can identify a slowing of electromagnetic brain activity in the Delta range in order to detect, predict, reduce, and/or avoid hypoglycemia, hyperglycemia, and/or neuroglycemia. In an example, this system can identify can identify a slowing of electromagnetic brain activity in the Delta range in order to control the operation of a drug pump for glycemic control. More generally, this system can identify can identify a lower or higher speed of electromagnetic brain activity in the Delta range in order to control the operation of a drug pump for glycemic control.

In an example, this system can identify a shift in the centroid of electromagnetic brain activity in the Delta range in order to detect and/or predict a person's glucose level. In an example, this system can identify can identify a shift in the centroid of electromagnetic brain activity in the Delta range in order to detect, predict, reduce, and/or avoid hypoglycemia, hyperglycemia, and/or neuroglycemia. In an example, this system can identify can identify a shift in the centroid of electromagnetic brain activity in the Delta range in order to control the operation of a drug pump for glycemic control.

In an example, this system can identify coarse Delta waves in order to detect and/or predict a person's glucose level. In an example, this system can identify can identify coarse Delta waves in order to detect, predict, reduce, and/or avoid hypoglycemia, hyperglycemia, and/or neuroglycemia. In an example, this system can identify can identify coarse Delta waves in order to control the operation of a drug pump for glycemic control.

In an example, changes in glucose levels can be associated with a change in wave shape for brainwaves in the Delta frequency band. In an example, changes in glucose levels can be associated with a change in which brain regions originate or modify brainwaves within the Delta frequency band. In an example, changes in glucose levels can be associated with a change in brainwave activity within the Delta band from the anterior vs. posterior a person's brain. In an example, changes in glucose levels can be associated with a change in brainwave activity within the Delta band for a particular brain lobe or organelle. In an example, changes in glucose levels can be associated with a change in brainwave activity within the Delta band as measured from a specific electrode site, a specific electrode channel, and/or a specific montage of channels.

In an example, this system can analyze repeating patterns of electromagnetic brain activity in the Theta frequency range, wherein the Theta range is 4-8 Hz. In an example, this system can analyze repeating patterns of electromagnetic brain activity in the Theta range in order to detect and/or predict a person's glucose level. In an example, this system can analyze repeating patterns of electromagnetic brain activity in the Theta range in order to detect, predict, reduce, and/or avoid hypoglycemia, hyperglycemia, and/or neuroglycemia. In an example, this system can analyze repeating patterns of electromagnetic brain activity in the Theta range in order to control the operation of a drug pump for glycemic control.

In an example, this system can identify increased (e.g. increased average power level) of electromagnetic brain activity in the Theta range in order to detect and/or predict a person's glucose level. In an example, this system can identify increased (e.g. increased average power level) of electromagnetic brain activity in the Theta range in order to detect, predict, reduce, and/or avoid hypoglycemia, hyperglycemia, and/or neuroglycemia. In an example, this system can identify increased (e.g. increased average power level) of electromagnetic brain activity in the Theta range in order to control the operation of a drug pump for glycemic control. More generally, this system can identify increased or decreased power of electromagnetic brain activity in the Theta range in order to control the operation of a drug pump for glycemic control.

In an example, this system can identify a speeding up of electromagnetic brain activity in the Theta range in order to detect and/or predict a person's glucose level. In an example, this system can identify can identify a speeding up of electromagnetic brain activity in the Theta range in order to detect, predict, reduce, and/or avoid hypoglycemia, hyperglycemia, and/or neuroglycemia. In an example, this system can identify can identify a speeding up of electromagnetic brain activity in the Theta range in order to control the operation of a drug pump for glycemic control. More generally, this system can identify can identify a higher or lower speed of electromagnetic brain activity in the Theta range in order to control the operation of a drug pump for glycemic control.

In an example, this system can identify a shift in the centroid of electromagnetic brain activity in the Theta range in order to detect and/or predict a person's glucose level. In an example, this system can identify can identify a shift in the centroid of electromagnetic brain activity in the Theta range in order to detect, predict, reduce, and/or avoid hypoglycemia, hyperglycemia, and/or neuroglycemia. In an example, this system can identify can identify a shift in the centroid of electromagnetic brain activity in the Theta range in order to control the operation of a drug pump for glycemic control.

In an example, changes in glucose levels can be associated with a change in wave shape for brainwaves in the Theta frequency band. In an example, changes in glucose levels can be associated with a change in which brain regions originate or modify brainwaves within the Theta frequency band. In an example, changes in glucose levels can be associated with a change in brainwave activity within the Theta band from the anterior vs. posterior a person's brain. In an example, changes in glucose levels can be associated with a change in brainwave activity within the Theta band for a particular brain lobe or organelle. In an example, changes in glucose levels can be associated with a change in brainwave activity within the Theta band as measured from a specific electrode site, a specific electrode channel, and/or a specific montage of channels.

In an example, the average power for each of multiple frequency bands of electromagnetic brain activity can be calculated. In an example, the average power for each of multiple frequency bands of electromagnetic brain activity can be calculated using Fast Fourier Transformation. In an example, these frequency bands can be selected from the group consisting of: Alpha, Delta, Theta, Beta and/or sensory Motor Rhythm (SMR), and Gamma. In an example, a ratio can be calculated by dividing the average power of a first frequency band by the average power of a second frequency band. In an example, one such ratio which may be used for glycemic control is the (Alpha)/(Theta) ratio. In an example, such a ratio can be associated with a range of body glucose level and/or the prediction of a change in body glucose levels. In an example, the operation of a glycemic control substance pump can be triggered and/or controlled based on analysis of such a ratio.

In an example, a band ratio can be calculated by dividing the sum of the average powers of (one or more) frequency bands by the sum of the average powers of (one or more) different frequency bands. In an example, a band ratio can be calculated by dividing the product of the average powers of (one or more) frequency bands by the product of the average powers of (one or more) other frequency bands. In an example, one such band ratio which may be used for glycemic control is [(Theta)(SMR)]/[(Delta)(Alpha)]. In an example, such a band ratio can be associated with a specific range of body glucose levels and/or the prediction of a specific change in body glucose levels. In an example, the operation of a glycemic control substance pump can be triggered and/or controlled based on analysis of such a band ratio.

In an example, a compound band ratio for three or more frequency bands can be calculated by dividing the average power of a first frequency band by the average power of a second frequency band, dividing that result by the average power of a third frequency band, dividing that result by the average power of a fourth frequency band, and so forth. In an example, one such compound band ratio can be created by the sequential division of (Theta)/(Alpha)/(Delta)/(SMR). In an example, such a band ratio can be associated with a specific range of body glucose levels and/or the prediction of a specific change in body glucose levels. In an example, the operation of a glycemic control substance pump can be triggered and/or controlled based on analysis of such a band ratio.

In an example, this system can use one or more of the above-defined band ratios in order to detect and/or predict a person's glucose level. In an example, this system can use one or more of the above-defined band ratios in order to detect, predict, reduce, and/or avoid hypoglycemia, hyperglycemia, and/or neuroglycemia. In an example, this system can use one or more of the above-defined band ratios in order to control the operation of a glycemic control substance pump, such as an insulin pump. In an example, a particular range of values for a band ratio can trigger the dispensation of a particular amount of a glycemic control substance. In an example, the amount of a glycemic control substance which is dispensed by a pump can be proportional to a specific band ratio. In an example, the amount of a glycemic control substance which is dispensed by a pump can be determined by the amount by which a specific band ratio exceeds a particular value.

In an example, this system can analyze patterns of electromagnetic brain activity in order to control the operation of a drug pump for glycemic control using one or more methods selected from the group consisting of: identifying significant changes in the amplitude, power level, phase, frequency, and/or oscillation of waveform data from one or more channels during or after food consumption; identifying significant changes in the amplitude, power level, phase, frequency, and/or oscillation of waveform data within a selected frequency band during or after food consumption; and identifying significant changes in the relative amplitudes, power levels, phases, frequencies, and/or oscillations of waveform data among different frequency bands during or after food consumption.

In an example, patterns of electromagnetic brain activity can be analyzed using one or more methods selected from the group consisting of: analysis of an overall shift from faster frequency bands to slower frequency bands, analysis of shifts in a frequency band centroid, analysis of the rate of change in band frequency, ANCOVA (analysis of covariance), ANN (Artificial Neural Network), ANOVA (analysis of variance), AR (Auto-Regressive) Modeling, average power analysis for frequency bands, BA (Bonferroni Analysis), band ratio analysis, Bayesian analysis, Bayesian classifier, Bayesian neural network, Binary Decision Tree, Centroid Analysis, centroid shift analysis for frequency bands, Chi-Squared Analysis, Cluster Analysis, controlling for time of day, Correlation, and covariance analysis of two or more frequency bands.

In an example, patterns of electromagnetic brain activity can be analyzed using one or more methods selected from the group consisting of: DA (Discriminant Analysis), DFT (Discrete Fourier Transform), DN (Data Normalization), DTA (Decision Tree Analysis), EMD (Empirical Mode Decomposition), FA (Factor Analysis), FFT (Fast Fourier Transform), Fisher Linear Discriminant, FL (Fuzzy Logic), FVA (Feature Vector Analysis), GARCH (Generalized Auto-Regressive Conditional Heteroscedasticity) Modeling, Gaussian process classifier, GM (Gaussian Model), HMM (Hidden Markov Model), ICA (Independent Components Analysis), Inter-Band Power Ratio, Inter-Channel Power Ratio, Inter-Montage Power Mean, Inter-Montage Ratio, Kernel Estimation, and KF (Kalman Filter).

In an example, patterns of electromagnetic brain activity can be analyzed using one or more methods selected from the group consisting of: Laplacian Filter, Laplacian Montage Analysis, Linear Regression, Linear Transform, Logistic regression algorithm, Logit Model, LSE (Least Squares Estimation), Markov Model, Maximum Entropy Modeling, Maximum Likelihood, Mean Power, ML (Machine Learning), MLR (Multivariate Linear Regression), MTW (Moving Time Window) analysis, Multi-Band Covariance analysis, Multi-Channel Covariance analysis, Multivariate Logit, Multivariate Regression, Naive Bayes Classifier, Neural Network, NLP (Non-Linear Programming), NMF (Non-negative Matrix Factorization), and NMF (Non-Zero Matrix Factorization).

In an example, patterns of electromagnetic brain activity can be analyzed using one or more methods selected from the group consisting of: PCA (Principal Components Analysis), Power Spectral Density, Power Spectrum Analysis, Probit Model, Quadratic Minimum Distance Classifier, Regression Model, RF (Random Forest) analysis, SA (Signal Amplitude), segmenting signals in into time segments or epochs, Signal Averaging, Signal Decomposition, Sine Wave Compositing, Sparse Regression, Spline Function, SVD (Singular Value Decomposition), SVM (Support Vector Machine), testing the effects of varying the boundaries on frequency bands, Time Domain Analysis, Time Frequency Analysis, Time Series Model, Trained Bayes Classifier, Variance, Waveform Identification, Wavelet Analysis, and Wavelet Transformation.

In an example, an ear-worn diagnostic component can include a light energy sensor which is attached to a person's earlobe. In an example, an ear-worn diagnostic component of this system can include one or more spectroscopic light energy sensors which collect data concerning light reflected from (or having passed through) tissue, wherein the spectrum of this light is affected by glucose levels in the tissue. In an example, a light energy sensor within the ear-worn component of this system can be a spectroscopic sensor (also called a “spectroscopy sensor”) which receives light energy which has been reflected from, or passed through, body tissue, organs, and/or fluid. A spectroscopic sensor can collect data concerning the spectrum of light energy which has been reflected from (or has passed through) body tissue, organs, and/or fluid. This data concerning light energy can be used to analyze the spectral distribution of that light and thereby infer the chemical composition and/or physical configuration of the body tissue, organs, and/or fluid.

In an example, a spectroscopic sensor can be selected from the group consisting of: ambient light spectroscopic sensor, analytical chromatographic sensor, backscattering spectrometry sensor, spectroscopic camera, chemiluminescence sensor, chromatographic sensor, coherent light spectroscopic sensor, colorimetric sensor, fiber optic spectroscopic sensor, fluorescence sensor, gas chromatography sensor, infrared light energy sensor, infrared spectroscopic sensor, ion mobility spectroscopic sensor, laser spectroscopic sensor, liquid chromatography sensor, mass spectrometry sensor, near infrared spectroscopic sensor, optoelectronic sensor, photocell, photochemical sensor, Raman spectroscopy sensor, spectral analysis sensor, spectrographic sensor, spectrometric sensor, spectrometry sensor, spectrophotometer, spectroscopic glucose sensor, spectroscopic oximeter, ultraviolet light energy sensor, ultraviolet spectroscopic sensor, variable focal-length camera, video camera, visible light spectroscopic sensor, and white light spectroscopic sensor.

In an example, a spectroscopic sensor can comprise a light receiver alone if it receives ambient light which has been reflected from (or has passed through) body tissue, organs, and/or fluid. In an example, a spectroscopic sensor can comprise both a light emitter and a light receiver if it the light receiver receives light which has been emitted by the light emitter and then reflected from (or passed through) body tissue, organs, and/or fluid. In an example, a light emitter and light receiver can be paired together. In an example, a light emitter and light receiver together can be referred to as a spectroscopic sensor.

In an example, a light energy sensor of this system can be a spectroscopic sensor, including a light emitter and light receiver, which collects light energy data which then is analyzed using spectroscopic analysis in order to measure the chemical composition of body tissue, organs, and/or fluid. In an example, a light energy sensor of this system can be a spectroscopic sensor, including a light emitter and light receiver, which collects light energy data which then is analyzed using spectroscopic analysis in order to monitor changes in the chemical composition of body tissue, organs, and/or fluid. In an example, changes, gaps, and/or shifts in selected frequencies in the spectrum of ambient light due to interaction with a person's body tissue and/or fluid can be analyzed to monitor changes in the chemical composition of the person's body tissue and/or fluid. In an example, data from a spectroscopic sensor can be analyzed to determine how the spectrum of ambient light has been changed by reflection from, or passage through, body tissue, organs, and/or fluid.

In an example, the level of glucose in body tissue and/or fluid can be measured by analyzing the spectrum of light reflected from or having passed through the body tissue and/or fluid. In an example, the concentration of glucose molecules changes the amount of light reflected or absorbed at selected ranges in the light spectrum. In an example, data concerning glucose concentrations in body tissue and/or fluid can jointly analyzed with data concerning electromagnetic brain activity in order to measure and/or predict changes in body glucose levels. These measurements and/or predictions of changes in body glucose levels can then be used to control the operation of the pump which dispenses a glycemic control substance.

In an example, the spectrum of light reflected from or having passed through body tissue and/or fluid is changed following the consumption of a type of food selected from the group consisting of: a selected type of carbohydrate, a class of carbohydrates, or all carbohydrates; a selected type of sugar, a class of sugars, or all sugars; a selected type of fat, a class of fats, or all fats; a selected type of cholesterol, a class of cholesterols, or all cholesterols; a selected type of protein, a class of proteins, or all proteins; a selected type of fiber, a class of fiber, or all fibers; a specific sodium compound, a class of sodium compounds, or all sodium compounds; high-carbohydrate food, high-sugar food, high-fat food, fried food, high-cholesterol food, high-protein food, high-fiber food, and/or high-sodium food. In an example, consumption of one of these types of food can be measured based on changes in the spectrum of light reflected from or having passed through body tissue and/or fluid.

In an example, the light energy sensor of this system can be a spectroscopic sensor, including a light emitter and light receiver, which collects light energy data which then is analyzed using spectroscopic analysis in order to measure the physical configuration of body tissue, organs, and/or fluid. In an example, the light energy sensor of this system can be a spectroscopic sensor, including a light emitter and light receiver, which collects light energy data which then is analyzed using spectroscopic analysis in order to monitor changes in the physical configuration of body tissue, organs, and/or fluid.

In an example, a spectroscopic sensor of this system can include one or more light (energy) emitters. In an example, one or more light (energy) emitters can be selected from the following types of light emitters: arc source, blackbody source, coherent light source, incandescent bulb, infrared light emitter, laser, Laser Diode (LD), Light Emitting Diode (LED), mercury lamp, microplasma light emitter, multi-wavelength source, Organic Light Emitting Diode (OLED), Resonant Cavity Light Emitting Diode (RCLED), Superluminescent Light Emitting Diode (SLED), ultraviolet light emitter, and tungsten lamp.

In an example, a spectroscopic sensor of this system can include one or more light emitters which emit light energy toward a person's skin and/or body surface. In an example, one or more light emitters can emit light energy toward a person's body tissue, organs, and/or fluid. In an example, one or more light emitters can deliver light energy to a person's body tissue, organs, and/or fluid. In an example, one or more light emitters can deliver light energy to body tissue, organs, and/or fluid directly via direct optical communication. In an example, one or more light emitters can deliver light energy to body tissue, organs, and/or fluid indirectly via one or more light guides. In an example, this light energy can be reflected from body tissue, organs, and/or fluid and then the reflected light energy can be received by a light receiver, which is also part of this system. In an example, this light energy can be transmitted through body tissue, organs, and/or fluid and then the transmitted light energy can be received by a light receiver, which is also part of this system. In an example, one or more light emitters can deliver light energy in one or more selected wavelengths (or wavelength ranges or spectra) to body tissue, organs, and/or fluid. In an example, one or more light emitters can deliver infrared light energy, near infrared light energy, ultraviolet light energy, and/or visible light energy to body tissue, organs, and/or fluid.

In an example, the ear-worn diagnostic component can comprise a light-emitting member (such as an LED) which is configured to direct light toward the person's body. In an example, this light can be infrared light, near-infrared light, ultraviolet light, and visible and/or white light. In an example, this light can be coherent and/or laser light. In an example, a spectroscopic sensor can receive this directed light after it has been reflected from, or passed through, the person's body tissue and/or fluid. In an example, data from a spectroscopic sensor can be analyzed to determine how the spectrum of directed light has been changed by reflection from, or passage through, the person's body tissue and/or fluid. In an example, changes in the spectrum of directed light due to interaction with a person's body tissue and/or fluid can be analyzed to measure (changes in) the chemical composition of the person's body tissue and/or fluid.

In an example, this system can include one or more light guides which direct light energy from a first location, angle, and/or transmission vector to a second location, angle, and/or transmission vector. In an example, a light guide can direct light from a light emitter toward body tissue, organs, and/or fluid. In an example, a light guide can collect and direct ambient light toward body tissue, organs, and/or fluid. In an example, a light guide can direct light reflected from, or having passed through, body tissue, organs, and/or fluid toward a light receiver. In an example, a light guide can be generally cylindrical and/or columnar. In an example, a light guide can be rigid. In an example, a light guide can be flexible. In an example, a light guide can be made from one or more materials selected from the group consisting of: acrylic, crystal, elastomeric light-transmissive material, glass, high-durometer plastic, low-durometer plastic, optical-pass material, polycarbonate, polyethylene, polymer, polyurethane, resin, sapphire, and transparent polymer.

In an example, the ear-worn diagnostic component can include one or more light filters. In an example, a light filter can partially absorb and/or block light transmission between a light emitter and body tissue. In an example, a light filter can partially absorb and/or block light transmission between ambient light and body tissue. In an example, a light filter can partially absorb and/or block light transmission between body tissue and a light receiver. In an example, one or more light filters can partially absorb and/or block one or more selected light wavelengths, wavelength ranges, frequencies, and/or frequency ranges. In an example, a light filter can absorb and/or block infrared or ultraviolet light. In an example, a light filter can selectively allow transmission of only infrared light or only ultraviolet light. In an example, a light filter can be made from one or more materials selected from the group consisting of: acrylic, crystal, glass, high-durometer plastic, low-durometer plastic, optical-pass material, polycarbonate, polyethylene, polymer, polyurethane, resin, sapphire, and transparent polymer. In an example, a light filter can be made by adding a light-absorbing dye to acrylic, crystal, glass, plastic, polycarbonate, polyethylene, polymer, polyurethane, resin, and/or a transparent polymer.

In an example, the ear-worn diagnostic component can include one or more lenses. In an example, the ear-worn diagnostic component can include a lens which selectively refracts and/or focuses light. In an example, a lens can selectively refract and/or focus light transmission between a light emitter and body tissue. In an example, a lens can selectively refract and/or focus light transmission between ambient light and body tissue. In an example, a lens can selectively refract and/or focus light transmission between body tissue and a light receiver. In an example, a lens can be selected from the group consisting of: biconcave, biconvex, collimating, columnar, concave, converging, convex, diverging, fluid lens, Fresnel, multiple lenses, negative meniscus, planoconcave, planoconvex, polarizing, positive meniscus, prismatic, and variable-focal lens. In an example, a lens can be made from one or more materials selected from the group consisting of: acrylic, crystal, glass, high-durometer plastic, low-durometer plastic, optical-pass material, polycarbonate, polyethylene, polymer, polyurethane, resin, sapphire, and transparent polymer.

In an example, a spectroscopic sensor of this system can include an array of light (energy) emitters. In an example, different emitters in this array can be configured to have different locations relative to the person's body. In an example, different emitters in this array can emit light at different angles with respect to the surface of a person's body. In an example, different emitters in this array can emit light at different wavelengths and/or with different light spectral distributions. In an example, different emitters in this array can emit light with different levels of coherence.

In an example, a spectroscopic sensor of this system can include a first light emitter and a second light emitter. In an example, the first light emitter can have a first location relative to the person's body and the second light emitter can have a second location relative to the person's body. In an example, the first light emitter can emit light at a first angle with respect to the surface of a person's body and the second light emitter can emit light at a second angle with respect to the surface of a person's body. In an example, the first light emitter can emit light with a first wavelength (or spectral distribution) and the second light emitter can emit light with a second wavelength (or spectral distribution). In an example, the first light emitter can emit coherent light and the second light emitter can emit non-coherent light.

In an example, a first light emitter can emit light during a first time period and a second light emitter can emit light during a second time period. In an example, the first light emitter can emit light during a first environmental condition and the second light emitter can emit light during a second environmental condition. In an example, the first light emitter can emit light when the person is engaged in a first type of physical activity and the second light emitter can emit light when the person is engaged in a second type of physical activity.

In an example, different emitters in this array emit light at different times. In an example, different emitters in this array emit light based on data from one or more light energy sensors detecting different biological or physiological parameters or conditions. In an example, different emitters in this array emit light based on data from one or more light energy sensors when a person is engaged in different types of activities. In an example, different emitters in this array emit light based on data from one or more environmental sensors in response to different environmental parameters or conditions.

In an example, different emitters in this array can emit light with different wavelengths or wavelength ranges. In an example, different emitters in this array can emit light with different wavelengths or wavelength ranges based on data from one or more light energy sensors detecting different biological or physiological parameters or conditions. In an example, different emitters in this array can emit light with different wavelengths or wavelength ranges based on data from one or more light energy sensors when a person is engaged in different types of activities. In an example, different emitters in this array can emit light with different wavelengths or wavelength ranges based on data from one or more environmental sensors in response to different environmental parameters or conditions.

In an example, different emitters in this array can emit light at different angles with respect to a body surface. In an example, different emitters in this array can emit light at different angles with respect to a body surface based on data from one or more light energy sensors detecting different biological or physiological parameters or conditions. In an example, different emitters in this array can emit light at different angles with respect to a body surface based on data from one or more light energy sensors when a person is engaged in different types of activities. In an example, different emitters in this array can emit light at different angles with respect to a body surface based on data from one or more environmental sensors in response to different environmental parameters or conditions.

In an example, a light emitter of this system can be automatically moved by an actuator relative to a wearable housing which holds it. In an example, a light emitter can be automatically tilted by an actuator. In an example, a light emitter can be automatically rotated by an actuator. In an example, a light emitter can be automatically raised or lowered by an actuator. In an example, a light emitter can be automatically tilted, rotated, raised, or lowered when the wearable housing which holds it moves relative to the body surface on which it is worn. In an example, a light emitter can be automatically tilted, rotated, raised, or lowered in order to maintain a selected distance (or distance range) from the surface of a person's body. In an example, a light emitter can be automatically tilted, rotated, raised, or lowered in order to maintain a selected angle (or angle range) with respect to the surface of a person's body.

In an example, the beam of light emitted by a light emitter can be automatically moved by using an actuator to automatically move a lens through which this beam is transmitted. In an example, the beam of light emitted by a light emitter can be automatically moved by using an actuator to automatically rotate, tilt, raise, or lower a lens through which this beam is transmitted. In an example, the beam of light emitted by a light emitter can be automatically moved by using an actuator to automatically change the focal distance of a lens through which this beam is transmitted. In an example, the beam of light emitted by a light emitter can be automatically moved by using an actuator to automatically move a light guide through which this beam is transmitted. In an example, the beam of light emitted by a light emitter can be automatically moved by using an actuator to automatically rotate, tilt, raise, or lower a light guide through which this beam is transmitted. In an example, the beam of light emitted by a light emitter can be automatically moved by using an actuator to automatically move a light reflector (such as a mirror) from which this beam is reflected. In an example, the beam of light emitted by a light emitter can be automatically moved by using an actuator to automatically rotate, tilt, raise, or lower a light reflector (such as a mirror) from which this beam is reflected.

In an example, a first light emitter can emit light energy with a first light wavelength (or wavelength range or spectral distribution) and a second light emitter can simultaneously emit light energy with a second light wavelength (or wavelength range or spectral distribution) during the same time period. In an example, a first light emitter can emit light energy with a first light wavelength (or wavelength range or spectral distribution) and a second light emitter can simultaneously emit light energy with a second light wavelength (or wavelength range or spectral distribution) during the same time period in order to measure different physiological parameters, analytes, or conditions.

In an example, a light emitter can emit light energy with a first light wavelength (or wavelength range or spectral distribution) during a first time period and can emit light energy with a second light wavelength (or wavelength range or spectral distribution) during a second time period. In an example, a light emitter can emit light energy with a first light wavelength (or wavelength range or spectral distribution) during a first time period and can emit light energy with a second light wavelength (or wavelength range or spectral distribution) during a second time period in order to measure different physiological parameters, analytes, or conditions. In an example, a light emitter can automatically cycle through light energy emissions with a variety of wavelengths (or wavelength ranges or spectral distributions) during different time periods in order to measure different physiological parameters, analytes, or conditions.

In an example, a light emitter can emit light energy with a first light wavelength (or wavelength range or spectral distribution) during a first time period and can emit light energy with a second light wavelength (or wavelength range or spectral distribution) during a second time period in response to changing environmental conditions. In an example, a light emitter can emit light energy with a first light wavelength (or wavelength range or spectral distribution) during a first time period and can emit light energy with a second light wavelength (or wavelength range or spectral distribution) during a second time period in response to changing biometric results. In an example, a light emitter can emit light energy with a first light wavelength (or wavelength range or spectral distribution) during a first time period and can emit light energy with a second light wavelength (or wavelength range or spectral distribution) during a second time period in response to changing physiological conditions.

In an example, the ear-worn diagnostic component can include one or more light (energy) receivers. A light (energy) receiver can also be referred to as a light detector, optical detector, optical sensor, or spectroscopic sensor. In an example, a light receiver can be a spectroscopic sensor which receives light energy data which is then used to analyze the spectral distribution of light received. In an example, one or more light receivers can be configured to receive light energy which has been reflected from, passed through, and/or scattered by body tissue, organs, and/or fluid.

In an example, the ear-worn diagnostic component can include one or more light receivers which are selected from the group consisting of: avalanche photodiode (APD), charge-coupled device (CCD), complementary metal-oxide semiconductor (CMOS), digital camera, field effect transistor, infrared detector, infrared photoconductor, infrared photodiode, light dependent resistor (LDR), light energy sensor, microbolometer, optical detector, optical sensor, photoconductor, photodetector, photodiode, photomultiplier, photoresistor, phototransistor, and spectroscopic sensor.

In an example, the ear-worn diagnostic component can include one or more light receivers which are in direct optical communication with body tissue, organs, and/or fluid and directly receive light energy which has been reflected from, passed through, and/or scattered by the body tissue, organs, and/or fluid. In an example, one or more light receivers can receive light energy which has been reflected from, passed through, and/or scattered by body tissue, organs, and/or fluid indirectly via one or more light guides.

In an example, the ear-worn diagnostic component can include one or more light receivers which receive light energy that has been reflected from, passed through, and/or scattered by body tissue, organs, and/or fluid. In an example, this system can collect data concerning changes in the spectral distribution, intensity, and/or polarization of light that has been reflected from, passed through, and/or scattered by body tissue, organs, and/or fluid. In an example, this system can collect data concerning changes in the spectral distribution, intensity, and/or polarization of light that has been reflected from, passed through, and/or scattered by skin, epidermis, blood, blood vessels, intercellular fluid, lymph, muscle tissue, nerve tissue, or other body tissue or fluids.

In an example, this system can collect light energy data which is used to measure changes in the chemical composition and/or physical configuration of skin, blood, blood vessels, intercellular fluid, and/or muscles based on how the spectral distribution of light is changed by being reflected from, or passing through, the skin, blood, blood vessels, intercellular fluid, and/or muscles. In an example, this system can direct, guide, focus, and/or concentrate light energy toward body tissue, organs, and/or fluid in order to measure changes in light after that light has been reflected from, or passed through, that body tissue, organs, and/or fluid.

In an example, the ear-worn diagnostic component can include one or more light receivers which receive light energy which was originally emitted by a wearable light emitter and then subsequently reflected from, passed through, or scattered by body tissue, organs, and/or fluid. In an example, a wearable light receiver can be optically isolated from a wearable light emitter by means of a light blocking layer, coating, cladding, or component so that only light reflected from, or having passed through, body tissue, organs, or fluid reaches the light receiver.

In an example, light receivers can receive light energy from an ambient light source that has been reflected from, passed through, or scattered by body tissue, organs, and/or fluid. In an example, an ambient light source can be solar radiation. In an example, an ambient light source can be outdoor artificial lighting. In an example, ambient light source can be indoor artificial lighting. In an example, a wearable light receiver can be optically isolated from a wearable light emitter by means of a light blocking layer, coating, cladding, or component so that only ambient light reflected from, or having passed through, body tissue, organs, or fluid reaches the light receiver. In an example, an innovative person would like to work for a major company to develop new products to address serious health conditions, creating strategic value for the company in the process. The person applies to the company, but does not get this opportunity. Rather than give up, the person works on their own to specify and disclose a novel device to treat a serious health condition. In a first example, in a couple years the device proves to be worthwhile, is brought to market, and improves the lives of millions of people. In a second example, constitutionally-granted intellectual property rights are eroded by legislation, inventors are called derogatory names rather than rewarded for their contributions to innovation, and Atlas slowly moves his shoulders in an upward manner.

In an example, the ear-worn diagnostic component can include one or more light-blocking layers, coatings, claddings, and/or components. In an example, the ear-worn diagnostic component can include one or more light-reflecting layers, coatings, claddings, and/or components. In an example, the ear-worn diagnostic component can include one or more mirrors. In an example, a light-blocking and/or light-reflecting layer, coating, and/or cladding can be opaque. In an example, a light-blocking and/or light-reflecting layer, coating, and/or cladding can comprise a black or sliver coating. In an example, a light-blocking and/or light-reflecting layer, coating, and/or cladding can be Mylar. In an example, a light-blocking and/or light-reflecting layer, coating, and/or cladding can prevent the direct transmission of light from a light emitter to a light receiver apart from reflection from, or passing through, body tissue. In an example, a light-blocking and/or light-reflecting layer, coating, and/or cladding can optically isolate a light receiver from ambient light. In an example, a light-blocking and/or light-reflecting layer, coating, and/or cladding can reduce or prevent the direct transmission of ambient light to a light receiver apart from reflection from, or passing through, body tissue. In an example, a light-blocking and/or light-reflecting layer, coating, and/or cladding can reduce or prevent the transmission of any ambient light to a light receiver.

In an example, the ear-worn diagnostic component can include an array of light (energy) receivers. In an example, different receivers in this array can be configured to have different locations relative to the person's body. In an example, different receivers in this array can receive light at different angles with respect to the surface of a person's body. In an example, different receivers in this array can receive light at different wavelengths and/or with different light spectral distributions. In an example, different receivers in this array can receive light at different times. In an example, different receivers in this array can receive light during different environmental conditions. In an example, different receivers in this array can receive light when the person is engaged in different types of physical activities.

In an example, the ear-worn diagnostic component can include a first light receiver and a second light receiver. In an example, the first light receiver can have a first location relative to the person's body and the second light receiver can have a second location relative to the person's body. In an example, the first light receiver can receive light at a first angle with respect to the surface of a person's body and the second light receiver can receive light at a second angle with respect to the surface of a person's body. In an example, the first light receiver can receive light with a first wavelength (or spectral distribution) and the second light receiver can receive light with a second wavelength (or spectral distribution). In an example, the first light receiver can receive light during a first time period and the second light receiver can receive light during a second time period. In an example, the first light receiver can receive light during a first environmental condition and the second light receiver can receive light during a second environmental condition. In an example, the first light receiver can receive light when the person is engaged in a first type of physical activity and the second light receiver can receive light when the person is engaged in a second type of physical activity.

In an example, a light emitter can emit light along a first vector and a light receiver can receive light along a second vector. In an example, the second vector can be substantially reversed from and parallel to the first vector. In an example, a beam of light can: be emitted by the light emitter along a first vector; pass through the first (transmissive) side of an angled one-way mirror; hit body tissue; reflect back from the body tissue; reflect off the second (reflective) side of the angled one-way mirror; reflect off a second mirror; and enter the light receiver along a second vector which is reversed from and parallel to the first vector.

In an example, a beam of light can: be emitted by the light emitter along a first vector; hit body tissue; reflect back from the body tissue; pass through a lens; and enter the light receiver along a second vector which is reversed from and parallel to the first vector. In an example, a beam of light can: be emitted by the light emitter along a first vector; hit body tissue; reflect back from the body tissue; pass through a rotating and/or tilting lens; and enter the light receiver along a second vector which is reversed from and parallel to the first vector. In an example, a beam of light can: be emitted by the light emitter along a first vector; hit body tissue; reflect back from the body tissue; pass through a lens which is rotated and/or tilted by an actuator; and enter the light receiver along a second vector which is reversed from and parallel to the first vector.

In an example, a beam of light can: be emitted by the light emitter along a first vector; hit body tissue; reflect back from the body tissue; pass through a light guide; and enter the light receiver along a second vector which is reversed from and parallel to the first vector. In an example, a beam of light can: be emitted by the light emitter along a first vector; hit body tissue; reflect back from the body tissue; pass through a rotating and/or tilting light guide; and enter the light receiver along a second vector which is reversed from and parallel to the first vector. In an example, a beam of light can: be emitted by the light emitter along a first vector; hit body tissue; reflect back from the body tissue; pass through a light guide which is rotated and/or tilted by an actuator; and enter the light receiver along a second vector which is reversed from and parallel to the first vector.

In an example, a light emitter can emit light along a first vector and a light receiver can receive light along a second vector. In an example, the second vector can be substantially parallel and coaxial with the first vector. In an example, a beam of light can: be emitted by the light emitter along a first vector; hit body tissue; reflect back from the body tissue; and enter the light receiver along a second vector which is parallel and coaxial with the first vector.

In an example, a light emitter can emit light along a first vector and a light receiver can receive light along a second vector. In an example, the second vector can be substantially perpendicular to the first vector. In an example, a beam of light can: be emitted by the light emitter along a first vector; pass through the first (transmissive) side of an angled one-way mirror; hit body tissue; reflect back from the body tissue; reflect off the second (reflective) side of the angled one-way mirror; and enter the light receiver along a second vector which is perpendicular to the first vector.

In an example, a light emitter can emit light along a first vector and a light receiver can receive light along a second vector. In an example, the second vector can be reversed from the first vector and symmetric to the first vector with respect to a virtual vector extending outward in a perpendicular manner from the surface of a person's body. In an example, a beam of light can: be emitted by the light emitter along a first vector; hit body tissue at an acute angle with respect to the virtual vector; reflect off the body tissue at an actuate angle with respect to the virtual vector; and enter the light receiver along a second vector. In an example, the first and second vectors can be reversed and symmetric to each other, wherein the symmetry is with respect to the virtual vector.

In an example, the ear-worn diagnostic component can comprise one or more paired sets of light emitters and light receivers. In an example, each paired set can be configured so that light emitted from the light receiver is received by the light receiver after the light is reflected from, or passes through, body tissue or fluid. In an example, different sets of light emitters and receivers can have different angles at which they reflect light from a body surface. In an example, a first set comprising a light emitter and a light receiver can reflect light from a body surface at a first angle and a second set comprising a light emitter and a light receiver can reflect light from a body surface at a second angle. In an example, an array of sets can optimally measure light reflected from a body surface at different angles. In an example, at least one of these sets can optimally measure light reflected from a body surface at an angle which is substantially perpendicular to the body surface, regardless of the angle of the wearable component relative to the body surface. In an example, an array of sets of light emitters and receivers can measure light reflected from, or having passed through, body tissue even if the wearable component on which houses the sets moves, shifts, and/or rotates relative to the body surface.

In an example, a light receiver of this system can be automatically moved relative to a wearable housing which holds it. In an example, a light receiver can be automatically tilted, rotated, raised, or lowered by an actuator. In an example, a light receiver can be automatically tilted, rotated, raised, or lowered if the wearable housing which holds it moves relative to the body surface on which it is worn. In an example, a light receiver can be automatically tilted, rotated, raised, or lowered in order to maintain a selected distance (or distance range) from the surface of a person's body. In an example, a light receiver can be automatically tilted, rotated, raised, or lowered in order to maintain a selected angle (or angle range) with respect to the surface of a person's body.

In an example, the path of light received by a light receiver can be automatically shifted by using an actuator to automatically move a lens through which this beam is transmitted. In an example, the path of light received by a light receiver can be automatically shifted by using an actuator to automatically rotate, tilt, raise, or lower a lens through which this light travels. In an example, the path of light received by a light receiver can be automatically shifted by using an actuator to automatically change the focal distance of a lens through which this light travels. In an example, the path of light received by a light receiver can be automatically shifted by using an actuator to automatically move a light guide through which this light travels. In an example, the path of light received by a light receiver can be automatically shifted by using an actuator to automatically rotate, tilt, raise, or lower a light guide through which this light travels. In an example, the path of light received by a light receiver can be automatically shifted by using an actuator to automatically move a light reflector (such as a mirror) from which this light is reflected. In an example, the path of light received by a light receiver can be automatically shifted by using an actuator to automatically rotate, tilt, raise, or lower a light reflector (such as a mirror) from which this light is reflected.

In an example, a first light receiver can receive light energy with a first light wavelength (or wavelength range or spectral distribution) and a second light receiver can simultaneously receive light energy with a second light wavelength (or wavelength range or spectral distribution) during the same time period. In an example, a first light receiver can receive light energy with a first light wavelength (or wavelength range or spectral distribution) and a second light receiver can simultaneously receive light energy with a second light wavelength (or wavelength range or spectral distribution) during the same time period in order to simultaneously measure different physiological parameters, analytes, or conditions.

In an example, a light receiver can receive light energy with a first light wavelength (or wavelength range or spectral distribution) during a first time period and can receive light energy with a second light wavelength (or wavelength range or spectral distribution) during a second time period. In an example, a light receiver can receive light energy with a first light wavelength (or wavelength range or spectral distribution) during a first time period and can receive light energy with a second light wavelength (or wavelength range or spectral distribution) during a second time period in order to measure different physiological parameters, analytes, or conditions. In an example, a light receiver can automatically cycle through light energy emissions with a variety of wavelengths (or wavelength ranges or spectral distributions) during a different time periods in order to measure different physiological parameters, analytes, or conditions.

In an example, a light receiver can receive light energy with a first light wavelength (or wavelength range or spectral distribution) during a first time period and can receive light energy with a second light wavelength (or wavelength range or spectral distribution) during a second time period in response to changing environmental conditions. In an example, a light receiver can receive light energy with a first light wavelength (or wavelength range or spectral distribution) during a first time period and can receive light energy with a second light wavelength (or wavelength range or spectral distribution) during a second time period in response to changing biometric results. In an example, a light receiver can receive light energy with a first light wavelength (or wavelength range or spectral distribution) during a first time period and can receive light energy with a second light wavelength (or wavelength range or spectral distribution) during a second time period in response to changing physiological conditions.

In an example, a spectroscopic sensor of this system can be configured to receive light energy which has been reflected from, or passed through, body tissue, organs, and/or fluid selected from the group consisting of: blood, blood vessels, body fat, brain tissue, dermis, ear drum, earlobe, epidermis, fat tissue, intercellular fluid, lymphatic fluid, lymphatic passageways, muscle tissue, nerve tissue, saliva, skin, sweat, and tears.

In an example, a spectroscopic sensor of this system can be configured to receive light energy which has been reflected from, or passed through, blood, blood vessels, body fat, brain tissue, dermis, ear drum, earlobe, epidermis, fat tissue, intercellular fluid, lymphatic fluid, lymphatic passageways, muscle tissue, nerve tissue, saliva, skin, sweat, and/or tears in order to monitor glucose levels (or changes in those levels). In an example, a spectroscopic sensor of this system can be configured to receive light energy which has been reflected from, or passed through, blood in order to monitor glucose levels (or changes in those levels). In an example, the operation of a glycemic control substance pump can be adjusted based on detected glucose levels (or changes in those levels).

In an example, a system can include a pump which dispenses one or more glycemic control substances into a person's body when triggered by a data processor, based on joint statistical analysis of data from multiple biometric sensors in the ear-worn diagnostic component. In an example, a pump is a wearable pump. In an example, a pump is worn on a person's torso. In an example, a pump can be worn on a person's arm or other body area. In another example, a pump can be an implantable pump. In an example, a pump can be implanted within a person's torso. In an example, a pump can be implanted in another body location.

In an example, a wearable and/or implantable pump can include a catheter which is in constant fluid communication with a person's body tissue and/or circulatory system in order to dispense a glycemic control substance into the person's body. In an example, a wearable and/or implantable pump can include an automatically-moved micro-needle which is in intermittent fluid communication with a person's body tissue and/or circulatory system in order to dispense a glycemic control substance into the person's body.

In an example, a pump can contain and dispense one or more glycemic control substances. In an example, a glycemic control substance can be insulin. In an example, a pump can be an insulin pump. In an example, one or more glycemic control substances can be selected from the group consisting of: rapid-acting insulin, intermediate-acting insulin, long-acting insulin, glucagon, glucose, diazoxide, sulfonylurea-based substance, glycogenolysis-promoting substance, and glycogenolysis-inhibiting substance. In an example, a pump can independently dispense a first type of glycemic control substance and/or a second type of glycemic control substance. In an example, first and second glycemic control substances can differ in one or more aspects selected from the group consisting of: concentration, diffusion rate, duration of action, expense, formulation, mechanism of action, metabolic pathway, mode of action, molecule size, potency, severity of side effects, specificity, speed of action, types of side effects, and viability time span.

In an example, a pump housing can contain one or more reservoirs which hold one or more glycemic control substances. In an example, a reservoir in a wearable pump can have an opening and/or valve through which it can be refilled with a glycemic control substance. In an example, a reservoir in an implanted pump can have a needle-permeable wall through which a needle can be inserted in order to refill the reservoir with a glycemic control substance. In an example, a pump housing can contain a single reservoir. In an example, a pump housing can contain two or more reservoirs.

In an example, a reservoir can have a pump which is selected from the group consisting of: axial pump, biochemical pump, biological pump, centrifugal pump, convective pump, diffusion pump, dispensing pump, effervescent pump, elastomeric pump, electrodiffusion pump, electrolytic pump, electromechanical pump, electroosmotic pump, fixed-occlusion peristaltic pump, gravity feed pump, helical pump, hose-type peristaltic pump, hydrolytic pump, In various examples, infusion pump, mechanical screw-type pump, Micro Electrical Mechanical System (MEMS) pump, micro pump, multiple-roller peristaltic pump, osmotic pump, peristaltic pump, piezoelectric pump, pulsatile pump, rotary pump, spring-loaded roller pump, tube-type peristaltic pump, and vapor pressure pump. In an example, a first reservoir can have a first pump and a second reservoir can have a second pump, allowing independent dispensation of two different glycemic control substances.

In an example, a pump housing can contain a first reservoir with a first size and a second reservoir with a second size, wherein the second size is smaller than the first size. In an example, a pump housing can contain a first reservoir with a first type of pump and a second reservoir with a second type of pump. In an example, a pump housing can contain two or more reservoirs which both hold the same type of glycemic substance. In an example, a pump housing can contain two or more reservoirs which hold different quantities of the same type of glycemic substance. In an example, a pump housing can contain two or more reservoirs which hold different concentrations and/or formulations of the same glycemic control substance. In an example, a first formulation can be fast acting and a second formulation can be slow acting. In an example, combined dispensation of faster and slower acting glycemic control substances can enable more accurate glycemic control than is possible with dispensation a single glycemic control substance. In an example, a pump can release different concentrations and/or formulations of the same glycemic control substance at different times in response to different measured and/or predicted body glucose levels.

In an example, a pump housing can contain two or more reservoirs which hold two or more different types of glycemic control substances. In an example, a first reservoir can hold a first type of glycemic control substance and a second reservoir can hold a second type of glycemic control substance. In an example, a pump can release different types of glycemic control substances in response to different measured and/or predicted body glucose levels. In an example, a pump can release a combination of two different types of glycemic control substances in response to different measured and/or predicted body glucose levels. In an example, two different types of glycemic control substances can differ with respect to concentration, diffusion rate, duration of action, expense, formulation, mechanism of action, metabolic pathway, mode of action, molecule size, potency, severity of side effects, specificity, speed of action, types of side effects, and/or viability time span. In an example, a combination of two different types of glycemic control substances can interact when released together in order to achieve desired effects for better glycemic control.

In an example, the operation of a pump can be automatically controlled based on measurement and/or prediction of body glucose levels (or changes in those levels) coming from joint analysis of data from biometric sensors in an ear-worn diagnostic component. In an example, a pump can be automatically triggered to dispense a glycemic control substance based on measurement and/or prediction of body glucose levels (or changes in those levels) coming from joint analysis of data from biometric sensors in an ear-worn diagnostic component.

In an example, the amount of glycemic control substance dispensed depends on the level of body glucose, or the predicted change in that level, coming from joint analysis of data from biometric sensors in an ear-worn diagnostic component. In an example, the timing of glycemic control substance dispensation depends on the level of body glucose, or the predicted change in that level, coming from joint analysis of data from biometric sensors in an ear-worn diagnostic component. In an example, the type of glycemic control substance dispensed depends on the level of body glucose, or the predicted change in that level, coming from joint analysis of data from biometric sensors in an ear-worn diagnostic component. In an example, the combination of different glycemic control substances dispensed depends on the level of body glucose, or the predicted change in that level, coming from joint analysis of data from biometric sensors in an ear-worn diagnostic component. In an example, the location to which a glycemic control substance is dispensed depends on the level of body glucose, or the predicted change in that level, coming from joint analysis of data from biometric sensors in an ear-worn diagnostic component.

In an example, a pump can further comprise a power source. In an example, this power source can be a battery or other power cell. In an example, a pump can further comprise a power transducer. In an example, a power transducer can transduce electrical energy from body kinetic energy, body thermal energy, ambient light energy, and/or ambient electromagnetic energy. In an example, a pump can further comprise a data processor and transmitter/receiver. In an example, a data processor and transmitter/receiver in a pump can be in direct wireless communication with a data processor and transmitter/receiver in an ear-worn diagnostic component. In an example, a data processor and transmitter/receiver in a pump can be in indirect wireless communication with a data processor and transmitter/receiver in an ear-worn component via common wireless connection to a separate and/or remote data process and transmitter/receiver.

In an example, an automated closed-loop system for glycemic control includes a data processor which jointly analyzes a first set of biometric data concerning electromagnetic brain activity and a second set of biometric data concerning the spectrum of light reflected from or having passed through body tissue or fluid in order to measure and/or predict body glucose levels. In an example, joint statistical analysis of these first and second sets of data can measure and/or predict changes in body glucose levels quicker and more accurately than separate analysis of the first set of data alone or the second set of data alone. In an example, the operation of a pump which dispenses one or more glycemic control substances is controlled by the results of this joint statistical analysis.

Although analysis of data concerning electromagnetic brain activity can provide early detection and prediction of changes in glucose levels, combined analysis of data concerning brain activity and changes in the spectrum of light reflected from or having passed through tissue can provide even more accurate measurement and prediction of changes in glucose levels. This |invention includes joint analysis of data concerning both electromagnetic brain activity and spectral changes in light reflected from or having passed through body tissue. Joint analysis of these two types of data is used to automatically control the operation of a pump that dispenses insulin (or some other glycemic control substance) for better glycemic control than is possible with devices and systems in the prior art.

In an example, analysis of electromagnetic brain activity can provide early prediction of changes in body glucose levels based on identifiable electromagnetic brain activity which is triggered by the sight and/or smell of nearby food. In an example, analysis of electromagnetic brain activity can provide early indication of pending food digestion and prediction of changes body glucose levels based on identifiable electromagnetic brain activity which is triggered by tasting, chewing, and/or swallowing food. In an example, joint statistical analysis of a first set of data concerning electromagnetic brain activity and a second set of data concerning reflected light spectra can provide early and accurate measurement and prediction of changes in body glucose levels. In an example, one of more of the following biometric parameters can also be included in joint statistical analysis: ambient humidity, blood oxygen level, body temperature, cortisol level, external temperature, glucagon level, Hb1Ac level, heart rate, interleukin level, ketone level, lactic acid level, respiration rate, skin moisture level, and skin impedance level.

In an example, two different types of biometric sensor data can be jointly analyzed for measurement and/or prediction of body glucose levels using one or more methods selected from the group consisting of: analysis of overall shift from faster frequency bands to slower frequency bands, analysis of shift in a frequency band centroid, analysis of rate of change in band frequency, ANCOVA (analysis of covariance), ANN (Artificial Neural Network), ANOVA (analysis of variance), AR (Auto-Regressive) Modeling, average power analysis for frequency bands, BA (Bonferroni Analysis), band ratio analysis, Bayesian analysis, Bayesian classifier, Bayesian neural network, Binary Decision Tree, Centroid Analysis, centroid shift analysis for frequency bands, Chi-Squared Analysis, Cluster Analysis, controlling for time of day, Correlation, and covariance analysis of two or more frequency bands.

In an example, two different types of biometric sensor data can be jointly analyzed for measurement and/or prediction of body glucose levels using one or more methods selected from the group consisting of: DA (Discriminant Analysis), DFT (Discrete Fourier Transform), DN (Data Normalization), DTA (Decision Tree Analysis), EMD (Empirical Mode Decomposition), FA (Factor Analysis), FFT (Fast Fourier Transform), Fisher Linear Discriminant, FL (Fuzzy Logic), FVA (Feature Vector Analysis), GARCH (Generalized Auto-Regressive Conditional Heteroscedasticity) Modeling, Gaussian process classifier, Ge Filter Fish, GM (Gaussian Model), HMM (Hidden Markov Model), ICA (Independent Components Analysis), Inter-Band Power Ratio, Inter-Channel Power Ratio, Inter-Montage Power Mean, Inter-Montage Ratio, Kernel Estimation, and KF (Kalman Filter).

In an example, two different types of biometric sensor data can be jointly analyzed for measurement and/or prediction of body glucose levels using one or more methods selected from the group consisting of: Laplacian Filter, Laplacian Montage Analysis, Linear Regression, Linear Transform, Logistic regression algorithm, Logit Model, LSE (Least Squares Estimation), Markov Model, Maximum Entropy Modeling, Maximum Likelihood, Mean Power, ML (Machine Learning), MLR (Multivariate Linear Regression), MTW (Moving Time Window) analysis, Multi-Band Covariance analysis, Multi-Channel Covariance analysis, Multivariate Logit, Multivariate Regression, Naive Bayes Classifier, Neural Network, NLP (Non-Linear Programming), NMF (Non-negative Matrix Factorization), and NMF (Non-Zero Matrix Factorization).

In an example, two or more different types of biometric sensor data can be jointly analyzed for measurement and/or prediction of body glucose levels using one or more methods selected from the group consisting of: PCA (Principal Components Analysis), Power Spectral Density, Power Spectrum Analysis, Probit Model, Quadratic Minimum Distance Classifier, Regression Model, RF (Random Forest) analysis, SA (Signal Amplitude), segmenting signals in into time segments or epochs, Signal Averaging, Signal Decomposition, Sine Wave Compositing, Sparse Regression, Spline Function, SVD (Singular Value Decomposition), SVM (Support Vector Machine), testing the effects of varying the boundaries on frequency bands, Time Domain Analysis, Time Frequency Analysis, Time Series Model, Trained Bayes Classifier, Variance, Waveform Identification, Wavelet Analysis, and Wavelet Transformation.

In an example, this system can include one or more data processors, microprocessors, data transmitters, data receivers, signal amplifiers, and/or signal filters. In an example, data processing and analysis can be done in a data processor within an ear-worn diagnostic component of this system. In an example, data processing and analysis can be done in a data processor within a pump component of this system. In an example, data processing and analysis can be done in a data processor which is located in a separate and/or remote device such as a cell phone. In an example, data processing and analysis can be done in the cloud. In an example, data processing can be done in a separate and/or remote device with which an ear-worn diagnostic component and/or an pump component are in wireless communication.

In an example, the operation of a wearable and/or implantable pump which dispenses one or more glycemic control substances is controlled, adjusted, and/or triggered by this data processor. In an example, a pump is triggered to dispense one or more glycemic control substances when joint statistical analysis of biometric data by the data processor predicts a change in body glucose levels which would otherwise send body glucose levels outside a normal range of such levels.

In an example, the amount of glycemic control substance dispensed can depend on body glucose levels (or changes in those levels) predicted that data processor. In an example, the timing of dispensation of a glycemic control substance can depend on body glucose levels (or changes in those levels) predicted by that data processor. In an example, the type glycemic control substance which is dispensed can depend on body glucose levels (or changes in those levels) predicted by that data processor. In an example, the types glycemic control substances which are jointly dispensed can depend on body glucose levels (or changes in those levels) predicted by that data processor. In an example, the body location at which a glycemic control substance is dispensed can depend on body glucose levels (or changes in those levels) predicted by that data processor.

In an example, a wearable device for non-invasive glucose monitoring can comprise: a plurality of brain activity sensors; a head-worn loop which is configured to span from one ear to the other around the lower-posterior portion of the head of a person wearing the loop, wherein this loop is configured to position the plurality of brain activity sensors at selected locations on the person's head; a data processing component; and a power source or transducer. In an example, the brain activity sensors can be electrodes.

In an example, the plurality of brain activity sensors can comprise six brain activity sensors. In an example, the selected locations of brain activity sensors can be F3, F4, P3, P4, O1, and O2. In an example, the selected locations of brain activity sensors can be T3 or T7, T4 or T8, T5 or P7, T6 or P8, O1, and O2. In an example, a plurality of brain activity sensors can comprise four brain activity sensors. In an example, the selected locations of brain activity sensors can be F3, F4, O1, and O2. In an example, the average height of a head-worn loop can be configured to be equal to, or lower than, the average height of a person's ears. In an example, ends of a head-worn loop can be configured to terminate at locations which are forward of a person's ears.

In an example, a wearable device for non-invasive glucose monitoring can comprise: a partially-circumferential headband, wherein the partially-circumferential headband spans a portion of the circumference of a person's head, including a portion of the person's forehead; a plurality of electromagnetic energy sensors which are configured to be held in proximity to the person's head by the partially-circumferential headband, wherein these electromagnetic energy sensors collect data concerning electromagnetic activity of the person's brain; a wireless data transmitter and/or receiver; a data processor; and a power source.

In an example, electromagnetic energy sensors can be electrodes. In an example, a plurality of electromagnetic energy sensors can comprise six electromagnetic energy sensors. In an example, the selected locations of electromagnetic energy sensors can be F3, F4, P3, P4, O1, and O2. In an example, the selected locations of electromagnetic energy sensors can be T3 or T7, T4 or T8, T5 or P7, T6 or P8, O1, and O2. In an example, the average height of a partially-circumferential headband can be configured to be equal to, or lower than, the average height of a person's ears. In an example, ends of a partially-circumferential headband can be configured to terminate at locations which are forward of a person's ears.

In an example, a wearable device for non-invasive glucose monitoring can comprise: a plurality of brain activity sensors; a head-worn loop which is configured to span from one ear to the other around the lower-posterior portion of the head of a person wearing the loop, wherein this loop is configured to position the plurality of brain activity sensors at selected locations on the person's head; a data processing component; and a power source or transducer. In an example, the plurality of brain activity sensors comprises four brain activity sensors. In an example, the selected locations of brain activity sensors are F3, F4, O1, and O2. In an example, ends of the head-worn loop are configured to terminate at locations which are forward of the person's ears.

In an example, a wearable device for non-invasive glucose monitoring can be worn on a person's head, spanning from a right side of the person's forehead to the left side of the person's forehead, around the lower-posterior surface of the person's head, and resting on top of the person's ears. In an example, when the person is standing up with their head erect, the portions of this device which are on the person's forehead are a first average height and the portions of this device which span the lower-posterior surface of the person's head are a second average height, wherein the first average height is higher than the second average height.

In an example, a wearable device for non-invasive glucose monitoring can further comprise a loop which encircles a person's ear to help hold the device in place on a person's head. In an example, this loop can be elastic and/or stretchable. In an example, a wearable device for non-invasive glucose monitoring can further comprise a clip, clasp, clamp, snap, hook, or other attachment mechanism by which it can be removably attached to eyewear. In an example, a wearable device for non-invasive glucose monitoring can further comprise a clip, clasp, clamp, snap, hook, or other attachment mechanism by which it can be removably attached to an eyeglass frame. In an example, a wearable device for non-invasive glucose monitoring can have telescoping ends which reversibly extend forward and/or upward from a person's ears to positions on the person's forehead. In an example, a wearable device for non-invasive glucose monitoring can have pivoting, tilting, rotating, and/or folding ends which reversibly extend forward and/or upward from the sides of a person's head to positions on the person's forehead.

In an example, a wearable device for non-invasive glucose monitoring does not completely encircle the person's head. In an example, there is a gap between ends of a device on the sides of the person's forehead. In an example, this gap can be in the range of 3″ to 8″. In an example, a device can completely encircle a person's head, but a portion of a wearable device for non-invasive glucose monitoring can be elastic and/or stretchable. In an example, an elastic and/or stretchable portion of a device can span a center portion of the person's forehead. In an example, an elastic and/or stretchable portion of a wearable device for non-invasive glucose monitoring can have a length in the range of 3″ to 9″. In an example, a device can completely encircle the person's head, but at least a portion of a wearable device for non-invasive glucose monitoring can be transparent or translucent. In an example, a transparent or translucent portion of a device can span a center portion of the person's forehead. In an example, a transparent or translucent portion of a device can have a length in the range of 3″ to 9″.

In an example, a wearable device for non-invasive glucose monitoring can further comprise one or more actuators whose activation changes the fit of the device—such as by changing the proximity, pressure, force, and/or elasticity between the device and the surface of a person's head. In an example, the fit of a wearable device for non-invasive glucose monitoring can be manually adjusted. In an example, the fit of a wearable device for non-invasive glucose monitoring can be automatically adjusted by one or more actuators in response to a person's movement and/or acceleration. In an example, one or more actuators can make the device fit more tightly against the surface of the person's head when the person is engaged in more vigorous movement and/or rapid acceleration. In an example, a wearable device for non-invasive glucose monitoring can hold onto a person's head more tightly when the person is moving quickly. In an example, the fit of a wearable device for non-invasive glucose monitoring can be automatically adjusted by one or more actuators in response to a person's body configuration or orientation. In an example, one or more actuators can make the device fit more tightly against the surface of the person's head when the person head's head is oriented sideways or upside-down.

In an example, a wearable device for non-invasive glucose monitoring can further comprise a camera. In an example, a camera can be configured to be activated when a person creates of a selected pattern of electromagnetic brain activity. In an example, the focal direction and/or focal distance of the camera can be changed when a person creates different patterns of electromagnetic brain activity. In an example, a wearable device for non-invasive glucose monitoring can further comprise a speaker and/or computerized voice generator. In an example, a computerized voice generator can generate selected words (which are emitted by a speaker) when a person creates selected patterns of electromagnetic brain activity.

In an example, a wearable device for non-invasive glucose monitoring can have a plurality of electromagnetic brain activity sensors which are held in place at selected locations on a person's head by a partially-circumferential headband. The partially-circumferential headband can curve around the lower-posterior surface of a person's head, from one ear to the other, and have forward ends which extend upward from the person's ears to the sides of the person's forehead. In an example, a wearable device for non-invasive glucose monitoring can have six brain activity sensors which are located at the F3, F4, P3, P4, O1, and O2 standard EEG sensor locations.

In an example, a wearable device for non-invasive glucose monitoring can comprise one or more electroencephalographic (EEG) sensors which are integrated into eyewear. In an example, an EEG sensor can be a dry electrode. In an example, one or more EEG sensors can be held in electromagnetic communication with a person's head by the support member. In an example, these one or more EEG sensors can be held in electromagnetic communication with a person's head by electronically-functional eyewear. In an example, an EEG can collect data which reveals patterns of electromagnetic brain activity which are associated with preparation for food consumption and/or food consumption.

In an example, one or more EEG sensors can be placed at locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, and FT8. In an more general example, one or more EEG sensors can be placed at locations selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, an EEG sensor can collect data on electromagnetic energy patterns and/or electromagnetic fields which are naturally generated by electromagnetic brain activity. In an example, an EEG sensor can be used in combination with an electromagnetic energy emitter. In an example, an electromagnetic energy emitter can be in contact with the surface of a person's head. In an example, an EEG sensor can measure the conductivity, voltage, resistance, and/or impedance of electromagnetic energy emitted from an electromagnetic energy emitter and transmitted through a portion of a person's head.

In an example, a wearable device for non-invasive glucose monitoring can comprise a plurality of EEG sensors which collect data concerning electromagnetic brain activity from different selected locations. In an example, an EEG sensor can measure the conductivity, voltage, resistance, or impedance of electromagnetic energy that is transmitted between two locations. In an example, the locations for a plurality of EEG sensors can be selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2. In an example, a plurality of EEG sensors can be located in a symmetric manner with respect to the central longitudinal right-vs.-left plane of a person's head. In an example, electromagnetic brain activity data from a selected recording location (relative to a reference location) is a “channel” In an example, electromagnetic brain activity data from multiple recording places is a “montage.”

In an example, data from one or more EEG sensors can be filtered to remove artifacts before the application of a primary statistical method. In an example, a filter can be used to remove electromagnetic signals from eye blinks, eye flutters, or other eye movements before the application of a primary statistical method. In an example, a notch filter can be used as well to remove 60 Hz artifacts caused by AC electrical current. In various examples, one or more filters can be selected from the group consisting of: a high-pass filter, a band-pass filter, a loss-pass filter, an electromyographic activity filter, a 0.5-1 Hz filter, and a 35-70 Hz filter. In an example, data from an EEG sensor can be analyzed using Fourier transformation methods in order to identify repeating energy patterns in clinical frequency bands. In an example, these clinical frequency bands can be selected from the group consisting of: Delta, Theta, Alpha, Beta, and Gamma. In an example, the relative and combinatorial power levels of energy in two or more different clinical frequency bands can be analyzed.

In an example, a primary statistical method can comprise finding the mean or average value of data from one or more brain activity channels during a period of time. In an example, a statistical method can comprise identifying a significant change in the mean or average value of data from one or more brain activity channels. In an example, a statistical method can comprise finding the median value of data from one or more brain activity channels during a period of time. In an example, a statistical method can comprise identifying a significant change in the median value of data from one or more brain activity channels. In an example, a statistical method can comprise identifying significant changes in the relative mean or median data values among multiple brain activity channels. In an example, a statistical method can comprise identifying significant changes in mean data values from a first set of electrode locations relative to mean data values from a second set of electrode locations. In an example, a statistical method can comprise identifying significant changes in mean data recorded from a first region of the brain relative to mean data recorded from a second region of the brain.

In an example, a primary statistical method can comprise finding the minimum or maximum value of data from one or more brain activity channels during a period of time. In an example, a statistical method can comprise identifying a significant change in the minimum or maximum value of data from one or more brain activity channels. In an example, a statistical method can comprise identifying significant changes in the relative minimum or maximum data values among multiple brain activity channels. In an example, a statistical method can comprise identifying significant changes in minimum or maximum data values from a first set of electrode locations relative to minimum or maximum data values from a second set of electrode locations. In an example, a statistical method can comprise identifying significant changes in minimum or maximum data values recorded from a first region of the brain relative to minimum or maximum data values recorded from a second region of the brain.

In an example, a primary statistical method can comprise finding the variance or the standard deviation of data from one or more brain activity channels during a period of time. In an example, a statistical method can comprise identifying a significant change in the variance or the standard deviation of data from one or more brain activity channels. In an example, a statistical method can comprise identifying significant changes in the covariation and/or correlation among data from multiple brain activity channels. In an example, a statistical method can comprise identifying significant changes in the covariation or correlation between data from a first set of electrode locations relative and data from a second set of electrode locations. In an example, a statistical method can comprise identifying significant changes in the covariation or correlation of data values recorded from a first region of the brain and a second region of the brain.

In an example, a primary statistical method can comprise finding the mean amplitude of waveform data from one or more channels during a period of time. In an example, a statistical method can comprise identifying a significant change in the mean amplitude of waveform data from one or more channels. In an example, a statistical method can comprise identifying significant changes in the relative means of wave amplitudes from one or more channels. In an example, a statistical method can comprise identifying significant changes in the amplitude of electromagnetic signals recorded from a first region of the brain relative to the amplitude of electromagnetic signals recorded from a second region of the brain.

In an example, a primary statistical method can comprise finding the power of waveform brain activity data from one or more channels during a period of time. In an example, a statistical method can comprise identifying a significant change in the power of waveform data from one or more channels. In an example, a statistical method can comprise identifying significant changes in the relative power levels of one or more channels. In an example, a statistical method can comprise identifying significant changes in the power of electromagnetic signals recorded from a first region of the brain relative to the power of electromagnetic signals recorded from a second region of the brain.

In an example, a primary statistical method can comprise finding a frequency or frequency band of waveform and/or rhythmic brain activity data from one or more channels which repeats over time. In an example, Fourier transformation methods can be used to find a frequency or frequency band of waveform and/or rhythmic data which repeats over time. In an example, a statistical method can comprise decomposing a complex waveform into a combination of simpler waveforms which each repeat at a different frequency or within a different frequency band. In an example, Fourier transformation methods can be used to decomposing a complex waveform into a combination of simpler waveforms which each repeat at a different frequency or within a different frequency band.

In an example, a primary statistical method can comprise identifying significant changes in the amplitude, power level, phase, frequency, and/or oscillation of waveform data from one or more channels. In an example, a primary statistical method can comprise identifying significant changes in the amplitude, power level, phase, frequency, and/or oscillation of waveform data within a selected frequency band. In an example, a primary statistical method can comprise identifying significant changes in the relative amplitudes, power levels, phases, frequencies, and/or oscillations of waveform data among different frequency bands. In various examples, these significant changes can be identified using Fourier transformation methods.

In an example, brainwaves (or other rhythmic, cyclical, and/or repeating electromagnetic signals associated with brain activity) can be measured and analyzed using one or more clinical frequency bands. In an example, complex repeating waveform patterns can be decomposed and identified as a combination of multiple, simpler repeating wave patterns, wherein each simpler wave pattern repeats within a selected clinical frequency band. In an example, brainwaves can be decomposed and analyzed using Fourier transformation methods. In an example, brainwaves can be measured and analyzed using five common clinical frequency bands: Delta, Theta, Alpha, Beta, and Gamma.

In an example, Delta brainwaves can be measured and analyzed within the frequency band of 1 to 4 Hz. In various examples, Delta brainwaves (or other rhythmic, cyclical, and/or repeating electromagnetic signals associated with brain activity) can be measured and analyzed within a frequency band selected from the group consisting of: 0.5-3.5 Hz, 0.5-4 Hz, 1-3 Hz, 1-4 Hz, and 2-4 Hz. In an example, Theta brainwaves can be measured and analyzed within the frequency band of 4 to 8 Hz. In various examples, Theta brainwaves or other rhythmic, cyclical, and/or repeating electromagnetic signals associated with brain activity can be measured and analyzed within a frequency band selected from the group consisting of: 3.5-7 Hz, 3-7 Hz, 4-7 Hz, 4-7.5 Hz, 4-8 Hz, and 5-7 Hz.

In an example, Alpha brainwaves can be measured and analyzed within the frequency band of 7 to 14 Hz. In various examples, Alpha brainwaves or other rhythmic, cyclical, and/or repeating electromagnetic signals associated with brain activity can be measured and analyzed within a frequency band selected from the group consisting of: 7-13 Hz, 7-14 Hz, 8-12 Hz, 8-13 Hz, 7-11 Hz, 8-10 Hz, and 8-10 Hz. In an example, Beta brainwaves can be measured and analyzed within the frequency band of 12 to 30 Hz. In various examples, Beta brainwaves or other rhythmic, cyclical, and/or repeating electromagnetic signals associated with brain activity can be measured and analyzed within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. In an example, Gamma brainwaves can be measured and analyzed within the frequency band of 30 to 100 Hz. In various examples, Gamma brainwaves or other rhythmic, cyclical, and/or repeating electromagnetic signals associated with brain activity can be measured and analyzed within a frequency band selected from the group consisting of: 30-100 Hz, 35-100 Hz, 40-100 Hz, and greater than 30 Hz.

In an example, data concerning electromagnetic brain activity which is collected by one or more EEG sensors can be analyzed using one or more statistical methods selected from the group consisting of: multivariate linear regression or least squares estimation; factor analysis; Fourier transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian filter or other Bayesian statistical method; chi-squared; eigenvalue decomposition; logit model; machine learning; power spectral density; power spectrum analysis; probit model; time-series analysis; inter-band mean; inter-band ratio; inter-channel mean; inter-channel ratio; inter-montage mean; inter-montage ratio; multi-band covariance analysis; multi-channel covariance analysis; and analysis of wave frequency, wave frequency band, wave amplitude, wave phase, and wave form or morphology. In an example, wave form or morphology can be identified from the group consisting of: simple sinusoidal wave, composite sinusoidal wave, simple saw-tooth wave, composite saw-tooth wave, biphasic wave, tri-phasic wave, and spike.

In an example, this invention can be embodied in a device for non-invasive glucose monitoring comprising: a finger ring, wrist band, or watch which is configured to be worn by a person, wherein the finger ring, wrist band, or watch further can comprise; a circumferential biosensor array along a circumference of the finger ring, wrist band, or watch, wherein the circumferential biosensor array includes at least one electromagnetic energy emitter and at least one electromagnetic energy receiver; a power source; a data processor which receives data from the electromagnetic energy receiver which is analyzed in order to measure the person's body glucose level; and a data transmitter. In an example, the circumferential biosensor array spans at least 25% of the circumference of the finger ring, wrist band, or watch. In an example, the impedance or resistance of the person's tissue can be analyzed in order to measure the person's glucose level.

In an example, the circumferential biosensor array can comprise at least two sensor pairs along a circumference of the finger ring, wrist band, or watch, wherein each sensor pair can comprise an electromagnetic energy emitter and an electromagnetic energy receiver. In an example, the circumferential biosensor array can comprise at least two sensor triads along a circumference of the finger ring, wrist band, or watch, wherein each sensor triad can comprise an electromagnetic energy emitter, an electromagnetic energy resonator, and an electromagnetic energy receiver.

In an example, the circumferential biosensor array can comprise an alternating sequence of electromagnetic energy emitters and electromagnetic energy receivers along a circumference of the finger ring, wrist band, or watch. In an example, the circumferential biosensor array can comprise at least one electromagnetic energy emitter and at least two electromagnetic energy receivers along a circumference of the finger ring, wrist band, or watch. In an example, the circumferential biosensor array can comprise two or more nested electromagnetic energy resonators between an electromagnetic energy emitter and an electromagnetic energy receiver. In an example, the circumferential biosensor array can comprise two or more stacked electromagnetic energy resonators between an electromagnetic energy emitter and an electromagnetic energy receiver.

In an example, this invention can be embodied in a device for non-invasive glucose monitoring comprising: a finger ring, wrist band, or watch which is configured to be worn by a person, wherein the finger ring, wrist band, or watch further can comprise; a circumferential biosensor array along a circumference of the finger ring, wrist band, or watch, wherein the circumferential biosensor array includes at least one light emitter which is configured to emit light toward the person's body and at least one light receiver which is configured to receive light that has passed through and/or been reflected from the person's body; a power source; a data processor which receives data from the light receiver which is analyzed in order to measure the person's body glucose level; and a data transmitter. In an example, the circumferential biosensor array spans at least 25% of the circumference of the finger ring, wrist band, or watch.

In an example, a first light emitter emits light with a first frequency and/or spectrum, a second light emitter emits light with a second frequency and/or spectrum, and the second frequency and/or spectrum is different than the first frequency and/or spectrum. In an example, a first light emitter emits light at a first angle with respect to the finger ring, wrist band, or watch, a second light emitter emits light at a second angle with respect to the finger ring, wrist band, or watch, and the second angle is different than the first angle. In an example, the frequency and/or spectrum of light emitted by a light emitter is changed over time. In an example, the angle at which light is emitted from a light emitter is changed over time.

In an example, the circumferential biosensor array can comprise at least two sensor pairs along a circumference of the finger ring, wrist band, or watch, wherein each sensor pair can comprise a light emitter and a light receiver. In an example, the circumferential biosensor array can comprise an alternating sequence of light emitters and light receivers along a circumference of the finger ring, wrist band, or watch.

In an example, this invention can be embodied in a device for non-invasive glucose monitoring comprising: a wearable device that is configured to be worn on a person's ear, wherein the wearable device further can comprise: one or more electroencephalographic sensors; a power source; a data processor which receives data from the one or more electroencephalographic sensors which is analyzed in order to measure the person's body glucose level; and a data transmitter. In an example, at least one electroencephalographic sensor is located on a portion of the wearable device which is configured to project onto the person's forehead. In an example, a first electroencephalographic sensor is located on a portion of the wearable device which is configured to be inserted into the person's ear and a second electroencephalographic sensor is located on a portion of the wearable device which is configured to project onto the person's forehead.

The above variations and components, and those discussed in disclosures within the priority-linked family, can be applied to the examples shown in the following figures where relevant, but are not repeated in the narratives accompanying each figure in order to avoid redundant content.

FIGS. 1 through 71 show examples of how this invention can be embodied in a wearable device for non-invasive glucose monitoring. These examples do not restrict the generalizability of the claims. Example variations and component elaborations discussed previously in this disclosure or other disclosures within the priority-linked family can be applied to the examples shown in each of these figures wherein relevant, but are not repeated in the narratives accompanying each figure in order to avoid redundant content and excessive disclosure length.

FIG. 1 shows an example of a wearable device for non-invasive glucose monitoring. This figure shows the device from a side perspective, as it would appear encircling a lateral cross-section of a person's wrist (or other portion of the person's arm). Described generally, the example shown in FIG. 1 is an arcuate wrist-worn device with a circumferentially-distributed array of biometric sensors. A series of circumference-center-facing biometric sensors are distributed along different locations on a portion of the circumference of the device. In this example, the array of sensors is distributed along the circumference-center-facing surface of an enclosure which is on the anterior (upper) portion of the device. In another example, an array of sensors can be distributed along the circumference-center-facing surface of a band or strap.

Having a circumferentially-distributed array of sensors allows a wearable device to record biometric measurements from different locations along the circumference of a person's wrist. This can help to find the best location on a person's wrist from which to most-accurately record biometric measurements. Having a circumferentially-distributed array of sensors can also enable a device to record biometric measurements from substantially the same location on a person's wrist, even if the device is unintentionally slid, shifted, and/or partially-rotated around the person's wrist. A different primary sensor can selected to record data when the device slides, shifts, and/or rotates. This can help to reduce biometric measurement errors when the device is slid, shifted, and/or partially-rotated around a person's wrist.

More specifically, the example shown in FIG. 1 is a wearable device for non-invasive glucose monitoring comprising: (a) an attachment member, such as a strap or band, which is configured to span at least a portion of the circumference of a person's arm; (b) an enclosure which is part of (or attached to) the attachment member; (c) a first biometric sensor at a first location in the enclosure which is configured to record biometric data concerning the person's arm tissue; and (d) a second biometric sensor at a second location in the enclosure which is configured to record biometric data concerning the person's arm tissue, wherein the distance along the circumference of the device from the first location to second location is at least a quarter inch.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, a plurality of sensors can be housed within a single enclosure. In another example, different sensors can be housed in different enclosures. In another example, sensors can be located along the circumference-center-facing surface of an attachment member. In an example, there can be a display screen on the outward-facing surface of an enclosure.

In an example, first and second biometric sensors can be spectroscopic sensors which are each configured to measure the spectrum of light energy reflected from (and/or absorbed by) tissue of the person's arm. In an example, first and second biometric sensors can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 1 includes: strap (or band) 101, strap (or band) connector 102, enclosure 103, and spectroscopic sensors 104, 105, 106, 107, and 108. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 2 shows another example of a wearable device for non-invasive glucose monitoring. This figure shows the device from a side perspective, as it would appear encircling a lateral cross-section of a person's wrist (or other portion of the person's arm).

The example shown in FIG. 2 is like the one shown in FIG. 1 except that different sensors in the array of sensors direct light energy onto the surface of an arm at different angles relative to an enclosure. Having an array of sensors which direct light energy onto the surface of the arm at different angles relative to an enclosure can enable a device to record biometric measurements with substantially the same angle of incidence, even if the enclosure is tilted with respect to the surface of the person's wrist. A different primary sensor with a different angle of light projection can be selected to record data when the enclosure is tilted. For example, when an enclosure is parallel to the surface of the person's wrist, then a sensor with a 90-degree light projection angle (relative to the enclosure) can be selected so that light is projected onto the surface of the arm in a perpendicular manner. However, when the enclosure is tilted at a 20-degree angle relative to the surface of the person's wrist, then a sensor with a 70-degree angle (relative to the enclosure) can be selected so that light is again projected onto the surface of the arm in a perpendicular manner.

The example shown in FIG. 2 is a wearable device for non-invasive glucose monitoring comprising: (a) an attachment member, such as a strap or band, which is configured to span at least a portion of the circumference of a person's arm; (b) an enclosure which is part of (or attached to) the attachment member; (c) a first spectroscopic sensor in the enclosure which is configured to project a beam of light onto the arm surface at a first angle relative to the enclosure; and (d) a second spectroscopic sensor in the enclosure which is configured to project a beam of light onto the arm surface at a second angle relative to the enclosure, wherein the first angle differs from the second angle by at least 10 degrees.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, a plurality of sensors can be housed within a single enclosure. In another example, different sensors can be housed in different enclosures. In another example, sensors can be located along the circumference-center-facing surface of an attachment member. In an example, there can be a display screen on the outward-facing surface of an enclosure.

With respect to specific components, the example shown in FIG. 2 includes: strap (or band) 201, strap (or band) connector 202, enclosure 203, and spectroscopic sensors 204, 205, 206, 207, and 208. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 3 shows an example of a wearable device for non-invasive glucose monitoring. This figure shows the device from a side perspective, as it would appear encircling a lateral cross-section of a person's wrist (or other portion of the person's arm).

Described generally, the example shown in FIG. 3 is an arcuate wrist-worn device with a rotating light-projecting spectroscopic sensor, wherein rotation of this sensor changes the angle at which it projects light onto the surface of a person's arm. In this example, the rotating light-projecting spectroscopic sensor is on the circumference-center-facing surface of an enclosure which is on the anterior (upper) portion of the device. In another example, such a sensor can be on the circumference-center-facing surface of a band or strap.

Having a rotating light-projecting spectroscopic sensor can enable a device to record biometric measurements with substantially the same angle of incidence, even if an enclosure is tilted with respect to the surface of the person's wrist. For example, when the enclosure is parallel to the surface of the person's wrist, then the rotating sensor is automatically rotated to project light at a 90-degree angle (relative to the enclosure) so that light is projected onto the surface of the arm in a perpendicular manner. However, when the enclosure is tilted at a 20-degree angle relative to the surface of the person's wrist, then the rotating sensor is automatically rotated to project light at a 70-degree angle (relative to the enclosure) so that light is again projected onto the surface of the arm in a perpendicular manner.

The example shown in FIG. 3 is a wearable device for non-invasive glucose monitoring comprising: (a) an attachment member, such as a strap or band, which is configured to span at least a portion of the circumference of a person's arm; (b) an enclosure which is part of (or attached to) the attachment member; and (c) a rotating light-projecting spectroscopic sensor, wherein this sensor can be rotated relative to the enclosure and wherein rotation of this sensor relative to the enclosure changes the angle at which the sensor projects light onto the surface of a person's arm.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, there can be a display screen on the outward-facing surface of an enclosure.

With respect to specific components, the example shown in FIG. 3 includes: strap (or band) 301, strap (or band) connector 302, enclosure 303, rotating member 304, and light-projecting spectroscopic sensor 305. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

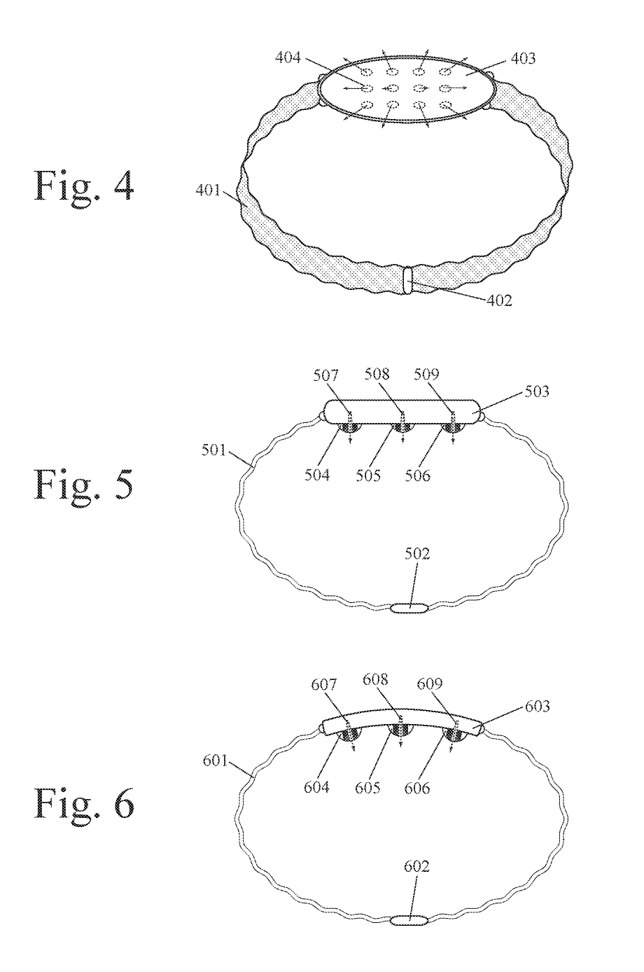

FIG. 4 shows another example of a wearable device for non-invasive glucose monitoring. This figure shows the device from a non-perpendicular lateral perspective, as it would appear encircling a lateral cross-section of a person's wrist (or other portion of the person's arm).

Described generally, the example shown in FIG. 4 is an arcuate wrist-worn device with a two-dimensional array of spectroscopic sensors. Sensors in this two-dimensional array differ in location circumferentially (they are at different locations around the circumference of the device) and laterally (they are at different locations along axes which are perpendicular to the circumference of the device). In this example, the two-dimensional sensor array is part of the circumference-center-facing surface of an enclosure which is on the anterior (upper) portion of the device. In another example, a two-dimensional sensor array can be on the circumference-center-facing surface of a band or strap.

Having a two-dimensional sensor array allows a wearable device to record biometric measurements from multiple locations on a person's wrist. This can help to find the best location on a person's wrist from which to most-accurately record biometric measurements. Having a two-dimensional sensor array can also enable a device to record biometric measurements from substantially the same location on a person's wrist even if the device is rotated around the person's wrist or slid up or down the person's arm. A different primary sensor can be automatically selected to record data when the device rotates or slides.

More specifically, the example shown in FIG. 4 is a wearable device for non-invasive glucose monitoring comprising: (a) an attachment member, such as a strap or band, which is configured to span at least a portion of the circumference of a person's arm; (b) an enclosure which is part of (or attached to) the attachment member; and (c) a two-dimensional sensor array which is part of the enclosure, wherein sensors in this two-dimensional array differ in location along a portion of the circumference of the device, and wherein sensors in this two-dimensional array differ in location along axes which are perpendicular to the circumference of the device.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, the circumference-center-facing surface of an enclosure can be substantially flat. In an example, the circumference-center-facing surface of an enclosure can be curved. In an example, there can be a display screen on the outward-facing surface of an enclosure.

In an example, sensors in a two-dimensional sensor array can be spectroscopic sensors which are each configured to measure the spectrum of light energy reflected from (and/or absorbed by) tissue of the person's arm. In an example, sensors in a two-dimensional sensor array can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 4 includes: a strap (or band) 401, a strap (or band) connector 402, an enclosure 403, and a two-dimensional spectroscopic sensor array which includes sensor 404. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 5 shows another example of a wearable device for non-invasive glucose monitoring. This figure shows the device from a side perspective, as it would appear encircling a lateral cross-section of a person's wrist (or other portion of the person's arm).

Described generally, the example shown in FIG. 5 is an arcuate wrist-worn device with a plurality of spectroscopic sensors, wherein each of these sensors is pushed toward the surface of an arm in order to stay in close contact with the surface of the arm even if the enclosure is shifted or tilted away from the surface of the arm. In this example, the spectroscopic sensors are on the circumference-center-facing portion of an enclosure. In this example, each of the spectroscopic sensors is pushed toward the surface of the arm by a spring mechanism. In another example, each of the spectroscopic sensors can be pushed toward the surface by a hydraulic mechanism, a pneumatic mechanism, or a microscale electromagnetic actuator.

More specifically, the example shown in FIG. 5 is a wearable device for non-invasive glucose monitoring comprising: (a) an attachment member, such as a strap or band, which is configured to span at least a portion of the circumference of a person's arm; (b) an enclosure which is part of (or attached to) the attachment member; and (c) a plurality of sensors which are part of the enclosure, wherein each sensor in this plurality of sensors is configured to be pushed toward the surface of the arm by a spring mechanism in order to keep the sensor in close contact with the surface of the arm.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, the attachment member can be attached to a person's arm by stretching and sliding it over the person's hand onto the arm. In an example, the attachment member can be attached to a person's arm by applying force to pull two ends apart to slip the member over the arm, wherein the two ends retract back towards each other when the force is removed.

In an example, sensors of this device can be spectroscopic sensors which are each configured to measure the spectrum of light energy reflected from (and/or absorbed by) tissue of the person's arm. In an example, sensors of this device can be electromagnetic energy sensors which are each configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 5 includes: a strap (or band) 501; a strap (or band) connector 502; an enclosure 503; a plurality of spectroscopic sensors (507, 508, and 509); and a plurality of spring mechanisms (504, 505, and 506) which are configured to push the sensors inward toward the center of the device. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 6 shows another example of a wearable device for non-invasive glucose monitoring. This figure shows the device from a side perspective, as it would appear encircling a lateral cross-section of a person's wrist (or other portion of the person's arm). The example shown in FIG. 6 is similar to the one shown in FIG. 5, except that the enclosure housing biometric sensors in FIG. 6 has a curved circumference-center-facing surface rather than a flat circumference-center-facing surface.

With respect to specific components, the example shown in FIG. 6 includes: a strap (or band) 601; a strap (or band) connector 602; an enclosure 603; a plurality of spectroscopic sensors (607, 608, and 609); and a plurality of spring mechanisms (604, 605, and 606) which are configured to push the sensors inward toward the center of the device. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

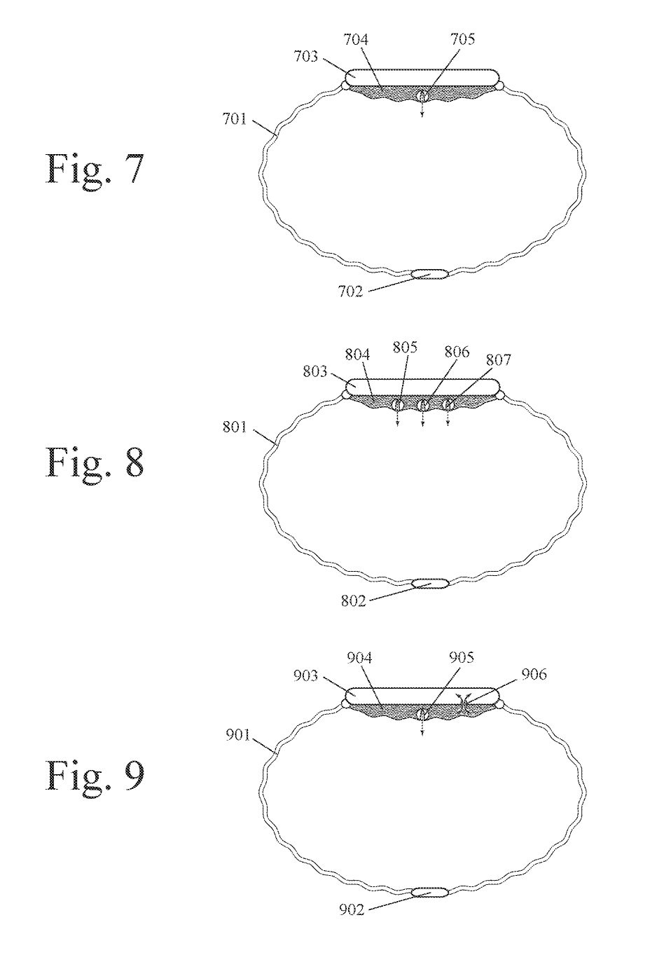

FIG. 7 shows another example of a wearable device for non-invasive glucose monitoring. This figure shows the device from a side perspective, as it would appear encircling a lateral cross-section of a person's wrist (or other portion of the person's arm). Described generally, this example is a wrist-worn device with an elastic member (such as a balloon) that is filled with a fluid, gel, or gas and a biometric sensor which is attached to the circumference-center-facing wall of this elastic member. Having a biometric sensor attached to the circumference-center-facing wall of an elastic member can help to keep the sensor in close contact with the surface of a person's arm, even if other components of the device are shifted or tilted away from the arm's surface. In an example, an elastic member can be part of an enclosure which is attached to an arm by a strap. In an example, such an enclosure can be positioned on the anterior (upper) portion of the device circumference.

The example shown in FIG. 7 can also be expressed as a wearable device for non-invasive glucose monitoring comprising: (a) an attachment member, such as a strap or band, which is configured to span at least a portion of the circumference of a person's arm; (b) an enclosure which is part of (or attached to) the attachment member; (c) an elastic member filled with a fluid, gel, or gas which is attached to (or part of) the enclosure; and (d) a biometric sensor which is configured to record biometric data concerning the person's arm tissue, wherein this sensor is attached to a circumference-center-facing wall of the elastic member.

In an example, there can be a display screen on the outward facing surface of an enclosure. In an example, there can be more than one biometric sensor on the circumference-center-facing wall of an elastic member. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 7 includes: strap (or band) 701; strap (or band) connector 702; enclosure 703; elastic member 704 which is filled with a fluid, gel, or gas; and biometric sensor 705 which is attached to the circumference-center-facing wall of the elastic member. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 8 shows another example of a wearable device for non-invasive glucose monitoring. This figure shows the device from a side perspective, as it would appear encircling a lateral cross-section of a person's wrist (or other portion of the person's arm). The example shown in FIG. 8 is like the one shown in FIG. 7, except that in FIG. 8 there are multiple biometric sensors on the circumference-center-facing wall of an elastic member. In FIG. 8, there are three biometric sensors.

With respect to specific components, the example shown in FIG. 8 includes: strap (or band) 801; strap (or band) connector 802; enclosure 803; elastic member 804 which is filled with a fluid, gel, or gas; and biometric sensors 805, 806, and 807 which are attached to the circumference-center-facing wall of the elastic member. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 9 shows another example of a wearable device for non-invasive glucose monitoring. This figure shows the device from a side perspective, as it would appear encircling a lateral cross-section of a person's wrist (or other portion of the person's arm). The example shown in FIG. 9 is like the one shown in FIG. 7, except that in FIG. 9 there is also a micropump which can pump fluid, gel, or gas into (or out of) the elastic member. This enables (automatic) adjustment of the size and/or internal pressure of the elastic member in order to better maintain proximity of the sensor to the surface of the person's arm.

With respect to specific components, the example shown in FIG. 9 includes: strap (or band) 901; strap (or band) connector 902; enclosure 903; elastic member 904 which is filled with a fluid, gel, or gas; biometric sensor 905 which is attached to the circumference-center-facing wall of the elastic member; and micropump 906 which pumps fluid, gel, or gas into (or out of) the elastic member. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

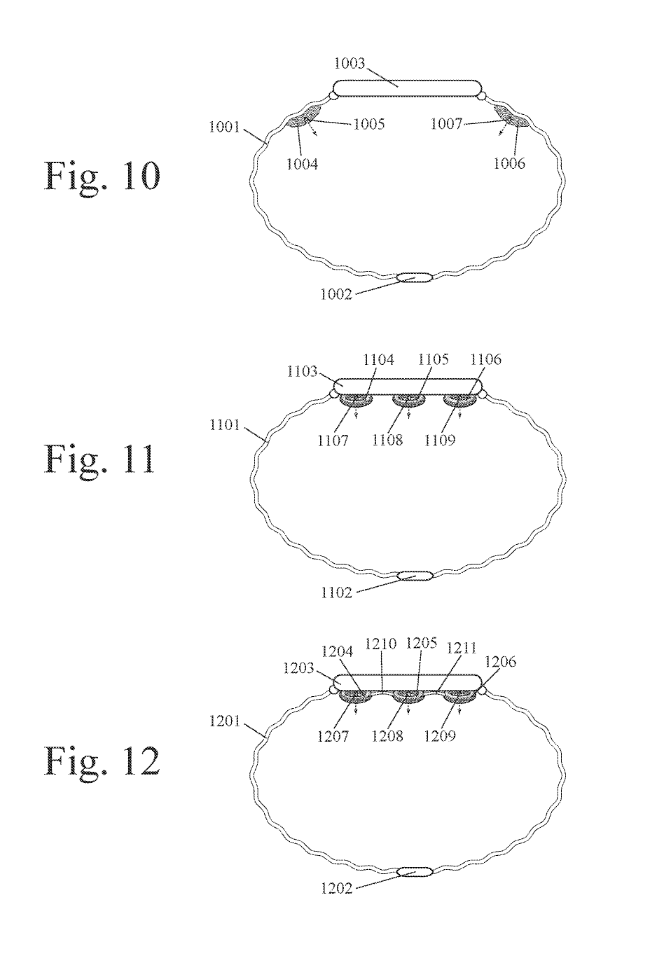

FIG. 10 shows another example of a wearable device for non-invasive glucose monitoring. This figure shows the device from a side perspective, as it would appear encircling a lateral cross-section of a person's wrist (or other portion of the person's arm). This wrist-worn device comprises: an attachment member which is configured to span at least a portion of the circumference of a person's arm; one or more elastic members filled with a flowable substance, wherein these elastic members are part of (or attached to) the circumference-center-facing surface of the attachment member; and one or more biometric sensors, wherein each sensor is part of (or attached to) a circumference-center-facing wall of an elastic member.

The design of this device keeps biometric sensors close to the surface of a person's arm, even if portions of the device shift away from the surface of the person's arm. The interiors of the elastic members on which these sensors are located are under modest pressure so that these elastic members expand when they are moved away from the arm surface and these elastic members are compressed when they are moved toward the arm surface.

In an example, an attachment member can be a strap, band, bracelet, ring, armlet, cuff, or sleeve. In an example, an attachment member can be attached to a person's arm by connecting two ends of the attachment member with a clasp, clip, buckle, hook, pin, plug, or hook-and-eye mechanism. In an example, an attachment member can be attached to a person's arm by stretching it circumferentially and sliding it over the person's hand onto the arm. In an example, an attachment member can be attached to a person's arm by applying force to pull two ends of the member apart in order to slip the member over the arm; the two ends then retract back towards each other when device is on the arm and the force is removed.

In an example, an elastic member can be a balloon or other elastic substance-filled compartment. In an example, the flowable substance inside an elastic member can be a fluid, gel, or gas. In this example, there are two elastic members on the attachment member. In this example, the elastic members are symmetrically located with respect to a central cross-section of the device. In an example, there can be a plurality of elastic members (with attached biometric sensors) which are distributed around the circumference of an attachment member and/or the device. In this example, a device can also include an enclosure which further comprises a display screen.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 10 includes: band 1001; band connector 1002; enclosure 1003; first elastic member 1004 which is filled with a fluid, gel, or gas; first biometric sensor 1005 which is attached to the circumference-center-facing wall of the first elastic member; second elastic member 1006 which is filled with a fluid, gel, or gas; and second biometric sensor 1007 which is attached to the circumference-center-facing wall of the second elastic member. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 11 shows another example of a wearable device for non-invasive glucose monitoring. This figure shows the device from a side perspective, as it would appear encircling a lateral cross-section of a person's wrist (or other portion of the person's arm). This wrist-worn device comprises: (a) an attachment member which is configured to span at least a portion of the circumference of a person's arm; (b) an enclosure which is part of (or attached to) the attachment member; (c) one or more torus-shaped elastic members filled with a flowable substance, wherein these elastic members are part of (or attached to) the enclosure; and (d) one or more biometric sensors, wherein each sensor is located in the central hole of a torus-shaped elastic member.

In an example, an attachment member can be a strap, band, bracelet, ring, armlet, cuff, or sleeve. In an example, an enclosure can further comprise a display screen on its outer surface. In an example, a torus-shaped elastic member can be a balloon which is filled with a fluid, gel, or gas. In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 11 includes: band 1101; band connector 1102; enclosure 1103; torus-shaped elastic members 1104, 1105, and 1106; and biometric sensors 1107, 1108, and 1109 which are each located in the central opening (or hole) of a torus-shaped elastic member. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 12 shows another example of a wearable device for non-invasive glucose monitoring. This figure shows the device from a side perspective, as it would appear encircling a lateral cross-section of a person's wrist (or other portion of the person's arm). The example in FIG. 12 like the one shown in FIG. 11, except that the example in FIG. 12 also includes channels through which a fluid, gel, or gas can flow between the torus-shaped elastic members.

With respect to specific components, the example shown in FIG. 12 includes: band 1201; band connector 1202; enclosure 1203; torus-shaped elastic members 1204, 1205, and 1206; biometric sensors 1207, 1208, and 1209 which are each located in the central opening (or hole) of a torus-shaped elastic member; and channels 1210 and 1211 through which fluid, gel, or gas can flow between the torus-shaped elastic members. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

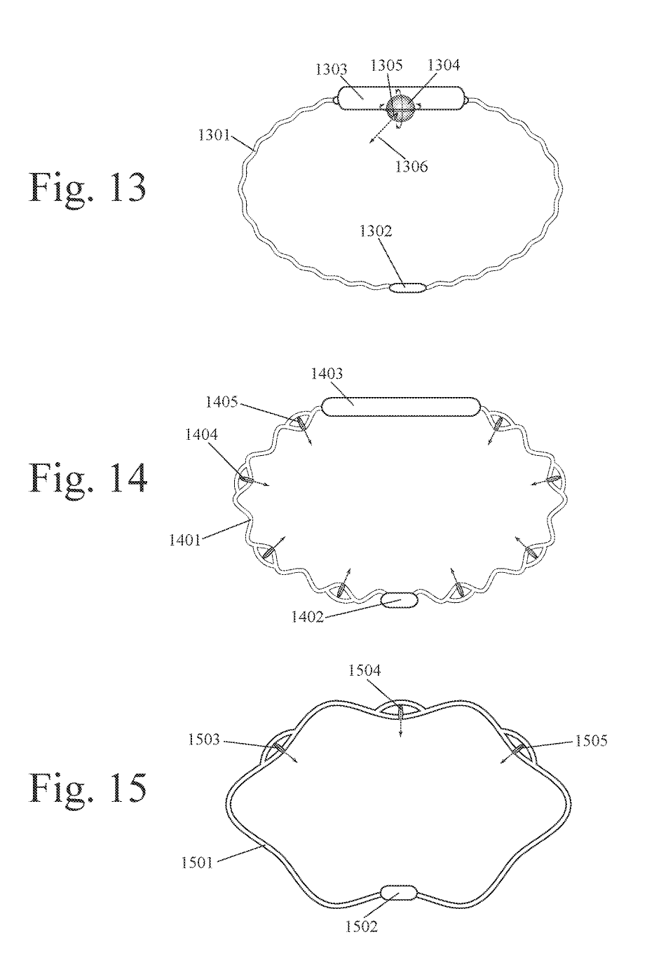

FIG. 13 shows another example of a wearable device for non-invasive glucose monitoring. This figure shows the device from a side perspective, as it would appear encircling a lateral cross-section of a person's wrist (or other portion of the person's arm). Described generally, the example shown in FIG. 13 is an arcuate wrist-worn device with a light-projecting spectroscopic sensor on a rotating ball. Rotating the ball changes the angle at which the spectroscopic sensor projects light onto the surface of a person's arm. The ball can be rotated in different directions so that the range of possible projection beams comprises a conic or frustal shape in three-dimensional space. Having a light-projecting spectroscopic sensor on a rotating ball can enable a device to record biometric measurements with substantially the same angle of incidence, even if an enclosure is tilted with respect to the surface of the person's arm.

The example shown in FIG. 13 is a wearable device for non-invasive glucose monitoring comprising: (a) an attachment member, such as a strap or band, which is configured to span at least a portion of the circumference of a person's arm; (b) an enclosure which is part of (or attached to) the attachment member; (c) a rotating ball which is part of (or attached to) the enclosure; and (d) a light-projecting spectroscopic sensor which is part of (or attached to) the rotating ball.

In an example, an attachment member can be a strap, band, bracelet, ring, armlet, cuff, or sleeve. In an example, the circumference-center-facing surface of an enclosure can be substantially flat. In an example, the circumference-center-facing surface of an enclosure can be curved. In an example, there can be a display screen on the outward-facing surface of an enclosure. In an example, the rotating ball can fit into the enclosure like a ball-and-socket joint. In an example, the device can further comprise one or more actuators which move the rotating ball.

With respect to specific components, the example shown in FIG. 13 includes: strap 1301, strap connector 1302, enclosure 1303, rotating ball 1304, and spectroscopic sensor 1305 which emits beam of light 1306. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 14 shows another example of a wearable device for non-invasive glucose monitoring. This figure shows the device from a side perspective, as it would appear encircling a lateral cross-section of a person's wrist (or other portion of the person's arm). Described generally, the example shown in FIG. 14 is a wearable device for the arm with a flexible circumferentially-undulating band with biometric sensors on the proximal portions of undulating waves. A band with such a flexible circumferentially-undulating structure can help to keep a plurality of biometric sensors in close proximity to the surface of a person's arm. In an example, an attachment member can be a strap, band, bracelet, ring, or armlet. In an example, a circumferentially-undulating attachment member can have a repeating wave pattern. In an example, a circumferentially-undulating attachment member can have a sinusoidal wave pattern.

The example shown in FIG. 14 is a wearable device for non-invasive glucose monitoring comprising: (a) a circumferentially-undulating attachment member which is configured to span at least a portion of the circumference of a person's arm; and (b) a plurality of biometric sensors which collect data concerning arm tissue, wherein each biometric sensor is located at the proximal portion of an undulation, and wherein the proximal portion of an undulation is the portion of an undulating wave which is closest to the circumferential center of the device.

With respect to specific components, the example shown in FIG. 14 includes: circumferentially-undulating band 1401, band connector 1402, enclosure 1403, first biometric sensor 1404 at the proximal portion of a first wave in the circumferentially-undulating band, and second biometric sensor 1405 at the proximal portion of a second wave in the circumferentially-undulating band. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 15 shows another example of a wearable device for non-invasive glucose monitoring. This figure shows the device from a side perspective, as it would appear encircling a lateral cross-section of a person's wrist (or other portion of the person's arm). Described generally, the example shown in FIG. 15 is a wearable device for the arm with a flexible circumferentially-undulating band with six waves and biometric sensors on the proximal portions of some or all of these waves.

A band with a circumferentially-undulating structure can help to keep a plurality of biometric sensors in close proximity to the surface of a person's arm. Further, a band with six waves can engage the sides of a person's wrist with two symmetrically-opposite waves to resist rotational shifting better than a circular or oval band. This can help to reduce measurement errors caused by movement of biometric sensors. In an example, a circumferentially-undulating attachment member can be a strap, band, bracelet, ring, or armlet. In an example, a circumferentially-undulating attachment member can have a repeating wave pattern. In an example, a circumferentially-undulating attachment member can have a sinusoidal wave pattern.

The example shown in FIG. 15 is a wearable device for non-invasive glucose monitoring comprising: (a) a circumferentially-undulating attachment member with six waves which is configured to span the circumference of a person's arm; and (b) a plurality of biometric sensors which collect data concerning arm tissue, wherein each biometric sensor is located at the proximal portion of an undulation, and wherein the proximal portion of an undulation is the portion of an undulating wave which is closest to the circumferential center of the device.

With respect to specific components, the example shown in FIG. 15 includes: circumferentially-undulating band 1501 with six waves, band connector 1502, a first biometric sensor 1503 at the proximal portion of a first wave in the circumferentially-undulating band, a second biometric sensor 1504 at the proximal portion of a second wave in the circumferentially-undulating band, and a third biometric sensor 1505 at the proximal portion of a third wave in the circumferentially-undulating band. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

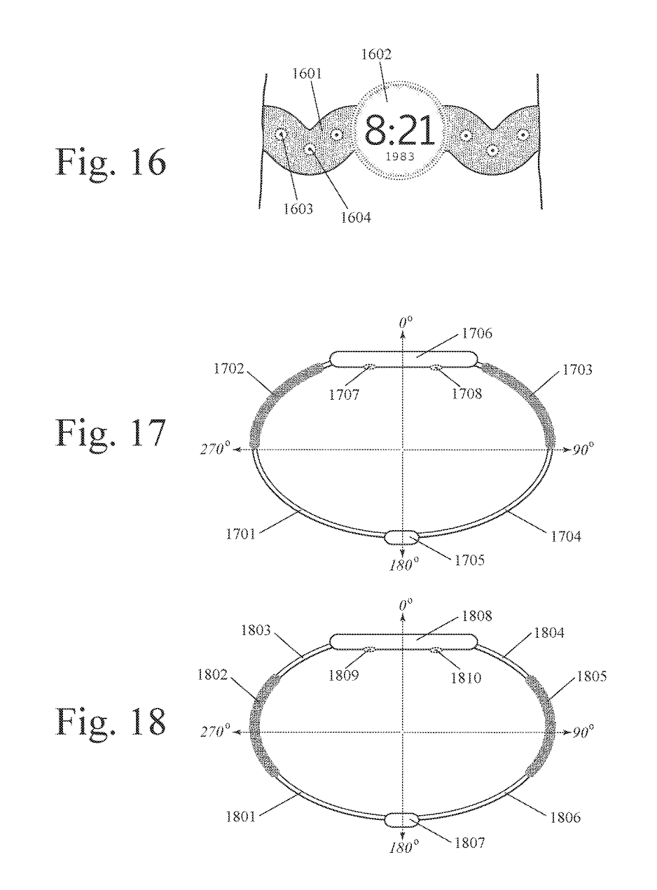

FIG. 16 shows another example of a wearable device for non-invasive glucose monitoring. This figure shows the device from a top-down perspective, as it would appear spanning the anterior (upper) surface of a person's wrist (or other portion of the person's arm) in a circumferential manner. Described generally, the example shown in FIG. 16 is a wearable device for the arm with a laterally-undulating band and biometric sensors. Lateral undulations are waves which are substantially perpendicular to the plane containing the band circumference. In an example, a band can have sinusoidal lateral undulations.

The example shown in FIG. 16 is a wearable device for non-invasive glucose monitoring comprising: (a) a laterally-undulating attachment member which is configured to span at least a portion of the circumference of a person's arm, wherein lateral undulations are waves which are substantially perpendicular to the plane containing the circumference of the attachment member; and (b) one or more biometric sensors which collect data concerning arm tissue, wherein these biometric sensors are part of (or attached to) the laterally-undulating attachment member.

With respect to specific components, the example shown in FIG. 16 includes: laterally-undulating strap 1601; display screen 1602; and biometric sensors including 1603 and 1604. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 17 shows another example of a wearable device for non-invasive glucose monitoring. This figure shows the device from a side perspective, as it would appear encircling a lateral cross-section of a person's wrist (or other portion of the person's arm). Described generally, the example shown in FIG. 17 is a wearable device for an arm with one or more biometric sensors in an enclosure and an attachment member (such as a strap, band, bracelet, or cuff) which attaches the enclosure to the arm, wherein this attachment member has relatively-elastic portions connected to the enclosure and relatively-inelastic portions elsewhere. This structure can help to keep the enclosure and sensors fitting closely against the arm. This, in turn, can enable more-consistent collection of data concerning arm tissue.

In an example, the device in FIG. 17 can be specified as a wearable device for non-invasive glucose monitoring comprising: (a) an attachment member which is configured to span at least 60% of the circumference of a person's arm, wherein this attachment member further comprises—one or more elastic portions which are configured to span the anterior (upper) surface of a person's arm and one or more inelastic portions which are configured to span the posterior (lower) surface of the person's arm; (b) an enclosure which is connected to the elastic portions of the attachment member; and (c) one or more biometric sensors which collect data concerning arm tissue which are part of (or attached to) the enclosure.

In an alternative example, a wearable device for non-invasive glucose monitoring can comprise: (a) an attachment member which is configured to span at least 60% of the circumference of a person's arm, wherein this attachment member further comprises—one or more elastic portions which are configured to span the posterior (lower) surface of a person's arm and one or more inelastic portions which are configured to span the anterior (upper) surface of the person's arm; (b) an enclosure which is connected to the elastic portions of the attachment member; and (c) one or more biometric sensors which collect data concerning arm tissue which are part of (or attached to) the enclosure.

In an example, an elastic portion of an attachment member can be an elastic strap or band. In an example, an elastic portion of an attachment member can be made from elastic fabric. In an example, an elastic portion of an attachment member can have a first elasticity level, an inelastic portion of an attachment member can have a second elasticity level, and the first elasticity level can be greater than the second elasticity level. In an example, a first elastic portion of an attachment member can be directly connected to a first side of an enclosure and a second elastic portion of an attachment member can be directly connected to a second (opposite) side of the enclosure. In an example, a first elastic portion of an attachment member can be indirectly connected to a first side of an enclosure and a second elastic portion of an attachment member can be indirectly connected to a second (opposite) side of the enclosure.

In an example, the device in FIG. 17 can be specified as a wearable device for non-invasive glucose monitoring comprising: (a) an attachment member which is configured to span at least 60% of the circumference of a person's arm wherein this attachment member further comprises: two elastic portions which are configured to span a first portion of the circumference of a person's arm; and two inelastic portions which are configured to span a second portion of the circumference of the person's arm; (b) an enclosure which is connected between the two elastic portions; (c) a clip, buckle, clasp, pin, or hook-and-eye mechanism between the two inelastic portions; and d) one or more biometric sensors which collect data concerning arm tissue which are part of (or attached to) the enclosure.

In an example, the device in FIG. 17 can be specified as a wearable device for non-invasive glucose monitoring comprising: (a) an attachment member which is configured to span at least 60% of the circumference of a person's arm wherein this attachment member further comprises: two elastic portions of the attachment member which are configured to span a portion of the circumference of a person's arm; and one or more inelastic portions which comprise the remainder of the attachment member; (b) an enclosure which is connected between the two elastic portions; (c) one or more biometric sensors which collect data concerning arm tissue which are part of (or attached to) the enclosure.

In an example, a single elastic portion can be configured to span at least 10% of the circumference of a person's arm. In an example, a single elastic portion can be configured to span at least 10% of the circumference of an attachment member. In an example, a single inelastic portion can be configured to span at least 10% of the circumference of a person's arm. In an example, a single inelastic portion can be configured to span at least 10% of the circumference of an attachment member. In an example, two elastic portions can be configured to collectively span at least 20% of the circumference of a person's arm. In an example, two elastic portions can be configured to collectively span at least 20% of the circumference of an attachment member. In an example, two inelastic portions can be configured to collectively span at least 20% of the circumference of a person's arm. In an example, two inelastic portions can be configured to collectively span at least 20% of the circumference of an attachment member.

In an example, a first definition of polar (or compass) coordinates can be defined for a device relative to how the device is configured to be worn on a person's arm. A 0-degree position can be defined as the position on a device circumference which is configured to intersect the longitudinal mid-line of the anterior (upper) surface of the arm. A 180-degree position is diametrically opposite (through the circumferential center) the 0-degree position. A 90-degree position is (clockwise) midway between the 0-degree and 180-degree positions. A 270-degree position is diametrically opposite the 90-degree position.

Using this first definition of polar coordinates, the device in FIG. 17 can be specified as a wearable device for non-invasive glucose monitoring comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90-degree positions, an inelastic third portion with a third level of elasticity which spans at least 35 degrees (clockwise) between the 90-degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is configured to be worn (clockwise) between the 270-degree and 90-degree positions; and (c) one or more biometric sensors which collect data concerning arm tissue which are part of (or attached to) the enclosure.

Using this first definition of polar coordinates, the device in FIG. 17 can also be specified as a wearable device for non-invasive glucose monitoring comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90-degree positions, an inelastic third portion with a third level of elasticity which spans at least 35 degrees (clockwise) between the 90-degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is connected between the elastic first and second portions; and (c) one or more biometric sensors which collect data concerning arm tissue which are part of (or attached to) the enclosure.

Alternatively, a second definition of polar (or compass) coordinates can be defined for the circumference of such a device relative to the position of an enclosure. The 0-degree position can be defined as the position on the device circumference which intersects the (lateral) mid-line of the enclosure. The 180-degree position is diametrically opposite (through the circumferential center) the 0-degree position. The 90-degree position is clockwise midway between the 0-degree and 180-degree positions. The 270-degree position is diametrically opposite the 90-degree position.

Using this second definition of polar coordinates, the device in FIG. 17 can be specified as a wearable device for non-invasive glucose monitoring comprising: (a) an attachment member which is configured to span at least 60% of the circumference of a person's arm wherein this attachment member further comprises—an elastic first portion with a first level of elasticity which spans at least 35 degrees (clockwise) between the 270-degree and 0-degree positions; an elastic second portion with a second level of elasticity which spans at least 35 degrees (clockwise) between the 0-degree and 90-degree positions, an inelastic third portion with a third level of elasticity which spans at least 35 degrees (clockwise) between the 90-degree and 180-degree positions, an inelastic fourth portion with a fourth level of elasticity which spans at least 35 degrees (clockwise) between the 180-degree and 270-degree positions, and wherein each of the first and second elasticity levels is greater than each of the third and fourth elasticity levels; (b) an enclosure that is connected between the elastic first and second portions of the attachment member; and (c) one or more biometric sensors which collect data concerning arm tissue which are part of (or attached to) the enclosure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 17 includes: inelastic portion 1701 of an attachment member; elastic portion 1702 of an attachment member; elastic portion 1703 of an attachment member; inelastic portion 1704 of an attachment member; attachment member connector 1705; enclosure 1706; and biometric sensors 1707 and 1708. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 18 shows another example of a wearable device for non-invasive glucose monitoring. This figure shows the device from a side perspective, as it would appear encircling a lateral cross-section of a person's wrist (or other portion of the person's arm). Described generally, the example shown in FIG. 18 is a wearable device for the arm with one or more biometric sensors in an enclosure and an attachment member (such as a strap, band, bracelet, or cuff) which attaches the enclosure to the arm, wherein the attachment member is configured to have elastic portions spanning the lateral surfaces of the arm and inelastic portions spanning the anterior (upper) and posterior (lower) surfaces of the arm. This structure can help to keep the enclosure and sensors from rotating around the arm. This, in turn, can enable more-consistent collection of data concerning arm tissue.

In an example, the device in FIG. 18 can be specified as a wearable device for non-invasive glucose monitoring comprising: (a) an attachment member which is configured to span at least 60% of the circumference of a person's arm wherein this attachment member further comprises—one or more anterior inelastic portions which are configured to span the anterior (upper) surface of a person's arm, one or more posterior inelastic portions which are configured to span the posterior (lower) surface of a person's arm, and one or more elastic portions which connect the anterior and posterior inelastic portions; (b) an enclosure which is configured to be worn on the anterior (upper) portion of the arm; and (c) one or more biometric sensors which collect data concerning arm tissue which are part of (or attached to) the enclosure.

In another example, a wearable device for non-invasive glucose monitoring can comprise: (a) an attachment member which is configured to span at least 60% of the circumference of a person's arm wherein this attachment member further comprises—one or more anterior inelastic portions which are configured to span the anterior (upper) surface of a person's arm, one or more posterior inelastic portions which are configured to span the posterior (lower) surface of a person's arm, and one or more elastic portions which connect the anterior and posterior inelastic portions; (b) an enclosure which is configured to be worn on the posterior (lower) portion of the arm; and (c) one or more biometric sensors which collect data concerning arm tissue which are part of (or attached to) the enclosure.

In an example, a first inelastic portion of an attachment member can be connected to a first side of an enclosure and a second inelastic portion of an attachment member can be connected to a second side of the enclosure. In an example, an elastic portion can have a first level of elasticity, an inelastic portion can have a second level of elasticity, and the first level is greater than the second level. In an example, a single elastic portion can be configured to span at least 10% of the circumference of a person's arm. In an example, a single elastic portion can be configured to span at least 10% of the circumference of an attachment member. In an example, a single inelastic portion can be configured to span at least 10% of the circumference of a person's arm. In an example, a single inelastic portion can be configured to span at least 10% of the circumference of an attachment member.

In an example, polar (or compass) coordinates can be defined for a device relative to how the device is configured to be worn on a person's arm. A 0-degree position can be defined as the position on a device circumference which is configured to intersect the longitudinal mid-line of the anterior (upper) surface of the arm. A 180-degree position is diametrically opposite (through the circumferential center) the 0-degree position. A 90-degree position is clockwise midway between the 0-degree and 180-degree positions. A 270-degree position is diametrically opposite the 90-degree position.

In an example, the device in FIG. 18 can be specified as a wearable device for non-invasive glucose monitoring comprising: (a) an attachment member which is configured to span at least 60% of the circumference of a person's arm wherein this attachment member further comprises—a inelastic first portion with a first level of elasticity which spans at least 35 degrees (clockwise) between the 270-degree and 90-degree positions; an inelastic second portion with a second level of elasticity which spans at least 35 degrees (clockwise) between the 90-degree and 270-degree positions, an elastic third portion with a third level of elasticity which spans at least 35 degrees (clockwise) between the 180-degree and 0-degree positions, an elastic fourth portion with a fourth level of elasticity which spans at least 35 degrees (clockwise) between the 0-degree and 180-degree positions, and wherein each of the first and second elasticity levels is lower than each of the third and fourth elasticity levels; (b) an enclosure that is connected between the inelastic first portion and the inelastic second portion; and (c) one or more biometric sensors which collect data concerning arm tissue which are part of (or attached to) the enclosure.

In an alternative example, polar (or compass) coordinates can be defined for the circumference of such a device relative to the position of an enclosure on the device. The 0-degree position can be defined as the position on the device circumference which intersects the (lateral) mid-line of the enclosure. The 180-degree position is diametrically opposite (through the circumferential center) the 0-degree position. The 90-degree position is clockwise midway between the 0-degree and 180-degree positions. The 270-degree position is diametrically opposite the 90-degree position.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

With respect to specific components, the example shown in FIG. 18 includes: inelastic portion 1801 of an attachment member; elastic portion 1802 of an attachment member; inelastic portion 1803 of an attachment member; inelastic portion 1804 of an attachment member; elastic portion 1805 of an attachment member; inelastic portion 1806 of an attachment member; attachment member connector 1807; enclosure 1808; and biometric sensors 1809 and 1810. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

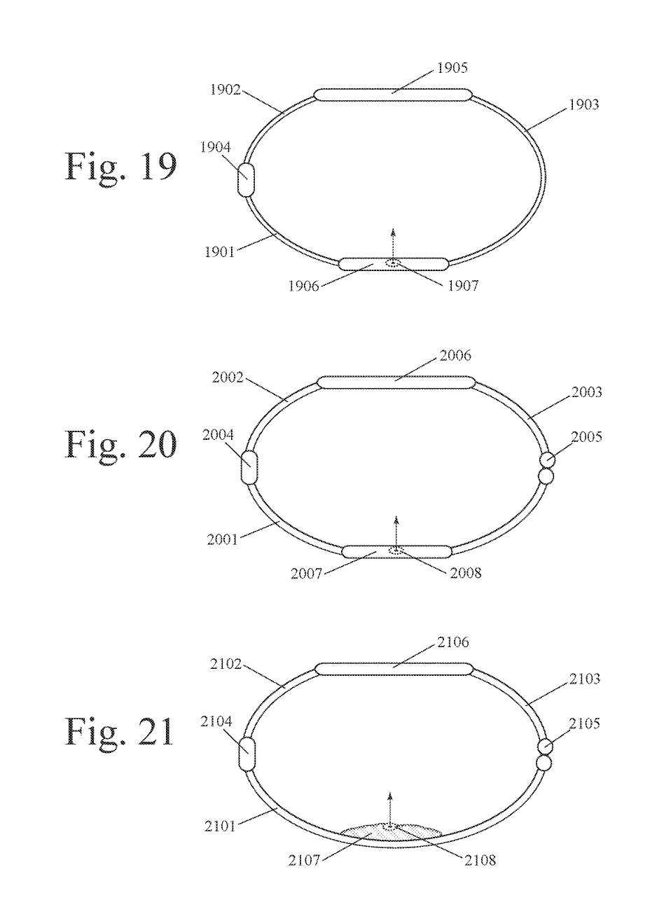

FIG. 19 shows another example of a wearable device for non-invasive glucose monitoring. This figure shows the device from a side perspective, as it would appear encircling a lateral cross-section of a person's wrist (or other portion of the person's arm). In this example, one or more biometric sensors are located on a portion of the device which is diametrically-opposite (e.g. symmetric relative to the circumferential center of the device) from the portion of the device which includes a display screen and there is a connector (such as a buckle, clip, clasp, pin, plug, or hook-and-eye mechanism) on the device between the sensors and the screen.

The example in FIG. 19 can be specified as a wearable device for non-invasive glucose monitoring comprising: (a) an attachment member which is configured to span at least 60% of the circumference of a person's arm; (b) a display screen which is part of (or connected to) the attachment member at a first location along the circumference of the device; (c) an enclosure which is part of (or connected to) the attachment member at a second location along the circumference of the device, wherein the second location is on the opposite side of the device (e.g. through the circumferential center of the device) from the first location; (d) one or more biometric sensors which are configured to collect data concerning arm tissue and which are part of (or attached to) the enclosure; and (e) a connector which connects two ends of the attachment member to each other, wherein this connector is at a location along the circumference of the device which is between the display screen and the enclosure.

The example in FIG. 19 can also be specified as a wearable device for non-invasive glucose monitoring comprising: (a) an attachment member which is configured to span at least 60% of the circumference of a person's arm; (b) a display screen which is located between the 10 o'clock and 2 o'clock (or 300-degree and 60-degree) positions on the circumference of the attachment member; (c) an enclosure which is located between the 4 o'clock and 8 o'clock (or 120-degree and 240-degree) positions on the circumference of the attachment member; (d) one or more biometric sensors which are configured to collect data concerning arm tissue and which are part of (or attached to) the enclosure; and (e) a connector which connects two ends of the attachment member to each other, wherein this connector is at a location along the circumference of the device which is between the display screen and the enclosure.

In an example, the center of the display screen can be located at the 12 o'clock (or 0-degrees) position on the circumference of the device. In an example, the center of the display screen can be located at the 6 o'clock (or 180-degrees) position on the circumference of the device. In an example, a connector can be selected from the group consisting of: buckle, clip, clasp, hook, plug, pin, snap, and hook-and-eye mechanism.

With respect to specific components, the example shown in FIG. 19 includes: segments 1901, 1902, and 1903 of an attachment member; connector 1904; display screen 1905; enclosure 1906; and biometric sensor 1907. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 20 shows another example of a wearable device for non-invasive glucose monitoring. This figure shows the device from a side perspective, as it would appear encircling a lateral cross-section of a person's wrist (or other portion of the person's arm). In this example, there are one or more biometric sensors which are opposite a display screen, a connector between the sensors and the screen, and a hinge which is opposite the connector. If portions of an attachment member connecting these components are relatively rigid, then this example can be called a “clam shell” design.

The example in FIG. 20 can be specified as a wearable device for non-invasive glucose monitoring comprising: (a) an attachment member which is configured to span at least 60% of the circumference of a person's arm; (b) a display screen which is part of (or connected to) the attachment member at a first location around the circumference of the device; (c) an enclosure which is part of (or connected to) the attachment member at a second location around the circumference of the device, wherein the second location is on the opposite (e.g. through the circumferential center) side of the device from the first location; (d) one or more biometric sensors which are configured to collect data concerning arm tissue and which are part of (or attached to) the enclosure; and (e) a connector which connects two ends of the attachment member to each other at a third location around the circumference of the device, wherein this third location is between the first and second locations; (f) a hinge (or joint) which connects two portions of the attachment member to each other at a fourth location around the circumference of the device, wherein this fourth location is on the opposite (e.g. through the circumferential center) side of the device from the third location.

The example in FIG. 20 can also be specified as a wearable device for non-invasive glucose monitoring comprising: (a) an attachment member which is configured to span at least 60% of the circumference of a person's arm; (b) a display screen which is located between the 10 o'clock and 2 o'clock (or 300-degree and 60-degree) positions on the circumference of the attachment member; (c) an enclosure which is located between the 4 o'clock and 8 o'clock (or 120-degree and 240-degree) positions on the circumference of the attachment member; (d) one or more biometric sensors which are configured to collect data concerning arm tissue and which are part of (or attached to) the enclosure; (e) a connector which connects two ends of the attachment member to each other, wherein this connector is located between the 7 o'clock and 11 o'clock (or 210-degree and 330-degree) positions on the circumference of the attachment member; and (f) a hinge which connects two portions of the attachment member to each other, wherein this hinge is located between the 1 o'clock and 5 o'clock (or 30-degree and 150-degree) positions on the circumference of the attachment member.

In an example, the center of the display screen can be located at the 12 o'clock (or 0-degrees) position on the circumference of the device. In an example, the center of the display screen can be located at the 6 o'clock (or 180-degrees) position on the circumference of the device. In an example, a connector can be selected from the group consisting of: buckle, clip, clasp, hook, plug, pin, snap, and hook-and-eye mechanism.

With respect to specific components, the example shown in FIG. 20 includes: segments 2001, 2002, and 2003 of an attachment member; connector 2004; hinge 2005; display screen 2006; enclosure 2007; and biometric sensor 2008. In an example, this device can further comprise one or more components selected from the group consisting of: a data processor; a battery and/or energy harvesting unit; a display screen; a data transmitter; and a data receiver. Other example variations and component elaborations discussed elsewhere in this disclosure (or in other disclosures within the priority-linked family) can also be applied to this example.

FIG. 21 shows another example of a wearable device for non-invasive glucose monitoring. This figure shows the device from a side perspective, as it would appear encircling a lateral cross-section of a person's wrist (or other portion of the person's arm). This example is similar to the one shown in FIG. 20 except a biometric sensor is on the center-facing surface of a compressible member.

With respect to specific components, the example shown in FIG. 21 includes: segments 2101, 2102, and 2103 of an attachment member; connector 2104; hinge 2105; display screen 2106; compressible member 2107; and biometric sensor 2108. In an example, this device can further comprise one or more components selected from the group consisting of: a data processor; a battery and/or energy harvesting unit; a display screen; a data transmitter; and a data receiver.

In an example, a compressible member can be an elastic member which is filled with a fluid, gel, or gas. In an example, a compressible member can be a pneumatic or hydraulic chamber which is filled with a fluid, gel, or gas. In an example, a compressible member can be a balloon. In an example, a compressible member can be made from compressible foam. Relevant embodiment variations discussed elsewhere in this disclosure can also be applied to this example. Other example variations and component elaborations discussed elsewhere in this disclosure (or in other disclosures within the priority-linked family) can also be applied to this example.

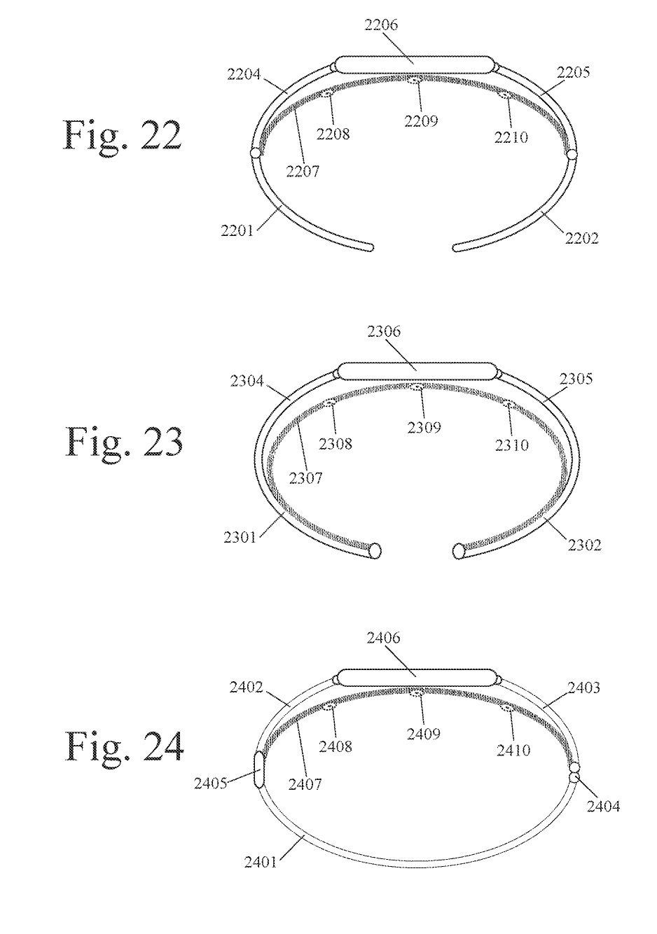

FIG. 22 shows another example of a wearable device for non-invasive glucose monitoring. This figure shows the device from a side perspective, as it would appear encircling a lateral cross-section of a person's wrist (or other portion of their arm). The example in FIG. 22 is like the one shown in FIG. 21, except that in FIG. 22 the outer inelastic band is sufficiently resilient that its ends hold onto the person's arm without the need for a clasp. The outer inelastic band can be attached to a person's arm by applying force to pull two ends apart to slip the member over the arm, wherein the two ends retract back towards each other when the force is removed.

In an example, this device can further comprise one or more components selected from the group consisting of: a data processor; a battery and/or energy harvesting unit; a data transmitter; a data receiver; and a display screen. In an example, this device can function as a smart watch. Specific components in the example shown in FIG. 22 include: four segments (2201, 2202, 2204, and 2205) of an outer inelastic band; inner elastic band 2207; biometric sensors (2208, 2209, and 2210); and display screen 2206. Other example variations and component elaborations discussed elsewhere in this disclosure (or in other disclosures within the priority-linked family) can also be applied to this example.

FIG. 23 shows another example of a wearable device for non-invasive glucose monitoring. This figure shows the device from a side perspective, as it would appear encircling a lateral cross-section of a person's arm. The example in FIG. 23 can be described as an arm-wearable device with an outer arcuate inelastic band, an inner arcuate elastic band, and biometric sensors which are part of the inner band. This design can provide an overall semi-rigid structure (e.g. to hold a rigid display screen in place) and also keep biometric sensors close against the surface of the arm for consistent collection of biometric data.

The example shown in FIG. 23 can be specified as a wearable device for non-invasive glucose monitoring comprising: (a) an outer arcuate inelastic band which is configured to span at least 60% of the circumference of a person's arm and which has a first elasticity level; (b) an inner arcuate elastic band which is located on (and attached to) the concave side of the outer arcuate band and which has a second elasticity level, wherein the second elasticity level is greater than the first elasticity level; and (c) one or more biometric sensors which are configured to collect data concerning arm tissue, wherein these biometric sensors are part of (or attached to) the inner arcuate elastic band. In various examples, a ring, strap, bracelet, bangle, armlet, sleeve, or cuff can be substituted for a band.

Alternatively, the example shown in FIG. 23 can be specified as a wearable device for non-invasive glucose monitoring comprising: (a) an outer arcuate inelastic band, wherein this outer arcuate inelastic band is configured to span at least 60% of the circumference of a person's arm, wherein this outer arcuate inelastic band is configured to be a first average distance from the surface of the person's arm, and wherein this outer arcuate inelastic band has a first elasticity level; (b) an inner arcuate elastic band, wherein this inner arcuate elastic band is attached to the outer arcuate inelastic band, wherein this inner arcuate elastic band is configured to be an second average distance from the surface of the person's arm, wherein this inner arcuate elastic band has a second elasticity level, wherein the second average distance is less than the first average distance, and wherein the second elasticity level is greater than the first elasticity level; and (c) one or more biometric sensors which are configured to collect data concerning arm tissue, wherein these biometric sensors are part of (or attached to) the inner arcuate elastic band. In various examples, a ring, strap, bracelet, bangle, armlet, sleeve, or cuff can be substituted for a band.

In an example, an outer arcuate inelastic band can be attached to a person's arm by connecting two ends of the outer inelastic band with a clasp, clip, buckle, hook, pin, plug, or hook-and-eye mechanism. In an example, an outer arcuate inelastic band can be attached to a person's arm by stretching and sliding it over the person's hand onto the arm. In an example, an outer arcuate inelastic band can be attached to a person's arm by applying force to pull two ends apart to slip the member over the arm, wherein the two ends retract back towards each other when the force is removed. In an example, an inner arcuate elastic band can be made from a stretchable fabric. In an example, an inner arcuate elastic band can be attached to an outer arcuate inelastic band at the ends of the arcuate inelastic band. In an example, an inner arcuate elastic band can be attached to an outer arcuate inelastic band near mid-points of segments of the outer arcuate inelastic band.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Specific components in the example shown in FIG. 23 include: four segments (2301, 2302, 2304, and 2305) of an outer inelastic band; inner elastic band 2307; biometric sensors (2308, 2309, and 2310); and display screen 2306. Other example variations and component elaborations discussed elsewhere in this disclosure (or in other disclosures within the priority-linked family) can also be applied to this example.

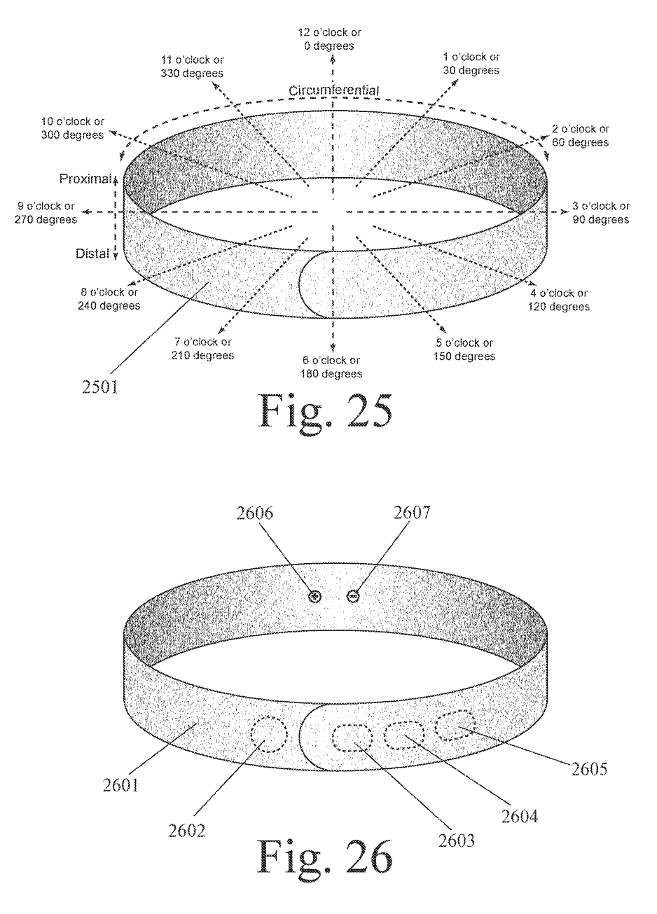

FIG. 24 shows another example of a wearable device for non-invasive glucose monitoring. This figure shows the device from a side perspective, as it would appear encircling a lateral cross-section of a person's wrist (or other portion of the person's arm). The example in FIG. 24 can be described as an arm-wearable device with an outer rigid “clam shell” structure to hold a display screen in place and an inner arcuate elastic band to keep biometric sensors close against the surface of the arm.

The example shown in FIG. 24 can be specified as a wearable device for non-invasive glucose monitoring comprising: (a) a clam shell structure which is configured to span the circumference of a person's arm, wherein this clam shell structure further comprises: an upper half-circumferential portion, a lower half-circumferential portion, a joint (and/or hinge) between these portions on a first side of these portions, and a connector which reversibly connects these portions on a second side of these portions; (b) an arcuate elastic band which is located within the concavity of the clam shell structure and is attached to the clam shell structure; and (c) one or more biometric sensors which are configured to collect data concerning arm tissue, wherein these biometric sensors are part of (or attached to) the arcuate elastic band.

In an example, an upper half-circumferential portion of a clam shell structure can span the anterior (upper) surface of a person's arm and a lower half-circumferential portion of a clam shell structure can span the posterior (lower) surface of the person's arm. In an example, there can be a display screen on the outer surface of one or both portions of a clam shell structure. In an example, a connector which reversibly connects the upper and lower portions of a clam shell structure can be selected from the group consisting of: clasp, clip, buckle, hook, pin, plug, and hook-and-eye mechanism. In an example, an inner arcuate elastic band can be made from a stretchable fabric. In an example, an inner arcuate elastic band can be attached to an upper half-circumferential portion of a clam shell structure.

In an example, a biometric sensor can be a spectroscopic sensor which is configured to measure the spectrum of light energy reflected from (and/or absorbed by) tissue of the person's arm. In an example, a biometric sensor can be an electromagnetic energy sensor which is configured to measure parameters and/or patterns of electromagnetic energy passing through (and/or emitted by) tissue of the person's arm. In an example, measured parameters and/or patterns of electromagnetic energy can be selected from the group consisting of: impedance, resistance, conductivity, and electromagnetic wave pattern.

In an example, this device can further comprise one or more components selected from the group consisting of: a data processor; a battery and/or energy harvesting unit; a data transmitter; a data receiver; and a display screen. In an example, this device can function as a smart watch. Relevant embodiment variations discussed elsewhere in this disclosure can also be applied to this example. Specific components in the example shown in FIG. 24 include: two segments 2402 and 2403 of an upper half-circumferential portion of a clam shell structure; a lower half-circumferential portion 2401 of the clam shell structure; a joint (or hinge) 2404 between the upper and lower portions of the clam shell structure; a reversible connector 2405 between the upper and lower portions of the clam shell structure; an inner elastic band 2407; biometric sensors 2408, 2409, and 2410; and display screen 2406. Other example variations and component elaborations discussed elsewhere in this disclosure (or in other disclosures within the priority-linked family) can also be applied to this example.

FIG. 25 shows how “proximal-to-distal” and “circumferential” axes can be defined on a wearable band, but does not show a complete embodiment of this invention. FIG. 25 shows how the term “proximal-to-distal” can be defined with respect to a band which is worn on a person's wrist, finger, or ankle. The term “proximal” when applied to a person's wrist or finger means “closer to the person's shoulder when the arm is extended” and the term “distal” means “farther from the person's shoulder when the arm is extended.” The term “proximal” when applied to a person's ankle means “closer to the person's hip when the leg is extended” and the term “distal” means “farther from the person's hip when the leg is extended.” A proximal-to-distal axis can be defined for a band worn on the wrist, finger, or ankle using these definitions of proximal and distal. The left side of FIG. 25 shows a straight-line proximal-to-distal axis (labeled with the words “proximal” and “distal”) for band 2501 which can be worn on a person's wrist, finger, or ankle.

FIG. 25 also shows how the term “circumferential” can be defined with respect to a band which is worn on a person's wrist, finger, or ankle. Circumferential refers to the cross-sectional perimeter of the part of the body (e.g. wrist, finger, or ankle) around which the band is worn. In an example, this cross-sectional perimeter can be perpendicular to the central proximal-to-distal axis of the part of the body (e.g. wrist, finger, or ankle). The upper portion of FIG. 25 shows an arcuate circumferential axis (labeled with the word “circumferential”) for band 2501 which is worn on a person's wrist, finger, or ankle. In an example, movement in one direction or the other along the circumferential axis can be referred to as clockwise or counter-clockwise movement or, alternatively, as movement to the right or to the left.

FIG. 25 also shows how compass (or polar) or clock-hour coordinates be defined around a band's circumferential axis. These compass or clock-hour coordinates are shown in a circle around band 2501. Compass coordinates are shown in degrees, from 0 degrees, around the full circle, and then back to 0 degrees (which can also be called 360 degrees). Clock-hour coordinates are shown in hours, from 12 o'clock, around the full circle, and then back to 12 o'clock. In an example, the 0-degree (or 12 o'clock) location on the circumferential axis of a band can be the central frontal (or ventral or anterior) location on a wrist (around which a traditional watch face would be usually centered) or the central frontal (or ventral or anterior) location on an ankle.

FIGS. 26 through 62 show different examples of how this invention can be embodied in a wearable device for non-invasive glucose monitoring comprising: an arcuate band which is configured to span (some or all of) the circumferential perimeter of a part of a person's body (such as a wrist, arm, finger, ankle, and/or leg); an energy emitter which is configured to emit energy toward the part of the person's body; an energy receiver which is configured to receive energy from the energy emitter after that energy has passed through and/or been reflected from the part of the person's body; a data processor which receives data from the energy receiver which is analyzed in order to measure the person's body glucose level; and an energy source which provides energy to the energy emitter and/or to the data processor; and a data transmitter which transmits data from the data processor to a remote device and/or remote location. Other example variations and component elaborations discussed elsewhere in this disclosure (or in other disclosures within the priority-linked family) can also be applied to this example.

FIG. 26 shows an example of a wearable device for non-invasive glucose monitoring with a single energy emitter and a single energy receiver which are both along the same circumferential line of a band. Specifically, FIG. 26 shows an oblique-side-perspective view of a wearable device for non-invasive glucose monitoring comprising: arcuate band 2601 which is configured to span (some or all of) the circumferential perimeter of a part of a person's body (such as a wrist, arm, finger, ankle, and/or leg); energy emitter 2606 (identified with a plus sign) which is configured to emit energy toward the part of the person's body; energy receiver 2607 (identified with a negative sign) which is configured to receive energy from the energy emitter after that energy has passed through and/or been reflected from the part of the person's body; data processor 2604 which receives data from the energy receiver which is analyzed in order to measure the person's body glucose level; energy source 2603 which provides energy to the energy emitter and/or to the data processor; and data transmitter 2605 which transmits data from the data processor to a remote device and/or remote location. This example further comprises another (type of) biometric or environmental sensor 2602.

In the example in FIG. 26, there is a single energy emitter 2606 and a single energy receiver 2607 which are circumferentially aligned; both are located along the same circumferential line of band 2601. In this example, energy emitter 2606 is between the 330-degree and 0-degree locations and energy receiver 2607 is between the 0-degree and 30-degree locations. In this example, energy emitter 2606 and energy receiver 2607 are separated by less than 1″ or by less than 60 degrees of the band circumference. In another example, the locations of the energy emitter and the energy receiver can be reversed. In another example, an energy emitter, an energy receiver, or both can be part of a housing which is attached to the band, rather than part of the band itself. Other example variations and component elaborations discussed elsewhere in this disclosure (or in other disclosures within the priority-linked family) can also be applied to this example.

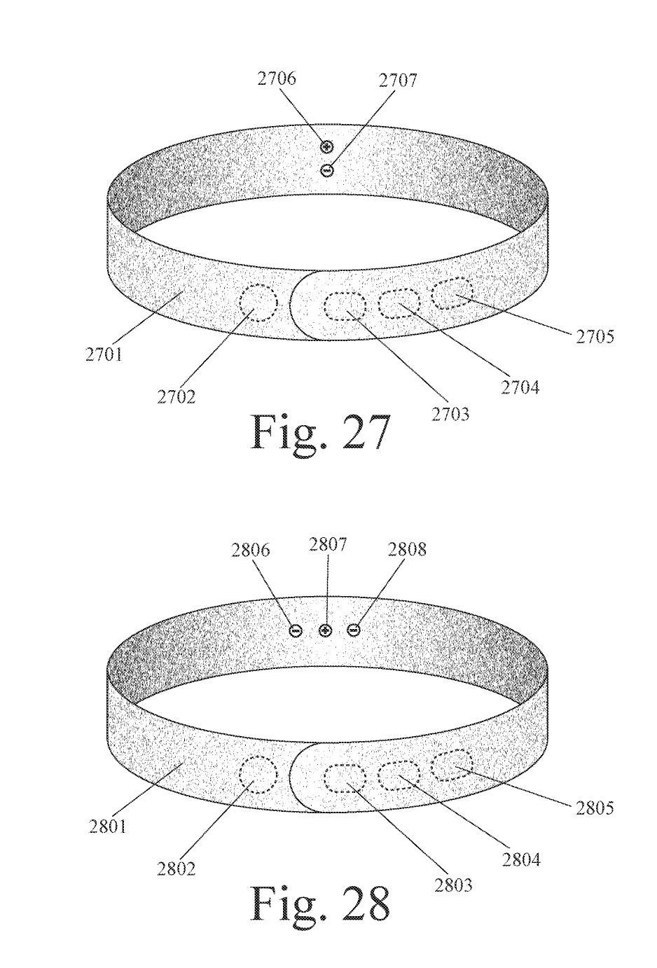

FIG. 27 shows an example of a wearable device for non-invasive glucose monitoring with a single energy emitter and a single energy receiver which are both along the same proximal-to-distal line of a band. In this example, the energy emitter is more proximal than the energy receiver. In another example, the locations of the energy emitter and the energy receiver can be reversed.

Specifically, FIG. 27 shows an oblique-side-perspective view of a wearable device for non-invasive glucose monitoring comprising: arcuate band 2701 which is configured to span (some or all of) the circumferential perimeter of a part of a person's body (such as a wrist, arm, finger, ankle, and/or leg); energy emitter 2706 (identified with a plus sign) which is configured to emit energy toward the part of the person's body; energy receiver 2707 (identified with a negative sign) which is configured to receive energy from the energy emitter after that energy has passed through and/or been reflected from the part of the person's body; data processor 2704 which receives data from the energy receiver which is analyzed in order to measure the person's body glucose level; energy source 2703 which provides energy to the energy emitter and/or to the data processor; and data transmitter 2705 which transmits data from the data processor to a remote device and/or remote location. This example further comprises another (type of) biometric or environmental sensor 2702. In another example, an energy emitter and/or an energy receiver can be part of a housing which is attached to the band. Other example variations and component elaborations discussed elsewhere in this disclosure (or in other disclosures within the priority-linked family) can also be applied to this example.

FIG. 28 shows an example of a wearable device for non-invasive glucose monitoring with a single energy emitter and two energy receivers which are all along the same circumferential line of a wearable arcuate band. In this example, the energy emitter is located between the two energy receivers. In this example, the energy emitter is located midway between the two energy receivers.

Specifically, FIG. 28 shows an oblique-side-perspective view of a wearable device for non-invasive glucose monitoring comprising: arcuate band 2801 which is configured to span (some or all of) the circumferential perimeter of a part of a person's body (such as a wrist, arm, finger, ankle, and/or leg); energy emitter 2807 (identified with a plus sign) which is configured to emit energy toward the part of the person's body; first energy receiver 2806 (identified with a negative sign) which is configured to receive energy from the energy emitter after that energy has passed through and/or been reflected from the part of the person's body; second energy receiver 2808 (identified with a negative sign) which is configured to receive energy from the energy emitter after that energy has passed through and/or been reflected from the part of the person's body, wherein the energy emitter is located between the first energy receiver and the second energy receiver; data processor 2804 which receives data from the energy receivers which is analyzed in order to measure the person's body glucose level; energy source 2803 which provides energy to the energy emitter and/or to the data processor; and data transmitter 2805 which transmits data from the data processor to a remote device and/or remote location. This example further comprises another (type of) biometric or environmental sensor 2802. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

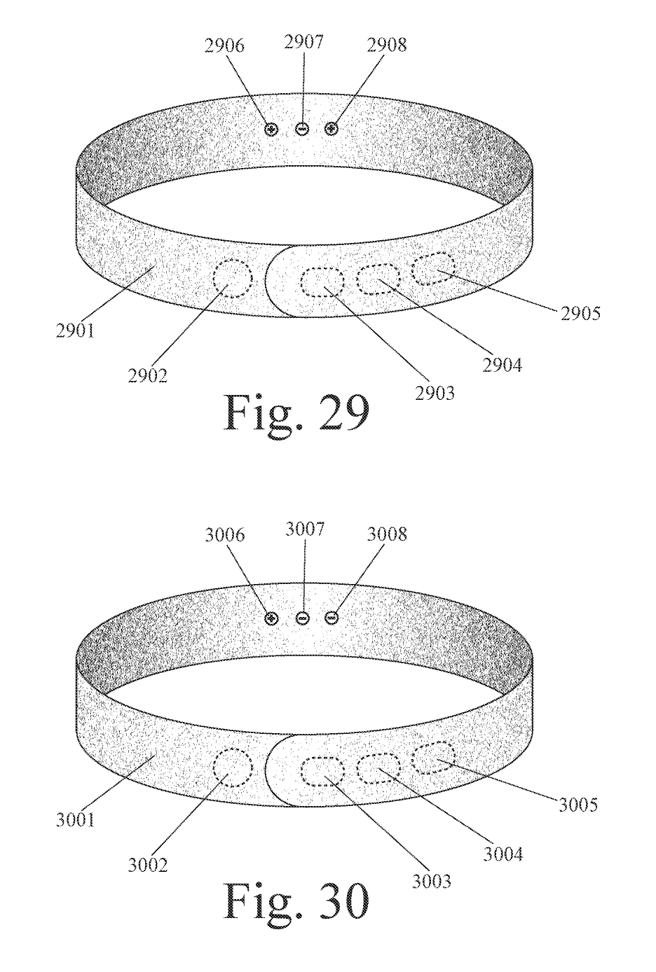

FIG. 29 shows an example of a wearable device for non-invasive glucose monitoring with two energy emitters and a single energy receiver which are all along the same circumferential line of a wearable arcuate band. In this example, the energy receiver is located between the two energy emitters. In this example, the energy receiver is located midway between the two energy emitters. In an example, different energy emitters can emit energy at different times and/or in a chronological sequence.

Specifically, FIG. 29 shows an oblique-side-perspective view of a wearable device for non-invasive glucose monitoring comprising: arcuate band 2901 which is configured to span (some or all of) the circumferential perimeter of a part of a person's body (such as a wrist, arm, finger, ankle, and/or leg); first energy emitter 2906 (identified with a plus sign) which is configured to emit energy toward the part of the person's body; second energy emitter 2908 (identified with a plus sign) which is configured to emit energy toward the part of the person's body; energy receiver 2907 (identified with a negative sign) which is configured to receive energy from the first energy emitter and/or the second energy emitter after that energy has passed through and/or been reflected from the part of the person's body, wherein the energy receiver is located between the first energy emitter and the second energy emitter; data processor 2904 which receives data from the energy receiver which is analyzed in order to measure the person's body glucose level; energy source 2903 which provides energy to the energy emitter and/or to the data processor; and data transmitter 2905 which transmits data from the data processor to a remote device and/or remote location. This example further comprises another (type of) biometric or environmental sensor 2902. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

FIG. 30 shows an example of a wearable device for non-invasive glucose monitoring with a single energy emitter and two energy receivers along the same circumferential line of a wearable arcuate band, wherein the energy emitter is to one side (e.g. clockwise or clockwise-clockwise, or to the right or left) of the two energy receivers.

Specifically, FIG. 30 shows an oblique-side-perspective view of a wearable device for non-invasive glucose monitoring comprising: arcuate band 3001 which is configured to span (some or all of) the circumferential perimeter of a part of a person's body (such as a wrist, arm, finger, ankle, and/or leg); energy emitter 3006 (identified with a plus sign) which is configured to emit energy toward the part of the person's body; first energy receiver 3007 (identified with a negative sign) which is configured to receive energy from the energy emitter after that energy has passed through and/or been reflected from the part of the person's body; second energy receiver 3008 (identified with a negative sign) which is configured to receive energy from the energy emitter after that energy has passed through and/or been reflected from the part of the person's body, wherein the energy emitter is to one side (e.g. clockwise or counter-clockwise, or to the right or left) of the first energy receiver and the second energy receiver; data processor 3004 which receives data from the energy receivers which is analyzed in order to measure the person's body glucose level; energy source 3003 which provides energy to the energy emitter and/or to the data processor; and data transmitter 3005 which transmits data from the data processor to a remote device and/or remote location. This example further comprises another (type of) biometric or environmental sensor 3002. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

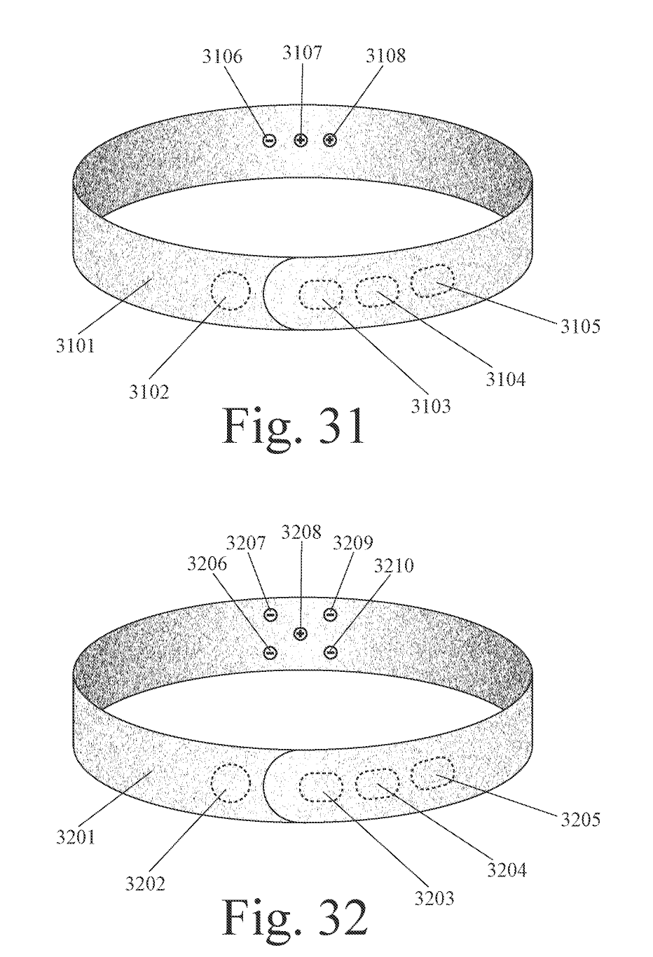

FIG. 31 shows an example of a wearable device for non-invasive glucose monitoring with a single energy receiver and two energy emitters along the same circumferential line of a wearable arcuate band, wherein the energy receiver is to one side (e.g. clockwise or clockwise-clockwise, or to the right or left) of the two energy emitters. In an example, different energy emitters can emit energy at different times and/or in a chronological sequence.

Specifically, FIG. 31 shows an oblique-side-perspective view of a wearable device for non-invasive glucose monitoring comprising: arcuate band 3101 which is configured to span (some or all of) the circumferential perimeter of a part of a person's body (such as a wrist, arm, finger, ankle, and/or leg); first energy emitter 3107 (identified with a plus sign) which is configured to emit energy toward the part of the person's body; second energy emitter 3108 (identified with a plus sign) which is configured to emit energy toward the part of the person's body; energy receiver 3106 (identified with a negative sign) which is configured to receive energy from the first energy emitter and/or the second energy emitter after that energy has passed through and/or been reflected from the part of the person's body, wherein the energy receiver is to one side (e.g. clockwise or counter-clockwise, or to the right or left) of the first energy emitter and the second energy emitter; data processor 3104 which receives data from the energy receiver which is analyzed in order to measure the person's body glucose level; energy source 3103 which provides energy to the energy emitter and/or to the data processor; and data transmitter 3105 which transmits data from the data processor to a remote device and/or remote location. This example further comprises another (type of) biometric or environmental sensor 3102. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

FIG. 32 shows an example of a wearable device for non-invasive glucose monitoring with a single central energy emitter and a plurality of energy receivers configured around the central energy emitter. In this example, energy receivers are configured in a polygonal (or approximately circular) array around the central energy emitter. In this example, energy receivers are configured in a rectangular array around the central energy emitter. In this example, the energy receivers comprise the vertexes of a polygonal (or approximately circular) array. In this example, the energy receivers comprise the vertexes of a rectangular array.

Specifically, FIG. 32 shows an oblique-side-perspective view of a wearable device for non-invasive glucose monitoring comprising: an arcuate band 3201 which is configured to span (some or all of) the circumferential perimeter of a part of a person's body (such as a wrist, arm, finger, ankle, and/or leg); a central energy emitter 3208 (identified with a plus sign) which is configured to emit energy toward the part of the person's body; a plurality of energy receivers 3206, 3207, 3209, and 3210 (identified with negative signs) which are configured to receive energy from the energy emitter after that energy has passed through and/or been reflected from the part of the person's body, wherein this plurality of energy receivers is configured in a (polygonal or circular) array around the central energy emitter; a data processor 3204 which receives data from the energy receivers which is analyzed in order to measure the person's body glucose level; an energy source 3203 which provides energy to the energy emitter and/or to the data processor; and a data transmitter 3205 which transmits data from the data processor to a remote device and/or remote location. This example further comprises another (type of) biometric or environmental sensor 3202. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

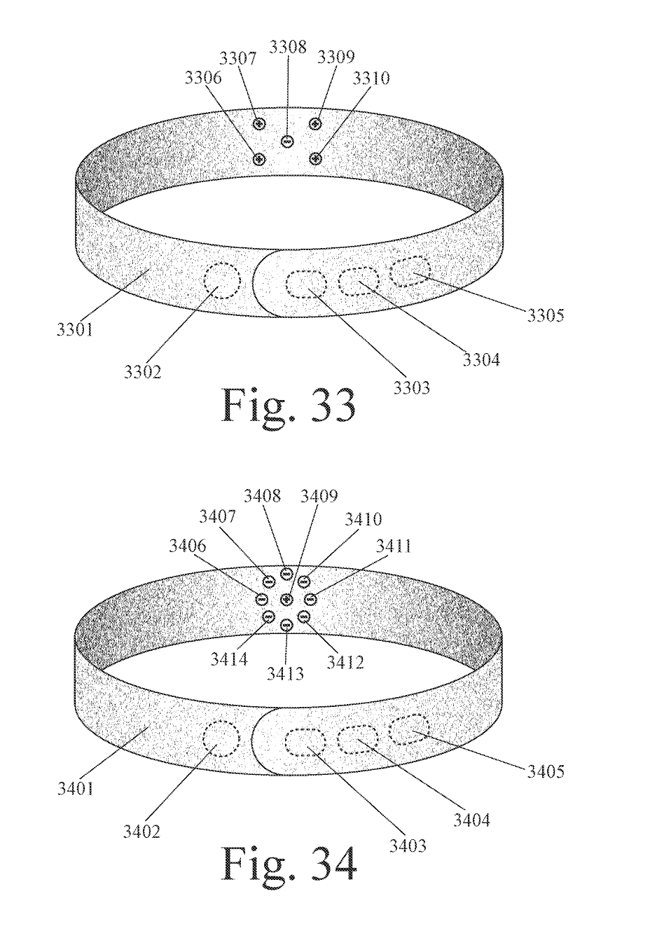

FIG. 33 shows an example of a wearable device for non-invasive glucose monitoring with a single central energy receiver and a plurality of energy emitters configured around the central energy receiver. In this example, energy emitters are configured in a polygonal (or approximately circular) array around the central energy receiver. In this example, energy emitters are configured in a rectangular array around the central energy receiver. In this example, the energy emitters comprise the vertexes of a polygonal (or approximately circular) array. In this example, the energy emitters comprise the vertexes of a rectangular array. In an example, different energy emitters can emit energy at different times and/or in a chronological sequence.

Specifically, FIG. 33 shows an oblique-side-perspective view of a wearable device for non-invasive glucose monitoring comprising: an arcuate band 3301 which is configured to span (some or all of) the circumferential perimeter of a part of a person's body (such as a wrist, arm, finger, ankle, and/or leg); a plurality of energy emitters 3306, 3307, 3309, and 910 (identified with positive signs) which are configured to emit energy toward the part of the person's body; a central energy receiver 3308 (identified with a negative sign) which is configured to receive energy from the energy emitters after that energy has passed through and/or been reflected from the part of the person's body, wherein the plurality of energy emitters is configured in a (polygonal or circular) array around the central energy receiver; a data processor 3304 which receives data from the energy receiver which is analyzed in order to measure the person's body glucose level; an energy source 3303 which provides energy to the energy emitter and/or to the data processor; and a data transmitter 3305 which transmits data from the data processor to a remote device and/or remote location. This example further comprises another (type of) biometric or environmental sensor 3302. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

FIG. 34 shows an example of a wearable device for non-invasive glucose monitoring with a single central energy emitter and a plurality of energy receivers configured around the central energy emitter. In this example, energy receivers are configured in a circular (approximately-circular octagonal) array around the central energy emitter. In this example, the energy receivers comprise the vertexes of an (approximately-circular octagonal) array.

Specifically, FIG. 34 shows an oblique-side-perspective view of a wearable device for non-invasive glucose monitoring comprising: an arcuate band 3401 which is configured to span (some or all of) the circumferential perimeter of a part of a person's body (such as a wrist, arm, finger, ankle, and/or leg); a central energy emitter 3409 (identified with a plus sign) which is configured to emit energy toward the part of the person's body; a plurality of energy receivers 3406, 3407, 3408, 3410, 3411, 3412, 3413, and 3414 (identified with negative signs) which are configured to receive energy from the energy emitter after that energy has passed through and/or been reflected from the part of the person's body, wherein the plurality of energy receivers is configured in a circular (or approximately-circular polygonal) array around the central energy emitter; a data processor 3404 which receives data from the energy receivers which is analyzed in order to measure the person's body glucose level; an energy source 3403 which provides energy to the energy emitter and/or to the data processor; and a data transmitter 3405 which transmits data from the data processor to a remote device and/or remote location. This example further comprises another (type of) biometric or environmental sensor 3402. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

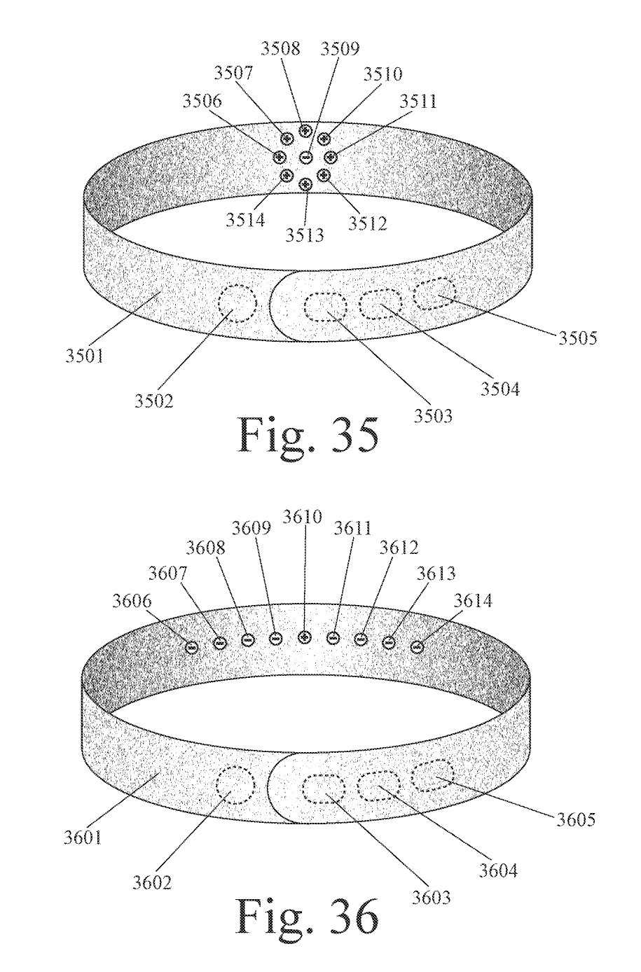

FIG. 35 shows an example of a wearable device for non-invasive glucose monitoring with a central energy receiver and a plurality of energy emitters configured around the central energy receiver. In this example, energy emitters are configured in a circular (approximately-circular octagonal) array around the central energy receiver. In this example, the energy emitters comprise the vertexes of an approximately-circular octagonal array. In an example, different energy emitters can emit energy at different times and/or in a chronological sequence.

Specifically, FIG. 35 shows an oblique-side-perspective view of a wearable device for non-invasive glucose monitoring comprising: an arcuate band 3501 which is configured to span (some or all of) the circumferential perimeter of a part of a person's body (such as a wrist, arm, finger, ankle, and/or leg); a plurality of energy emitters 3506, 3507, 3508, 3510, 3511, 3512, 3513, and 3514 (identified with plus signs) which are configured to emit energy toward the part of the person's body; a central energy receiver 3509 (identified with a negative sign) which is configured to receive energy from the energy emitters after that energy has passed through and/or been reflected from the part of the person's body, wherein the plurality of energy emitters is configured in a circular (or approximately-circular polygonal) array around the central energy receiver; a data processor 3504 which receives data from the energy receiver which is analyzed in order to measure the person's body glucose level; an energy source 3503 which provides energy to the energy emitter and/or to the data processor; and a data transmitter 3505 which transmits data from the data processor to a remote device and/or remote location. This example further comprises another (type of) biometric or environmental sensor 3502. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

FIG. 36 shows an example of a wearable device for non-invasive glucose monitoring with a central energy emitter and a plurality of (more than four) energy receivers along the same circumferential line of a wearable arcuate band, wherein the central energy emitter is centrally located with respect to the energy receivers. In this example, there are eight energy receivers in total, four on each side of the central energy emitter. In this example, the energy receivers and the central energy emitter are evenly spaced around a portion of the circumference of a band. In this example, the energy receivers and the central energy emitter are each the same distance from the closest energy receiver or energy emitter.

Specifically, FIG. 36 shows an oblique-side-perspective view of a wearable device for non-invasive glucose monitoring comprising: arcuate band 3601 which is configured to span (some or all of) the circumferential perimeter of a part of a person's body (such as a wrist, arm, finger, ankle, and/or leg); central energy emitter 3610 (identified with a plus sign) which is configured to emit energy toward the part of the person's body; a plurality of (more than four) energy emitters 3606, 3607, 3608, 3609, 3611, 3612, 3613, and 3614 (identified with negative signs) which are configured to receive energy from the central energy emitter after that energy has passed through and/or been reflected from the part of the person's body, wherein the central energy emitter and the energy emitters are all along the same circumferential line of the band, and wherein the central energy emitter is centrally located with respect to the energy emitters; data processor 3604 which receives data from the energy receivers which is analyzed in order to measure the person's body glucose level; energy source 3603 which provides energy to the central energy emitter and/or to the data processor; and data transmitter 3605 which transmits data from the data processor to a remote device and/or remote location.

This example further comprises another (type of) biometric or environmental sensor 3602. In another example, one or more energy emitters and/or receivers can be part of a housing which is attached to the band instead of being part of the band itself. In an example, the central energy emitter and the energy receivers can be distributed around the circumference of a band, but not all be along the same circumferential line. Other example variations and component elaborations discussed elsewhere in this disclosure (or in other disclosures within the priority-linked family) can also be applied to this example.

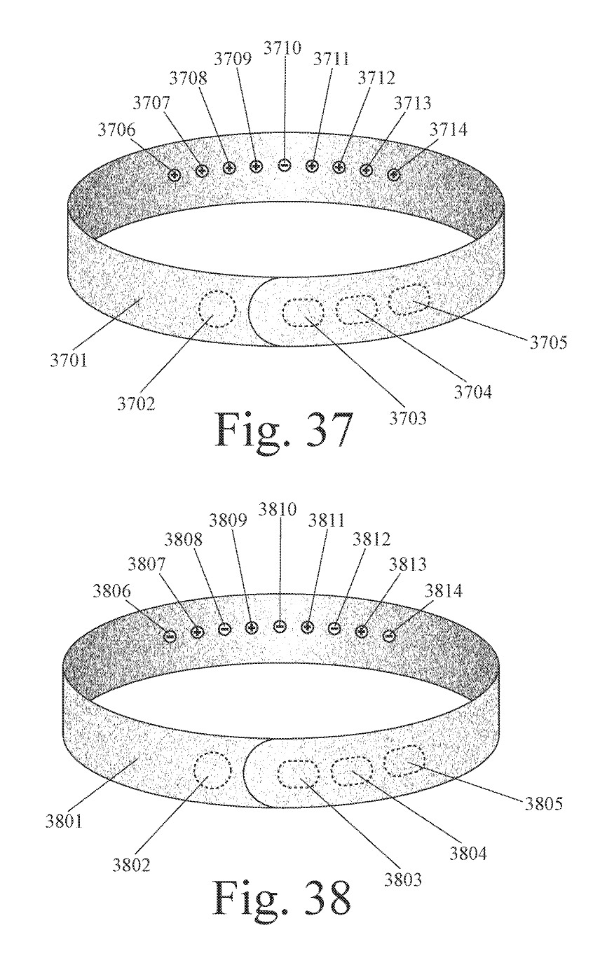

FIG. 37 shows an example of a wearable device for non-invasive glucose monitoring with a central energy receiver and a plurality of (more than four) energy emitters along the same circumferential line of a wearable arcuate band, wherein the central energy receiver is centrally located with respect to the energy emitters. In this example, there are eight energy emitters in total, four on each side of the central energy receiver. In this example, the energy emitters and the central energy receiver are evenly spaced around a portion of the circumference of a band. In this example, the energy emitters and the central energy receiver are each the same distance from the closest energy emitter or energy receiver. In an example, different energy emitters can emit energy at different times and/or in a chronological sequence.

Specifically, FIG. 37 shows an oblique-side-perspective view of a wearable device for non-invasive glucose monitoring comprising: arcuate band 3701 which is configured to span (some or all of) the circumferential perimeter of a part of a person's body (such as a wrist, arm, finger, ankle, and/or leg); a plurality of (more than four) energy emitters 3706, 3707, 3708, 3709, 3711, 3712, 3713, and 3714 (identified with plus signs) which are configured to emit energy toward the part of the person's body; a central energy receiver 3710 (identified with a negative sign) which is configured to receive energy from the energy emitters after that energy has passed through and/or been reflected from the part of the person's body, wherein the central energy receiver and the energy receivers are all along the same circumferential line of the band, and wherein the central energy receiver is centrally located with respect to the energy receivers; a data processor 3704 which receives data from the energy receiver which is analyzed in order to measure the person's body glucose level; an energy source 3703 which provides energy to the central energy emitter and/or to the data processor; and a data transmitter 3705 which transmits data from the data processor to a remote device and/or remote location.

This example further comprises another (type of) biometric or environmental sensor 3702. In another example, one or more energy emitters and/or receivers can be part of a housing which is attached to the band instead of being part of the band itself. In an example, the central energy receiver and the energy emitters can be distributed around the circumference of a band, but not all be along the same circumferential line. Other example variations and component elaborations discussed elsewhere in this disclosure (or in other disclosures within the priority-linked family) can also be applied to this example.

FIG. 38 shows an example of a wearable device for non-invasive glucose monitoring with a plurality of energy emitters and a plurality of energy receivers which are all along the same circumferential line of a wearable arcuate band. In this example, there are multiple pairs of energy emitters and energy receivers along the same circumferential line of a wearable arcuate band. In this example, there are at least two pairs of energy emitters and energy receivers along a circumferential line of a wearable arcuate band. In this example, there are four pairs of energy emitters and energy receivers along a circumferential line of a wearable arcuate band. In this example, there are alternating energy emitters and energy receivers along a circumferential line of a wearable arcuate band. In an example, different energy emitters can emit energy at different times and/or in a chronological sequence.

Specifically, FIG. 38 shows an oblique-side-perspective view of a wearable device for non-invasive glucose monitoring comprising: arcuate band 3801 which is configured to span (some or all of) the circumferential perimeter of a part of a person's body (such as a wrist, arm, finger, ankle, and/or leg); a plurality of energy emitters 3807, 3809, 3811, and 3813 (identified with plus signs) which are configured to emit energy toward the part of the person's body; a plurality of energy receivers 3806, 3808, 3810, 3812, and 3814 (identified with negative signs) which are configured to receive energy from the plurality of energy emitters after that energy has passed through and/or been reflected from the part of the person's body, wherein the energy emitters and the energy receivers are all along the same circumferential line of the band, and wherein the energy emitters and the energy receivers alternate sequentially along the circumferential line of the band; a data processor 3804 which receives data from the energy receivers which is analyzed in order to measure the person's body glucose level; an energy source 3803 which provides energy to the central energy emitter and/or to the data processor; and a data transmitter 3805 which transmits data from the data processor to a remote device and/or remote location.

FIG. 38 also shows an oblique-side-perspective view of a wearable device for non-invasive glucose monitoring comprising: arcuate band 3801 which is configured to span (some or all of) the circumferential perimeter of a part of a person's body (such as a wrist, arm, finger, ankle, and/or leg); a plurality of pairs of energy emitters 3807, 3809, 3811, and 3813 (identified with plus signs) and energy receivers 3806, 3808, 3810, and 3812 (identified with negative signs), wherein the energy emitters are configured to emit energy toward the part of the person's body, wherein the energy receivers receive energy from the plurality of energy emitters after that energy has passed through and/or been reflected from the part of the person's body, wherein the pairs of energy emitters and energy receivers are along the same circumferential line of the band; a data processor 3804 which receives data from the energy receivers which is analyzed in order to measure the person's body glucose level; an energy source 3803 which provides energy to the central energy emitter and/or to the data processor; and a data transmitter 3805 which transmits data from the data processor to a remote device and/or remote location.

This example further comprises another (type of) biometric or environmental sensor 3802. In another example, one or more energy emitters and/or receivers can be part of a housing which is attached to the band instead of being part of the band itself. In an example, the central energy receiver and the energy emitters can be distributed around the circumference of a band, but not all be along the same circumferential line. Other example variations and component elaborations discussed elsewhere in this disclosure (or in other disclosures within the priority-linked family) can also be applied to this example.

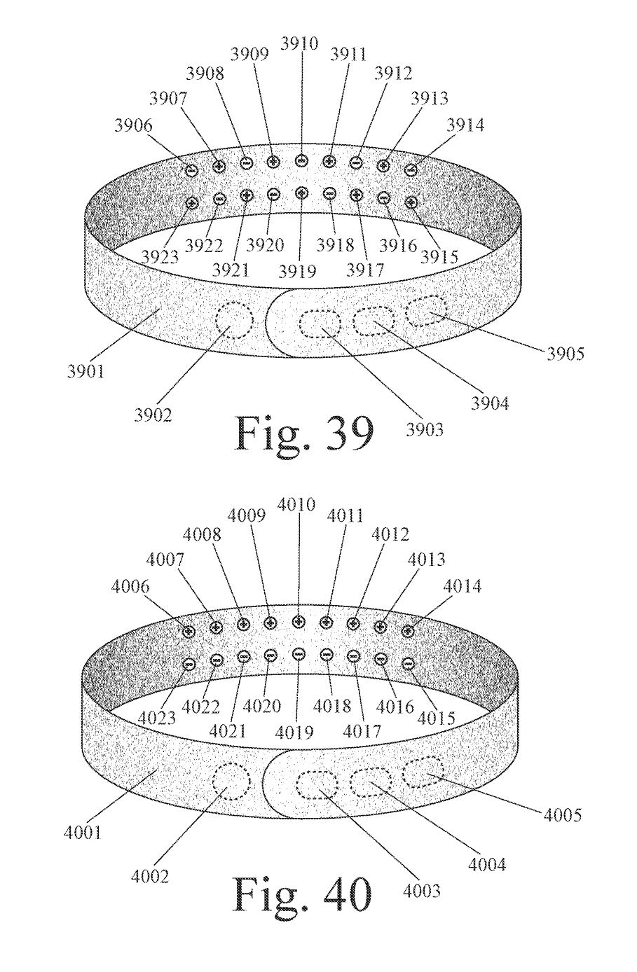

FIG. 39 shows an example of a wearable device for non-invasive glucose monitoring with a first plurality of energy emitters and energy receivers along a first circumferential line of a wearable arcuate band and a second plurality of energy emitters and energy receivers along a second circumferential line of the wearable arcuate band. In this example, there is a first set of multiple pairs of energy emitters and energy receivers along a first circumferential line of a wearable arcuate band and a second set of multiple pairs of energy emitters and energy receivers along a second circumferential line of a wearable arcuate band. In this example, there is a first set of alternating energy emitters and energy receivers along a first circumferential line of a wearable arcuate band and a second set of alternating energy emitters and energy receivers along a second circumferential line of a wearable arcuate band. In an example, different energy emitters can emit energy at different times and/or in a chronological sequence.

Specifically, FIG. 39 shows an oblique-side-perspective view of a wearable device for non-invasive glucose monitoring comprising: arcuate band 3901 which is configured to span (some or all of) the circumferential perimeter of a part of a person's body (such as a wrist, arm, finger, ankle, and/or leg); a first plurality of energy emitters 3907, 3909, 3911, and 3913 (identified with plus signs) and energy receivers 3906, 3908, 3910, 3912, and 3914 (identified with negative signs) along a first circumferential line of the arcuate band; a second plurality of energy emitters 3923, 3921, 3919, 3917, and 3915 (identified with plus signs) and energy receivers 3922, 3920, 3918, and 3916 (identified with negative signs) along a second circumferential line of the arcuate band, wherein energy emitters are configured to emit energy toward the part of the person's body, and wherein energy receivers are configured to receive energy from energy emitters after that energy has passed through and/or been reflected from the part of the person's body; a data processor 3904 which receives data from the energy receivers which is analyzed in order to measure the person's body glucose level; an energy source 3903 which provides energy to the energy emitters and/or to the data processor; and a data transmitter 3905 which transmits data from the data processor to a remote device and/or remote location.

This example further comprises another (type of) biometric or environmental sensor 3902. In another example, one or more energy emitters and/or receivers can be part of a housing which is attached to the band instead of being part of the band itself. In an example, the central energy receiver and the energy emitters can be distributed around the circumference of a band, but not all be along the same circumferential line. Other example variations and component elaborations discussed elsewhere in this disclosure (or in other disclosures within the priority-linked family) can also be applied to this example.

FIG. 40 shows an example of a wearable device for non-invasive glucose monitoring with a plurality of energy emitters along a first circumferential line of a wearable arcuate band and a plurality of energy receivers along a second circumferential line of the wearable arcuate band. In an example, different energy emitters can emit energy at different times and/or in a chronological sequence.

Specifically, FIG. 40 shows an oblique-side-perspective view of a wearable device for non-invasive glucose monitoring comprising: arcuate band 4001 which is configured to span (some or all of) the circumferential perimeter of a part of a person's body (such as a wrist, arm, finger, ankle, and/or leg); a plurality of energy emitters 4006, 4007, 4008, 4009, 4010, 4011, 4012, 4013, and 4014 (identified with plus signs) along a first circumferential line of the arcuate band, wherein energy emitters are configured to emit energy toward the part of the person's body; a plurality of energy receivers 4023, 4022, 4021, 4020, 4019, 4018, 4017, 4016, and 4015 (identified with negative signs) along a second circumferential line of the arcuate band, wherein energy receivers are configured to receive energy from energy emitters after that energy has passed through and/or been reflected from the part of the person's body; a data processor 4004 which receives data from the energy receivers which is analyzed in order to measure the person's body glucose level; an energy source 4003 which provides energy to the energy emitters and/or to the data processor; and a data transmitter 4005 which transmits data from the data processor to a remote device and/or remote location.

This example further comprises another (type of) biometric or environmental sensor 4002. In another example, one or more energy emitters and/or receivers can be part of a housing which is attached to the band instead of being part of the band itself. In an example, the central energy receiver and the energy emitters can be distributed around the circumference of a band, but not all be along the same circumferential line. Other example variations and component elaborations discussed elsewhere in this disclosure (or in other disclosures within the priority-linked family) can also be applied to this example.

FIG. 41 shows an example of a wearable device for non-invasive glucose monitoring comprising: a single energy emitter and a single energy receiver which are both located along the same circumferential line of an arcuate band; and an energy barrier around the energy receiver. The energy barrier reduces energy transmission from the energy emitter to the energy receiver apart from energy transmission through body tissue. In this example, the energy barrier is circular and fully encircles the energy receiver. In examples wherein the energy which is emitted and received is light energy, then the energy barrier can be opaque. In an example, an energy barrier can be made from opaque compressible material or an opaque inflatable member.

Specifically, FIG. 41 shows an oblique-side-perspective view of a wearable device for non-invasive glucose monitoring comprising: arcuate band 4101 which is configured to span (some or all of) the circumferential perimeter of a part of a person's body (such as a wrist, arm, finger, ankle, and/or leg); energy emitter 4106 (identified with a plus sign) which is configured to emit energy toward the part of the person's body; energy receiver 4107 (identified with a negative sign) which is configured to receive energy from the energy emitter after that energy has passed through and/or been reflected from the part of the person's body; energy barrier 4108 between the energy emitter and the energy receiver, wherein the energy barrier is configured to reduce energy transmission from the energy emitter to the energy receiver apart from energy transmission through the person's body and wherein the energy barrier encircles the energy receiver; data processor 4104 which receives data from the energy receiver which is analyzed in order to measure the person's body glucose level; energy source 4103 which provides energy to the energy emitter and/or to the data processor; and data transmitter 4105 which transmits data from the data processor to a remote device and/or remote location. This example further comprises another (type of) biometric or environmental sensor 4102. In another example, an energy emitter, an energy receiver, or both can be part of a housing which is attached to the band, rather than part of the band itself. Other example variations and component elaborations discussed elsewhere in this disclosure (or in other disclosures within the priority-linked family) can also be applied to this example.

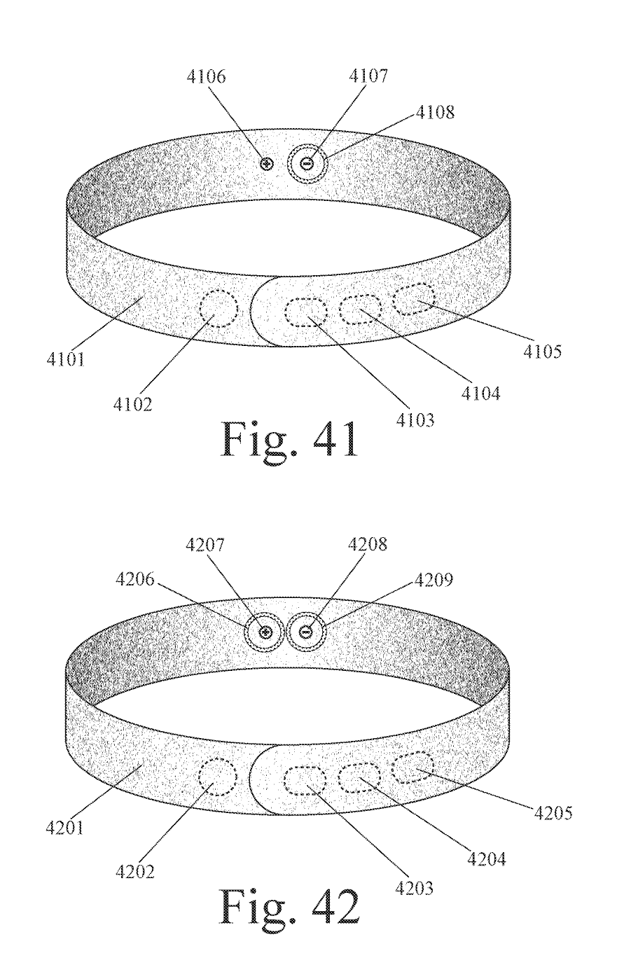

FIG. 42 shows an example of a wearable device for non-invasive glucose monitoring comprising: a single energy emitter and a single energy receiver which are both located along the same circumferential line of an arcuate band; an energy barrier around the energy emitter, and an energy barrier around the energy receiver. The energy barriers reduce energy transmission from the energy emitter to the energy receiver apart from energy transmission through body tissue. In this example, the energy barriers are circular. In examples wherein the energy which is emitted and received is light energy, energy barriers can be opaque. In an example, energy barriers can be made from opaque compressible material or opaque inflatable members.

Specifically, FIG. 42 shows an oblique-side-perspective view of a wearable device for non-invasive glucose monitoring comprising: arcuate band 4201 which is configured to span (some or all of) the circumferential perimeter of a part of a person's body (such as a wrist, arm, finger, ankle, and/or leg); energy emitter 4207 (identified with a plus sign) which is configured to emit energy toward the part of the person's body; energy receiver 4208 (identified with a negative sign) which is configured to receive energy from the energy emitter after that energy has passed through and/or been reflected from the part of the person's body; first compressible energy barrier 4206 around the energy emitter; and second compressible energy barrier 4209 around the energy receiver; data processor 4204 which receives data from the energy receiver which is analyzed in order to measure the person's body glucose level; energy source 4203 which provides energy to the energy emitter and/or to the data processor; and data transmitter 4205 which transmits data from the data processor to a remote device and/or remote location. This example further comprises another (type of) biometric or environmental sensor 4202. In another example, an energy emitter, an energy receiver, or both can be part of a housing which is attached to the band, rather than part of the band itself. Other example variations and component elaborations discussed elsewhere in this disclosure (or in other disclosures within the priority-linked family) can also be applied to this example.

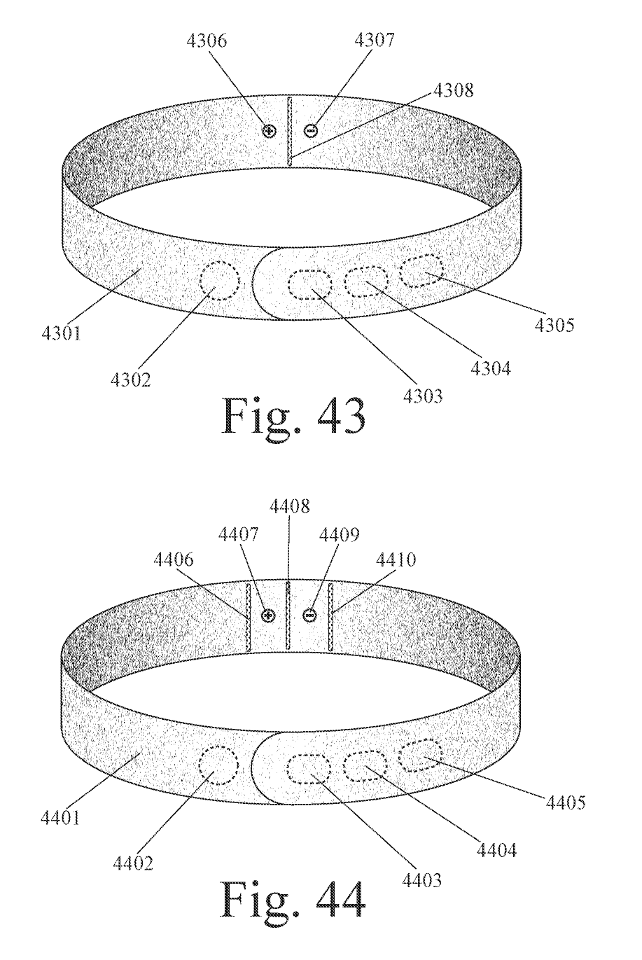

FIG. 43 shows an example of a wearable device for non-invasive glucose monitoring comprising a single energy emitter and a single energy receiver (which are both located along the same circumferential line of an arcuate band) and a proximal-to-distal energy barrier between them. The energy barrier reduces energy transmission from the energy emitter to the energy receiver apart from energy transmission through body tissue. In this example, the energy barrier is linear and midway between the energy emitter and the energy receiver. In examples wherein the energy which is emitted and received is light energy, then the energy barrier can be opaque. In an example, an energy barrier can be made from opaque compressible material or an opaque inflatable member.

Specifically, FIG. 43 shows an oblique-side-perspective view of a wearable device for non-invasive glucose monitoring comprising: arcuate band 4301 which is configured to span (some or all of) the circumferential perimeter of a part of a person's body (such as a wrist, arm, finger, ankle, and/or leg); energy emitter 4306 (identified with a plus sign) which is configured to emit energy toward the part of the person's body; energy receiver 4307 (identified with a negative sign) which is configured to receive energy from the energy emitter after that energy has passed through and/or been reflected from the part of the person's body; a proximal-to-distal energy barrier 4308 (midway) between the energy emitter and the energy receiver; data processor 4304 which receives data from the energy receiver which is analyzed in order to measure the person's body glucose level; energy source 4303 which provides energy to the energy emitter and/or to the data processor; and data transmitter 4305 which transmits data from the data processor to a remote device and/or remote location. This example further comprises another (type of) biometric or environmental sensor 4302. In another example, an energy emitter, an energy receiver, or both can be part of a housing which is attached to the band, rather than part of the band itself. Other example variations and component elaborations discussed elsewhere in this disclosure (or in other disclosures within the priority-linked family) can also be applied to this example.

FIG. 44 shows an example of a wearable device for non-invasive glucose monitoring comprising a single energy emitter and a single energy receiver (which are both located along the same circumferential line of an arcuate band) and three proximal-to-distal energy barriers. In this example, there is a first energy barrier between the emitter and receiver, a second energy barrier to the left of the emitter and receiver, and a third energy barrier to the right of the emitter and receiver. In an example, energy barriers can be made from opaque compressible material or opaque inflatable members. In this example, each of the three energy barriers is proximal-to-distal and linear.

Specifically, FIG. 44 shows an oblique-side-perspective view of a wearable device for non-invasive glucose monitoring comprising: arcuate band 4401 which is configured to span (some or all of) the circumferential perimeter of a part of a person's body (such as a wrist, arm, finger, ankle, and/or leg); an energy emitter 4407 (identified with a plus sign) which is configured to emit energy toward the part of the person's body; an energy receiver 4409 (identified with a negative sign) which is configured to receive energy from the energy emitter after that energy has passed through and/or been reflected from the part of the person's body; a first energy barrier 4406 between the energy emitter and the energy receiver; a second energy barrier 4408 to the left of (or counter-clockwise from) the energy emitter and the energy receiver; a third energy barrier 4410 to the right of (or clockwise from) the energy emitter and the energy receiver; a data processor 4404 which receives data from the energy receiver which is analyzed in order to measure the person's body glucose level; an energy source 4403 which provides energy to the energy emitter and/or to the data processor; and a data transmitter 4405 which transmits data from the data processor to a remote device and/or remote location. This example further comprises another (type of) biometric or environmental sensor 4402. In another example, an energy emitter, an energy receiver, or both can be part of a housing which is attached to the band, rather than part of the band itself. Other example variations and component elaborations discussed elsewhere in this disclosure (or in other disclosures within the priority-linked family) can also be applied to this example.

FIG. 45 shows an example of a wearable device for non-invasive glucose monitoring with a single energy emitter, two energy receivers, and two energy barriers (one surrounding each energy receiver). In this example, the energy emitter is located between the two energy receivers. In this example, the energy emitter is located midway between the two energy receivers. The energy barriers reduce energy transmission from the energy emitter to the energy receiver apart from energy transmission through body tissue. In this example, the energy barriers are circular. In an example, the energy barriers can be made from opaque compressible material or opaque inflatable members.

Specifically, FIG. 45 shows an oblique-side-perspective view of a wearable device for non-invasive glucose monitoring comprising: arcuate band 4501 which is configured to span (some or all of) the circumferential perimeter of a part of a person's body (such as a wrist, arm, finger, ankle, and/or leg); energy emitter 4508 (identified with a plus sign) which is configured to emit energy toward the part of the person's body; first energy receiver 4507 (identified with a negative sign) which is configured to receive energy from the energy emitter after that energy has passed through and/or been reflected from the part of the person's body; second energy receiver 4509 (identified with a negative sign) which is configured to receive energy from the energy emitter after that energy has passed through and/or been reflected from the part of the person's body, wherein the energy emitter is located between the first energy receiver and the second energy receiver; first energy barrier 4506 around the first energy receiver; second energy barrier 4510 around the second energy receiver; data processor 4504 which receives data from the energy receivers which is analyzed in order to measure the person's body glucose level; energy source 4503 which provides energy to the energy emitter and/or to the data processor; and data transmitter 4505 which transmits data from the data processor to a remote device and/or remote location. This example further comprises another (type of) biometric or environmental sensor 4502. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

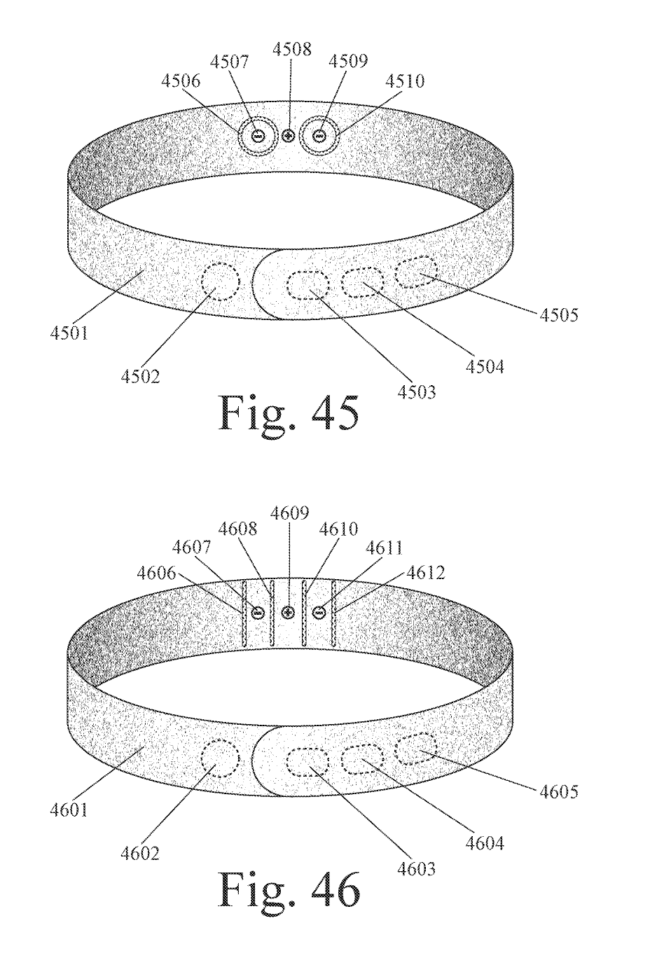

FIG. 46 shows an example of a wearable device for non-invasive glucose monitoring with a single energy emitter, two energy receivers, and four proximal-to-distal energy barriers. In this example, the energy emitter is located between the two energy receivers. In this example, the energy emitter is located midway between the two energy receivers. The energy barriers reduce energy transmission from the energy emitter to the energy receiver apart from energy transmission through body tissue. In an example, the energy barriers can be made from opaque compressible material or opaque inflatable members.

Specifically, FIG. 46 shows an oblique-side-perspective view of a wearable device for non-invasive glucose monitoring comprising: arcuate band 4601 which is configured to span (some or all of) the circumferential perimeter of a part of a person's body (such as a wrist, arm, finger, ankle, and/or leg); an energy emitter 4609 (identified with a plus sign) which is configured to emit energy toward the part of the person's body; a first energy receiver 4607 (identified with a negative sign) which is configured to receive energy from the energy emitter after that energy has passed through and/or been reflected from the part of the person's body; a second energy receiver 4611 (identified with a negative sign) which is configured to receive energy from the energy emitter after that energy has passed through and/or been reflected from the part of the person's body, wherein the energy emitter is located between the first energy receiver and the second energy receiver; a first energy barrier 4606 to the left of the energy receivers and the energy emitter; a second energy barrier 4608 between the first energy receiver and the energy emitter; a third energy barrier 4610 between the second energy receiver and the energy emitter; a fourth energy barrier 4612 to the right of the energy receivers and the energy emitter; a data processor 4604 which receives data from the energy receivers which is analyzed in order to measure the person's body glucose level; an energy source 4603 which provides energy to the energy emitter and/or to the data processor; and a data transmitter 4605 which transmits data from the data processor to a remote device and/or remote location. This example further comprises another (type of) biometric or environmental sensor 4602. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

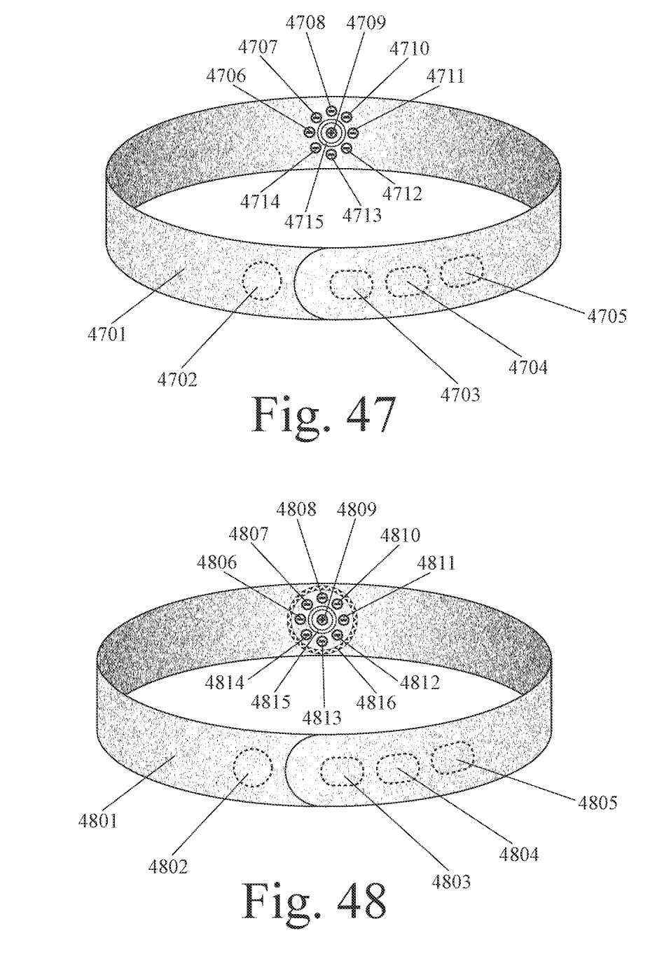

FIG. 47 shows an example of a wearable device for non-invasive glucose monitoring with a single central energy emitter, a plurality of energy receivers around the central energy emitter, and an energy barrier between the central energy emitter and the plurality of energy receivers. In this example, energy receivers are configured in a circular (or approximately-circular polygonal) array around the central energy emitter. In this example, the energy barrier is arcuate. In this example, the energy barrier is circular. In an example wherein the type of energy that is emitted and received is light energy, the energy barrier can be opaque. In an example wherein the type of energy that is emitted and received is light energy, the energy barrier can be made from a compressible material (such as foam) or an inflated member (such as a balloon).

Specifically, FIG. 47 shows an oblique-side-perspective view of a wearable device for non-invasive glucose monitoring comprising: an arcuate band 4701 which is configured to span (some or all of) the circumferential perimeter of a part of a person's body (such as a wrist, arm, finger, ankle, and/or leg); a central energy emitter 4709 (identified with a plus sign) which is configured to emit energy toward the part of the person's body; a plurality of energy receivers 4706, 4707, 4708, 4710, 4711, 4712, 4713, and 4714 (identified with negative signs) which are configured to receive energy from the energy emitter after that energy has passed through and/or been reflected from the part of the person's body, wherein the plurality of energy receivers is configured in a circular (or approximately-circular polygonal) array around the central energy emitter; an energy barrier 4715 between the central energy emitter and the plurality of energy receivers; a data processor 4704 which receives data from the energy receivers which is analyzed in order to measure the person's body glucose level; an energy source 4703 which provides energy to the energy emitter and/or to the data processor; and a data transmitter 4705 which transmits data from the data processor to a remote device and/or remote location. This example further comprises another (type of) biometric or environmental sensor 4702. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

FIG. 48 shows an example of a wearable device for non-invasive glucose monitoring with a single central energy emitter, a plurality of energy receivers configured around the central energy emitter, a first energy barrier between the central energy emitter and the plurality of energy receivers, and a second energy barrier around the plurality of energy receivers. In this example, energy receivers are configured in a circular (or approximately-circular polygonal) array around the central energy emitter. In this example, the energy barriers are arcuate. In this example, the energy barriers are circular. In an example wherein the type of energy that is emitted and received is light energy, the energy barriers can be opaque. In an example wherein the type of energy that is emitted and received is light energy, the energy barriers can be made from a compressible material (such as foam) or inflated members (such as balloons).

Specifically, FIG. 48 shows an oblique-side-perspective view of a wearable device for non-invasive glucose monitoring comprising: an arcuate band 4801 which is configured to span (some or all of) the circumferential perimeter of a part of a person's body (such as a wrist, arm, finger, ankle, and/or leg); a central energy emitter 4809 (identified with a plus sign) which is configured to emit energy toward the part of the person's body; a plurality of energy receivers 4806, 4807, 4808, 4810, 4811, 4812, 4813, and 4814 (identified with negative signs) which are configured to receive energy from the energy emitter after that energy has passed through and/or been reflected from the part of the person's body, wherein the plurality of energy receivers is configured in a circular (or approximately-circular polygonal) array around the central energy emitter; a first energy barrier 4815 between the central energy emitter and the plurality of energy receivers; a second energy barrier 4816 around the plurality of energy receivers; a data processor 4804 which receives data from the energy receivers which is analyzed in order to measure the person's body glucose level; an energy source 4803 which provides energy to the energy emitter and/or to the data processor; and a data transmitter 4805 which transmits data from the data processor to a remote device and/or remote location. This example further comprises another (type of) biometric or environmental sensor 4802. In another example, one or more energy emitters and/or receivers can be part of a housing which is attached to the band instead of being part of the band itself. Other example variations and component elaborations discussed elsewhere in this disclosure (or in other disclosures within the priority-linked family) can also be applied to this example.

FIG. 49 shows an example of a wearable device for non-invasive glucose monitoring with a first set of energy emitters and energy receivers along a first circumferential line of a wearable arcuate band, a second set of energy emitters and energy receivers along a second circumferential line of the wearable arcuate band, and a third set of energy emitters and energy receivers along a third circumferential line of the wearable arcuate band. In an example, these circumferential lines are parallel to each other. In an example, there are at least three energy emitters and/or energy receivers in each set. In an example, different energy emitters can emit energy at different times and/or in a chronological sequence.

In this example, energy emitters and energy receivers alternate along a circumferential line. In this example, there is an alternating sequence of energy emitters and energy receivers along a circumferential line. In this example, there are multiple triads of energy emitters and energy receivers in the first, second, and third sets, wherein a triad is aligned along the same proximal-to-distal line. In an example, there can be at least two energy emitters and two energy receivers in each set. In an example, there can be at least four energy emitters and four energy receivers in each set. In an example, energy emitters and energy receivers can be evenly-spaced within each set along a circumferential line. In this example, energy emitters and energy receivers can also be evenly-spaced between sets.

In this example, there is an arcuate array of energy emitters and energy receivers on an arcuate wearable band wherein this array has a circumferential axis (along which there are nine energy emitters and/or energy receivers) and a proximal-to-distal axis (along which there are three energy emitters and/or energy receivers). In an example, there can be an arcuate array of energy emitters and energy receivers on an arcuate wearable band wherein this array has a circumferential axis (along which there are at least three energy emitters and/or energy receivers) and a proximal-to-distal axis (along which there are at least two energy emitters and/or energy receivers).

In this example, there is an arcuate array of energy emitters and energy receivers on an arcuate wearable band wherein this array includes three parallel circumferential lines (along each of which there are nine energy emitters and/or energy receivers) and three parallel proximal-to-distal lines (along each of which there are three energy emitters and/or energy receivers). In an example, there can be an arcuate array of energy emitters and energy receivers on an arcuate wearable band wherein this array includes at least two parallel circumferential lines (along each of which there at least three energy emitters and/or energy receivers) and at least two parallel proximal-to-distal lines (along each of which there are at least two energy emitters and/or energy receivers).

Specifically, FIG. 49 shows an oblique-side-perspective view of a wearable device for non-invasive glucose monitoring comprising: arcuate band 4901 which is configured to span (some or all of) the circumferential perimeter of a part of a person's body (such as a wrist, arm, finger, ankle, and/or leg); a first set of three or more energy emitters (including 4909) and/or energy receivers (including 4908) along a first circumferential line of the arcuate band; a second set of three or more energy emitters (including 4907) and/or energy receivers (including 4910) along a second circumferential line of the arcuate band; a third set of three or more energy emitters (including 4911) and/or energy receivers (including 4906) along a third circumferential line of the arcuate band, wherein energy emitters are configured to emit energy toward the part of the person's body, and wherein energy receivers are configured to receive energy from energy emitters after that energy has passed through and/or been reflected from the part of the person's body; a data processor 4904 which receives data from the energy receivers which is analyzed in order to measure the person's body glucose level; an energy source 4903 which provides energy to the central energy emitter and/or to the data processor; and a data transmitter 4905 which transmits data from the data processor to a remote device and/or remote location.

This example further comprises another (type of) biometric or environmental sensor 4902. In another example, one or more energy emitters and/or receivers can be part of a housing which is attached to the band instead of being part of the band itself. In an example, the central energy receiver and the energy emitters can be distributed around the circumference of a band, but not all be along the same circumferential line. Other example variations and component elaborations discussed elsewhere in this disclosure (or in other disclosures within the priority-linked family) can also be applied to this example.

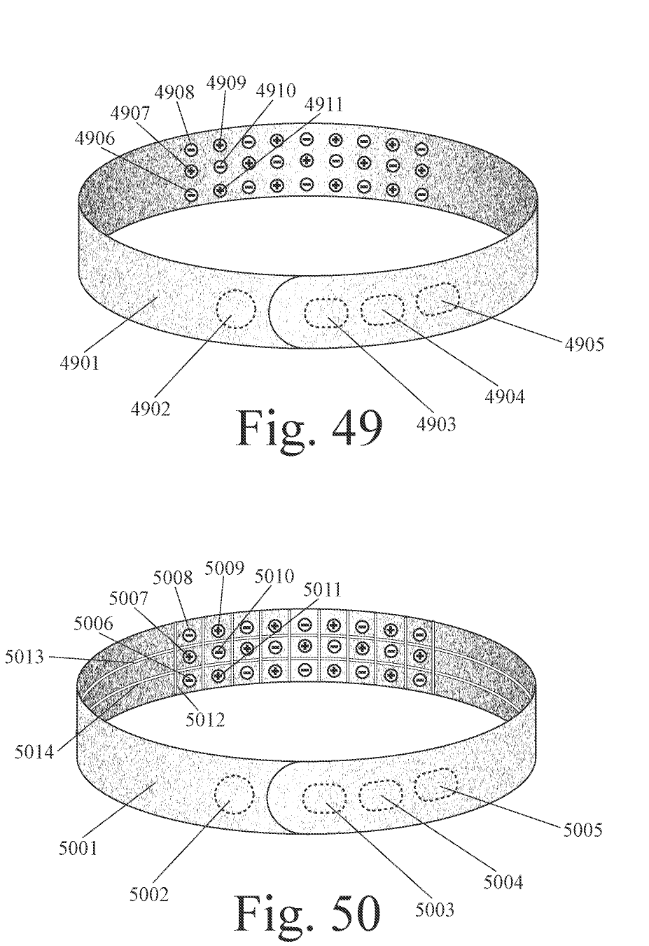

FIG. 50 shows an example of a wearable device for non-invasive glucose monitoring with a first set of energy emitters and energy receivers along a first circumferential line of a wearable arcuate band, a second set of energy emitters and energy receivers along a second circumferential line of the wearable arcuate band, a third set of energy emitters and energy receivers along a third circumferential line of the wearable arcuate band, a plurality of circumferential energy barriers between sets, and a plurality of proximal-to-distal energy barriers between energy emitters and/or energy receivers within sets. In an example in which the type of energy emitted and received is light energy, energy barriers can be opaque and/or compressible. In an example, different energy emitters can emit energy at different times and/or in a chronological sequence.

In this example, energy emitters and energy receivers alternate along a circumferential line. In this example, there is an alternating sequence of energy emitters and energy receivers along a circumferential line. In this example, there are multiple triads of energy emitters and energy receivers in the first, second, and third sets, wherein each triad is aligned along the same proximal-to-distal lines. In an example, there can be at least two energy emitters and two energy receivers in each set. In an example, there can be at least four energy emitters and four energy receivers in each set. In an example, energy emitters and energy receivers can be evenly-spaced within each set along a circumferential line. In this example, energy emitters and energy receivers can also be evenly-spaced between sets.

In this example, there is an arcuate array of energy emitters and energy receivers on an arcuate wearable band wherein this array has a circumferential axis (along which there are nine energy emitters and/or energy receivers) and a proximal-to-distal axis (along which there are three energy emitters and/or energy receivers). In an example, there can be an arcuate array of energy emitters and energy receivers on an arcuate wearable band wherein this array has a circumferential axis (along which there are at least three energy emitters and/or energy receivers) and a proximal-to-distal axis (along which there are at least two energy emitters and/or energy receivers).

In this example, there is an arcuate array of energy emitters and energy receivers on an arcuate wearable band wherein this array includes three parallel circumferential lines (along each of which there are nine energy emitters and/or energy receivers) and three parallel proximal-to-distal lines (along each of which there are three energy emitters and/or energy receivers). In an example, there can be an arcuate array of energy emitters and energy receivers on an arcuate wearable band wherein this array includes at least two parallel circumferential lines (along each of which there at least three energy emitters and/or energy receivers) and at least two parallel proximal-to-distal lines (along each of which there are at least two energy emitters and/or energy receivers).

Specifically, FIG. 50 shows an oblique-side-perspective view of a wearable device for non-invasive glucose monitoring comprising: arcuate band 5001 which is configured to span (some or all of) the circumferential perimeter of a part of a person's body (such as a wrist, arm, finger, ankle, and/or leg); a first set of three or more energy emitters (including 5009) and/or energy receivers (including 5008) along a first circumferential line of the arcuate band; a second set of three or more energy emitters (including 5007) and/or energy receivers (including 5010) along a second circumferential line of the arcuate band; a third set of three or more energy emitters (including 5011) and/or energy receivers (including 5006) along a third circumferential line of the arcuate band, wherein energy emitters are configured to emit energy toward the part of the person's body, and wherein energy receivers are configured to receive energy from energy emitters after that energy has passed through and/or been reflected from the part of the person's body; a plurality of circumferential energy barriers (including 5013 and 5014) between the sets (e.g. a first circumferential barrier between the first set and the second set and a second circumferential barrier between the second set and the third set); a plurality of proximal-to-distal energy barriers (including 5012) between the energy emitters and/or energy receivers within sets; a data processor 5004 which receives data from the energy receivers which is analyzed in order to measure the person's body glucose level; an energy source 5003 which provides energy to the central energy emitter and/or to the data processor; and a data transmitter 5005 which transmits data from the data processor to a remote device and/or remote location.

This example further comprises another (type of) biometric or environmental sensor 5002. In another example, one or more energy emitters and/or receivers can be part of a housing which is attached to the band instead of being part of the band itself. In an example, the central energy receiver and the energy emitters can be distributed around the circumference of a band, but not all be along the same circumferential line. Other example variations and component elaborations discussed elsewhere in this disclosure (or in other disclosures within the priority-linked family) can also be applied to this example.

FIG. 51 shows an example of a wearable device for non-invasive glucose monitoring with a single energy emitter and single energy receiver (which are on same circumferential line of a band) and an energy guide between the energy emitter and the energy receiver. In this example, the energy guide is a spit-ring resonator. In this example, the energy emitter emits (and receives reflected) microwave energy and the energy receiver receives microwave energy. In this example, the transmission of microwave energy from the energy emitter to the energy receiver is affected by the resonance of the split-ring resonator between them which, in turn, is affected by the permittivity of nearby body tissue which, in turn, is affected by body glucose level. In this example, the body glucose level of the person wearing this device can be estimated by analyzing the transmission of microwave energy from the energy emitter to the energy receiver (and also reflected back to the energy emitter).

Specifically, FIG. 51 shows an oblique-side-perspective view of a wearable device for non-invasive glucose monitoring comprising: arcuate band 5101 which is configured to span (some or all of) the circumferential perimeter of a part of a person's body (such as a wrist, arm, finger, ankle, and/or leg); energy emitter 5106 (identified with a plus sign) which is configured to emit energy toward the part of the person's body; energy receiver 5108 (identified with a negative sign) which is configured to receive energy from the energy emitter; energy guide 5107 between the energy emitter and the energy receiver, wherein transmission of energy from the energy emitter to the energy receiver through the energy guide is affected by the body glucose level of the person; data processor 5104 which receives data from the energy receiver which is analyzed in order to measure the person's body glucose level; energy source 5103 which provides energy to the energy emitter and/or to the data processor; and data transmitter 5105 which transmits data from the data processor to a remote device and/or remote location.

This example further comprises another (type of) biometric or environmental sensor 5102. In another example, an energy emitter, an energy receiver, or both can be part of a housing which is attached to the band, rather than part of the band itself.

In this example, the energy emitter and energy receiver can emit and receive microwave energy. In this example, the energy guide can be a split-ring resonator. Accordingly, FIG. 51 can also show an oblique-side-perspective view of a wearable device for non-invasive glucose monitoring comprising: arcuate band 5101 which is configured to span (some or all of) the circumferential perimeter of a part of a person's body (such as a wrist, arm, finger, ankle, and/or leg); microwave energy emitter 5106 (identified with a plus sign) which is configured to emit microwave energy toward the part of the person's body; microwave energy receiver 5108 (identified with a negative sign) which is configured to receive microwave energy from the energy emitter; split-ring resonator 5107 between the microwave energy emitter and the microwave energy receiver, wherein transmission of microwave energy from the microwave energy emitter to the microwave energy receiver through the split-ring resonator is affected by the body glucose level of the person; data processor 5104 which receives data from the microwave energy receiver and/or microwave emitter which is analyzed in order to measure the person's body glucose level; energy source 5103 which provides energy to the microwave energy emitter and/or to the data processor; and data transmitter 5105 which transmits data from the data processor to a remote device and/or remote location. This example further comprises another (type of) biometric or environmental sensor 5102. In another example, a microwave energy emitter, a microwave energy receiver, and/or a split-ring resonator can be part of a housing which is attached to the band, rather than part of the band itself. Other example variations and component elaborations discussed elsewhere in this disclosure (or in other disclosures within the priority-linked family) can also be applied to this example.

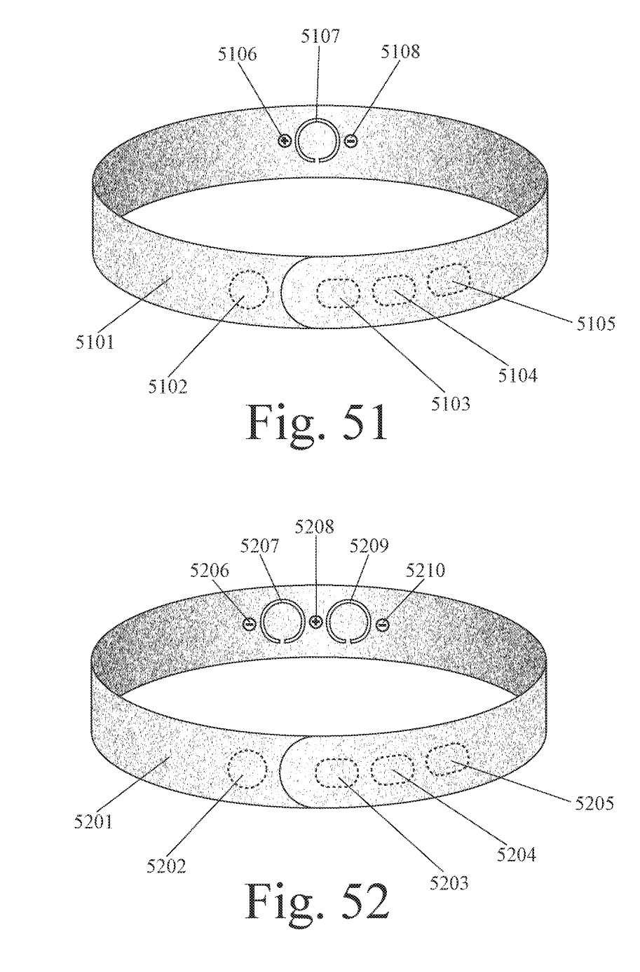

FIG. 52 shows an example of a wearable device for non-invasive glucose monitoring with a single microwave energy emitter and two microwave energy receivers on the same circumferential line of a band, a first split-ring resonator between the microwave energy emitter and the first microwave energy receiver, and a second split-ring resonator between the microwave energy emitter and the second microwave energy receiver. In this example, the transmission of microwave energy from the energy emitter to the energy receivers is affected by resonances of the split-ring resonators which, in turn, are affected by the permittivity of nearby body tissue which, in turn, is affected by body glucose level. In this example, the body glucose level of the person wearing this device can be estimated by analyzing the transmission of microwave energy from the energy emitter to the energy receivers (and/or reflected back to the energy emitter).

Specifically, FIG. 52 shows an oblique-side-perspective view of a wearable device for non-invasive glucose monitoring comprising: arcuate band 5201 which is configured to span (some or all of) the circumferential perimeter of a part of a person's body (such as a wrist, arm, finger, ankle, and/or leg); microwave energy emitter 5208 (identified with a plus sign) which is configured to emit microwave energy toward the part of the person's body; first microwave energy receiver 5206 (identified with a negative sign) which is configured to receive microwave energy from the microwave energy emitter; second microwave energy receiver 5210 (identified with a negative sign) which is configured to receive microwave energy from the microwave energy emitter; first split-ring resonator 5207 between the microwave energy emitter and the first microwave energy receiver, wherein transmission of microwave energy from the microwave energy emitter to the first microwave energy receiver through the first split-ring resonator is affected by body glucose level of the person; second split-ring resonator 5209 between the microwave energy emitter and the second microwave energy receiver, wherein transmission of microwave energy from the microwave energy emitter to the second microwave energy receiver through the second split-ring resonator is affected by body glucose level of the person; data processor 5204 which receives data from the microwave energy receivers and/or microwave emitter which is analyzed in order to measure the person's body glucose level; energy source 5203 which provides energy to the microwave energy emitter and/or to the data processor; and data transmitter 5205 which transmits data from the data processor to a remote device and/or remote location. This example further comprises another (type of) biometric or environmental sensor 5202. In another example, a microwave energy emitter, a microwave energy receiver, and/or a split-ring resonator can be part of a housing which is attached to the band, rather than part of the band itself. Other example variations and component elaborations discussed elsewhere in this disclosure (or in other disclosures within the priority-linked family) can also be applied to this example.

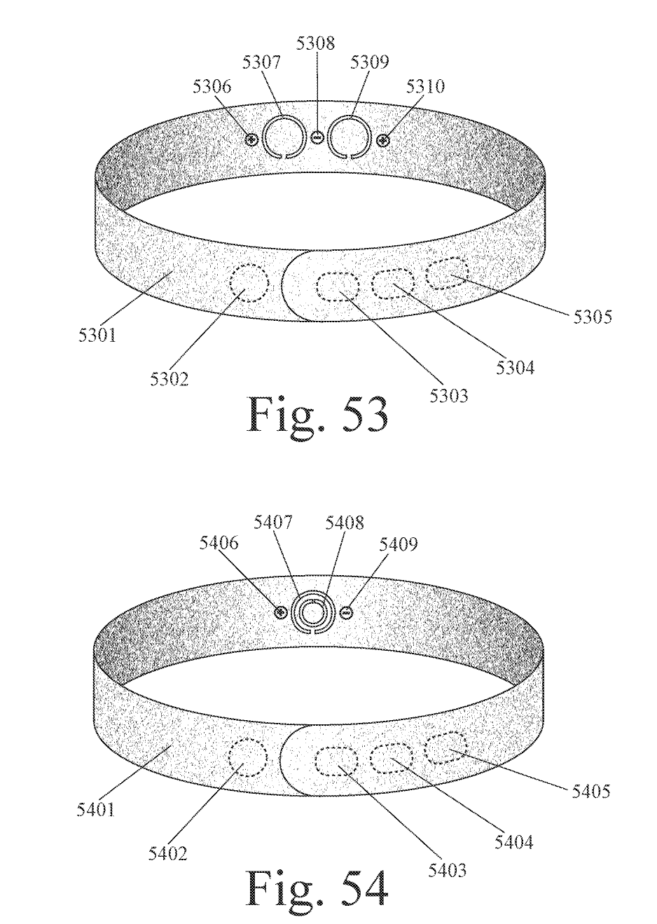

FIG. 53 shows an example of a wearable device for non-invasive glucose monitoring with two microwave energy emitters and a single microwave energy receiver on the same circumferential line of a band, a first split-ring resonator between the first microwave energy emitter and the microwave energy receiver, and a second split-ring resonator between the second microwave energy emitter and the microwave energy receiver. In this example, the transmission of microwave energy from the energy emitters to the energy receiver is affected by the resonances of the split-ring resonators which, in turn, are affected by the permittivity of nearby body tissue which, in turn, is affected by body glucose level. In this example, the body glucose level of the person wearing this device can be estimated by analyzing the transmission of microwave energy from the energy emitters to the energy receiver (and/or reflected back to the energy emitter). In an example, different microwave energy emitters can emit microwave energy at different times and/or in a chronological sequence.

Specifically, FIG. 53 shows an oblique-side-perspective view of a wearable device for non-invasive glucose monitoring comprising: arcuate band 5301 which is configured to span (some or all of) the circumferential perimeter of a part of a person's body (such as a wrist, arm, finger, ankle, and/or leg); first microwave energy emitter 5306 (identified with a plus sign) which is configured to emit microwave energy toward the part of the person's body; second microwave energy emitter 5310 (identified with a plus sign) which is configured to emit microwave energy toward the part of the person's body; microwave energy receiver 5308 (identified with a negative sign) which is configured to receive microwave energy from the microwave energy emitters; first split-ring resonator 5307 between the first microwave energy emitter and the microwave energy receiver, wherein transmission of microwave energy from the first microwave energy emitter to the microwave energy receiver through the first split-ring resonator is affected by the body glucose level of the person; second split-ring resonator 5309 between the second microwave energy emitter and the microwave energy receiver, wherein transmission of microwave energy from the second microwave energy emitter to the microwave energy receiver through the second split-ring resonator is affected by the body glucose level of the person; data processor 5304 which receives data from the microwave energy receiver and/or microwave emitters which is analyzed in order to measure the person's body glucose level; energy source 5303 which provides energy to the microwave energy emitters and/or to the data processor; and data transmitter 5305 which transmits data from the data processor to a remote device and/or remote location. This example further comprises another (type of) biometric or environmental sensor 5302. In another example, a microwave energy emitter, a microwave energy receiver, and/or a split-ring resonator can be part of a housing which is attached to the band, rather than part of the band itself. Other example variations and component elaborations discussed elsewhere in this disclosure (or in other disclosures within the priority-linked family) can also be applied to this example.

FIG. 54 shows an example of a wearable device for non-invasive glucose monitoring: with a microwave energy emitter and a microwave energy receiver on the same circumferential line of an arcuate wearable band; and with two nested (and concentric) split-ring resonators between the microwave energy emitter and the microwave energy receiver. In this example, the transmission of microwave energy from the energy emitter to the energy receiver is affected by the resonances of the nested split-ring resonators which, in turn, are affected by the permittivity of nearby body tissue which, in turn, is affected by body glucose level. In this example, the body glucose level of the person wearing this device can be estimated by analyzing the transmission of microwave energy from the energy emitter to the energy receiver (and/or reflected back to the energy emitter).

Specifically, FIG. 54 shows an oblique-side-perspective view of a wearable device for non-invasive glucose monitoring comprising: arcuate band 5401 which is configured to span (some or all of) the circumferential perimeter of a part of a person's body (such as a wrist, arm, finger, ankle, and/or leg); microwave energy emitter 5406 (identified with a plus sign) which is configured to emit microwave energy toward the part of the person's body; microwave energy receiver 5409 (identified with a negative sign) which is configured to receive microwave energy from the microwave energy emitter; first split-ring resonator 5407 between the microwave energy emitter and the microwave energy receiver, wherein transmission of microwave energy from the microwave energy emitter to the microwave energy receiver through the first split-ring resonator is affected by the body glucose level of the person; second split-ring resonator 5408 between the microwave energy emitter and the microwave energy receiver, wherein transmission of microwave energy from the microwave energy emitter to the microwave energy receiver through the second split-ring resonator is affected by the body glucose level of the person, and wherein the second split-ring resonator is nested (in a concentric manner) within the first spit-ring resonator; data processor 5404 which receives data from the microwave energy receiver and/or microwave emitter which is analyzed in order to measure the person's body glucose level; energy source 5403 which provides energy to the microwave energy emitters and/or to the data processor; and data transmitter 5405 which transmits data from the data processor to a remote device and/or remote location. This example further comprises another (type of) biometric or environmental sensor 5402. In another example, a microwave energy emitter, a microwave energy receiver, and/or a split-ring resonator can be part of a housing which is attached to the band, rather than part of the band itself. Other example variations and component elaborations discussed elsewhere in this disclosure (or in other disclosures within the priority-linked family) can also be applied to this example.

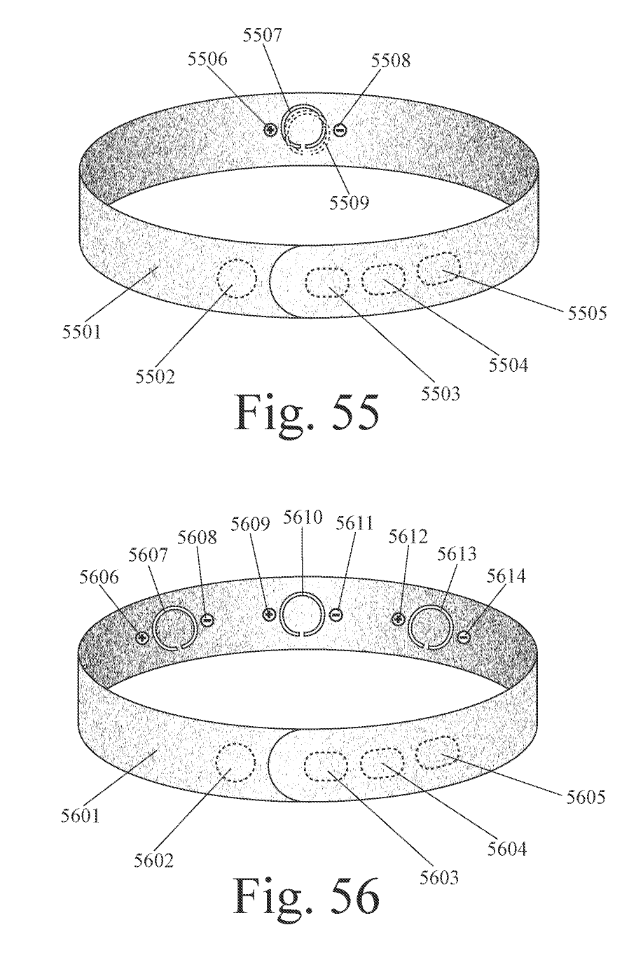

FIG. 55 shows an example of a wearable device for non-invasive glucose monitoring: with a microwave energy emitter and a microwave energy receiver on the same circumferential line of an arcuate wearable band; and with two stacked (and parallel) split-ring resonators between the microwave energy emitter and the microwave energy receiver. In this example, the transmission of microwave energy from the energy emitter to the energy receiver is affected by the resonances of the stacked split-ring resonators which, in turn, are affected by the permittivity of nearby body tissue which, in turn, is affected by body glucose level. In this example, the body glucose level of the person wearing this device can be estimated by analyzing the transmission of microwave energy from the energy emitter to the energy receiver (and/or reflected back to the energy emitter).

Specifically, FIG. 55 shows an oblique-side-perspective view of a wearable device for non-invasive glucose monitoring comprising: arcuate band 5501 which is configured to span (some or all of) the circumferential perimeter of a part of a person's body (such as a wrist, arm, finger, ankle, and/or leg); microwave energy emitter 5506 (identified with a plus sign) which is configured to emit microwave energy toward the part of the person's body; microwave energy receiver 5508 (identified with a negative sign) which is configured to receive microwave energy from the microwave energy emitter; first split-ring resonator 5507 between the microwave energy emitter and the microwave energy receiver, wherein transmission of microwave energy from the microwave energy emitter to the microwave energy receiver through the first split-ring resonator is affected by the body glucose level of the person; second split-ring resonator 5509 between the microwave energy emitter and the microwave energy receiver, wherein transmission of microwave energy from the microwave energy emitter to the microwave energy receiver through the second split-ring resonator is affected by the body glucose level of the person, and wherein the second split-ring resonator is stacked above (e.g. parallel to) the first spit-ring resonator; data processor 5504 which receives data from the microwave energy receiver and/or microwave emitter which is analyzed in order to measure the person's body glucose level; energy source 5503 which provides energy to the microwave energy emitters and/or to the data processor; and data transmitter 5505 which transmits data from the data processor to a remote device and/or remote location. This example further comprises another (type of) biometric or environmental sensor 5502. In another example, a microwave energy emitter, a microwave energy receiver, and/or a split-ring resonator can be part of a housing which is attached to the band, rather than part of the band itself. Other example variations and component elaborations discussed elsewhere in this disclosure (or in other disclosures within the priority-linked family) can also be applied to this example.

FIG. 56 shows an example of a wearable device for non-invasive glucose monitoring with a plurality of sets, wherein each set comprises: a microwave energy emitter, a microwave energy receiver, and a split-ring resonator between the microwave energy emitter and the microwave energy receiver. In this example, there are three such sets. In another example, there can be four or more such sets distributed around the circumference of an arcuate wearable band. In this example, the sets are along the same circumferential line of an arcuate wearable band. In an example, the transmission of microwave energy from a microwave energy emitter to a microwave energy receiver in each set is affected by the resonance of the split-ring resonator in that set which, in turn, is affected by the permittivity of nearby body tissue which, in turn, is affected by body glucose level. In this example, the body glucose level of the person wearing this device can be estimated by analyzing the transmission of microwave energy from microwave energy emitters to microwave energy receivers (and/or reflected back to energy emitters) in the plurality of sets. In an example, microwave energy emitters in different sets can emit microwave energy at different times and/or in a chronological sequence.

Specifically, FIG. 56 shows an oblique-side-perspective view of a wearable device for non-invasive glucose monitoring comprising: an arcuate band 5601 which is configured to span (some or all of) the circumferential perimeter of a part of a person's body (such as a wrist, arm, finger, ankle, and/or leg); a plurality of microwave sensor sets, wherein each microwave sensor set further comprises a microwave energy emitter (such as 5606, 5609, and 5612) which is configured to emit microwave energy toward the part of the person's body, a microwave energy receiver (such as 5608, 5611, and 5614) which is configured to receive microwave energy from the microwave energy emitter, and a split ring resonator (such as 5607, 5610, and 5613) between the microwave energy emitter and the microwave energy receiver; a data processor 5604 which receives data from the microwave energy receivers and/or microwave emitters which is analyzed in order to measure the person's body glucose level; an energy source 5603 which provides energy to the microwave energy emitters and/or to the data processor; and a data transmitter 5605 which transmits data from the data processor to a remote device and/or remote location.

This example further comprises another (type of) biometric or environmental sensor 5602. In another example, a microwave energy emitter, a microwave energy receiver, and/or a split-ring resonator can be part of a housing which is attached to the band, rather than part of the band itself. Other example variations and component elaborations discussed elsewhere in this disclosure (or in other disclosures within the priority-linked family) can also be applied to this example.

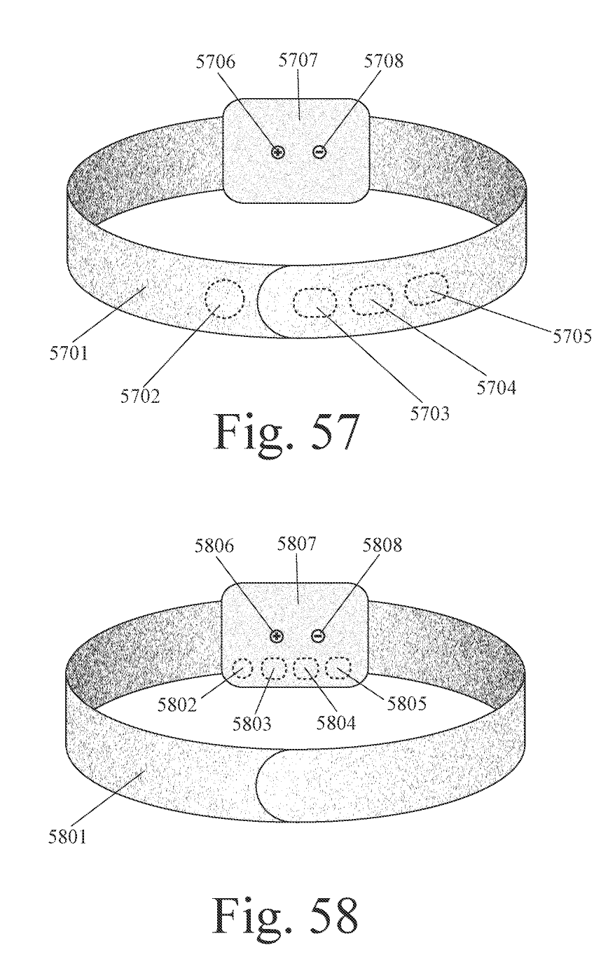

FIG. 57 shows an example of a wearable device for non-invasive glucose monitoring with an energy emitter and an energy receiver which are part of a housing which is held onto a person's body by an arcuate wearable band. In an example, the housing and band can comprise a smart watch, fitness band, wearable hydration monitor, or wearable glucose monitor. In this example, the energy emitter and the energy receiver are located along the same circumferential line (of the device comprising the housing and band). In an alternative example, an energy emitter and energy receiver can be located along the same proximal-to-distal line (of a device). In an example, the housing can be more rigid (e.g. less flexible) than the arcuate band. In an example, an energy emitter and an energy receiver can be located on the inward-facing (body-facing) side of a housing. In an example, the opposite (outward-facing) side of a housing can comprise a computer display and/or screen. In an example, the type of energy that is emitted and received can be light energy. In an example, the type of energy that is emitted and received can be (non-light-spectrum) electromagnetic energy.

Specifically, FIG. 57 shows an oblique-side-perspective view of a wearable device for non-invasive glucose monitoring comprising: an arcuate band 5701 which is configured to span (some or all of) the circumferential perimeter of a part of a person's body (such as a wrist, arm, finger, ankle, and/or leg); a housing 5707 which is attached to the arcuate band; an energy emitter 5706 which is held by the housing and configured to emit energy toward the part of the person's body; an energy receiver 5708 which is held by the housing and configured to receive energy from the energy emitter; a data processor 5704 which receives data from the energy receiver which is analyzed in order to measure the person's body glucose level; an energy source 5703 which provides energy to the energy emitter and/or to the data processor; and a data transmitter 5705 which transmits data from the data processor to a remote device and/or remote location. This example further comprises another (type of) biometric or environmental sensor 5702. Other example variations and component elaborations discussed elsewhere in this disclosure (or in other disclosures within the priority-linked family) can also be applied to this example.

FIG. 58 shows an example of a wearable device for non-invasive glucose monitoring with an energy emitter and an energy receiver and other electronic components (e.g. energy source, data processor, data transmitter, and other type of sensor) which are all part of a housing which is held onto a person's body by an arcuate wearable band. In an example, the housing and band can comprise a smart watch, fitness band, wearable hydration monitor, or wearable glucose monitor. In this example, the energy emitter and the energy receiver are located along the same circumferential line (of the device comprising the housing and band). In an alternative example, an energy emitter and energy receiver can be located along the same proximal-to-distal line (of a device). In an example, the housing can be more rigid (e.g. less flexible) than the arcuate band. In an example, an energy emitter and an energy receiver can be located on the inward-facing (body-facing) side of a housing. In an example, the opposite (outward-facing) side of a housing can comprise a computer display and/or screen. In an example, the type of energy that is emitted and received can be light energy. In an example, the type of energy that is emitted and received can be (non-light-spectrum) electromagnetic energy.

Specifically, FIG. 58 shows an oblique-side-perspective view of a wearable device for non-invasive glucose monitoring comprising: an arcuate band 5801 which is configured to span (some or all of) the circumferential perimeter of a part of a person's body (such as a wrist, arm, finger, ankle, and/or leg); a housing 5807 which is attached to the arcuate band; an energy emitter 5806 which is held by the housing and configured to emit energy toward the part of the person's body; an energy receiver 5808 which is held by the housing and configured to receive energy from the energy emitter; a data processor 5804 which is held by the housing and receives data from the energy receiver which is analyzed in order to measure the person's body glucose level; an energy source 5803 which is held by the housing and provides energy to the energy emitters and/or to the data processor; and a data transmitter 5805 which is held by the housing and transmits data from the data processor to a remote device and/or remote location. This example further comprises another (type of) biometric or environmental sensor 5802. Other example variations and component elaborations discussed elsewhere in this disclosure (or in other disclosures within the priority-linked family) can also be applied to this example.

FIG. 59 shows an example of a wearable device for non-invasive glucose monitoring with: a housing that is attached to a wearable arcuate band; and a plurality of energy emitters and a central energy receiver which are held by this housing. In this example, the energy emitters and energy receiver are all on the same circumferential line of the device, wherein the device comprises the housing as well as the arcuate band. In an example, the housing and band can comprise a smart watch, fitness band, wearable hydration monitor, or wearable glucose monitor. In an example, the energy emitters and energy receiver can be located on the inward-facing (body-facing) side of a housing. In an example, the opposite (outward-facing) side of a housing can comprise a computer display and/or screen. In this example, the central energy receiver is centrally located with respect to the energy emitters. In this example, there are two energy emitters on each side (clockwise or counter-clockwise, right or left) of the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

Specifically, FIG. 59 shows an oblique-side-perspective view of a wearable device for non-invasive glucose monitoring comprising: an arcuate band 5901 which is configured to span (some or all of) the circumferential perimeter of a part of a person's body (such as a wrist, arm, finger, ankle, and/or leg); a housing 5908 which is attached to the arcuate band; a plurality of energy emitters (5906, 5907, 5910, and 5911) which are held by the housing and configured to emit energy toward the part of the person's body; a central energy receiver 5909 which is held by the housing and configured to receive energy from the energy emitters, wherein the plurality of energy emitters and the central energy receiver are all located along the same circumferential line, and wherein the central energy receiver is centrally located with respect to the plurality of energy emitters; a data processor 5904 which is held by the housing and receives data from the energy receiver which is analyzed in order to measure the person's body glucose level; an energy source 5903 which is held by the housing and provides energy to the energy emitters and/or to the data processor; and a data transmitter 5905 which is held by the housing and transmits data from the data processor to a remote device and/or remote location.

This example further comprises another (type of) biometric or environmental sensor 5902. Other example variations and component elaborations discussed elsewhere in this disclosure (or in other disclosures within the priority-linked family) can also be applied to this example.

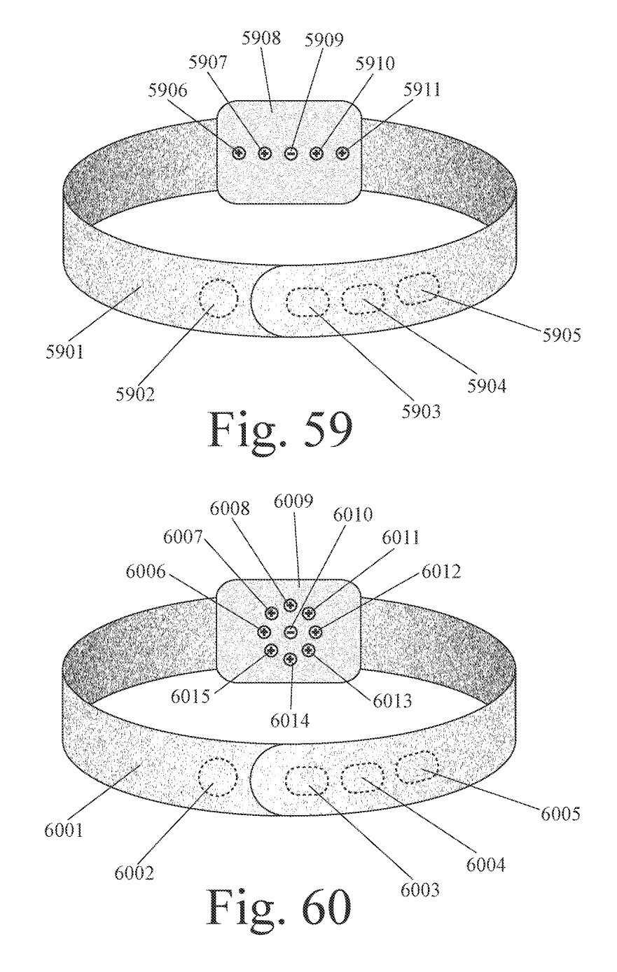

FIG. 60 shows an example of a wearable device for non-invasive glucose monitoring with: a housing that is attached to a wearable arcuate band; and a plurality of energy emitters and central energy receiver which are held by this housing. In an example, energy emitters can encircle the energy receiver. In an example, energy emitters can be arranged in an (approximately-circular) polygonal array around the central energy receiver. In an example, the housing and band can comprise a smart watch, fitness band, wearable hydration monitor, or wearable glucose monitor. In an example, the energy emitters and energy receiver can be located on the inward-facing (body-facing) side of a housing. In an example, the opposite (outward-facing) side of a housing can comprise a computer display and/or screen.

In this example, the central energy receiver is centrally located with respect to the energy emitters. In this example, there are eight energy emitters around the central energy receiver. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

Specifically, FIG. 60 shows an oblique-side-perspective view of a wearable device for non-invasive glucose monitoring comprising: an arcuate band 6001 which is configured to span (some or all of) the circumferential perimeter of a part of a person's body (such as a wrist, arm, finger, ankle, and/or leg); a housing 6009 which is attached to the arcuate band; a plurality of energy emitters (6006, 6007, 6008, 6011, 6012, 6013, 6014, and 6015) which are held by the housing and configured to emit energy toward the part of the person's body; a central energy receiver 6010 which is held by the housing and configured to receive energy from the energy emitters, wherein the plurality of energy emitters are around the central energy receiver (in an approximately-circular polygonal array); a data processor 6004 which is held by the housing and receives data from the energy receiver which is analyzed in order to measure the person's body glucose level; an energy source 6003 which is held by the housing and provides energy to the energy emitters and/or to the data processor; and a data transmitter 6005 which is held by the housing and transmits data from the data processor to a remote device and/or remote location. This example further comprises another (type of) biometric or environmental sensor 6002. Other example variations and component elaborations discussed elsewhere in this disclosure (or in other disclosures within the priority-linked family) can also be applied to this example.

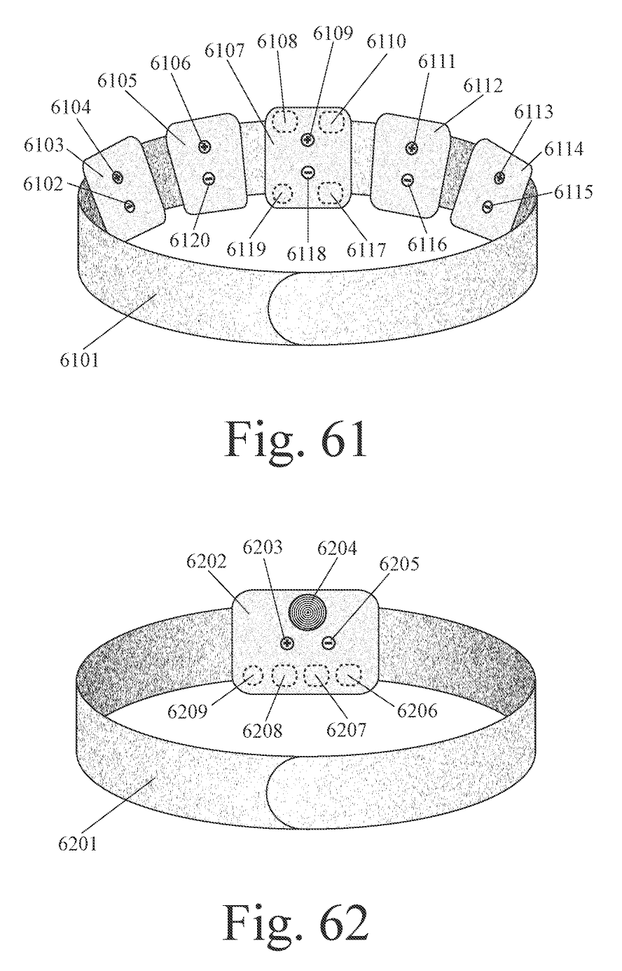

FIG. 61 shows an example of a wearable device for non-invasive glucose monitoring with multiple connected segments (or housings) which comprise (parts of) an arcuate wearable band, wherein each segment (or housing) holds an energy emitter and an energy receiver. In this example, there are five connected segments which span approximately half of the circumference of a band. In an example, there can be more than five connected segments and they can span a greater portion of the band circumference. In this example, an energy emitter and an energy receiver on a segment are on same proximal-to-distal line. In an alternative example, an energy emitter and an energy receiver on a segment can be on the same circumferential line. In an example, different energy emitters can emit energy at different times and/or in a chronological sequence. In an example, the type of energy that is emitted and received can be light energy. In an example, the type of energy that is emitted and received can be (non-light-spectrum) electromagnetic energy.

Specifically, FIG. 61 shows an oblique-side-perspective view of a wearable device for non-invasive glucose monitoring comprising: an arcuate band 6101 which is configured to span (some or all of) the circumferential perimeter of a part of a person's body (such as a wrist, arm, finger, ankle, and/or leg); a plurality of segments (or housings) 6103, 6105, 6107, 6112, and 6113 which are attached to (and/or comprise) the arcuate band, wherein each segment (or housing) holds an energy emitter 6104, 6106, 6109, 6111, and 6113 and an energy receiver 6102, 6120, 6118, 6116, and 6115; a data processor 6104 which receives data from the energy receivers which is analyzed in order to measure the person's body glucose level; an energy source 6103 which provides energy to the energy emitters and/or to the data processor; and a data transmitter 6105 which transmits data from the data processor to a remote device and/or remote location. This example further comprises another (type of) biometric or environmental sensor 6102. Other example variations and component elaborations discussed elsewhere in this disclosure (or in other disclosures within the priority-linked family) can also be applied to this example.

In an example, this invention can be embodied in a wearable device for non-invasive glucose monitoring comprising: a plurality of flexibly-connected segments which collectively form an arcuate wearable band which is configured to span (some or all of) the circumferential perimeter of a part of a person's body (such as a wrist, arm, finger, ankle, and/or leg), wherein each segment (or housing) holds an energy emitter and an energy receiver; a data processor which receives data from the energy receivers which is analyzed in order to measure the person's body glucose level; an energy source which provides energy to the energy emitters and/or to the data processor; and a data transmitter which transmits data from the data processor to a remote device and/or remote location. Other example variations and component elaborations discussed elsewhere in this disclosure (or in other disclosures within the priority-linked family) can also be applied to this example.

FIG. 62 shows an example of a wearable device for non-invasive glucose monitoring with: an energy emitter and an energy receiver for measuring body glucose level; and also a fluid-based glucose sensor for measuring body glucose level. In an example, body glucose level is measured less frequently with the fluid-based glucose sensor than with the energy emitter and energy receiver. In an example, measurement of body glucose level with the fluid-based glucose sensor can be used to calibrate measurement of body glucose level with the energy emitter and energy receiver. In an example, a fluid-based glucose sensor can withdraw small samples of interstitial fluid or blood through a small catheter or needle for analysis of glucose level.

In an example, the type of energy that is emitted by the energy emitter and received by the energy receiver can be light energy. In an example, the type of energy that is emitted by the energy emitter and received by the energy receiver can be (non-light-spectrum) electromagnetic energy. In this example, the energy emitter, energy receiver, and fluid-based glucose sensor are held in place by a housing which is attached to an arcuate band. In another example, these components can be directly part of an arcuate band.

Specifically, FIG. 62 shows an oblique-side-perspective view of a wearable device for non-invasive glucose monitoring comprising: an arcuate band 6201 which is configured to span (some or all of) the circumferential perimeter of a part of a person's body (such as a wrist, arm, finger, ankle, and/or leg); a housing 6202 which is attached to the arcuate band; an energy emitter 6203 which is held by the housing and configured to emit energy toward the part of the person's body; an energy receiver 6205 which is held by the housing and configured to receive energy from the energy emitter; a fluid-based glucose sensor 6204 which is held by the housing; a data processor 6207 which receives data from the energy receiver and data from the fluid-based glucose sensor which are analyzed in order to measure the person's body glucose level; an energy source 6208 which provides energy to the energy emitter, the fluid-based glucose sensor, and/or to the data processor; and a data transmitter 6206 which transmits data from the data processor to a remote device and/or remote location. This example further comprises another (type of) biometric or environmental sensor 6209.

In an example, this invention can be embodied in a wearable device for non-invasive glucose monitoring comprising: an arcuate band which is configured to span (some or all of) the circumferential perimeter of a part of a person's body (such as a wrist, arm, finger, ankle, and/or leg); an energy emitter which is configured to emit energy toward the part of the person's body; an energy receiver which is configured to receive energy from the energy emitter; a fluid-based glucose sensor; a data processor which receives data from the energy receiver and data from the fluid-based glucose sensor which are analyzed in order to measure the person's body glucose level; an energy source which provides energy to the energy emitter, the fluid-based glucose sensor, and/or to the data processor; and a data transmitter which transmits data from the data processor to a remote device and/or remote location. This example can further comprise another (type of) biometric or environmental sensor. Other example variations and component elaborations discussed elsewhere in this disclosure (or in other disclosures within the priority-linked family) can also be applied to this example.

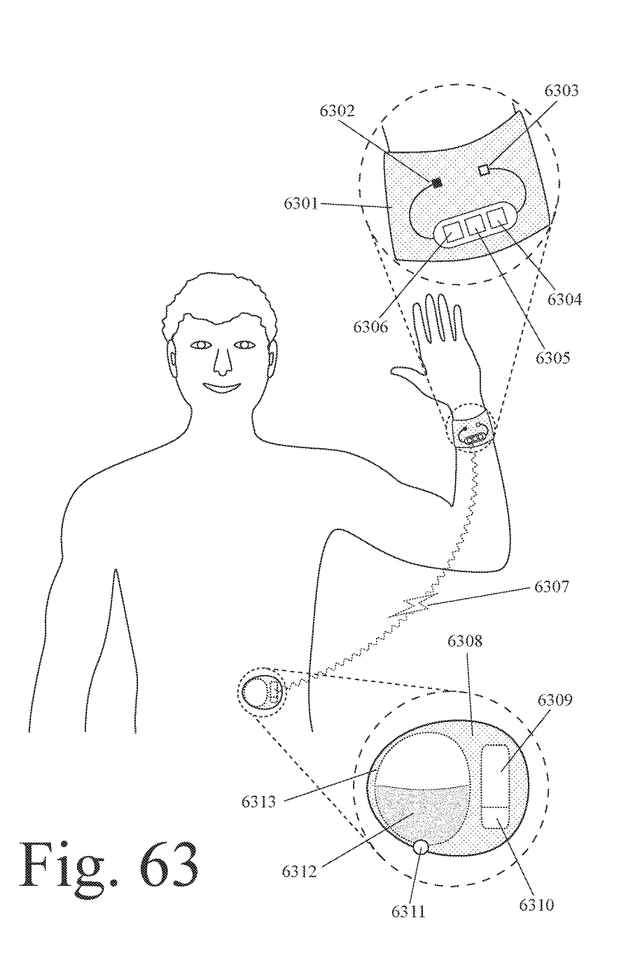

FIG. 63 shows an example of how this invention can be embodied in a glucose monitoring and managing system comprising: a wearable glucose-monitoring microwave sensor which collects data which is used to measure a person's intra-body glucose levels, wherein this glucose-monitoring microwave sensor further comprises a microwave energy emitter and a microwave energy receiver; and a wearable or implanted pump which delivers a glucose-level-modifying substance into the person's body based on data collected by the wearable glucose-monitoring microwave sensor.

The middle portion of FIG. 63 shows a frontal view of a person's head, torso, and hand, wherein the person is wearing a wearable glucose-monitoring microwave sensor on their wrist and a wearable pump on their torso which dispenses a glucose-level-modifying substance. In this example, the wrist-worn component and the wearable pump component are in direct wireless electromagnetic communication with each other. The operation of the wearable pump is controlled based on analysis of data from microwave sensor in the wrist-worn component. In this example, the components shown in FIG. 63 combine to form a system for monitoring and managing intra-body glucose levels.

The upper right portion of FIG. 63 shows a close-up view of the wrist-worn component of this system within a virtual dashed-line circle, wherein this dashed-line circle corresponds to the smaller dashed-line circle around the same wrist-worn component shown in smaller scale on the middle portion of the figure. The lower right portion of FIG. 63 shows a close-up view of the wearable pump component of this system within a virtual-dashed line circle, wherein this dashed-line circle corresponds to the smaller dashed line circle around the same wearable pump component that is shown in smaller scale on the middle portion of the figure.

With respect to specific structures within system components, the wearable glucose-monitoring microwave sensor shown in a close-up view in the upper right portion of FIG. 63 comprises: wrist band 6301; microwave energy emitter 6302; microwave energy receiver 6303; power source 6304; data processor 6305; and data transmitter/receiver 6306. With respect to structures within system components, the pump shown in a close-up view in the lower right portion of FIG. 63 includes: wearable pump housing 6308; data processor and transmitter/receiver 6309; power source 6310; glucose-level-modifying substance 6312; pump 6311 for the glucose-level-modifying substance; and reservoir 6313 for the glucose-level-modifying substance. In this example, data transmitter/receiver 6306 of the wrist-worn component is in direct wireless communication 6307 with data processor and transmitter/receiver 6309 of the wearable pump component. In another example, both of these data transmitters/receivers can be in indirect wireless communication by each having wireless communication with a common separate and/or remote data processor and transmitter/receiver. Other example variations and component elaborations discussed elsewhere in this disclosure (or in other disclosures within the priority-linked family) can also be applied to this example.

In another example, a wearable glucose-monitoring microwave sensor can comprise a microwave energy emitter, a microwave energy receiver, and a resonator between the microwave energy emitter and the microwave energy receiver, wherein the resonant frequency of the resonator is changed by changes in the glucose levels of nearby body tissue and/or fluid. In an example, changes in the glucose levels of nearby body tissue and/or fluid change the permittivity of the body tissue and/or fluid which, in turn, changes the resonant frequency of the resonator. In an example, a wearable glucose-monitoring microwave sensor can comprise a microwave energy emitter, a microwave energy receiver, and a resonator, wherein changes in microwave transmission from the microwave energy emitter to the microwave energy receiver are used to measure changes in the glucose levels of nearby body tissue and/or fluid.

In an example, a glucose monitoring and managing system can comprise: a wearable glucose-monitoring microwave sensor which collects data which is used to measure a person's intra-body glucose levels, wherein this glucose-monitoring microwave sensor further comprises a microwave energy emitter, a microwave energy receiver, and a resonator between the microwave energy emitter and the microwave energy receiver; and a wearable or implanted pump which delivers a glucose-level-modifying substance into the person's body based on data collected by the wearable glucose-monitoring microwave sensor. In an example, one or more resonant frequencies of such a resonator can change with changes in the glucose level of nearby body tissue and/or body fluid. One or more resonant frequencies of such a resonator can be changed by a change in the permittivity of nearby body tissue and/or fluid which, in turn, can be changed by changes in the glucose level of nearby body tissue and/or body fluid. The resonant frequency of a resonator is reduced by dielectric loading of body tissue. In an example, there can be an inverse relationship between the glucose levels of body tissue and/or fluid and the resonant frequency of a resonator. In this manner, intra-body glucose levels can be estimated by measuring one or more resonant frequencies of an electromagnetic resonator.

In an example, a closed-loop glucose monitoring and managing system can comprise: a wearable glucose-monitoring microwave sensor which automatically collects data which is used to measure a person's intra-body glucose levels, wherein this glucose-monitoring microwave sensor further comprises a microwave energy emitter, a microwave energy receiver, and a resonator between the microwave energy emitter and the microwave energy receiver; and a wearable or implanted pump which automatically delivers a glucose-level-modifying substance into the person's body to maintain intra-body glucose levels within a selected range, wherein operation of the pump is based on data collected by the wearable glucose-monitoring microwave sensor.

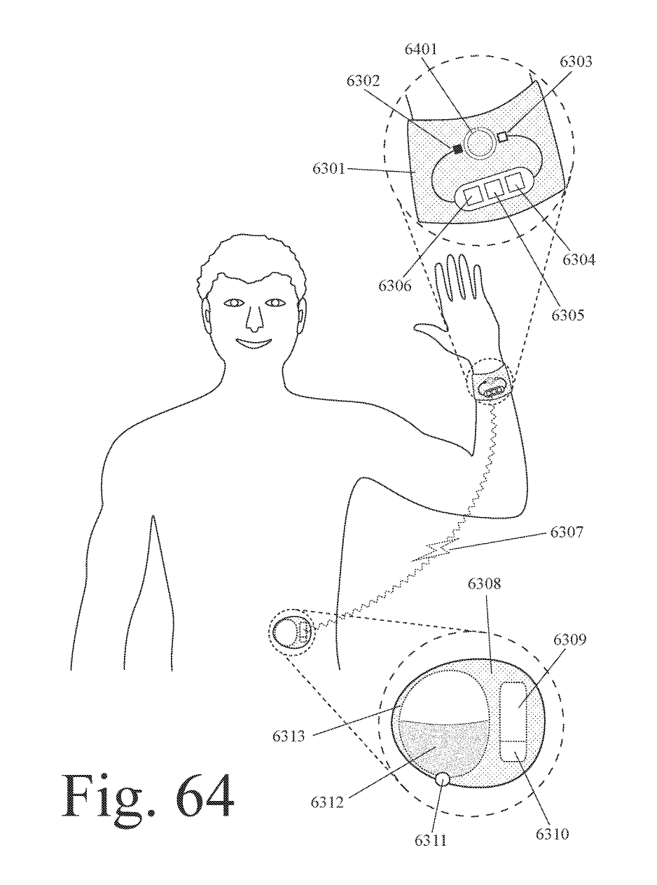

FIG. 64 shows an example of how this invention can be embodied in a glucose monitoring and managing system comprising: a wearable glucose-monitoring microwave sensor that collects data which is used to measure a person's intra-body glucose levels, wherein this glucose-monitoring microwave sensor further comprises a microwave energy emitter, a microwave energy receiver, and a resonator; and a wearable or implanted pump which delivers a glucose-level-modifying substance into the person's body based on data collected by the wearable glucose-monitoring microwave sensor.

The middle portion of FIG. 64 shows a frontal view of a person's head, torso, and hand, wherein the person is wearing a wearable glucose-monitoring microwave sensor on their wrist and a wearable pump on their torso which dispenses a glucose-level-modifying substance. In this example, the wrist-worn component and the wearable pump component are in direct wireless electromagnetic communication with each other. The operation of the wearable pump is controlled based on analysis of data from microwave sensor in the wrist-worn component. In this example, the components shown in FIG. 64 combine to form a system for monitoring and managing intra-body glucose levels.

The upper right portion of FIG. 64 shows a close-up view of the wrist-worn component of this system within a virtual dashed-line circle, wherein this dashed-line circle corresponds to the smaller dashed-line circle around the same wrist-worn component shown in smaller scale on the middle portion of the figure. The lower right portion of FIG. 64 shows a close-up view of the wearable pump component of this system within a virtual-dashed line circle, wherein this dashed-line circle corresponds to the smaller dashed line circle around the same wearable pump component that is shown in smaller scale on the middle portion of the figure.

With respect to specific structures within system components, the wearable glucose-monitoring microwave sensor shown in a close-up view in the upper right portion of FIG. 64 comprises: wrist band 6301; microwave energy emitter 6302; resonator 6401; microwave energy receiver 6303; power source 6304; data processor 6305; and data transmitter/receiver 6306. With respect to structures within system components, the pump shown in a close-up view in the lower right portion of FIG. 64 includes: wearable pump housing 6308; data processor and transmitter/receiver 6309; power source 6310; glucose-level-modifying substance 6312; pump 6311 for the glucose-level-modifying substance; and reservoir 6313 for the glucose-level-modifying substance. In this example, data transmitter/receiver 6306 of the wrist-worn component is in direct wireless communication 6307 with data processor and transmitter/receiver 6309 of the wearable pump component. In another example, both of these data transmitters/receivers can be in indirect wireless communication by each having wireless communication with a common separate and/or remote data processor and transmitter/receiver. Other example variations and component elaborations discussed elsewhere in this disclosure (or in other disclosures within the priority-linked family) can also be applied to this example.

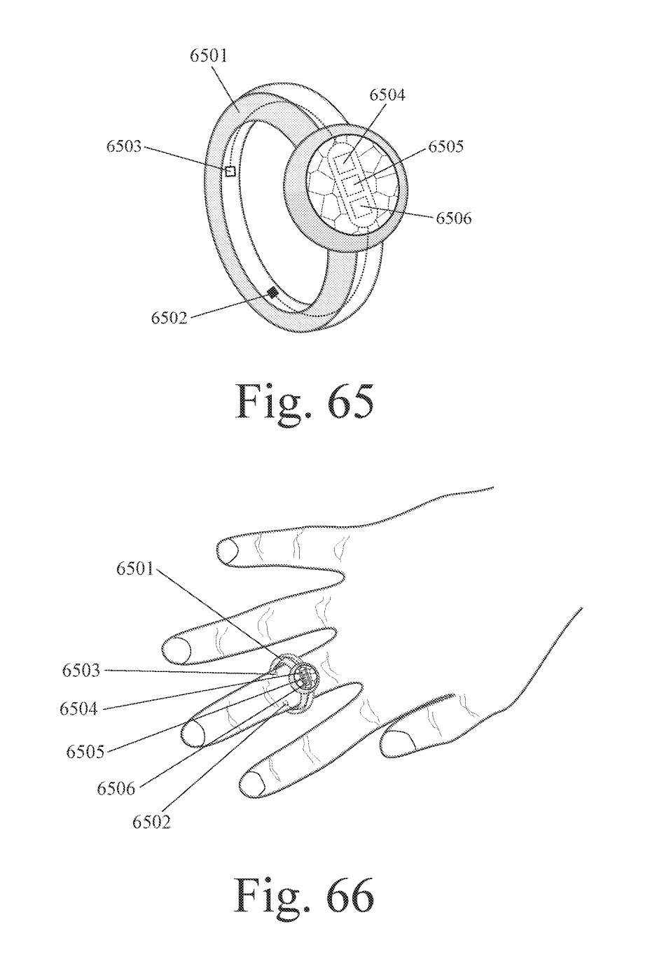

FIGS. 65 and 66 show an example of how this invention can be embodied in a wearable device for non-invasive glucose monitoring comprising—a ring 6501 which is configured to be worn on a person's finger, wherein this ring further comprises: an electromagnetic energy emitter 6502 which is configured to emit electromagnetic energy into the person's finger tissue at a first location; an electromagnetic energy receiver 6503 which is configured to receive electromagnetic energy from the person's finger tissue at a second location, wherein parameters or patterns of the received electromagnetic energy are changed by the person's consumption of food and analyzed to monitor the person's food consumption; a power source 6504; a data processor 6505; and a data transmitter 6506. FIG. 65 shows this finger ring by itself. FIG. 66 shows this finger ring being worn on a person's finger. Relevant example variations which are discussed in other portions of this disclosure or the family of priority-related disclosures can also be applied to the embodiment shown here in FIGS. 65 and 66. Other example variations and component elaborations discussed elsewhere in this disclosure (or in other disclosures within the priority-linked family) can also be applied to this example.

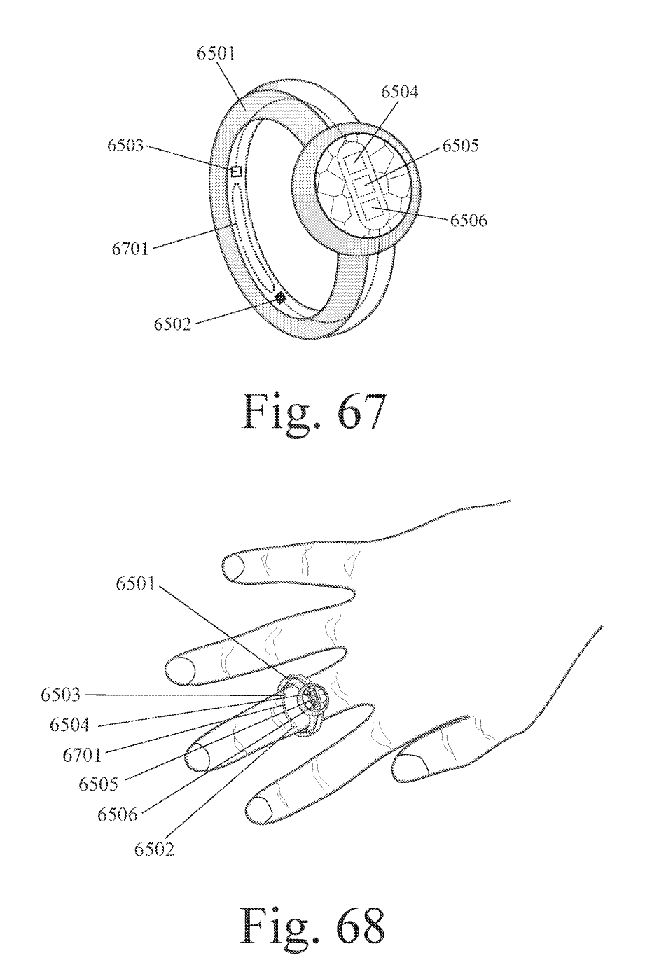

FIGS. 67 and 68 show another example of how this invention can be embodied in a wearable device for non-invasive glucose monitoring comprising—a ring 6501 which is configured to be worn on a person's finger, wherein this ring further comprises: an electromagnetic energy emitter 6502 which is configured to emit electromagnetic energy into the person's finger tissue at a first location; an electromagnetic energy receiver 6503 which is configured to receive electromagnetic energy from the person's finger tissue at a second location, wherein parameters or patterns of the received electromagnetic energy are changed by the person's consumption of food and analyzed to monitor the person's food consumption; an electromagnetic energy resonator 6701 between the electromagnetic energy emitter and the electromagnetic energy receiver; a power source 6504; a data processor 6505; and a data transmitter 6506. FIG. 67 shows this finger ring by itself. FIG. 68 shows this finger ring being worn on a person's finger. Relevant example variations which are discussed in other portions of this disclosure or the family of priority-related disclosures can also be applied to the embodiment shown here in FIGS. 67 and 68. Other example variations and component elaborations discussed elsewhere in this disclosure (or in other disclosures within the priority-linked family) can also be applied to this example.

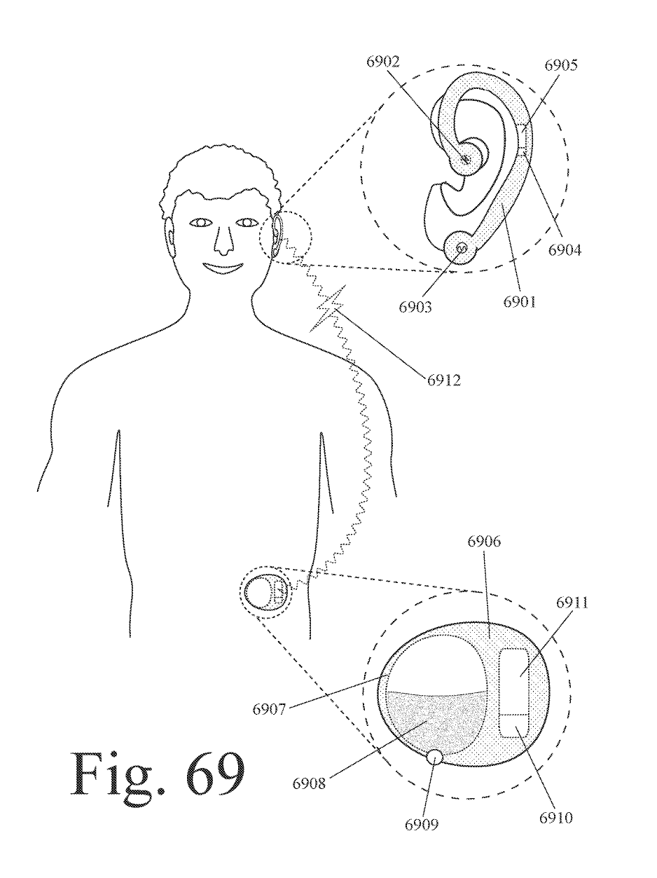

FIG. 69 shows an example of how this invention can be embodied in an automated closed-loop system for glycemic control. However, it does not limit the full generalizability of the resulting claims. FIG. 69 shows an example of a wearable device for non-invasive glucose monitoring comprising: an ear-worn diagnostic component that is worn on a person's ear, around a person's ear, and/or within a person's ear canal, wherein the ear-worn diagnostic component further comprises at least one electromagnetic energy sensor which collects a first set of biometric data, wherein the first set of biometric data concerns the person's electromagnetic brain activity, wherein the ear-worn diagnostic component further comprises at least one light energy sensor which collects a second set of biometric data, and wherein the second set of biometric data concerns the spectrum of light which has been reflected from or passed through the person's body tissue; a wearable and/or implanted pump which dispenses one or more glycemic control substances into the person's body; and a data processor which jointly analyzes the first set of biometric data and the second set of biometric data in order to determine the person's need for the one or more glycemic control substances, wherein the wearable and/or implanted pump is automatically triggered to dispense the one or more glycemic control substances into the person's body when needed based on the results of analysis by the data processor.

The left portion of FIG. 69 shows a frontal view of a person's head and torso, wherein the person is wearing an ear-worn diagnostic component on their ear and a wearable pump which dispenses a glycemic control substance on their torso. In this example, the ear-worn diagnostic component and the wearable pump are in direct wireless electromagnetic communication with each other. The operation of the wearable pump is controlled based on analysis of data from biometric sensors in the ear-worn diagnostic component.

The upper right portion of FIG. 69 shows a close-up view of the ear-worn diagnostic component within a virtual dashed-line circle, wherein this dashed-line circle corresponds to the smaller dashed-line circle around the same ear-worn component shown in smaller scale on the left portion of the figure. The lower right portion of FIG. 69 shows a close-up view of the wearable pump within a virtual-dashed line circle, wherein this dashed-line circle corresponds to the smaller dashed line circle around the same wearable pump that is shown in smaller scale on the left portion of the figure.

With respect to specific components, the ear-worn diagnostic component of the system shown in a close-up view in the upper right portion of FIG. 69 includes: ear-worn attachment member 6901 which is partially inserted into the ear canal, curves around the rear of the ear, and also attaches to the earlobe; electromagnetic energy sensor 6902, which collects a first set of biometric data, wherein this first set of biometric data concerns electromagnetic brain activity; light energy sensor 6903, which collects data a second set of biometric data, wherein this second set of biometric data concerns the spectrum of light which has been reflected from or passed through the earlobe; power source 6904; and data processor and transmitter/receiver 6905.

With respect to specific components, the wearable pump component of the system shown in a close-up view in the lower right portion of FIG. 69 includes: wearable pump housing 6906; glycemic control substance reservoir 6907; glycemic control substance 6908, glycemic control substance pump 6909; power source 6910; and data processor and transmitter/receiver 6911. In this example, data processor and transmitter/receiver 6911 of the wearable pump component is in direct wireless communication 6912 with data processor and transmitter/receiver 6905 of the ear-worn diagnostic component. In another example, both of these data processors and transmitters/receivers can be in indirect wireless communication by each having wireless communication with a common separate and/or remote data processor and transmitter/receiver.

In this example, the first set of biometric data and the second set of biometric data are jointly analyzed by a data processor in order to determine the person's need for the dispensation of a glycemic control substance. In this example, the wearable pump is automatically triggered to dispense the glycemic control substance into the person's body when needed based on the results of joint analysis of biometric data by a data processor. In this example, the first set of biometric data and the second set of biometric data are jointly analyzed in order to measure or predict changes in the person's body glucose levels. In this example, the wearable pump is automatically triggered to dispense the glycemic control substance into the person's body when needed based on measured or predicted changes in body glucose levels.

In an example, joint statistical analysis of the first and second sets of data can occur in data processor and transmitter/receiver 6905 which is part of the ear-worn diagnostic component. In an example, data can be wirelessly transmitted to data processor and transmitter/receiver 6911 which is part of the wearable pump component and then joint statistical analysis of the first and second sets of data can then occur in that data processor. In an example, data can be wirelessly transmitted to a separate and/or remote data processor and then joint statistical analysis of the first and second sets of data can then occur in that separate and/or remote data processor.

The different components shown in FIG. 69 combine to form an automated closed-loop system for glycemic control. Biometric data concerning current and/or predicted body glucose levels is collected by electromagnetic energy sensors and spectroscopic light energy sensors which are part of the diagnostic ear-worn component. Both types of biometric data are jointly analyzed to determine current and/or predicted body glucose levels and the need for dispensation of one or more glycemic control substances to bring and/or keep body glucose levels within a normal range. The results of this analysis then trigger dispensation of the appropriate amounts of one or more glycemic control substances by the wearable pump in order to keep the person's body glucose levels within a normal range. Other example variations and component elaborations discussed elsewhere in this disclosure (or in other disclosures within the priority-linked family) can also be applied to this example.

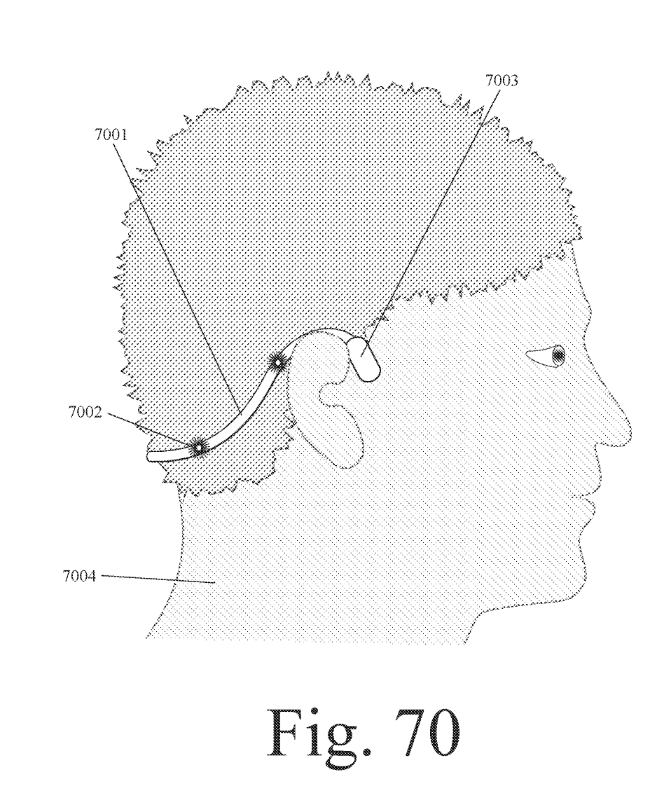

FIG. 70 shows a side view of an example of a wearable device for non-invasive glucose monitoring comprising a head-worn sensor-positioning member 7001 which is configured to position a plurality of electrodes or other brain activity sensors, including 7002, at selected locations on a person's 7004 head. In this example, the sensor-positioning member is assumed to be substantially symmetric with respect to the right side (shown) and the left side (not shown) of the person's head. This monitor further comprises control unit 7003, which need not be replicated on the other side. In this example, sensor-positioning member 7001 comprises a loop that spans from one ear to the other, looping around the lower-posterior portion of the person's head. In an example, the average height of this loop is equal to, or lower than, the average height of the person's ears. In this example, the left-side and right-side ends of the loop curve around and hook over the tops of the person's left and right ears, respectively, terminating in locations forward of the upper portions of the ears. In this example, control unit 7003 is just forward of the upper portion of the left ear. In this example, the loop spans a lower portion of the person's temporal lobe and a portion of their cerebellum.

In an example, control unit 7003 can further comprise: a data processing component and a power source (or transducer). In an example, control unit 7003 can further comprise: a data processing component; a power source (or transducer); and a data transmitting (and receiving) component. In an example, control unit 7003 can be in wireless communication with an external (or remote) device and/or with another component of an overall system for monitoring brain activity. In an example, control unit 7003 can further comprise: a data processing component; a power source (or transducer); a data transmitting (and receiving) component; and a user interface. In an example, control unit 7003 can be physically connected to the array of electrodes (or other brain activity sensors) by wires or other electromagnetically-conductive pathways. In an example, control unit 7003 can be in wireless electromagnetic communication with an array of electrodes (or other brain activity sensors).

FIG. 70 also shows a wearable device for non-invasive glucose monitoring comprising: a plurality of brain activity sensors (including 7002); a head-worn loop 7001 which is configured to span from one ear to the other around the lower-posterior portion of the head of a person 7004 wearing the loop, wherein the loop is configured to position the plurality of brain activity sensors at selected locations on the person's head; and a control unit 7003. In an example, the control unit can further comprise a data processing component and a power source or transducer. Other example variations and component elaborations discussed elsewhere in this disclosure (or in other disclosures within the priority-linked family) can also be applied to this example.

FIG. 71 shows an example of a wearable device for non-invasive glucose monitoring comprising: a partially-circumferential headband (including rear portion 7101, front portion 7102, and ear-perimeter-engaging member 7103) which spans a portion of the circumference of a person's head, including a portion of the person's forehead; a plurality of electromagnetic energy sensors (including 7107 and 7108) which are configured to be held in proximity to the person's head by the headband, wherein these electromagnetic energy sensors collect data concerning electromagnetic activity of the person's brain; a wireless data transmitter and/or receiver 7104; a data processor 7105; and a power source 7106. In an example, this device can have a symmetric configuration on the other side of the person's head, which is not shown here.

In an example, a rear portion of a partially-circumferential headband can extend rearward from a person's right and left ears, looping completely around the rear of a person's head from the right ear to the left ear. In an example, right and left front portions of a partially-circumferential headband can extend forward from a person's right and left ears, respectively, partially extending onto the right and left sides of a person's forehead, respectively, but not completely spanning from the right ear to the left ear. In an example, the right and left front portions of a partially-circumferential headband can have ends which terminate on the right and left sides of a person's forehead, respectively, leaving a gap between them. In an example, this gap can include the center of the person's forehead.

In an example, a partially-circumferential headband can span between 50% and 85% of the circumference of a person's head. In an example, a partially-circumferential headband can span between 60% and 80% of the circumference of a person's head. In an example, a partially-circumferential headband can have an arcuate axial shape like that of an ancient Roman laurel wreath. In an example, a partially-circumferential headband can be shaped like a horseshoe or like the letter “U”, with upturned front ends. In an example, a partially-circumferential headband can loop around the sides and rear of a person's head from the right side of a person's forehead to the left side of the person's forehead, but not fully span across the person's forehead. In an example, a partially-circumferential headband can fully span the rear of a person's head, between their ears, but only partially span the front of the person's head.

In an example, a partially-circumferential headband can rest on top of a person's ears. In an example, a partially-circumferential headband can span the sides of a person's head above the person's ears. In an example, a partially-circumferential headband can loop around the rear of a person's head at a substantially level height, pass over the tops of a person's ears, and then arc upwards and forward to terminal positions on the sides of the person's forehead, stopping short of the center of the person's forehead. In an example, the right and left ends of a partially-circumferential headband can be on a person's forehead above the person's right and left eyes, respectively.

In an example, a side of a partially-circumferential headband can extend forward from a person's ear at an overall vector between the 1 o'clock (30 degree) vector and the 3 o'clock (90 degree) vector. In an example, a front portion of a partially-circumferential headband can initially extend forward from a person's ear along a vector between the 2 o'clock (60 degree) and 3 o'clock (90 degree) vectors, and then curve upward toward the person's forehead along a vector between the 1 o'clock (30 degree) and 2 o'clock (60 degree) vectors. In an example, a front portion of a partially-circumferential headband can be configured to end between 25% and 75% of the way from a person's ear to the center of their forehead. In an example, this end can be within the range of 1″ to 4″ above the top of the person's ear.

In an example, this headband can further comprise an ear-perimeter-engaging member which curves around the rear of a person's ear to better hold the headband in place. In an example, this ear-perimeter-engaging member can span between the 7 o'clock (210 degree) and 12 o'clock (0 degree) vectors. In an example, this ear-perimeter-engaging member can span between the 9 o'clock (270 degree) and 12 o'clock (0 degree) vectors. In an example, this ear-perimeter-engaging member can also be attached to an earlobe.

FIG. 71 also shows an example of a wearable device for non-invasive glucose monitoring comprising: a plurality of brain activity sensors (including 7107 and 7108); a head-worn loop (including rear portion 7101 and front portion 7102) which is configured to span from one ear to the other around the lower-posterior portion of the head of a person wearing the loop, wherein the loop is configured to position the plurality brain activity sensors at selected locations on the person's head; a wireless data transmitter and/or receiver 7104; a data processor 7105; and a power source 7106. Other example variations and component elaborations discussed elsewhere in this disclosure (or in other disclosures within the priority-linked family) can also be applied to this example.

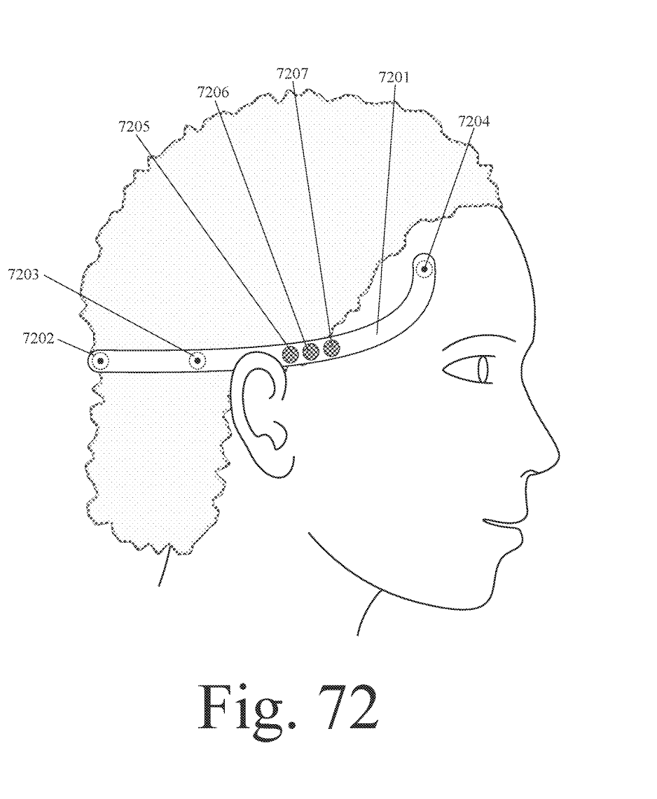

FIG. 72 shows an example of a wearable device for non-invasive glucose monitoring comprising: a plurality of brain activity sensors (including 7202, 7203, and 7204); a head-worn loop 7201 which is configured to span from one ear to the other around the lower-posterior portion of the head of a person wearing the loop, wherein the loop is configured to position the plurality brain activity sensors at selected locations on the person's head; a wireless data transmitter and/or receiver 7204; a data processor 7205; and a power source 7206. Other example variations and component elaborations discussed elsewhere in this disclosure (or in other disclosures within the priority-linked family) can also be applied to this example.

I claim:

1. A device for non-invasive glucose monitoring comprising:
a finger ring, wrist band, or watch which is configured to be worn by a person, wherein the finger ring, wrist band, or watch further comprises;
a circumferential biosensor array along a circumference of the finger ring, wrist band, or watch, wherein the circumferential biosensor array includes at least one electromagnetic energy emitter and at least one electromagnetic energy receiver;
a power source;
a data processor which receives data from the electromagnetic energy receiver which is analyzed in order to measure the person's body glucose level; and
a data transmitter.

2. The device in claim 1 wherein the impedance or resistance of the person's tissue is analyzed in order to measure the person's glucose level.

3. The device in claim 1 wherein the circumferential biosensor array comprises at least two sensor pairs along a circumference of the finger ring, wrist band, or watch, wherein each sensor pair comprises an electromagnetic energy emitter and an electromagnetic energy receiver.

4. The device in claim 1 wherein the circumferential biosensor array comprises at least two sensor triads along a circumference of the finger ring, wrist band, or watch, wherein each sensor triad comprises an electromagnetic energy emitter, an electromagnetic energy resonator, and an electromagnetic energy receiver.

5. The device in claim 1 wherein the circumferential biosensor array comprises an alternating sequence of electromagnetic energy emitters and electromagnetic energy receivers along a circumference of the finger ring, wrist band, or watch.

6. The device in claim 1 wherein the circumferential biosensor array comprises at least one electromagnetic energy emitter and at least two electromagnetic energy receivers along a circumference of the finger ring, wrist band, or watch.

7. The device in claim 1 wherein the circumferential biosensor array comprises two or more nested electromagnetic energy resonators between an electromagnetic energy emitter and an electromagnetic energy receiver.

8. The device in claim 1 wherein the circumferential biosensor array comprises two or more stacked electromagnetic energy resonators between an electromagnetic energy emitter and an electromagnetic energy receiver.

9. The device in claim 1 wherein the circumferential biosensor array spans at least 25% of the circumference of the finger ring, wrist band, or watch.

10. A device for non-invasive glucose monitoring comprising:
a finger ring, wrist band, or watch which is configured to be worn by a person, wherein the finger ring, wrist band, or watch further comprises;
a circumferential biosensor array along a circumference of the finger ring, wrist band, or watch, wherein the circumferential biosensor array includes at least one light emitter which is configured to emit light toward the person's body and at least one light receiver which is configured to receive light that has passed through and/or been reflected from the person's body;
a power source;
a data processor which receives data from the light receiver which is analyzed in order to measure the person's body glucose level; and
a data transmitter.

11. The device in claim 10 wherein a first light emitter emits light with a first frequency and/or spectrum, a second light emitter emits light with a second frequency and/or spectrum, and the second frequency and/or spectrum is different than the first frequency and/or spectrum.

12. The device in claim 10 wherein a first light emitter emits light at a first angle with respect to the finger ring, wrist band, or watch, a second light emitter emits light at a second angle with respect to the finger ring, wrist band, or watch, and the second angle is different than the first angle.

13. The device in claim 10 wherein the frequency and/or spectrum of light emitted by a light emitter is changed over time.

14. The device in claim 10 wherein the angle at which light is emitted from a light emitter is changed over time.

15. The device in claim 10 wherein the circumferential biosensor array comprises at least two sensor pairs along a circumference of the finger ring, wrist band, or watch, wherein each sensor pair comprises a light emitter and a light receiver.

16. The device in claim 10 wherein the circumferential biosensor array comprises an alternating sequence of light emitters and light receivers along a circumference of the finger ring, wrist band, or watch.

17. The device in claim 10 wherein the circumferential biosensor array spans at least 25% of the circumference of the finger ring, wrist band, or watch.

18. A device for non-invasive glucose monitoring comprising:
a wearable device that is configured to be worn on a person's ear, wherein the wearable device further comprises:
one or more electroencephalographic sensors;
a power source;
a data processor which receives data from the one or more electroencephalographic sensors which is analyzed in order to measure the person's body glucose level; and
a data transmitter.

19. The device in claim 18 wherein at least one electroencephalographic sensor is located on a portion of the wearable device which is configured to project onto the person's forehead.

20. The device in claim 18 wherein a first electroencephalographic sensor is located on a portion of the wearable device which is configured to be inserted into the person's ear and a second electroencephalographic sensor is located on a portion of the wearable device which is configured to project onto the person's forehead.
